(19) United States
(12) Patent Application Publication
(10) Pub. No.: US 2016/0369237 A1
(43) Pub. Date: Dec. 22, 2016

(54) METHODS TO ACCELERATE THE ISOLATION OF NOVEL CELL STRAINS FROM PLURIPOTENT STEM CELLS AND CELLS OBTAINED THEREBY

(71) Applicant: Advanced Cell Technology, Inc., Marlborough, MA (US)

(72) Inventors: Michael D. West, Mill Valley, CA (US); Geoffrey Sargent, San Lorenzo, CA (US); James Teruo Murai, San Bruno, CA (US); Steven Kessler, Belmont, CA (US); Karen Chapman, Mill Valley, CA (US)

(21) Appl. No.: 15/247,786

(22) Filed: Aug. 25, 2016

Related U.S. Application Data
(63) Continuation of application No. 11/604,047, filed on Nov. 21, 2006, now abandoned.
Application No. 11/604,047 is a continuation-in-part of application No. PCT/US06/13519, filed on Apr. 11, 2006.
(60) Provisional application No. 60/791,400, filed on Apr. 11, 2006.
(60) Provisional application No. 60/738,912, filed on Nov. 21, 2005.

(51) Int. Cl. C12N 5/0775; C12N 5/071; A61K 35/407; A61K 35/30; A61K 35/39; C12N 5/0793; A61K 35/34

(52) U.S. Cl. CPC C12N 5/0662; C12N 5/0619; C12N 5/067; A61K 35/34; A61K 35/30; A61K 35/39; A61K 35/407; C12N 2506/02

(57) ABSTRACT

This invention generally relates to methods to differentiate pluripotent stem cells, such as embryonic stem, embryonic germ, or embryo-derived cells, to obtain subpopulations of cells from heterogeneous mixtures of cells wherein the subpopulation of cells possess reduced differentiation potential compared to the original pluripotent stem cells and where the subpopulation is capable of being propagated.

### FIELD OF THE INVENTION

This invention generally relates to methods to accelerate the isolation of novel cell strains from pluripotent stem cells and cells obtained by such methods. Specifically, this invention relates to methods to differentiate pluripotent stem cells, such as human embryonic stem (“hES”) cells, human embryonic germ (“hEG”) cells, human embryo-derived (“hED”) cells and human embroyonal carcinoma cells (human EC cells), to obtain subpopulations of cells from heterogeneous mixtures of cells, wherein the subpopulation of cells possess reduced differentiation potential compared to the original pluripotent stem cells and where the subpopulation is capable of being propagated. This invention also provides novel compositions of such subpopulation of cells and methods to propagate said cells. More particularly, the invention relates to a two-step method wherein said pluripotent stem cells are first exposed to conditions that induce a heterogeneity of differentiation potential in said stem cells, and next a plating/propagation step allowing single cells or an oligoclonal cluster of similar cells with reduced breadth of differentiation potential than the original stem cells and that resulted from the original stem cells to expand in number while exposed to a combination of culture environments that determine conditions that promote propagation from one or a small cluster of cells. Said single cell or oligoclonal cell-derived populations of cells with a more restricted breadth of differentiation potential and cells capable of proliferation from the second step are characterized and formulated for use in research and therapy, and for the production of bioactive materials such as cell extracts, conditioned medium and extracellular matrix.

### BACKGROUND OF THE INVENTION

Advances in stem cell technology, such as the isolation and propagation in vitro of embryonic stem cells (“ES” cells including human ES cells (“hES” cells)) and related totipotent stem cells including but not limited to, EG, EC, or ED cells (including human EG, EC or ED cells), constitute an important new area of medical research. hES cells have a demonstrated potential to be propagated in the undifferentiated state and then to be induced subsequently to differentiate into any and all of the cell types in the human body, including complex tissues. In addition, many of these early stem cells are naturally telomerase positive in the undifferentiated state, thereby allowing the cells to be expanded extensively and subsequently genetically modified and clonally expanded. Since the telomere length of many of these cells is germ-line in length, differentiated cells derived from these immortal lines will naturally repress the expression of the catalytic component of telomerase (hTERT) and thereby become mortal though the long initial telomere length allows for cells with long replicative capacity compared to fetal or adult-derived tissue. This has led to the suggestion that many diseases resulting from the dysfunction of cells may be amenable to treatment by the administration of hES-derived cells of various differentiated types (Thomson et al., Science 282:1145-1147 (1998)). Nuclear transfer studies have demonstrated that it is possible to transform a somatic differentiated cell back to a totipotent state such as that of embryonic stem cells (“ES”) (Cibelli et al., Nature Biotech 16:642-646 (1998)) or embryo-derived (“ED”) cells. The development of technologies to reprogram somatic cells back to a totipotent ES cell state, such as by the transfer of the genome of the somatic cell to an enucleated oocyte and the subsequent culture of the reconstructed embryo to yield ES cells, often referred to as somatic cell nuclear transfer (“SCNT”), offers a method to transplant ES-derived somatic cells with a nuclear genotype of the patient (Lanza et al., Nature Medicine 5:975-977 (1999)).

In addition to SCNT, other techniques exist to address the problem of transplant rejection, including the use of gynogenesis and androgenesis (see U.S. application No. 60/161,987, filed Oct. 28, 1999; Ser. No. 09/697,297, filed Oct. 27, 2000; Ser. No. 09/995,659, filed Nov. 29, 2001; Ser. No. 10/374,512, filed Feb. 27, 2003; PCT application no. PCT/US00/29551, filed Oct. 27, 2000; the disclosures of which are incorporated by reference in their entirety). In the case of a type of gynogenesis designated parthenogenesis, pluripotent stem cells may be manufactured without antigens foreign to the gamete donor and therefore useful in manufacturing cells that can be transplanted without rejection. In addition, parthenogenic stem cell lines can be assembled into a bank of cell lines homozygous or hemizygous in the HLA region to reduce the complexity of a stem cell bank in regard to HLA haplotypes.

Nevertheless, there remains a need for providing a means to direct the differentiation of totipotent or pluripotent stem cells into the many desired cell lineages present in the developing and developed mammalian body, under conditions which are compatible in either a general laboratory setting or in a good manufacturing processes (“GMP”) cell manufacturing facility where there is adequate documentation as to the purity and genetic normality of the cells.

Furthermore, there still remains a need to describe methods to identify cells derived from such pluripotent stem cells that are capable of being propagated in vitro, methods to identify culture conditions for propagating cells derived from pluripotent stem cells, precise definition relating to the materials that have come into physical contact with the cells, precise definition of the presence or absence of pathogens in such cells, and evidence as to whether any undifferentiated or other cell types, such as fibroblastic cells, contaminate the cell formulation derived from such cells planned for therapeutic use, and methods to identify such purified populations of cells that are capable of expansion in number in a target tissue and/or stable engraftment. Also, there is a need to derive cells from pluripotent stem cells, such derived cells that are more differentiated than the parent pluripotent stem cells but are still progenitor cells that can differentiate further.

Furthermore, while there are numerous publications relating to the differential expression of genes, including but not limited to, differentiation-related genes such as homeobox-containing genes, in mouse and avian species, such data do not necessarily apply to other species such as hES-derived cells, and such published results often result from histological studies of limited tissues and whole tissues where it is not possible to determine precisely what cell types differentially express particular genes in the course of development. As a result, there is a need to determine what genes and combinations of genes provide useful markers of defined and clonal differentiation pathways in various species including avian and mammalian species such as human. Such markers would allow the correct identification of cells derived from pluripotent stem cells such as hES cells.

One of the major recurrent problems with culturing mammalian differentiated cell types in vitro is the preservation of a pure culture of the differentiated cell type without having the culture overgrown with fibroblastic or other contaminating cell types. See, Ian Freshney, Culture of Animal Cells: A Manual of Basic Technique (5th Ed.), New York: Wiley Publishing, 2005, p. 217. Because heterogeneous cultures of immortal organisms, such as bacteria or yeast cells, could be made homogeneous through means to isolate a population of cells from a single parent cell, efforts have been made to isolate populations of human and other mammalian cells of various types from a single parent cell (clonogenic growth). However, the traditional microbiological approach to the problem of culture heterogeneity, by isolating pure cell strains using cloning, has limited success in most primary cultures from fetal or adult tissue because of the poor cloning efficiencies. However, the cloning of primary cultures has been shown to be successful for certain cell types, for example, in Sertoli cells (Zwain et al., Mol Cell Endocrinol., 80(1-3):115-26 (1991)), juxtaglomerular (Muirhead et al., Methods Enzymol., 191:152-67 (1990)) and glomerular (Troyer & Kreisberg, Methods Enzymol., 191:141-52 (1990)) cells from kidney, oval cells from liver (Suh et al., Tissue Eng., 9(3):411-20 (2003)), satellite cells from skeletal muscle (Zeng et al., Poult Sci., 81(8):1191-8 (2002); McFarland et al., Comp Biochem Physiol C Toxicol Pharmacol., 134(3):341-51 (2003); Hashimoto et al., Development, 131(21):5481-90 (2004)) and separation of different lineages from adult stem cell populations has been reported (Young et al., Anat Rec A Discov Mol Cell Evol Biol., 276(1):75-102 (2004)). Therefore, while the generation of clonogenic populations of cells has demonstrated its usefulness in generating a limited number of differentiated cell types free of contaminating cells, there still remains a need to describe methods for propagating cell types and culture systems, such as the early embryonic cell lineages derived from hES, hEG, human EC or hED cells.

In addition, a further problem with culturing human cells is the inability to expand the number of cells in the cell cultures to generate enough cells to be of practical and therapeutic applicability. This stems from the observation that most human cell clones from fetal or adult tissue sources senescence relatively early such as when still replicating in the original colony or shortly thereafter (i.e. can only survive for a limited number of generations thereby limiting many applications such as scale-up in the manufacturing process) (see, e.g., Smith et al., Proc. Natl Acad. Sci., USA, v. 75(3), pp. 1253-1356 (1978)).

In addition, most cells derived from fetal or adult sources are not capable of being propagated at low densities, such as when deriving cultures from a single parent cell or from a small number of similar cells (oligoclonal). At low densities, the cells do not receive sufficient mitogenic signals to allow for extensive propagation. Therefore, even if the cells had sufficient replicative lifespan to generate a useful culture of cells, the cultivation of many somaticcells at low density is nevertheless nonpermissive for growth and for uncharacterized cell types, such as hES-derived cell lines, there is no way of knowing which, if any, hES-derived cells are capable of propagation clonally or oligoclonally in vitro. In some cases, growth of some cell types can nevertheless be achieved at clonal densities by culturing the cells under specific conditions, such as in low ambient oxygen, on mitotically inactivated feeder cells, or with the addition of conditioned medium. However, such techniques have only been reported useful in generating stable cell lines for a few cell types and success for any novel cell type is still highly uncertain.

While methods have been described to accomplish genetic selection, by the introduction of transgenes into pluripotent stem cells, wherein the expression of said transgene is dependent upon a differentiation-specific promoter sequence and said transgene imparts an ability to select a particular differentiated cell type from a mixture of heterogeneous cells (see, e.g., U.S. Pat. Nos. 5,733,727 and 6,015,671), such genetic selection techniques do not in themselves necessarily lead to purified populations of cells capable of being propagated in vitro nor do they provide the methods to accomplish such propagation. In addition, novel methods that do not result in genetically modified cells would be useful in simplifying the development of cell-based therapies.

Furthermore, patterns for the expression of various growth factors, receptors, extracellular matrix components in the developing animal have been described. For example, Ford-Perriss et al., Clinical & Experimental Pharm. & Physiol. 28:493-503 (2001) describe the expression of growth factors such as members of the FGF family of growth factors in the developing mammalian CNS, yet the role of these and many other factors in the differentiation of pluripotent stem cells in vitro, or in the cultivation of cells derived from a single cell or a small number of cells committed to a common cell fate that was itself differentiated from or is in the process of differentiating from pluripotent stem cells has not been described.

Finally, while there are descriptions of numerous cell types obtained from pluripotent stem cells such as human embryonic stem cells, there has been no description of a method to obtain cells from hES, hEG, human EC or hED cells, wherein said cells display a prenatal gene expression phenotype consistent with cells and tissues of animals in their embryonic stage of development, which are normally progressively lost in further fetal development and in the subsequent adult animal. While animals models and molecular studies have revealed that there are different gene expression patterns in fetal vs. adult tissues, prior attempts via gene therapy to alter the pattern of gene expression in cells to more closely mimic that of the early prenatal state have not resulted in satisfactory results. Therefore, there remains a need to describe a means for identifying and propagating such cells from pluripotent stem cells. The identification of the prenatal patterns of gene expression in such cells will provide useful markers for subsequent identification of these cells that may be capable of regenerating tissue, i.e., capable of stromal/epithelial interactions that can be organize tissue, including but not limited to, innervation (such as neural axon outgrowth) and vascularization.

In summary, while numerous techniques to increase the frequency of a desired cell type in a complex mixture of cell types differentiated from pluripotent stem cells have been reported, there remains a problem of the preservation of the culture of a particular cell type, in particular, properties useful in facilitating the transplantation of such cells into organs and tissues including, but not limited to, properties unique to embryonic cells and tissues. In addition, there remains a need to identify novel means of generating uniform populations of cells with limited or even unitary differentiation potential from pluripotent stem cells, such as hES cells, means to identify said cells capable of being propagated in vitro, and methods of generating and propagating such culture.

### SUMMARY OF THE INVENTION

This invention solves the problems described above. This invention generally relates to methods to differentiate pluripotent stem cells, such as human embryonic stem cells (“hES”), human embryonic germ (“hEG”) cells, human embryonal carcinoma (“EC”) cells and human embryo-derived (“hED”) cells, to obtain subpopulations of cells from heterogeneous mixtures of cells, wherein the subpopulation of cells possess reduced differentiation potential compared to the original pluripotent stem cells and where the subpopulation is capable of being propagated. This invention also provides novel compositions of such subpopulation of cells and methods to propagate such cells.

More particularly, the invention relates to a two-step method wherein pluripotent stem cells are first exposed to conditions that induce a heterogeneity of differentiation potential in said stem cells, and next a plating/propagation step allowing single cells or an oligoclonal cluster of similar cells with reduced differentiation potential than the original stem cells and that resulted from the original stem cells to expand in number while exposed to a combination of culture environments. Said single cell-derived populations of cells with a more restricted breadth of differentiation potential and cells capable of proliferation from the second step are characterized and formulated for use in research and therapy, and for the production of growth factors, cell extracts, conditioned medium, and extracellular matrix of said cells are formulated and used for research and therapy.

This invention provides a method for deriving desired cell types (“derived cells”) from pluripotent stem cells such as hES, hEG, human EC or hED cells (parent population). The derived cells possess reduced differentiation potential when compared to the pluripotent stem cells from which they were derived (parent pluripotent stem cell population). The derived cells comprise cells that have the ability to differentiate further, i.e., they are not terminally differentiated cells. In certain embodiments, the method of this invention comprises the steps of: (1)(a) selecting all or a subset of differentiation conditions that may result in the differentiation of said parent pluripotent stem cells into a heterogeneous population of cells, wherein a plurality of said cells may be more differentiated than said parent pluripotent stem cells; (1)(b) exposing said parent pluripotent stem cells to said all or a subset of differentiation conditions from step (1)(a) for various time periods resulting in a heterogeneous population of cells comprising cells with reduced differentiation potential than said parent pluripotent stem cells, wherein a plurality of said cells may have reduced differentiation potential than said parent pluripotent stem cells; (2)(a) culturing said heterogeneous population of cells from step (1)(b) in culture conditions wherein said single cells proliferate and the single cells and/or their progeny may be isolated as a clonal or oligoclonal culture of cells; wherein said heterogeneous population of cells may optionally be disaggregated to single cells prior to culturing, and (2)(b) propagating said clonal population of cells of step (2)(a), resulting in said derived cells, wherein said cells are more uniform in differentiation potential and have reduced differentiation potential compared to the parent pluripotent stem cell population. In certain embodiments, the cells in steps (2)(a) and (2)(b) are grown in the same medium, including the differentiation conditions, as the medium used in step (1)(b) to differentiate the parent pluripotent stem cells. Using the same, or substantially the same medium and growth factors has the advantage that cells capable of proliferating clonally or oligoclonally are expanded in step 1 (b) increasing the number of propagating clones in steps 2 (a) and 2 (b). The resulting cells are “derived cells.” In certain embodiments of this method, the heterogeneous population of cells from step (1) (b) are obtained by allowing said parent pluripotent stem cells to differentiate for various periods of time without disaggregation, i.e., for the cells to incubate in the differentiation conditions for various time periods before optionally disaggregating them. In a further embodiment of this method, the heterogeneous population of cells from step (1)(b) are obtained by allowing said parent pluripotent stem cells to differentiate for various periods of time without disaggregation and further, comprising the step of producing embryoid bodies using a variety of culture conditions for various time periods. In further embodiments of this method, the embryoid bodies are differentiated for various time periods. In certain embodiments of this method, the disaggregating step is performed by trypsinizing the heterogeneous population of cells. In certain other embodiments of this method, the heterogeneous population of cells from step (1)(b) is plated in step (2)(a) at limiting dilution or at low density and subsequently removed using cloning cylinders, to arrive at individual cultures each of which originated from a single cell or small number of cells (oligoclonal). In further embodiments of this method, the limiting dilution is performed in multiwell dishes. In certain other embodiments of this method, the heterogeneous population of cells from step (1)(b) are plated in juxtaposition with feeder or inducer cells. In certain other embodiments of this method, the heterogeneous population of cells from step (1)(b) are plated as single isolated cells at low density in a semisolid media in step (2)(a). In certain other embodiments of this method, the heterogeneous population of cells from step (1)(b) are cultured in hanging drop culture. In certain other embodiments of this method, the heterogeneous population of cells from step (1)(b) are cultured as single isolated cells at low density in hanging drop culture in step (2)(a) and cultured in step (2)(b) as cell aggregates. In certain other embodiments of this method, the heterogeneous population of cells from step (1)(b) are cultured in step (2)(a) at low cellular density such that colonies of proliferating cells derived from a single cell can be easily identified and isolated using cloning cylinders or other similar means well known in the art and subsequently propagated in step (2)(b). In certain embodiments of this method, the pluripotent stem cells are differentiated in vitro, in vivo, or in ovo. In certain embodiments of this method, the heterogeneous population of cells forms a multicellular aggregate, such as an embryoid body. In certain embodiments of this method, the method of this invention further comprises the step of disaggregating the multicellular aggregate into single cells, by, for example, trypsinizing the multicellular aggregate. In certain embodiments of this method, the cells contained in a plurality of wells of step (1)(b) are documented by genotype or phenotype prior to step (2)(a), such as by photography, by immunocytochemistry or by hybridization of probes with RNA or cDNA transcript. In certain embodiments, the heterogeneous population of cells is not disaggregated prior to plating but clonal or oligoclonal growth originates from the original heterogeneous aggregate. In certain embodiments, the single cells and/or their progeny may be isolated as an oligoclonal population of cells, each of which have similar characteristics (as it is known that like cells often share morphology and have common cell adhesion molecules and adhere together). In certain embodiments, the pluripotent stem cells form embryoid bodies prior to being exposed to differentiation conditions. The parent cells may be pluripotent or may be totipotent.

This invention also provides a method for deriving desired cell types (“derived cells”) from parent pluripotent stem cells comprising the steps of:

(1) exposing said parent pluripotent stem cells in various differentiation conditions for various time periods resulting in a heterogeneous population of cells comprising cells with reduced differentiation potential than said parent pluripotent stem cells, wherein a plurality of said cells may have reduced differentiation potential than said parent pluripotent stem cells;
(2)(a) culturing said heterogeneous population of cells from step (1) in culture conditions wherein said single or small number of cells proliferate and the progeny of said single cells may be isolated as a clonal or oligoclonal culture of cells; wherein said heterogeneous population of cells comprising cells with reduced differentiation potential than the parent population may optionally be disaggregated to single cells prior to culturing, and
(2)(b) propagating said clonal population of cells of step (2)(a), resulting in said derived cells, wherein said cells are more uniform in differentiation potential and have reduced differentiation potential compared to the parent pluripotent stem cell population. The derived cells comprise cells that have the ability to differentiate further, i.e., they are not terminally differentiated cells. The parent cells may be pluripotent or may be totipotent. In certain embodiments, the cells in steps (2) (a) and (2) (b) are grown in the same medium, including the differentiation conditions, as the medium used in step (1) to differentiate the parent pluripotent stem cells. In certain embodiments of this method, the heterogeneous population of cells from step (1) are obtained by allowing said parent pluripotent stem cells to differentiate for various periods of time without disaggregation, i.e., for the cells to incubate in the differentiation conditions for various time periods before optionally disaggregating them. In a further embodiment of this method, the heterogeneous population of cells from step (1) are obtained by allowing said parent pluripotent stem cells to differentiate for various periods of time without disaggregation and further, comprising the step of producing embryoid bodies using a variety of culture conditions for various time periods. In further embodiments of this method, the embryoid bodies are differentiated for various time periods. In certain embodiments of this method the disaggregating step is performed by trypsinizing the heterogeneous population of cells. In certain other embodiments of this method, the heterogeneous population of cells from step (1) is plated in step (2)(a) at limiting dilution or at low density allowing isolation using cloning cylinders, to arrive at individual cultures each of which originated from a single cell or each of which originated from an oligoclonal number of cells. In further embodiments of this method, the limiting dilution is performed in multiwell dishes. In certain other embodiments of this method, the heterogeneous population of cells from step (2)(a) is plated in juxtaposition with feeder or inducer cells. In certain other embodiments of this method, the heterogeneous population of cells from step (1) are plated as single isolated cells at low density in a semisolid media in step (2)(a). In certain other embodiments of this method, the heterogeneous population of cells from step (1)(b) are cultured in hanging drop culture. In certain other embodiments of this method, the heterogeneous population of cells from step (1) are cultured as single isolated cells at low density in hanging drop culture in step (2)(a) and cultured in step (2)(b) as cell aggregates. In certain other embodiments of this method, the heterogeneous population of cells from step (1) are cultured in step (2)(a) at low cellular density such that colonies of proliferating cells derived from a single cell can be easily identified and isolated using cloning cylinders or other similar means well known in the art and subsequently propagated in step (2)(b). In certain embodiments of this method, the pluripotent stem cells are differentiated in vitro, in vivo, or in ovo. In certain embodiments of this method, the heterogeneous population of cells forms a multicellular aggregate, such as an embryoid body. In certain embodiments of this method, the method of this invention further comprises the step of disaggregating the multicellular aggregate into single cells, by, for example, trypsinizing the multicellular aggregate. In certain embodiments of this method, the cells contained in a plurality of wells of step (2)(a) are documented by genotype or phenotype prior to step (2)(b), such as by photography, by immunocytochemistry or by hybridization of probes with RNA or cDNA transcript. In certain embodiments, the heterogeneous population of cells is not disaggregated prior to plating. In certain embodiments, the single cells and/or their progeny may be isolated as an oligoclonal population of cells, each of which have similar characteristics (as it is known that like cells stick together). In certain embodiments, the pluripotent stem cells first form embryoid bodies prior to being exposed to differentiation conditions.

In another embodiment of the invention, cells from the first differentiation step, but prior to the clonal or oligoclonal propagation step, are placed in growth media similar to or identical to that in which they will be clonally or oligoclonally expanded in order to increase the number of cells capable of propagating in the medium of the second step. This enrichment step allows an increased number and more predictable number of cells to proliferate in the final clonal or oligoclonal medium of the second step. In some cases where the medium of the initial differentiation step is identical to or similar to the medium in which the cells will be clonally or oligoclonally expanded, the enrichment step may also increase the number of proliferating cells such that the heterogeneous mixture may be cryopreserved and in the event that the clonal or oligoclonal isolation yielded useful cell types, the cryopreserved heterogeneous mixture of cells may be thawed and used as a source of cells for clonal or oligoclonal isolation again. Therefore, in one embodiment, the enrichment step is part of the initial differentiation step in that the culture medium of the first differentiation step is identical to, or similar to, that of the second clonal or oligoclonal propagation step. Alternatively, the enrichment step may be a separate step. The cells may be initially differentiated in one medium, then the heterogeneous mixture of cells can be transferred at normal cell culture densities to a different medium of the second clonal or oligoclonal expansion step. The cells are cultivated in that medium in a separate step. After a period of time of 2-30 days (preferably 5-14 days) that allows for the percentage of cells capable of being propagated in the medium to be increased, the heterogeneous mixture of cells is then clonally or oligoclonally expanded as described herein.

The methods of this invention is to accelerate the isolation of novel cells strains (cell lines) from pluripotent stem cells. In certain embodiments, the methods of this invention are directed to the isolation of a large number of cell lines that are in various states of differentiation or are differentiating. Some of these derived cells are terminally differentiated. Thus, it is an object of this invention to produce and isolate a large number of cell lines from pluripotent stem cells. Some of such cell lines are progenitors cells of various developmental lineages. Thus, in certain embodiments of this invention, it is a goal to isolate and propagate as many of the heterogeneous population of cells comprising cells with reduced differentiation potential than the starting parent pluripotent stem cells as possible.

In certain embodiments of this invention, the parent pluripotent stem cells or embryoid bodies derived therefrom are exposed to a variety of differentiating conditions. In certain embodiments of this invention, the plating step is performed at various time intervals after exposing to the differentiating conditions.

In certain embodiments of this invention, the pluripotent stem cells are ES cells, EG cells, EC cells or ED cells. In certain embodiments, the starting pluripotent stem cells are teratomas. One way to form teratomas is as follows: human or non-human ES cells may be injected into an animal to induce three dimensional growth, including but not limited to, immunocompromized animals such as nude mice, or into SPF embryonated chick eggs. In certain embodiments of this invention, the pluripotent stem cells are human cells. In other embodiments, the pluripotent stem cells are non-human cells, such as mouse cells, non-human primate cells, rat cells, non-human mammalian cells such as bovine, porcine, equine, canine, or feline cells, etc.

In certain embodiments of this invention, the pluripotent stem cells are genetically modified such that the MHC genes are deleted (“nullizygotes” for MHC). In certain other embodiments of this invention, the pluripotent stem cells are genetically modified such that the MHC genes are first deleted and then alleles of the MHC gene family are restored such that these stem cells are hemizygous or homozygous for one allele of the MHC gene family.

In certain embodiments of this invention, the pluripotent stem cells are derived from the direct differentiation of embryonic cells (such as morula cells or inner mass cells) without the derivation of embryonic stem cell line.

In certain embodiments of this invention, the pluripotent stem cells are derived from blastomeres. For example, blastomere, morula, or ICM cells can be plated in step 1a as are the other pluripotent stem cells of the present invention, and then clonal or oligoclonal cells isolated by following steps 1 (b) through 2 (b) as described herein where the pluripotent cells of the embryo yield clonal or oligoclonal cell lines without the intermediate step of ES cell line derivation.

In certain embodiments of this invention, the pluripotent stem cells are derived from the reprogramming of somatic cell through the exposure of said somatic cell to the cytoplasm of an undifferentiated cell.

In certain embodiments of this invention, the derived cells are endodermal cells, ectodermal cells or mesodermal cells, or cells of neural crest origin (the latter often designated ectodermal). In other embodiments of this invention, the derived cells are neuroglial precursor cells including definitive ectoderm and primitive neuroepithelium. In other embodiments of this invention, the derived cells are definitive endodermal cells such as hepatic cells or hepatic precursor cells, foregut, midgut, or hindgut endoderm, lung, pancreatic beta, or other endodermal precursor cells. In other embodiments of this method, the derived cells are chondrocyte, bone, or syovial precursor cells. In yet other embodiments of this invention, the derived cells are myocardial or myocardial precursor cells. In yet other embodiments of this invention, the derived cells are smooth muscle or skeletal muscle precursor cells including, but not limited to, somatic muscle precursor cells, muscle satellite stem cells and myoblast cells. In yet other embodiments of this invention, the derived cells are precursors of the branchial arches including those of the first branchial arch, such as mandibular mesenchyme, tooth, gingival fibroblast or gingival fibroblast precursor cells. In yet another embodiments of the invention, the derived cells are those of the intermediate mesoderm and precursors of kidney cells. In yet other embodiments of this invention, the derived cells are dermal fibroblasts with prenatal patterns of gene expression leading to scarless regeneration following wounding. In yet other embodiments of this invention, the derived cells are retinal precursor cells. In yet other embodiments of this invention, the derived cells are hemangioblasts.

This invention also provide isolated cells derived by the methods described above. This invention also contemplates genetically modifying these isolated cells.

In certain embodiments, the cells derived by the methods of this invention could be used as feeders or inducers on which other cells can be clonally expanded. In certain embodiments, the cell lines of this invention could be used as feeders or inducers in the first differentiation step (with or without the step of enrichment).

In certain embodiments, the cell lines made by the methods of this invention may be incorporated into devices and this invention provides such devices. Many of the cell lines made by the methods of this invention secrete factor(s) that may be useful therapeutically. Such cells could be mitotically inactivated, and the mitotically inactivated cells may be applied to a number of matrices to make a tissue engineered construct where the cells survive for a period of time secreting the factor(s) and then die. In certain embodiments, the cells are irradiated to inactivate them. A typical irradiation protocol for this purpose (giving cells in a free state) would involve exposing the cells to 20 to 50 Gy (2000 to 5000 rads; sometimes up to 100 Gy) from a Cs-137 or C0-60 source. In certain embodiments, a practical device configuration for releasing secreted factors would involve cell encapsulation. Another way to inactivate cells is by treating the cells with mitomycin C. The cells can be encapsulated (or microencapsulated) collectively or as clusters or individually in porous implantable polynmeric capsules. These can be made of a variety of substances, including but not limited to, polysaccharide hydrogels, chitosans, calcium or barium alginates, layered matrices of alginate and polylysine, poly(ethylene glycol) (PEG) polymers, polyacrylates (e.g., hydroxyethyl methacrylate methyl methacrylate), silicon, or polymembranes (e.g., acrylonitrile-co-vinyl chloride) in capillary-like, tube-like or bag-like configurations. Among the requirements for therapeutic utility are chemical definability, the ability to validate structure, stability, resistance to protein absorption, lack of toxicity, permeability to oxygen and nutrients as well as to the released therapeutic compounds, and resistance to antibodies or cellular attack. See, e.g., Orive et al. (2003) Nature Medicine 9(1):104-107 and Methods of Tissue Engineering, Eds Atalla, A. and Lanza, R. P. Academic Press, 2002.

### BRIEF DESCRIPTION OF THE DRAWINGS

FIG. 1 shows a schematic drawing illustrating one experimental design for performing the differentiation step of pluripotent stem cells by subjecting said pluripotent stem cells to a variety or combination of differentiation conditions over time, leading to a heterogeneous population of cells, herein referred to as candidate cultures. In order to identify the individual candidate cultures (“CC”), each CC is assigned a reference position number (such as CC1-CC90).

FIG. 2 shows a schematic drawing illustrating one experimental design for performing the propagation step of the candidate cultures identified from FIG. 1. Under the propagation step, the individual candidate cultures are disaggregated to produce single cells and then subjected to an array of combinations of propagation conditions that promote cellular differentiation or propagation.

FIG. 3 shows colony growth visualized with crystal violet staining after two weeks of growth. FIG. 3A depicts the entire plate of colonies. Colonies that were removed from the plate with cloning cylinders were identified by the circular markings. FIG. 3B depicts colonies that were determined to be too close together to be separated. FIG. 3C depicts the typical colonies that were subsequently chosen for isolation. These discrete colonies were characterized as colonies with uniformly circular boundaries that were at this or greater distances apart from each other. See Example 13.

FIG. 4 depicts a representative phase contrast photograph of single cell-derived populations of cells (ACTC 2017, ACTC 2026 and ACTC 20230) in their primary colonies (P0) and after the fourth passage (P4). See Example 13.

FIG. 5 depicts a phase contrast photograph of dermal progenitor candidate Clone 8 (ACTC51/B2).

FIG. 6 depicts the relative pattern of gene expression of 17 different cell clones derived from Series 1 as described in Example 17. The cell clone numbers 1-17 along the horizontal axis represent the following cell lines: (1) ACTC61 or B30, (2) ACTC54 or B17, (3) ACTC52 or B29, (4) ACTC56 or B6, (5) 4-1, (6) 4-3, (7) B-10, (8) ACTC51 or B2, (9) ACTC53 or B7, (10) ACTC57 or B25, (11) ACTC58 or B11, (12) ACTC55 or B3, (13) ACTC50 or 326, (14) ACTC64 or 6-1, (15) ACTC62 or 2-2, (16) ACTC63 or 2-1, and (17) ACTC60 or B-28. The cell clones in FIGS. 7-16, 18, 21 and 23 represent the same Series 1 cell lines. The expression of the following genes in each of the 17 cell clones were measured in FIG. 6: (a) dermo-1 (TWIST2), (b) dermatopontin (DPT), (c) PRRX2, (d) PEDF (SERPINF1), (e) AKR1C1, (f) collagen VI/alpha 3 (COL6A3), (g) microfibril-associated glycoprotein 2 (MAGP2), (h) GLUT5, (i) WISP2, (j) CHI3L1, (k) Odd-Skipped Related 2 (OSR2), (1) angiopoietin-like 2 (ANGPTL2), (m) RGMA, (n) EPHA5, (o) smooth muscle Actin Gamma 2 (ACTG2), (p) fibulin-1 (FBLN1), (q) LOXL4, (r) CD44 (the receptor for hyaluronic acid which promotes scarless wound repair), and (s) ADPRT (housekeeping gene for purposes of normalization). Values shown in the vertical axis of each of the histograms of the 17 cell clones of Series 1 represent the mean normalized relative fluorescent units (RFU) of the gene of interest. Values of approximately 100 RFU represents nonspecific background signal. The expression of these genes may be useful as markers to identify dermal fibroblast progenitor cells.

FIG. 7 depicts the relative expression of the SOX11 gene in the 17 different cell clones derived from Series 1 as described in Example 17. FIG. 7 illustrates that cell clone 1 of Series 1 as compared to some other cell clones of Series 1 express higher levels of the SOX11 gene. Values shown represent the normalized relative fluorescent units (RFU). See Example 18.

FIG. 8 depicts the relative expression of the CPE gene in the 17 different cell clones derived from Series 1 as described in Example 17. FIG. 8 illustrates that cell clones 1, 2, 4, 5, 6 and 7 of Series 1 express higher levels of the CPE gene as compared to some other cell clones of Series 1. Values shown represent the normalized relative fluorescent units (RFU). See Example 18.

FIG. 9 depicts the relative expression of the CPZ gene in the 17 different cell clones derived from Series 1 as described in Example 17. FIG. 9 illustrates that cell clones 8, 9, 10, 11, 13 and 14 of Series 1 express higher levels of the CPZ gene as compared to some other cell clones of Series 1. Values shown represent the normalized relative fluorescent units (RFU). See Example 18.

FIG. 10 depicts the relative expression of the C3 gene in the 17 different cell clones derived from Series 1 as described in Example 17. FIG. 10 illustrates that cell clones 8, 9, 10 and 12 of Series 1 express higher levels of the C3 gene compared to some other clones of Series 1. Values shown represent the normalized relative fluorescent units (RFU). See Example 18.

FIG. 11 depicts the relative expression of the MASP1 gene in the 17 different cell clones derived from Series 1 as described in Example 17. FIG. 11 illustrates that cell clones 8, 10, 11, 14, 15 and 16 of Series 1 express higher levels of the MASP1 gene as compared to some other clones of Series 1. Values shown represent the normalized relative fluorescent units (RFU). See Example 18.

FIG. 12 depicts the relative expression of the BF gene in the 17 different cell clones derived from Series 1 as described in Example 17. FIG. 12 illustrates that cell clones 10, 12, 13 and 14 of Series 1 express higher levels of the BF gene as compared to some other clones of Series 1. Values shown represent the normalized relative fluorescent units (RFU). See Example 18.

FIG. 13 depicts the relative expression of the FGFR3 gene in the 17 different cell clones derived from Series 1 as described in Example 17. FIG. 13 illustrates that cell clone 1 of Series 1 express higher levels of the FGFR3 gene as compared to some other clones of Series 1. Values shown represent the normalized relative fluorescent units (RFU). See Example 18.

FIG. 14 depicts the relative expression of the MYL4 gene in the 17 different cell clones derived from Series 1 as described in Example 17. FIG. 14 illustrates that cell clone 4 of Series 1 express higher levels of the MYL4 gene as compared to some other clones of Series 1. Values shown represent the normalized relative fluorescent units (RFU). See Example 18.

FIG. 15 depicts the relative expression of the MYH3 gene in the 17 different cell clones derived from Series 1 as described in Example 17. FIG. 15 illustrates that cell clone 9 of Series 1 express higher levels of the MYH3 gene as compared to some other clones of Series 1. Values shown represent the normalized relative fluorescent units (RFU). See Example 18.

The clones referred to above are described in Example 17. Series 1 refers to the cell lines generated in Example 17.

FIG. 16 depicts the relative mRNA expression levels of various genes in the 17 cell clones derived from Series 1, as compared to the housekeeping ADPRT gene. The following gene markers were expressed: (a) actin gamma 2, (b) smooth muscle actin (ACTA2), (c) the endothelial receptor for angiopoietin-1 (TEK), (d) PLAP1, (e) tropomyosin-1 (TPM-1), (f) calponin-1 (CNN1), (g) dysferlin, (h) the unidentified gene L0051063, (i) the oxidized low-density (lectin-like) receptor-1 (OLR1), (j) LRP2 binding protein (Lrp2bp), (k) MAGP2, (1) LOXL4, (m) MaxiK), and (n) ADPRT (shown for purposes of normalization). The expression of these genes may be useful as markers to identify smooth muscle progenitor cells. Based on the relative expression patterns illustrated in FIG. 16, cell clones 15-17 of Series 1 express unique markers of novel embryonic smooth muscle cell strains. Cell clones 15-17 and details relating to the markers are described in Example 21.

FIG. 17 depicts a phase contrast photographs of smooth muscle clonogenic cell lines produced from hES cell line ACTS. Clone 15 (ACTC62/2-2), clone 16 (ACTC63/2-1) and clone 17 (ACTC60/B-28) of Series 1 are shown after thawing at passage number 7. See Example 21.

FIG. 18 depicts the expression of HOX and other developmentally-regulated segmentation genes in identifying cell types in hES-derived cell clones 1-17 of Series 1. The expression of the following gene markers were measured in FIG. 18: (a) Dlx1, (b) Dlx2, (c) HOXD1, (d) HOXA2, (e) HOXA5, (f) HOXC6, (g) HOXD8, (h) HOXC10, (i) HOXA11 and (j) HOXD11. See Example 22.

FIG. 19 is a photograph of a representative clonogenic colony of candidate cells expressing a prenatal pattern of dermal fibroblast gene expression derived from embryoid bodies.

FIG. 20 is a photograph of a representative clonogenic colony of candidate epidermal keratinocyte cells expressing a prenatal pattern of gene expression derived from embryoid bodies as described in Example 24.

FIG. 21 depicts the relative pattern of gene expression of 17 different cell clones derived from Series 1 as described in Example 17, as compared to the standard housekeeping ADPRT gene. The expression of the following genes were measured: (a) HOXA2, (b) HOXB-2, (c) SOX11, (d) ID4, (e) FOXC1, (f) Cadherin-6, (g) PTN, (h) SLITRK3 and (i) CRYAB. The expression of the housekeeping ADPRT gene is depicted in (j) (shown for purposes of normalization). The expression of these genes may be useful as markers to identify cranial neural crest progenitor cells. See Example 25.

FIG. 22 depicts a phase contrast photograph of single cell-derived cranial neural crest cells (clone 1; also referred to as ACTC61/B30) of Series 1 at passage 7 derived from the human ES cell line ACTS. See Example 25.

FIG. 23 depicts the relative expression of the VEGFC gene in the 17 different cell clones derived from Series 1 as described in Example 17.

In FIGS. 6-16, 18, 21 and 23, the y-axis represents relative units and clones 1-17 of Series 1 (see examples 17, 18, 21, 22, 25 and 26) are shown in the x-axis.

FIG. 24 illustrates a robotic platform which may be used to perform the methods of the invention.

### DETAILED DESCRIPTION OF THE INVENTION

Table of Abbreviations

| | |
|---|---|
| AFP | Alpha fetoprotein |
| BMP | Bone Morphogenic Protein |
| BRL | Buffalo rat liver |
| BSA | Bovine serum albumin |
| CD | Cluster Designation |
| cGMP | Current Good Manufacturing Processes |
| CNS | Central Nervous System |
| DMEM | Dulbecco's modified Eagle's medium |
| DMSO | Dimethyl sulphoxide |
| DPBS | Dulbecco's Phosphate Buffered Saline |
| EC | Embryonal carcinoma |
| ECM | Extracellular Matrix |
| ED Cells | Embryo-derived cells; hED cells are |
| human | ED cells |
| EDTA | Ethylenediamine tetraacetic acid |
| ES Cells | Embryonic stem cells; hES cells are |
| human | ES cells |
| FACS | Fluorescence activated cell sorting |
| FBS | Fetal bovine serum |
| GMP | Good Manufacturing Practices |
| hED Cells | Human embryo-derived cells |
| EG Cells | Embryonic germ cells; hEG cells are |
| human | EG cells |
| EC cells | Embryonal carcinoma cells; hEC cells are |
| | human embyronal carcinoma cells |
| HSE | Human skin equivalents are mixtures of |
| | cells and biological or synthetic |
| | matrices manufactured for testing purposes |
| | or for therapeutic application in |
| | promoting wound repair. |
| ICM | Inner cell mass of the mammalian |
| | blastocyst-stage embryo. |
| LOH | Loss of Heterozygosity |
| MEM | Minimal essential medium |
| NT | Nuclear Transfer |
| PBS | Phosphate buffered saline |
| PS fibroblasts | Pre-scarring fibroblasts are |
| | fibroblasts derived from the skin of |
| | early gestational skin or derived from ED |
| | cells that display a prenatal pattern of |
| | gene expression in that they promote the |
| | rapid healing of dermal wounds without |
| | scar formation. |
| RA | Retinoic acid |
| RFU | Relative Fluorescence Units |
| SCNT | Somatic Cell Nuclear Transfer |
| SFM | Serum-Free Medium |
| SPF | Specific Pathogen-Free |
| SV40 | Simian Virus 40 |
| Tag | Large T-antigen |
| T-EDTA | Trypsin EDTA |

### DEFINITIONS

The term “analytical reprogramming technology” refers to a variety of methods to reprogram the pattern of gene expression of a somatic cell to that of a more pluripotent state, such as that of an ES, ED, EC or EG cell, wherein the reprogramming occurs in multiple and discrete steps and does not rely simply on the transfer of a somatic cell into an oocyte and the activation of that oocyte (see U.S. application No. 60/332,510, filed Nov. 26, 2001; Ser. No. 10/304,020, filed Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application No. 60/818,813, filed Jul. 5, 2006, PCT/US06/30632, filed Aug. 3, 2006, the disclosure of each of which is incorporated by reference herein).

The term “cellular reconstitution” refers to the transfer of a nucleus of chromatin to cellular cytoplasm so as to obtain a functional cell.

The term “cytoplasmic bleb” refers to the cytoplasm of a cell bound by an intact, or permeabilized, but otherwise intact plasma membrane but lacking a nucleus.

The term “pluripotent stem cells” refers to animal cells capable of differentiating into more than one differentiated cell type. Such cells include hES cells, hED cells, hEG cells, human EC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “embryonic stem cells” (ES cells) refers to cells derived from the inner cell mass of blastocysts, blastomeres, or morulae that have been serially passaged as cell lines while maintaining an undifferentiated state (e.g. express TERT, OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. The term “human embryonic stem cells” (hES cells) refers to human ES cells.

The term “colony in situ differentiation” refers to the differentiation of colonies of hES, hEG, human EC or hED cells in situ without removing or disaggregating the colonies from the culture vessel in which the colonies were propagated as undifferentiated stem cell lines. Colony in situ differentiation does not utilize the intermediate step of forming embryoid bodies, though embryoid bodies or other aggregation techniques such as the use of spinner culture may nevertheless follow a period of colony in situ differentiation.

The term “direct differentiation” refers to process of differentiating blastomere cells, morula cells, ICM cells, ED cells, or somatic cells reprogrammed to an undifferentiated state directly without the intermediate state of propagating undifferentiated stem cells such as hES cells as undifferentiated cell lines.

The term “human embryo-derived” (“hED”) cells (hEDC) refer to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, and mesoderm and their derivatives, but excluding hES cells that have been passaged as cell lines. The hED cells may be derived from fertilization of an egg cell with sperm or DNA, nuclear transfer, chromatin transfer, parthenogenesis, analytical reprogramming technology, or by means to generate hES cells with homozygosity in the HLA region.

The term “human embryonic germ cells” (hEG cells) refer to pluripotent stem cells derived from the primordial germ cells of fetal tissue or maturing or mature germ cells such as oocytes and spermatogonial cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA (see U.S. application No. 60/161,987, filed Oct. 28, 1999; Ser. No. 09/697,297, filed Oct. 27, 2000; Ser. No. 09/995,659, filed Nov. 29, 2001; Ser. No. 10/374,512, filed Feb. 27, 2003; PCT application no. PCT/US/00/29551, filed Oct. 27, 2000; the disclosures of which are incorporated herein in their entirety).

The term “histotypic culture” refers to cultured cells that are aggregated to create a three-dimensional structure with tissue-like cell density such as occurs in the culture of some cells over a layer of agar or such as occurs when cells are cultured in three dimensions in a collagen gel, sponge, or other polymers such as are commonly used in tissue engineering.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from the cells that do not share these common characteristics and are allowed to proliferate, generating a population of cells that are essentially entirely derived from the original population of similar cells.

The term “differentiated cells” when used in reference to cells made by methods of this invention from pluripotent stem cells refer to cells having reduced potential to differentiate when compared to the parent pluripotent stem cells. These differentiated cells of this invention comprise cells that could differentiate further (i.e., they may not be terminally differentiated).

The term “organotypic culture” refers to cultured cells that are aggregated to create a three-dimensional structure with tissue-like cell density such as occurs in the culture of some cells over a layer of agar, cultured as teratomas in an animal, otherwise grown in a three dimensional culture system but wherein said aggregated cells contain cells of different cell lineages, such as, by way of nonlimiting examples, the combination of epidermal keratinocytes and dermal fibroblasts or the combination of parenchymal cells with their corresponding tissue stroma, or epithelial cells with mesenchymal cells.

The term embryonal carcinoma (“EC”) cells, including human EC cells, are embryonal carcinoma cells such as TERA-1, TERA-2, NTera-2. EC cells are well known in the art.

This invention provides methods for the derivation of cells that are derived from a single (clonal) cell or a small number of similar cells (oligoclonal) differentiated, or in the process of differentiating, from pluripotent stem cells, wherein said single cells or oligoclonal cells are propagated to produce a population of cells—a population being two or more cells, under propagation conditions identified by means of screening a panel of conditions including, but not limited to, combinations of growth factors, extracellular components, conditioned media, hormones, ion concentrations, and co-culture with inducing or feeder cell types. This invention also provides formulation and use of the cells derived from the methods of this invention as well as engineered tissues made of such cells. Certain embodiments of this invention are described in the summary of the invention section and will not be repeated in this detailed description section.

The cells of this invention are differentiated from, or in the process of differentiating from, pluripotent stem cells, which could be any pluripotent stem cells. In some embodiments, the pluripotent stem cells include hES, hEG, hEC and hED cells, as well as pluripotent stem cells derived from the developing embryo such as those of the first eight weeks of human embryonic development including, but not limited to, pluripotent endodermal, mesodermal, or ectodermal progenitor cells. In some embodiments, the pluripotent stem cells may be derived from human or nonhuman embryonic or fetal tissues.

While techniques to differentiate hES cells into several differentiated states have been described, and whereas the use of clonogenic assays have been described for use in assaying the proliferative potential of bone marrow hematopoietic and stromal cells, for purifying some mixtures of cells, or otherwise characterizing said cells, the present invention uniquely describes the novel method of deriving populations of two or more, preferably one hundred or more cells, from a single (clonal) cell or a small number of similar cells (oligoclonal) differentiated from, or in the process of differentiating from, embryonic pluripotent stem cells such as hES, hEG, hEC, hED cells or other pluripotent embryonic stem cells such as primitive endoderm, mesoderm, or ectodermal cells, wherein the resulting single cell-derived population of cells can be documented not to have contaminating cells from the original pluripotent stem cells, wherein the resulting single cell-derived or oligoclonal population of cells are isolated from a heterogeneous population and can be used in cell therapy, research, for the isolation of novel extracts with therapeutic utility, or for the derivation of ligands that specifically bind to said cells.

The present invention also provides a means of identifying single cell-derived populations of cells of this invention capable of scalability. This invention also provides methods for identifying conditions for the propagation of said cells, for characterizing the differentiated state of said cells, and for identifying single cell-derived populations of cells capable of being stably engrafted after transplantation.

In one aspect of the invention, the method provides a means of identifying single cell-derived populations of cells of this invention with a pattern of gene expression corresponding to that of an animal of the same species in the prenatal state in vivo, as well as identifying conditions for the propagation of said cells.

In one aspect of the invention, the method provides a means of identifying the single cell-derived populations of cells of this invention using flow cytometry or analogous affinity-based cell sorting technology such as magnetic bead sorting and the further characterization of these cells' gene expression, phenotype and stability. The resulting suspension of sorted cells may then be plated at a density of a single cell per well for colony formation and subsequent clonal expansion. In some case, the cell plating step may be accomplished using an automated cell deposition device (“ACDU”). The use of flow cytometry is particularly useful where said cell of this invention is rarely present in the original heterogenous mixture of cells or where said cell of this invention has only limited capacity to proliferate after clonal or oligoclonal isolation. Moreover, a larger number of starting cells can be isolated to increase the final yield.

In another aspect of the invention, the complexity of the initial heterogenous mixture of cells that result from the first step may be reduced to concentrate cell types of interest by sorting cells using antigens that are expected to be on the desired cell type or by genetically modifying the parent pluripotent stem cells with expression DNA constructs that comprise a promoter and a marker gene such as GFP, such that the particular gene is expressed in the cell type or family of cell types that is desired, allowing such cells to be identified and isolated.

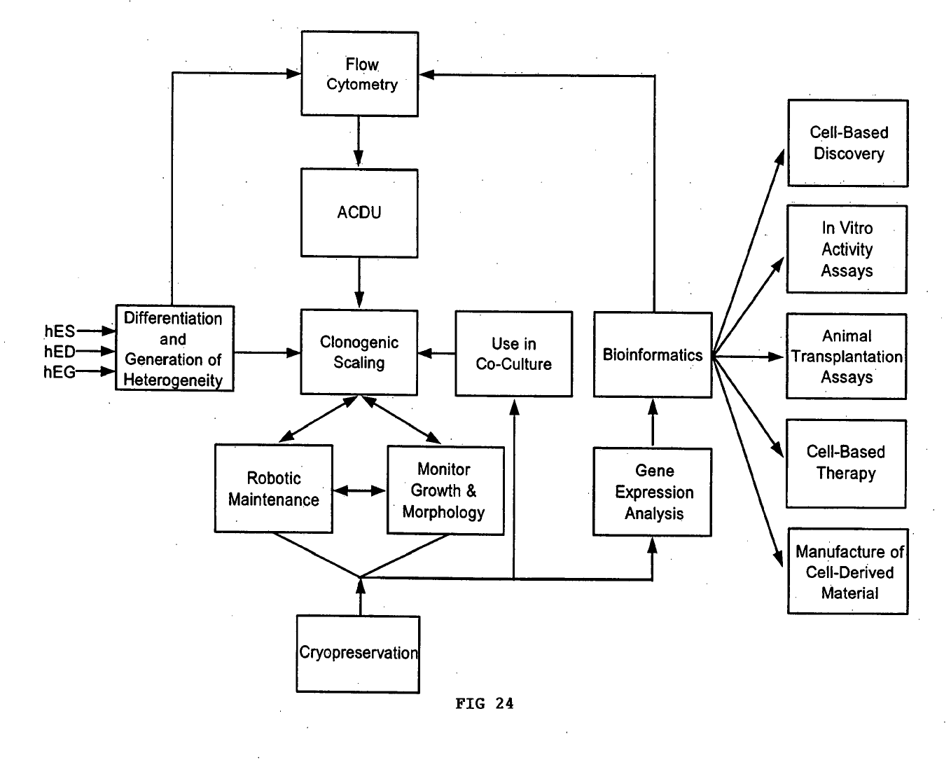

In another aspect of the invention, the methods of the invention may be automated, for example, by using robotic manipulation. In certain embodiments, cells may be expanded clonally or oligoclonally via robotic means in a variety of media, extracellular matrices, or co-cultured cells. In certain embodiments, robotic automation may also be used to monitor cell growth. In certain other embodiments, robotic automation may be used to culture and propogate cells made by methods of this invention, for example, passaging, feeding, and cryopreserving said cells, with generated information being stored in a computer database. This enables the reproducible production of desired cell types and may be useful in a research setting where a large number of culture conditions are assayed. Robotic automation of the methods of this invention may also be useful in personalized medicine where the robotic platform is combined with the cells from a patient and wherein each patient has customized differentiated cells produced. Components of such a robotic platform is illustrated in FIG. 24.

In one aspect of the invention, the method comprises the steps of deriving differentiated or differentiating cells by differentiating pluripotent stem cells for varying periods of time in vitro, in vivo, or in ovo, with or without an intermediate step of forming multicellular aggregates such as embryoid bodies, and distributing the differentiated cells in cell culture conditions wherein the cells are cultured attached to a substrate at such a low density that subsequent cultures are composed of colonies of cells derived from what was originally a single cell. In the case where multicellular aggregates such as embryoid bodies are formed, there may be a step to separate the aggregates into single cells, such as by trypsinizing the aggregates.

In another aspect of the invention, the method comprises the steps of deriving cells differentiated at various periods of time from pluripotent stem cells (such as hES cells), and culturing such differentiated or differentiating cells at low density in a semisolid media such that subsequent culture can identify colonies of cells derived from what was originally a single cell, wherein said differentiated or differentiating cells are cultured in combinations of various culture media (including, but not limited to, media conditioned in the presence of various cell types), growth factors, ambient gas concentrations, and extracellular matrices.

In certain embodiments, the differentiated cells or differentiating cells made by the methods of this invention are derived from a single cell that is documented by photography or other means of identification, such immunocytochemical or hybridization of probes with RNA or cDNA transcripts, to be a cell of a certain differentiated state such that it is not an ES cell in order to reduce the potential of transplanting undesired cells, such as undifferentiated cells including ES cells into the animal or human in need of cell-based therapy. The lack of contaminating ES cells in the differentiated cell or differentiating cell cultures made by the methods of this invention eliminates the potential risk of tumor-forming ES cells. It has previously been known that ES-derived cells may have the capability to form tumors, as evidenced by the existence of cancer stem cells. In contrast, the lack of contaminating ES cells in the differentiated cell or differentiating cell cultures made by the methods of this invention eliminates such tumor-forming ES cells. To confirm this, for example, the tumor-forming ability of hES-derived clonal cell lines of Series 1 generated by the methods of this invention was compared with hES cells. When hES-derived clonal cell lines of Series 1 of the present invention or hES cells were injected intramuscularly or subcutaneously into the rear legs of SCID mice, large teratomas (approximately one cm) were observed only in hES-injected mice at the site of injection three months later. However, no evidence of tumors were observed in the animals injected with hES-derived clonal cell lines of Series 1 of the present invention. No signs of malignancy, edema, erythema, or other pathology were observed at the site of injection or in any of the analyzed tissues in animals injected with hES-derived clonal cell lines of Series 1.

In another aspect of the invention, the method comprises deriving 100 or more cells from a single differentiated cell, or a cell in the process of differentiating, said cell resulting from differentiating a pluripotent stem cell, such as a hES cell, wherein the pluripotent stem cell is genetically modified to delete genes from the MHC gene family or cells wherein genes of the MHC gene family are first removed and then alleles of the MHC gene family are restored such as to make hemizygous or homozygous stem cells (see U.S. application Ser. No. 10/445,195, filed May 27, 2003; 60/729,173, filed Oct. 20, 2005, the disclosures of which are incorporated by reference).

In another aspect of the invention, the method comprises the derivation of 100 cells or more from a single differentiated cell differentiated from a pluripotent stem cell, or from a cell in the process of differentiating from a pluripotent stem cell such as a hED cell, wherein the pluripotent stem cell is derived from the direct differentiation of an embryonic cell or cells without the derivation of a human ES cell line.

In another aspect of the invention, the method comprises the derivation of 100 cells or more from a single differentiated cell or a cell in the process of differentiating from a pluripotent stem cell such as a hES cell wherein the hES cell line is derived from a single blastomere. The pluripotent embryonic stem cells can also be generated from a single blastomere removed from an embryo without interfering with the embryo's normal development to birth. See U.S. application No. 60/624,827, filed Nov. 4, 2004; 60/662,489, filed Mar. 14, 2005; 60/687,158, filed Jun. 3, 2005; 60/723,066, filed Oct. 3, 2005; 60/726,775, filed Oct. 14, 2005; Ser. No. 11/267,555 filed Nov. 4, 2005; PCT application no. PCT/US05/39776, filed Nov. 4, 2005, 60/797,449, filed May 3, 2006 and 60/798,065, filed May 4, 2006, the disclosures of which are incorporated by reference; see also Chung et al., Nature, Oct. 16, 2005 (electronically published ahead of print) and Chung et al., Nature V. 439, pp. 216-219 (2006), the disclosures of each of which are incorporated by reference).

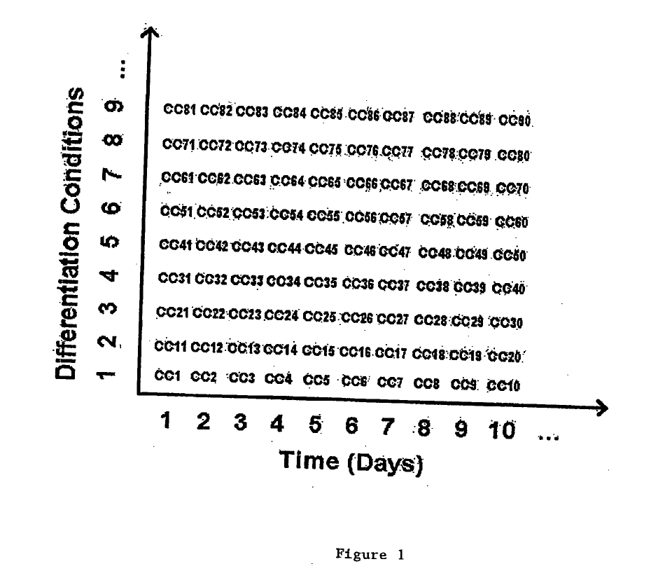

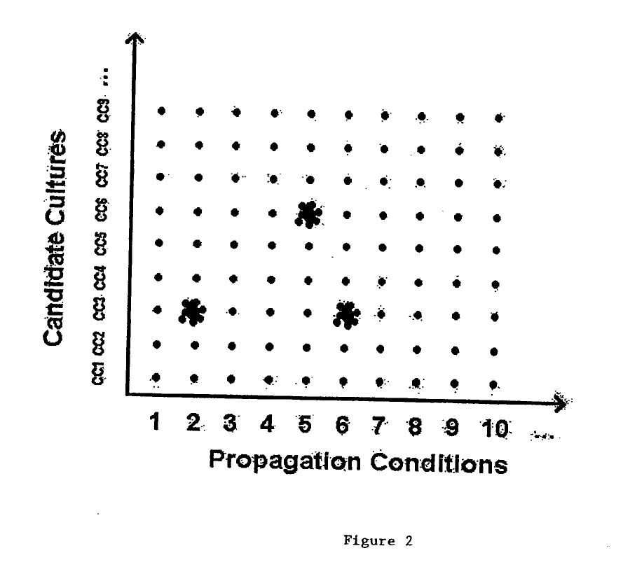

The present invention thus provides novel methods for the culture of mammalian pluripotent stem cell-derived cells from a single cell by first performing a differentiation step. In this differentiation step, pluripotent stem cells are differentiated under a variety or combination of different conditions leading to a heterogeneous populations of cells herein referred to as candidate cultures (“CC”) (see FIG. 1). These candidate cultures may be identified, such as with bar coding and identified as candidate cultures (in the case of FIG. 1 as candidate cultures 1-90 (CC1-90)). In a second step (see FIG. 2), said candidate cultures are disaggregated so as to produce single cells that are separated such that when the cells from the candidate cultures are exposed to culture conditions that promote cellular proliferation or propagation, said single cells from the candidate culture may proliferate and expand in cell number in a manner allowing said proliferating cells to be later retrieved for use. To produce single cells, the cells may be plated at limiting dilution or at low density in cloning cylinders. To produce oligoclonal cells, the cells may be plated at a higher density such that clusters of related cells are isolated based on morphology of by sampling of the cluster and testing by PCR for markers of interest. Cells of interest may also be picked from among the cells plated at low density wherein clonal derivation is nearly certain. The conditions to promote differentiation in step one to generate candidate cultures, and the conditions to promote propagation are chosen so as to make an array of combinations of conditions to screen for many possible candidate cultures and many possible propagation conditions.

The propagated single cell-derived cells of this invention have utility, for example, in research in cell biology, for the production of ligands for differentiation antigens, for the production of growth factors, for drug discovery as feeder cells to obtains other such cells or as feeder cells for totipotent or pluripotent stem cells (such as hES cells) and for cell-based therapy and transplantation in human and veterinary medicine.

In one embodiment of the invention, the pluripotent stem cells are differentiated under a variety or combination of different conditions, such as those conditions listed, for example, in Table I. The differentiation condition may include members of the EGF family of ligands; members of the EGF receptor/ErbB receptor family; members of the FGF ligand family; members of the FGF Receptor family; FGF regulators; Hedgehog family proteins; Hedgehog Regulators; members of the IGF family of ligands; IGF-I Receptor (CD221); members of the insulin growth factor-like binding protein (IGFBP) family of proteins; members of the Receptor Tyrosine Kinase family to sequester certain ligands; members of the proteoglycan family and proteoglycan regulators; members of the SCF, Flt-3 Ligand & M-CSF family; members of the Activin family; members of the BMP (Bone Morphogenetic Protein) family; members of the GDF (Growth Differentiation Factor) family; members of the GDNF Family of Ligands; members of the TGF-beta family of proteins; other TGF-beta Superfamily Ligands; members of the TGF-beta superfamily of receptors; modulators of the TGF-beta superfamily; members of the VEGF/PDGF family of factors; members of the family of Dickkopf proteins & Wnt inhibitors; members of the Frizzled family of factors and related proteins; members of the Wnt family of ligands; other Wnt-related Molecules; other factors known to influence the growth or differentiation of cells; members of the steroid family of hormones; members of the extracellular/membrane family of proteins; extracellular matrix proteins, ambient oxygen conditions; animal serum conditions; members of the interleukin family of proteins; members of the protease family of proteins; any one of the amino acids; members of the prostaglandin family; members of the retinoid receptor agonists/antagonists; a variety of different commercial cell culture media such as those listed in Table I; or miscellaneous inducers.

In another embodiment of the invention, the pluripotent stem cells are differentiated under a variety or combination of different conditions, such as any compounds or agents that belong to the family of teratogens listed, for example, but not limited to those, in Table IV. Tetratogens refer to any agents or compounds known to affect differentiation in vivo.

In certain embodiments of the invention, the various culture conditions that may be used in the first differentiation step or the subsequent propagation step include but not limited to: plating the cells directly on culture vessel wall, such as a dish, multiwell dish, flask, or roller bottle; attaching the cells to beads, microcarriers or disks, or solid or hollow fibers; encapsulating the cells in gels such as alginates; or culturing the cells in semisolid media as is well known in the art for the culture of hematopoietic and other bone marrow-derived cells grown in suspension; culturing the cells in ovo, such as in juxtaposition with SPF chicken unfertilized eggs or fertilized SPF eggs in juxtaposition with avian embryonic cells; culturing the cells in microdrops, in hanging drops, as cell aggregates analogous to mammospheres and neurospheres; plating the cells on tissue culture substrates with added ECM components, incubating the cells to extracts in solution, in vesicles such as liposomes, or RNA extracts, including micro RNA extracts from differentiated cells such as, but not limited to, those listed in Table II, or differentiating cells such as, but not limited to, those listed in Table III; culturing the cells in various media including, but not limited to: defined media, media with animal sera, conditioned media with cells of defined cell types, including stromal cells, parenchymal cells, media conditioned with tissue, including embryonic and fetal anlagen or media conditioned in the heterogeneous culture from which the single cells were originally isolated, or conditioned medium obtained from the original culture of differentiated cells prior to trypsinization or such conditioned medium at 10% or 50% of the medium.

In another embodiment of the invention, the cells can be co-cultured with inducing cells on one layer, said inducing cells including stromal cells, parenchymal cells, embryonic and fetal anlagen or single cell-derived colonies on another layer.

In another embodiment of the invention, the single cell-derived or oligoclonal derived cells may be used as feeders or inducer cells for cell derivation of new cell types. The single cell or oligoclonal-derived feeder/inducer cell lines may be cultured in a variety of conditions and combined with a heterogenous mixture of candidate cells. The single cell or oligoclonal-derived feeder/inducer cells may also be mitotically inactivated using, for example, mitomycin C or ionizing radiation.

The complete media used in the isolation of single cell-derived cells may be defined medium without sera or other uncharacterized ingredient such as D-MEM/F-12 (1:1), and with insulin, transferrin, epidermal growth factor, leutinizing hormone or follicle stimulating hormone, somatomedin and growth hormone with HEPES buffer added to 15 mM to compensate for the loss of the buffering capacity of serum.

Conditions may be used to promote the growth of cells at clonal densities such as culturing the cells in an oxygen partial pressure less than that of the ambient atmosphere, such as 1-10% oxygen, preferably 3-5% oxygen, culturing the cells in media lacking phenol red, culturing the cells with the addition of agents useful in metabolizing the toxic effects of oxygen such as the addition of 0.1 nM-10 μM selenium, preferably 1.0 nM-1 μM selenium, 10⁻⁵-10⁻⁷ M N-acetyl cysteine, (preferably 10⁻⁵ M), and/or 500 U/mL of catalase.

In another embodiment of the invention, cells from the first differentiation step but prior to the clonal or oligoclonal propagation step, are placed in growth media similar to or identical to that in which they will be clonally or oligoclonally expanded in order to increase the number of cells capable of propagating in the medium of the second step. This enrichment step allows an increased number and more predictable number of cells to proliferate in the final clonal or oligoclonal medium of the second step. In some cases where the medium of the initial differentiation step is identical to or similar to the medium in which the cells will be clonally or oligoclonally expanded, the enrichment step may also increase the number of proliferating cells such that the heterogeneous mixture may be cryopreserved and in the event that the clonal or oligoclonal isolation yielded useful cell types, the cryopreserved heterogeneous mixture of cells may be thawed and used as a source of cells for clonal or oligoclonal isolation again. Therefore, in one embodiment, the enrichment step is part of the initial differentiation step in that the culture medium of the first differentiation step is identical to, or similar to, that of the second clonal or oligoclonal propagation step. Alternatively, the enrichment step may be a separate step. The cells may be initially differentiated in one medium, then the heterogeneous mixture of cells can be transferred at normal cell culture densities to a different medium of the second clonal or oligoclonal expansion step. The cells are cultivated in that medium in a separate step. After a period of time of 2-30 days (preferably 5-14 days) that allows for the percentage of cells capable of being propagated in the medium to be increased, the heterogeneous mixture of cells is then clonally or oligoclonally expanded as described herein.

In another embodiment of the invention, the enrichment step may be effected or facilitated by physical separation of various subsets of the heterogeneous mixture of cells from the first differentiation step and/or the enrichment step. These subsets may, for example, represent cells at one or more lineages or stages of maturation or differentiation. One way to achieve this is to react the cells with a ligand or ligands such as, but not limited to, antibodies useful to positively select or purify specific cell types, or to delete the heterogeneous mixture of cells of specific cell types. A person of ordinary skill in the art can be guided in this effort by the gene expression profile of cells disclosed in this application. This gene expression profile data can yield useful information on the cell surface gene expression of antigens or other molecules such as differentiation or lineage markers for which antibodies or other ligands to such markers are available. For example, the isolation of RNA with subsequent gene expression analysis can yield a profile of the expression of transcripts related to cell surface antigens, and these can be useful in purifying the heterogeneous mixture of cells of step 1 (a) and 1 (b) using affinity methods known in the art, to increase the frequency of cells of a desired type for subsequent clonal isolation in steps 2 (a) and 2 (b) or the direct use of the cells without clonal or oligoclonal isolation. According, such antigens and markers are useful in the identification and purification of cells made by the method of this invention as is understood by one skilled in the art. For example, in the case of cell clone ACTC60 (or B-28) of Series 1, ligands to CD13 (ANPEP), CD42c (GP1BB), CD49a (ITGA1), CD49d (ITGA4), and CD202b (TEK) may be useful in the identification and purification of this cell clone.

In another embodiment of the invention, the first differentiation step may be mediated by siRNA or other similar techniques (i.e. ribozymes, antisense). The use of siRNA (including miRNAs that naturally regulate cell differentiation and are known in the art) in the first differentiation step may provide a means of steering the differentiation of the pluripotent stem cells to make a heterogeneous population of cells that are biased in some direction, for example, to become endoderm, mesoderm or ectoderm. For example, transfection of embryonic stem cells with OCT4- or Nanog-targeted RNAi is sufficient to induce differentiation towards extraembryonic lineages (Hough et al. Stem Cells. 2006 Feb. 2; Epub). RNAi has been shown to work in a number of cells, including mammalian cells, such as ES cells.

In another embodiment of the invention, the initial pluripotent stem cells may express the catalytic component of telomerase reverse transcriptase (hTERT) (such as when the cells are ES cell lines) and telomere length may be maintained in cultures of said stem cells such that differentiated derived cells made according to the present invention have relatively long proliferative lifespans allowing for clonal, even up to five serial clonal isolations. In addition, since the cells express TERT, telomere length may be increased through the addition of agents to the culture that increase mean telomere length in said cells. The increased in mean telomere length in the TERT-expressing pluripotent stem cell such as an ES cell, then leads to an increased proliferative lifespan of the telomerase negative derived cells. This leads to the repression of telomerase activity when said cells undergo differentiation and said cells are able to retain an increased proliferative lifespan when compared to normal somatic cells of that species.

Pluripotent stem cells that are naturally expressing the catalytic component of telomerase reverse transcriptase (hTERT) and normally repress that expression when the pluripotent stem cells differentiate may be treated with exogenous agents to increase the mean telomere length in the pluripotent stem cells. The differentiated cells from said stem cells will display an increased replicative lifespan when compared to their normal counterparts. Such agents may include, but are not limited to, inhibitors of DNA cytosine-C5-methyltransferase 3 beta (DNMT3B; accession number NM_175849.1) using, for example, siRNA constructs targeting the mRNA transcripts of that gene, or small molecule inhibitors of the enzyme. The knockout of DNA3B in tumor cells has been reported to increase the mean telomere length in those cells, but the inhibition of that enzyme would not necessarily be expected in any normal cell type such as pluripotent stem cells with germ-line telomere length. Additional molecular targets to transiently increase mean telomere length include, for example, modulators of poly (ADP-ribose) polymerase (ADPRT; accession number NM_001618.2), TERF1, TERF2, and the exogenous addition of estrogen or telomeric oligonucleotides.

In certain embodiments of the invention, the pluripotent stem cells may be transfected with a DNA construct such that hTERT or the TERT gene of another species is constitutively activated or inducibly by an extrinsic activator as is well known in the art. In some embodiments, the TERT gene may be derived from mammalian species other than human, including, but not limited to, equine, canine, porcine, bovine, and ovine sources; rodent sources such as mouse or rat; or avian sources. The differentiated cell clones generated according to the present invention may then be constitutively immortal or conditionally immortal. Such cells will be useful where the expansion of said cells would normally erode telomere length below a desired level.

In another embodiment of the invention, the first differentiation step may be mediated by reprogramming the expression profile of a cell to convert it into that of a desired cell type. For example, the pluripotent stem cells can be reprogrammed by incubating the nucleus or chromatin mass from said pluripotent stem cells with a reprogramming media (e.g., a cell extract) under conditions that allow nuclear or cytoplasmic components such as transcription factors to be added to, or removed from, the nucleus or chromatin mass (see U.S. application Ser. No. 10/910,156, filed Aug. 2, 2004 (US publication no. 20050014258, published Jan. 20, 2005); see also U.S. application No. 60/705,625, filed Aug. 3, 2005; U.S. application No. 60/729,173, filed Oct. 20, 2005; U.S. application No. 60/818,813, filed Jul. 5, 2006). The added transcription factors may promote the expression of mRNA or protein molecules found in cells of the desired cell type, and the removal of transcription factors that would otherwise promote expression of mRNA or protein molecules found in said pluripotent stem cells. If desired, the chromatin mass may then be incubated in an interphase reprogramming media (e.g., an interphase cell extract) to reform a nucleus that incorporates desired factors from either reprogramming media. The nucleus or chromatin mass is then inserted into a recipient cell or cytoplast, forming a reprogrammed cell of the desired cell type. In another embodiment, a permeabilized cell is incubated with a reprogramming media (e.g., a cell extract) to allow the addition or removal of factors from the cell, and then the plasma membrane of the permeabilized cell is resealed to enclose the desired factors and restore the membrane integrity of the cell. If desired, the steps of any of these methods may be repeated one or more times or different reprogramming methods may be performed sequentially to increase the extent of reprogramming, resulting in a greater alteration of the mRNA and protein expression profile in the reprogrammed cell. Furthermore, reprogramming medias may be made representing combinations of cell functions (e.g., medias containing extracts or factors from multiple cell types) to produce unique reprogrammed cells possessing characteristics of multiple cell types.

Although human cells are preferred for use in the invention, the cells to be used in the method of the invention are not limited to cells from human sources. Cells from other mammalian species including, but not limited to, equine, canine, porcine, bovine, and ovine sources; or rodent species such as mouse or rat; or cells from other species such as avian, in particular SPF chicken ES-derived or embryo-derived cells, may be used.

In addition, cells that are spontaneously, chemically or virally transfected or recombinant cells or genetically engineered cells may also be used in this invention. For those embodiments that incorporate more than one cell type, chimeric mixtures of normal cells from two or more sources; mixtures of normal and genetically modified or transfected cells; or mixtures of cells of two or more species or tissue sources may be used.

In addition, clonal or oligoclonal cells isolated according to the invention may be modified to artificially inhibit cell cycle inhibitory factors or otherwise stimulate the cells to replicate rapidly through means well known in the art. Said artificial stimulation of the cell cycle may be made reversible through means well known in the art, including but not limited to, the use of inducible promoters, temperature sensitive promoters, RNAi, the transcient delivery of proteins into the cells or by other means known in the art. Any method known in the art to overcome cell cycle inhibition may be used with the invention. By way of nonlimiting example, the retinoblastoma and p53 pathways may be inhibited, such as by the use of T-antigen, the adenovirus proteins E1A and E1B, or the papillomavirus proteins E6 and E7. In certain embodiments, protein agents may be modified with protein transduction domains as described herein. By way of nonlimiting example, pluripotent stem cells such as ES, EG, EC or ED cells may be transfected with a construct that leads to an inducible SV40 T-antigen such as a temperature sensitive T-antigen. As a result, cells can be allowed to differentiate into an initial heterogeneity of cell types and then clonally or oligoclonally expanded under conditions wherein the SV40 T-antigen gene is induced to stimulate the proliferation of the cells. When sufficient numbers of cells are obtained, the expression of SV40 T-antigen may be downregulated by reversing the steps that led to the activation of the gene, or by the physical removal of the gene or genes using recombinase technology as is well known in the art, such as through the use of the CRE recombinase system or the use of FLP recombinase.

In certain embodiments, SV40 T-antigen may be added during the first differentiation step or at the beginning of the clonal or oligoclonal expansion/propagation step. In certain embodiments, the import of SV40 T-antigen may be improved by delivery with liposomes, electroporation, or by permeabilization (see U.S. Patent Application No. 20050014258, herein incorporated by reference). For example, cells may be permeabilized using any standard procedure, such as permeabilization with digitonin or Streptolysin O. Briefly, cells are harvested using standard procedures and washed with PBS. For digitonin permeabilization, cells are resuspended in culture medium containing digitonin at a concentration of approximately 0.001-0.1% and incubated on ice for 10 minutes. For permeabilization with Streptolysin O, cells are incubated in Streptolysin O solution (see, for example, Maghazachi et al., 1997) for 15-30 minutes at room temperature. After either incubation, the cells are washed by centrifugation at 400×g for 10 minutes. This washing step is repeated twice by resuspension and sedimentation in PBS. Cells are kept in PBS at room temperature until use. Alternatively, the cells can be permeabilized while placed on coverslips to minimize the handling of the cells and to eliminate the centrifugation of the cells, thereby maximizing the viability of the cells.

Delivery of T-antigen or other proteins may be accomplished indirectly by transfecting transcriptionally active DNA into living cells (such as the cells of this invention) where the gene is expressed and the protein is made by cellular machinery. Several methods are known to one of skill in the art to effectively transfect plasmid DNA including calcium phosphate coprecipitation, DEAE dextran facilitated transfection, electroporation, microinjection, cationic liposomes and retroviruses. Any method known in the art may be used with this invention to deliver T-antigen or other proteins into cells.

In certain embodiments, protein is delivered directly into cells of this invention, thereby bypassing the DNA transfection step. Several methods are known to one of skill in the art to effectively deliver proteins into cells including microinjection, electroporation, the construction of viral fusion proteins, and the use of cationic lipids.

Electroporation may be used to introduce foreign DNA into mammalian (Neumann, E. et al. (1982) EMBO J. 1, 841-845), plant and bacterial cells, and may also be used to introduce proteins (Marrero, M. B. et al. (1995) J. Biol. Chem. 270, 15734-15738; Nolkrantz, K. et al. (2002) Anal. Chem. 74, 4300-4305; Rui, M. et al. (2002) Life Sci. 71, 1771-1778). Cells (such as the cells of this invention) suspended in a buffered solution of the purified protein of interest are placed in a pulsed electrical field. Briefly, high-voltage electric pulses result in the formation of small (nanometer-sized) pores in the cell membrane. Proteins enter the cell via these small pores or during the process of membrane reorganization as the pores close and the cell returns to its normal state. The efficiency of delivery is dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines remain refractory even to electroporation unless partially activated.

Microinjection was first used to introduce femtoliter volumes of DNA directly into the nucleus of a cell (Capecchi, M. R. (1980) Cell 22, 470-488) where it can be integrated directly into the host cell genome, thus creating an established cell line bearing the sequence of interest. Proteins such as antibodies (Abarzua, P. et al. (1995) Cancer Res. 55, 3490-3494; Theiss, C. and Meller, K. (2002) Exp. Cell Res. 281, 197-204) and mutant proteins (Naryanan, A. et al. (2003) J. Cell Sci. 116, 177-186) can also be directly delivered into cells via microinjection to determine their effects on cellular processes first hand. Microinjection has the advantage of introducing macromolecules directly into the cell, thereby bypassing exposure to potentially undesirable cellular compartments such as low-pH endosomes. All of these techniques can be used on the cells of this invention or the parent pluripotent cells.

Several proteins and small peptides have the ability to transduce or travel through biological membranes independent of classical receptor- or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. The small protein transduction domains (PTDs) from these proteins can be fused to other macromolecules, peptides or proteins to successfully transport them into a cell (Schwarze, S. R. et al. (2000) Trends Cell Biol. 10, 290-295). Sequence alignments of the transduction domains from these proteins show a high basic amino acid content (Lys and Arg) which may facilitate interaction of these regions with negatively charged lipids in the membrane. Secondary structure analyses show no consistent structure between all three domains. The advantages of using fusions of these transduction domains is that protein entry is rapid, concentration-dependent and appears to work with difficult cell types (Fenton, M. et al. (1998) J. Immunol. Methods 212, 41-48.). All of these techniques can be used on the cells of this invention or the parent pluripotent cells.

Liposomes have been rigorously investigated as vehicles to deliver oligonucleotides, DNA (gene) constructs and small drug molecules into cells (Zabner, J. et al. (1995) J. Biol. Chem. 270, 18997-19007; Feigner, P. L. et al. (1987) Proc. Natl. Acad. Sci. USA 84, 7413-7417). Certain lipids, when placed in an aqueous solution and sonicated, form closed vesicles consisting of a circularized lipid bilayer surrounding an aqueous compartment. These vesicles or liposomes can be formed in a solution containing the molecule to be delivered. In addition to encapsulating DNA in an aqueous solution, cationic liposomes can spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Feigner, J. H. et al. (1994) J. Biol. Chem. 269, 2550-2561). The cationic liposome strategy has also been applied successfully to protein delivery (Zelphati, O. et al. (2001) J. Biol. Chem. 276, 35103-35110). Because proteins are more heterogeneous than DNA, the physical characteristics of the protein such as its charge and hydrophobicity will influence the extent of its interaction with the cationic lipids. All of these techniques can be used on the cells of this invention or the parent pluripotent cells.

In certain embodiments Pro-Ject Protein Transfection Reagent may be used. Pro-Ject Protein Transfection Reagent utilizes a unique cationic lipid formulation that is noncytotoxic and is capable of delivering a variety of proteins into numerous cell types. The protein being studied is mixed with the liposome reagent and is overlayed onto cultured cells. The liposome:protein complex fuses with the cell membrane or is internalized via an endosome. The protein or macromolecule of interest is released from the complex into the cytoplasm free of lipids (Zelphati, O. and Szoka, Jr., F. C. (1996) Proc. Natl. Acad. Sci. USA 93, 11493-11498) and escaping lysosomal degradation. The noncovalent nature of these complexes is a major advantage of the liposome strategy as the delivered protein is not modified and therefore is less likely to lose its activity. All of these techniques can be used on the cells of this invention or the parent pluripotent cells.

In certain embodiments, the nuclear localization sequence of SV40 T-antigen may be modified. Protein transduction domains (PTD), covalently or non-covalently linked to T-antigen, allow the translocation of T-antigen across the cell membranes so the protein may ultimately reach the nuclear compartments of the cells. PTDs that may be fused with a Tag protein include the PTD of the HIV transactivating protein (TAT) (Tat 47-57) (Schwarze and Dowdy (2000) Trends Pharmacol. Sci. 21: 45-48; Krosl et al. (2003) Nature Medicine 9: 1428-1432). For the HIV TAT protein, the amino acid sequence conferring membrane translocation activity 5 corresponds to residues 47-57 (YGRKKRRQRRR) (Ho et al. (2001) Cancer Research 61: 473-477; Vives et al. (1997) J. Biol Chem. 272: 16010-16017). This sequence alone can confer protein translocation activity. The TAT PTD may also be the nine amino acids peptide sequence RKKRRQRRR (Pauk et al. (2002) Mol Cells 30:202-8). The TAT PTD sequences may be any of the peptide sequences disclosed in Ho et al. (2001) Cancer Research 61: 473-477, including YARKARRQARR, YARZLAARQARA, YARAARRAARR, and RARAARRAARA. Other proteins that contain PTDs that may be fused with Tag include the herpes simplex virus 1 (HSV-1) DNA-binding protein VP22 and the Drosophila Antennapedia (Antp) transcription factor (Schwarze et al. (2000) Trends Cell Biol 10: 290-295). For Antp, amino acids 43-58 (RQIKIWFQNRRMKWM) represent the protein transduction domain, and for HSV VP22 the PTD is represented by the residues DAATATRGRSAASRPTERPRAPARSASRPRRPVE. Alternatively, HeptaARG (RRRRRRR) or artificial peptides that confer transduction activity may be used as a PTD. The PTD may be a PTD peptide that is duplicated or multimerized; including one or more of the TAT PTD peptide YARAAARQARA, or a multimer consisting of three of the TAT PTD peptide YARARARQARA. Techniques for making fusion genes encoding fusion proteins are well known in the art. The joining of various DNA fragments coding for different polypeptide sequences may be performed in accordance with conventional techniques. The fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & 20 Sons: 1992). A fusion gene coding for a purification leader sequence, such as a poly-(His) sequence, may be linked to the N-terminus or C-terminus of the desired portion of the Tag polypeptide or Tag-fusion protein allowing the fusion protein be purified by affinity chromatography using a metal resin. The purification leader sequence can then be subsequently removed by treatment with enterokinase to provide the purified Tag polypeptide (e.g., see Hochuli, E. et al (1987), J. Chromatog. 411:177-184). T antigen that is provided in the media may be excreted by another cell type. The other cell type may be a feeder layer, such as a mouse stromal cell layer transduced to express secretable T antigen. For example, T antigen may be fused to or engineered to comprise a signal peptide, or a hydrophobic sequence that facilitates export and secretion of the protein. Alternatively, T antigen, as a fusion protein covalently or linked to a PTD or as a protein or a fusion protein non-covalently linked to a PTD, may be added directly to the media. In certain embodiments, cell lines are created that secrete the TAT-T antigen fusion protein (see Derer, W. et al. (2001) The FASEB Journal, Published online). Conditioned medium from TAT-T antigen secreting cell lines is subsequently added to recipient cell lines to promote cell growth.

Human embryo-derived (hED) cells are cells that are derived from human embryos such as human preimplantation embryos, postimplantation embryos (such as aborted embryonic tissue) or pluripotent cell lines such as ES cell lines derived from human preimplantation embryos. Human zygotes, 2 or more cell premorula stage such as blastomeres, morula stage, compacting morula, blastocyst embryo inner cell masses, or cells from developing embryos all contain pluripotent cells. Such cells may be differentiated using techniques described herein to yield the initial heterogeneous population of cells of the first step. Because such culture conditions may induce the direct differentiation of the cells without allowing the propagation of a hES cell line, the probability of a hES cell contaminating the resulting clonal or oligoclonal cultures are reduced.

The single cells of this invention (made by the methods of this invention) may be used as the starting point for deriving various differentiated cell types. The single cells of this invention may be the precursors of any cell or tissue lineage.

In another embodiment of the invention, the clonal or oligoclonal populations may be derived from embryonic tissues. For example, embryonic tissue may be dissected and the cells disaggregated. Such disaggregated cells may be then be used as the starting parent pluripotent cells of the methods of this invention.

There have been numerous attempts in the prior art to differentiate embryonic stem cells, embryonal carcinoma cells, and embryonic germ cells into various cell types. These methods have been only marginally been successful due to problems with culturing and characterizing the complex mixture of cell types originating out of differentiating ES, EC, and EG cultures in vitro. It has not been possible to preserve a pure culture of the differentiated cell type without having the culture overgrown with fibroblastic or other contaminating cell types. See, Ian Freshney, Culture of Animal Cells: A Manual of Basic Technique (5th Ed.), New York: Wiley Publishing, 2005, p. 217. The methods of the present application can overcome those difficulties due in part to the unexpected clonogenicity of ES, EC EG, and ED-derived cells. In addition, while ES cell lines such as human ES cell lines originate from cultures of ICM cells, it is not therefore obvious that observations made with ES cell lines apply to ED cells, especially those made by direct differentiation from the embryo without the generation of an ES cell line. For example, while the ICM of the preimplantation embryo contains totipotential cells capable of differentiating into all somatic cell lineages and the germ-line, many efforts have been made in the past to generate ES cell lines that retain the totipotency of the ICM and can still contribute to the germ-line. Such ES cell lines would therefore, like mouse ES cells, be useful in introducing heritable genetic modifications into animals. Nevertheless, other than mouse ES cells, mammalian cultured ICM cells generally lose the ability to contribute to the germ-line when introduced into the blastocyst and are therefore not equivalent to the ICM. Therefore, it would not be obvious to one skilled in the art that ED cells cultured without the generation of an ES cell line would differentiate or propagate in the same manner as ES cells. However, in the present invention, it is disclosed that totipotential cells of preimplantation embryos, including zygotes, blastomeres, cells from the morula staged embryo, cells from the inner cell mass, and cells from the embryonic disc are in fact equivalent to ES cell lines and can simply be substituted for ES cell in the present invention.

In one embodiment of the application, any methods of differentiating, propagating, identifying, isolating, or using stem cells known in the art (for example, U.S. Pat. Nos. 6,953,799, 7,029,915, 7,101,546, 7,129,034, 6,887,706, 7,033,831, 6,989,271, 7,132,286, 7,132,287, 6,844,312, 6,841,386, 6,565,843, 6,908,732, 6,902,881, 6,602,680, 6,719,970, 7,112,437, 6,897,061, 6,506,574, 6,458,589, 6,774,120, 6,673,606, 6,602,711, 6,770,478, 6,610,535, 7,045,353, 6,903,073, 6,613,568, 6,878,543, 6,670,397, 6,555,374, 6,261,841, 6,815,203, 6,967,019, 7,022,666, 6,423,681, 6,638,765, 7,041,507, 6,949,380, 6,087,168, 6,919,209, 6,676,655, 6,761,887, 6,548,299, 6,280,718, 6,656,708, 6,255,112, 6,413,773, 6,225,119, 6,056,777, 6,962,698, 6,936,254, 6,942,995, 6,924,142, 6,165,783, 6,093,531, 6,379,953, 6,022,540, 6,586,243, 6,093,557, 5,968,546, 6,562,619, 5,914,121, 6,251,665, 6,228,640, 5,948,623, 5,766,944, 6,783,775, 6,372,262, 6,147,052, 5,928,945, 6,096,540, 6,709,864, 6,322,784, 5,827,740, 6,040,180, 6,613,565, 5,908,784, 5,854,292, 6,790,826, 5,677,139, 5,942,225, 5,736,396, 5,648,248, 5,610,056, 5,695,995, 6,248,791, 6,051,415, 5,939,529, 5,922,572, 6,610,656, 6,607,913, 5,844,079, 6,686,198, 6,033,906, 6,340,668, 6,020,197, 5,766,948, 5,369,030, 6,001,654, 5,955,357, 5,700,691, 5,498,698, 5,733,878, 5,384,331, 5,981,165, 6,464,983, 6,531,445, 5,849,686, 5,197,985, 5,246,699, 6,177,402, 5,488,040, 6,667,034, 5,635,386, 5,126,325, 5,994,518, 5,032,507, 5,847,078, 6,004,548, 5,529,982, 4,342,828, 7,105,344, 7,078,230, 7,074,911, 7,053,187, 7,041,438, 7,030,292, 7,015,037, 7,011,828, 6,995,011, 6,969,608, 6,967,102, 6,960,444, 6,929,948, 6,878,542, 6,867,035, 6,866,843, 6,833,269, 6,828,144, 6,818,210, 6,800,480, 6,787,355, 6,777,231, 6,777,230, 6,749,847, 6,737,054, 6,706,867, 6,677,306, 6,667,391, 6,642,048, 6,638,501, 6,607,720, 6,576,464, 6,555,318, 6,545,199, 6,534,052, RE37,978, 6,461,865, 6,432,711, 6,399,300, 6,372,958, 6,369,294, 6,342,356, 6,337,184, 6,331,406, 6,271,436, 6,245,566, 6,235,970, 6,235,969, 6,215,041, 6,204,364, 6,194,635, 6,171,824, 6,090,622, 6,015,671, 5,955,290, 5,945,577, 5,914,268, 5,874,301, 5,866,759, 5,865,744, 5,843,422, 5,830,510, 5,795,569, 5,766,581, 5,733,727, 5,725,851, 5,712,156, 5,688,692, 5,656,479, 5,602,301, 5,370,870, 5,366,888, and 5,332,672, and U.S. patent publication nos. 20060251642, 20060217301, 20060216820, 20060193769, 20060161996, 20060134784, 20060134782, 20060110828, 20060104961, 20060088890, 20060079488, 20060078989, 20060068496, 20060062769, 20060024280, 20060015961, 20060009433, 20050244969, 20050244386, 20050233447, 20050221483, 20050164377, 20050153425, 20050149998, 20050142102, 20050130147, 20050118228, 20050106211, 20050054102, 20050032207, 20040260079, 20040228899, 20040193274, 20040152189, 20040151701, 20040141946, 20040121464, 20040110287, 20040052768, 20040028660, 20040028655, 20040018178, 20040009595, 20030203003, 20030175680, 20030161819, 20030148510, 20030082155, 20030040111, 20030040023, 20030036799, 20030032187, 20030032183, 20030031657, 20020197240, 20020164307, 20020098584, 20020098582, 20020090714, 20020022259, 20020019018, 20010046489, 20010024824, and 20010016203) are used in combination with the methods of the present application in differentiating, propagating, identifying, isolating, or using directly differentiated embryo-derived cells (i.e., substituting ED cells for ES cells and directly differentiating the ED cells). In certain embodiments, only the initial differentiation procedure from the prior art is used in combination with the present methods. In certain embodiments, ED cells are directly differentiated in the manner disclosed in the art for ES cells and following differentiation, cells are plated resulting in isolating a number of individual cultures of cells or a number of individual cultures of cells that are oligoclonal, wherein one or more of said cultures comprise cells with reduced differentiation potential than the starting pluripotent stem cells and wherein each of said individual cultures having only one cell may be propagated into a pure clonal culture of cells and wherein each of said individual cultures of cells having cells that are oligoclonal may be propagated into a larger number of cells, and one or more (or all) of said individual cultures of cells is propagated. To summarize, ED cells are differentiated in step 1 of this invention according to the methods in the art and then the heterogenous population of cells so generated are cultured and propagated according to step 2 of this invention.

In another aspect of the invention, the methods of this invention result in the derivation of endodermal cells from a single cell differentiated or in the process of differentiating from pluripotent stem cells such as, but not limited to, hES, hEG, hEC or hED cells.

In another aspect of the invention, the methods of this invention result in the derivation of mesodermal cells from a single cell differentiated or in the process of differentiating from pluripotent stem cells such as, but not limited to, hES, hEG, hEC or hED cells.

In another aspect of the invention, the methods of this invention result in the derivation of ectodermal cells from a single cell differentiated or in the process of differentiating from pluripotent stem cells such as, but not limited to, hES, hEG, hEC or hED cells.

In another aspect of the invention, the methods of this invention result in the derivation of neuroglial precursor cells from a single cell differentiated or in the process of differentiating from pluripotent stem cells such as, but not limited to, hES, hEG, hEC or hED cells.

In another aspect of the invention, the methods of this invention result in the derivation of hepatic cells or hepatic precursor cells from a single cell differentiated or in the process of differentiating from pluripotent stem cells such as, but not limited to, hES, hEG, hEC or hED cells.

In another aspect of the invention, the methods of this invention result in the derivation of chondrocyte or chondrocyte precursor cells from a single cell differentiated or in the process of differentiating from pluripotent stem cells such as, but not limited to, hES, hEG, hEC or hED cells.

In another aspect of the invention, the methods of this invention result in the derivation of myocardial or myocardial precursor cells from a single cell differentiated or in the process of differentiating from pluripotent stem cells such as, but not limited to, hES, hEG, hEC or hED cells. Such myocardial precursor cells may also be produced by direct differentiation as described herein.

In another aspect of the invention, the methods of this invention result in the derivation of gingival fibroblast or gingival fibroblast precursor cells from a single cell differentiated or in the process of differentiating from pluripotent stem cells such as, but not limited to, hES, hEG, hEC or hED cells.

In another aspect of the invention, the methods of this invention result in the derivation of pancreatic beta cells or pancreatic beta precursor cells from a single cell differentiated or in the process of differentiating from pluripotent stem cells such as, but not limited to, hES, hEG, hEC or hED cells.

In another aspect of the invention, the methods of this invention result in the derivation of retinal precursor cells with from a single cell differentiated or in the process of differentiating from pluripotent stem cells such as, but not limited to, hES, hEG, hEC or hED cells.

In another aspect of the invention, the methods of this invention result in the derivation of hemangioblasts from a single cell differentiated or in the process of differentiating from pluripotent stem cells such as, but not limited to, hES, hEG, hEC or hED cells.

In another aspect of the invention, the methods of this invention result in the derivation of dermal fibroblasts with prenatal patterns of gene expression from a single cell differentiated or in the process of differentiating from pluripotent stem cells such as, but not limited to, hES, hEG, hEC or hED cells.

Dermal fibroblasts derived according to the invention can be grown on a biocompatible substratum and engrafted on the neodermis of artificial skin covering a wound. Autologous keratinocytes may also be cultivated on a commercially available membrane such as Laserskin™ using the methods provided in this invention.

In another embodiment of the present invention, it is possible to simplify burn treatment further and to save lives of patients having extensive burns where sufficient autologous skin grafts cannot be repeatedly harvested in a short period of time. The dead skin tissue of a patient with extensive burns can be excised within about three to seven days after injury. The wound can be covered with any artificial skin, for example Integra™, or any dermal equivalent thereof, and dermal keratinocytes or dermal fibroblasts produced according to the methods of this invention or derived from said cells may thereafter be engrafted on the neodermis of the artificial skin, with resultant lower rejection and infection incidences.

Epidermolysis bullosa (“EB”) is a group of heritable diseases that result in a loss of mechanical strength in the skin, in particular, separation of the epidermis from the dermis (blistering). EB patients have fragile skin which can blister even from mild, such as skin-to-skin, contact. These patients suffer from constant pain and scarring, which, in the worse forms, leads to eventual disfigurement, disability and often early death. EB patients lack anchors that hold the layers of their skin together and as a consequence, any activity that rubs or causes pressure produces a painful sore that has been compared to a second-degree burn. One of the forms of EB is lethal in the first weeks or months of life. Some are more long-term and cause pain and mutilation throughout the patient's lifetime. Infection is a serious, ongoing concern and no treatment for EB has been effective. To date, parents' only hope has been to attempt to protect the child's skin with gauze and ointments, to prevent and protect the wounds and healthy skin. The manifestation of the disease is highly variable depending on the locus of the mutation. Traditionally, there are three categories: the simplex form with separation within the keratinocytes, the junctional forms with separation the lamina lucida of the basement membrane, and the dystrophic forms with separation in the papillary dermis. There is now evidence of another variant at the level of hemidesmosomes and the basal cell/lamina lucida interface (Uitto et al., Am J Med Genet C Semin Med Genet 131C:61-74 (2004)). Accordingly, dermal keratinocytes or dermal fibroblasts produced according to the methods of this invention or derived from said cells may be engrafted onto wound sites of EB patients to lower the incidence of infection and prevent further blistering.

The cells produced according to the methods of this invention or derived from said cells may also be combined with biological or synthetic matrices as is well known in the art. For example, dermal fibroblasts may be combined with collagen, including collagen that has been cross-linked by chemical or physical methods, and/or with other extracellular matrix components such as fibronectin, fibrin, proteoglycans, among others. The cells may be used in combination with hyaluronan (HA).

Some embodiments of the invention provide a matrix for implantation into a patient. In some embodiments, the matrix is seeded with a population of keratinocytes or dermal fibroblast cells derived according to the methods of this invention. The matrix may contain or be pre-treated with one or more bioactive factors including, for example, drugs, anti-inflammatory agents, antiapoptotic agents, and growth factors. The seeded or pre-treated matrices can be introduced into a patient's body in any way known in the art, including but not limited to, implantation, injection, surgical attachment, transplantation with other tissue, injection, and the like. The matrices of the invention may be configured to the shape and/or size of a tissue or organ in vivo. The scaffolds of the invention may be flat or tubular or may comprise sections thereof. The scaffolds of the invention may also be multilayered.

To form a bilayer tissue construct comprising a cell-matrix construct and a second cell layer thereon, the method of this invention additionally comprises the step of: culturing cells of a second type on a surface of the formed tissue-construct to produce a bilayered or multilayered tissue construct.

An extracellular matrix-producing cell type for use in the invention may be any cell type capable of producing and secreting extracellular matrix components and organizing the extracellular matrix components to form a cell-matrix construct. More than one extracellular matrix-producing cell type may be cultured to form a cell-matrix construct. Cells of different cell types or tissue origins may be cultured together as a mixture to produce complementary components and structures similar to those found in native tissues. For example, the extracellular matrix-producing cell type may have other cell types mixed with it to produce an amount of extracellular matrix that is not normally produced by the first cell type. Alternatively, the extracellular matrix-producing cell type may also be mixed with other cell types that form specialized tissue structures in the tissue but do not substantially contribute to the overall formation of the matrix aspect of the cell-matrix construct, such as in certain skin constructs of the invention. All cells are either produced by methods of this invention or derived from said cells.

While any extracellular matrix-producing cell type may be used in accordance with this invention, the preferred cell types for use in this invention are derived from mesenchyme. More preferred cell types are fibroblasts, stromal cells, and other supporting connective tissue cells, most preferably human dermal fibroblasts found in human dermis for the production of a human dermal construct. Fibroblast cells, generally, produce a number of extracellular matrix proteins, primarily collagen. There are several types of collagens produced by fibroblasts, however, type I collagen is the most prevalent in vivo. Human fibroblast cell strains can be derived from a number of sources, including, but not limited to, neonate male foreskin, dermis, tendon, lung, umbilical cords, cartilage, urethra, corneal stroma, oral mucosa, and intestine. The human cells may include but need not be limited to, fibroblasts, but may include: smooth muscle cells, chondrocytes and other connective tissue cells of mesenchymal origin. It is preferred, but not required, that the origin of the matrix-producing cell used in the production of a tissue construct be derived from a tissue type that it is to resemble or mimic after employing the culturing methods of the invention. For instance, in the embodiment where a skin-construct is produced, the preferred matrix-producing cell is a fibroblast, preferably of dermal origin. In another preferred embodiment, fibroblasts isolated by microdissection from the dermal papilla of hair follicles can be used to produce the matrix alone or in association with other fibroblasts. In the embodiment where a corneal-construct is produced, the matrix-producing cell is derived from corneal stroma. Cell donors may vary in development and age. Cells may be derived from donor tissues of embryos, neonates, or older individuals including adults. Embryonic progenitor cells such as mesenchymal stem cells may be used in the invention and induced to differentiate to develop into the desired tissue. All cells are either produced by methods of this invention or derived from said cells.

Recombinant or genetically-engineered cells may be used in the production of the cell-matrix construct to create a tissue construct that acts as a drug delivery graft for a patient needing increased levels of natural cell products or treatment with a therapeutic. The cells may produce and deliver to the patient via the graft recombinant cell products, growth factors, hormones, peptides or proteins for a continuous amount of time or as needed when biologically, chemically, or thermally signaled due to the conditions present in the patient. Either long or short-term gene product expression is desirable, depending on the use indication of the cultured tissue construct. Long term expression is desirable when the cultured tissue construct is implanted to deliver therapeutic products to a patient for an extended period of time. Conversely, short term expression is desired in instances where the cultured tissue construct is grafted to a patient having a wound where the cells of the cultured tissue construct are to promote normal or near-normal healing or to reduce scarification of the wound site. Once the wound has healed, the gene products from the cultured tissue construct are no longer needed or may no longer be desired at the site. Cells may also be genetically engineered to express proteins or different types of extracellular matrix components which are either “normal” but expressed at high levels or modified in some way to make a graft device comprising extracellular matrix and living cells that is therapeutically advantageous for improved wound healing, facilitated or directed neovascularization, or minimized scar or keloid formation. These procedures are generally known in the art, and are described in Sambrook et al, Molecular Cloning, A Laboratory Manual, Cold Spring Harbor Press, Cold Spring Harbor, N.Y. (1989), incorporated herein by reference. All of the above-mentioned types of cells are included within the definition of a “matrix-producing cell” as used in this invention.

Human skin equivalents (“HSE”) using biological matrices are well known in the art and may include the use of hydrated collagen gels as described by Smola et al., J Cell Biol, 122:417-29 (1993). In brief, 4 mg/mL collagen solutions are mixed at 4° C. with fibroblasts to reach a final density of 1×10⁵ cells/mL. The collagen/cell suspension is then placed on a membrane such as a filter membrane and incubated for 15 min at 37° C. in a humidified incubator to allow polymerization. Then the gel is placed in culture media of various compositions known in the art and allowed to contract and stabilize over time. All cells are either produced by methods of this invention or derived from said cells.

In addition, synthetic matrices comprising synthetic polymers may be used. Synthetic polymers include polyether urethane and polyglycan, co-polymers such as Polyactive â, Isotis NV, Bilthoven, the Netherlands), consisting of poly(ethyleneglycol-terephthatlate) (55%)/poly(butylene-terephthalate) (45%) (PEGT/PBT) copolymer and polyethylene glycol. All cells are either produced by methods of this invention or derived from said cells.

Pre-scarring (“PS”) fibroblasts may be seeded into biological or synthetic matrices at a concentration that promotes the rapid healing of wounds and/or reduces scar formation. Such concentrations range from 1.0×10⁵ to 1×10⁷ cells/cm². All cells are either produced by methods of this invention or derived from said cells.

Other tissue such as diaphragmatic tissue may also be used. All cells and tissues are either produced by methods of this invention or derived from said cells.

In another aspect of the invention, the methods of this invention result in the derivation of neural crest cells from a single cell differentiated or in the process of differentiating from pluripotent stem cells such as, but not limited to, hES, hEG, hEC or hED cells.

Neural crest cells derived according to the invention include neural crest cells of the forebrain or midbrain origin with no Hox gene expression as well as neural crest cells with Hox gene expression including Hoxa-1 through Hoxa-13, Hoxb-1 through Hoxb9, Hoxc-4 through Hoxc-13, and Hoxd-1 through Hoxd-13 corresponding to regions in the hindbrain, cervical, thoracic, and lumbar regions such as hindbrain cranial, vagal, cardiac, and trunk neural crest.

Such varieties of neural crest cells may be pluripotent stem cells that have a propensity to differentiate into a unique constellation of cell types, though there is some plasticity here, so that given the right environmental cues, neural crest cells of one type can differentiate into the cell types normally formed by another neural crest cell type. For example, cranial neural crest cells with no Hox gene expression normally become cells and tissues including: dental mesenchyme, detal papilla, odontoblasts, dentine matrix, pulp, cementum, periodontal ligaments, chondrocytes in Meckel's cartilage, the bone of the mandible, the articulating disk of the termporomandibular joint and the branchial arch nerve ganglion, the meningens and frontal bones and suture mesenchyme of the cranium.

Generally, cranial neural crest cells have the potential to differentiate into melanocytes, nerve ganglia such as peripheral nerve ganglia such as sensory nerves and the cranial nerves, glia including Schwann cells, smooth muscle cells, cells of the ear including the bones of the middle ear, and connective tissues of the face and neck including the dermis and cells of the anterior chamber of the eye such as the endothelial cells of the cornea and cells of the lens, thymus, and parathyroid gland. The migratory nature of neural crest progenitors makes the cells particularly useful in integrating into diseased dermis such as that of EB and producing normal COL7A1 useful in the treatment of the disease.

Cardiac neural crest cells are capable of differentiating into aorticopulmonary septum, conotruncal cushions, SA node, AV node, and other conduction fibers of the heart, and derivatives of the 3rd, 4th, and 6th branchial arches.

Neural crest cells from the trunk are capable of differentiating into many of the cell types observed in cranial neural crest cells, but can also become adrenomedullary cells.

In another aspect of the invention, the methods of this invention result in the derivation of elastogenic fibroblasts with prenatal patterns of gene expression from a single cell differentiated or in the process of differentiating from pluripotent stem cells such as, but not limited to, hES, hEG, hEC or hED cells. Such cells may be useful, for example, for the treatment of aging and sagging skin, vocal cords and the lung where age-related elastolysis may lead to disease or dysfunction.

In another aspect of the invention, the methods of this invention result in the derivation of lung connective tissue cells with prenatal patterns of gene expression that is highly elastogenic from a single cell differentiated or in the process of differentiating from pluripotent stem cells such as, but not limited to, hES, hEG, hEC or hED cells.

In another aspect of the invention, the method comprises the derivation of 100 cells or more from a single differentiated cell or a cell in the process of differentiating from a pluripotent stem cell such as a hES cell, wherein the pluripotent stem cell is derived from the reprogramming of a somatic cell through the exposure of the somatic cell to the cytoplasm of an undifferentiated cell (see U.S. application No. 60/624,827, filed Jun. 30, 1999; Ser. No. 09/736,268, filed Dec. 15, 2000; Ser. No. 10/831,599, filed Apr. 30, 2004; PCT application no. PCT/US02/18063, filed Jun. 30, 2000; U.S. application No. 60/314,657, filed Aug. 27, 2001; Ser. No. 10/228,316, filed Aug. 27, 2002; Ser. No. 10/487,963, filed Feb. 26, 2004; Ser. No. 11/055,454, filed Feb. 9, 2005; PCT application no. PCT/US02/26798, filed Aug. 27, 2002; the disclosures of which are incorporated by reference; see also U.S. application No. 60/705,625, filed Aug. 3, 2005; U.S. application No. 60/729,173, filed Oct. 20, 2005; U.S. application No. 60/818,813, filed Jul. 5, 2006; and PCT/US06/30632, filed Aug. 3, 2006, the disclosures of which are incorporated by reference).

In particular, the reprogrammed cells may be differentiated into cells with a dermatological prenatal pattern of gene expression that is highly elastogenic or capable of regeneration without causing scar formation, by methods of this invention. Dermal fibroblasts of mammalian fetal skin, especially corresponding to areas where the integument benefits from a high level of elasticity, such as in regions surrounding the joints, are responsible for synthesizing de novo the intricate architecture of elastic fibrils that function for many years without turnover. In addition, early embryonic skin is capable of regenerating without scar formation. Cells from this point in embryonic development made from the reprogrammed cells of the present invention are useful in promoting scarless regeneration of the skin including forming normal elastin architecture. This is particularly useful in treating the symptoms of the course of normal human aging, or in actinic skin damage, where there can be a profound elastolysis of the skin resulting in an aged appearance including sagging and wrinkling of the skin.

In another embodiment of the invention, the reprogrammed cells are exposed to inducers of differentiation to yield other therapeutically-useful cells such as retinal pigment epithelium, hematopoietic precursors and hemangioblastic progenitors as well as many other useful cell types of the endoderm, mesoderm, and endoderm, by methods of this invention. Such inducers include but are not limited to: cytokines such as interleukin-alpha A, interferon-alpha A/D, interferon-beta, interferon-gamma, interferon-gamma-inducible protein-10, interleukin-1-17, keratinocyte growth factor, leptin, leukemia inhibitory factor, macrophage colony-stimulating factor, and macrophage inflammatory protein-1 alpha, 1-beta, 2, 3 alpha, 3 beta, and monocyte chemotactic protein 1-3, 6kine, activin A, amphiregulin, angiogenin, B-endothelial cell growth factor, beta cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropoietin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocyte macrophage colony stimulating factor, GRO-alpha/MGSA, GRO-beta, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, insulin-like growth factor II, nerve growth factor, neurotophin-3,4, oncostatin M, placenta growth factor, pleiotrophin, rantes, stem cell factor, stromal cell-derived factor 1B, thrombopoietin, transforming growth factor—(alpha, beta1,2,3,4,5), tumor necrosis factor (alpha and beta), vascular endothelial growth factors, and bone morphogenic proteins, enzymes that alter the expression of hormones and hormone antagonists such as 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine, leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopressin, extracellular matrix components such as fibronectin, proteolytic fragments of fibronectin, laminin, tenascin, thrombospondin, and proteoglycans such as aggrecan, heparan sulphate proteoglycan, chontroitin sulphate proteoglycan, and syndecan. Other inducers include cells or components derived from cells from defined tissues used to provide inductive signals to the differentiating cells derived from the reprogrammed cells of the present invention. Such inducer cells may derive from human, nonhuman mammal, or avian, such as specific pathogen-free (SPF) embryonic or adult cells.

In another embodiment of the invention, the cells with a prenatal pattern of gene expression made by methods of this invention are genetically modified to enhance a therapeutic effect, either before or after going through methods of this invention (i.e., either the parent pluripotent stem cells or the cells derived from methods of this invention). Such modifications may include the upregulation of expression of platelet-derived growth factor (PDGF) to improve wound repair when the modified cells are introduced into a wound. Such modifications may also include the up or down-regulation of one of a number of extracellular signaling molecules including, but not limited to, growth factors, cytokines, extracellular matrix components, nucleic acids encoding the foregoing, steroids, and morphogens or neutralizing antibodies to such factors. Such inducers include but are not limited to: cytokines such as interleukin-alpha A, interferon-alpha A/D, interferon-beta, interferon-gamma, interferon-gamma-inducible protein-10, interleukin-1-17, keratinocyte growth factor, leptin, leukemia inhibitory factor, macrophage colony-stimulating factor, and macrophage inflammatory protein-1 alpha, 1-beta, 2, 3 alpha, 3 beta, and monocyte chemotactic protein 1-3, 6kine, activin A, amphiregulin, angiogenin, B-endothelial cell growth factor, beta cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropioetin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocyte macrophage colony stimulating factor, GRO-alpha/MGSA, GRO-beta, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, insulin-like growth factor II, nerve growth factor, neurotophin-3,4, oncostatin M, placenta growth factor, pleiotrophin, rantes, stem cell factor, stromal cell-derived factor 1B, thrombopoietin, transforming growth factor—(alpha, beta1,2,3,4,5), tumor necrosis factor (alpha and beta), vascular endothelial growth factors, and bone morphogenic proteins, enzymes that alter the expression of hormones and hormone antagonists such as 17B-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine, leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopressin, extracellular matrix components such as fibronectin, proteolytic fragments of fibronectin, laminin, tenascin, thrombospondin, and proteoglycans such as aggrecan, heparan sulphate proteoglycan, chontroitin sulphate proteoglycan, and syndecan.

The present invention also provides for methods for direct differentiation of these cells from embryos without making ES cell lines (ED cells). Direct differentiation refers, for example, to methods of making downstream stem cells from an embryo without making ES cells (see U.S. patent publication no. 20050265976, published Dec. 1, 2005, and international patent publication no. WO0129206, published Apr. 26, 2001, the disclosures of which are hereby incorporated by reference). Also, direct differentiation may be accomplished from other pluripotent cells such as NT-derived, parthenote-derived, morula or blastomere-derived, cells that are homozygous in the HLA, those put into the gene trap system (see U.S. application Ser. No. 10/227,282, filed Aug. 26, 2002 and Ser. No. 10/685,693, filed October 2003, the disclosures of which are incorporated herein by reference), those made by dedifferentiating using cytoplasmic transfer (see U.S. application Ser. No. 10/831,599, filed Apr. 23, 2004; Ser. No. 10/228,316, filed Aug. 27, 2002; and Ser. No. 10/228,296, filed Aug. 27, 2002, the disclosures of which are incorporated herein by reference). All of these pluripotent cells may be used as the starting cells of the methods of this invention.

The present invention also provides for methods for the treatment of dermatological diseases or disorders, and one such method is the derivation of dermal cells with prenatal patterns of gene expression which may be derived according to the methods of this invention. Specifically this may be done by culturing embryo-derived cells, NT-derived, parthenote-derived, morula or blastomere-derived cells according to the methods of this invention.

The present invention also provides for a method of conducting a pharmaceutical business by establishing regional centers comprising the cells of the present invention. In one aspect of the invention, the method comprises the derivation from a subject of populations of two or more, preferably one hundred or more cells from a single cell differentiated or in the process of differentiating from pluripotent stem cells such as, but not limited to, hES, hEG, hEC or hED cells, wherein the resulting single cell-derived population of cells can be documented not to have contaminating cells from the original parent pluripotent stem cells (such as ES, EG, EC or ED cells), wherein the resulting single cell-derived population of cells are isolated from a heterogeneous population from said subject and can be used in cell therapy in said subject.

The present invention also provides for a method of conducting a pharmaceutical business wherein the single or oligoclonal-derived populations of cells generated by the methods of the invention are marketed to healthcare providers, researchers or directly to subjects in need of such cells. One aspect provides a method for conducting a pharmaceutical business, comprising marketing to healthcare providers, researchers or to patients in need of such single or oligoclonal-derived populations of cells, the benefits of using any of the cells described herein in the treatment of a disease or disorder. A related aspect provides a method for conducting a pharmaceutical business, comprising: (a) manufacturing any of the cells described herein; and (b) marketing to healthcare providers, researchers or to patients in need of such cells the benefits of using the cells in the treatment of a disease or disorder. In some embodiments, the rights to develop and market such single or oligoclonal-derived populations of cells or to conduct such manufacturing steps may be licensed to a third party for consideration. In certain embodiments of the invention, the cells are marketed along with other factors including, but not limited to, the extracellular matrix, and the gene expression profile of said cells.

In certain embodiments, the methods of the invention could be performed in a high throughput format using techniques known to one skilled in the art (see, e.g., Meldrum (2000) Genome Research Vol. 10, Issue 8, 1081-1092). The automation of the steps of the procedure using robotics could further enhance the number of conditions that can be tested. For example, 96-well microtiter plates or higher well densities such as 384- and 1536-well formats can be utilized for tissue culture techniques. Also of potential use in this invention are automated spotting, colony-picking robots or liquid handling devices. Most of these devices use an X-Y-Z robot arm (one that can move in three dimensions) mounted on an anti-vibration table. The robot arm may hold nozzles in case of non-contact spotting. In contact spotting, the robot arm may hold pins. Nozzles or pins are dipped into a first microtiter plate to pick up the test media component or cells to be delivered. The tips in case of pins are then moved to the solid support surface and allowed to touch the surface only minimally; the solution is then transferred. The pins are then washed and moved to the next set of wells and test media. This process is repeated until hundreds or thousands of test conditions are tested. One example of a robotic platform is the CellMate robotic platform.

In certain embodiments, to obtain cultures with single cells or oligoclonal clusters of multiple cells, the cells (such as the population of heterogeneous population of cells) are plated at limiting dilution. Limiting dilution may be performed as is known to one skilled in the art (Moretta et al., J Immunol. (1985) 134(4):2299-304). In certain embodiments, limiting dilution is performed such that most wells have a single cell. In other embodiments, limiting dilution is performed such that most wells have a single oligoclonal clusters of multiple cells.

Cells and compositions obtained from the methods of this invention may be tested for the capacity to be scaled up in roller bottles before being designated a product candidate.

### Applications

The disclosed methods for the culture of animal cells and tissues are useful in generating cells or progeny thereof in mammalian and human cell therapy, such as, but not limited to, generating human cells useful in treating dermatological, retinal, cardiac, neurological, endocrinological, muscular, skeletal, articular, hepatic, neurological, renal, gastrointestinal, pulmonary, and blood and vascular cell disorders in humans and nonhuman animals.

In certain embodiments of the invention, single cell-derived and oligoclonal cell-derived cells, derived by methods of this invention, are utilized in research and treatment of disorders relating to cell biology, cell-based drug discovery and in cell therapy. The single cell-derived cell populations derived using the methods of the present invention may already have received the requisite signals to be directed down a differentiation pathway. For example, some paraxial or somatopleuric single cell-derived populations of cells may express genes consistent with dermal fibroblast gene expression, in particular, a prenatal pattern of gene expression useful in promoting scarless wound repair and in promoting elastogenesis. Such cells include for example, those cells listed in Table II, including but not limited to: cells of the heart; cells of the musculo-skeletal system; cells of the nervous tissue; cells of the respiratory system; cells of the endocrine system; cells of the vascular system; cells of the hematopoietic system; cells of the integumentary system; cells of the urinary system; or cells of the gastrointestinal system. Such cells may be stably grafted in a histocompatible host when the cells are grafted into the tissue into which the cells would normally differentiate. Such final differentiated tissues are well known from the art of embryology and by way of nonlimiting example, some are listed in Table III. Such tissues include for example (as listed in Table III), but not limited to: endoderm-embryonic tissues; mesoderm-embryonic tissues; ectoderm-embryonic tissues; or extraembryonic cells.

In certain embodiments of the invention, single cell-derived and oligoclonal cell-derived cells are introduced into the tissues in which they normally reside in order to exhibit therapeutic utility. For example, the clonogenic populations of cells derived by methods of this invention may be introduced into the tissues including but not limited to the tissues listed in Table II.

In certain embodiments of the invention, single cell-derived and oligoclonal cell-derived cells, derived by methods of this invention, are utilized in inducing the differentiation of other pluripotent stem cells. The generation of single cell-derived populations of cells capable of being propagated in vitro while maintaining an embryonic pattern of gene expression is useful in inducing the differentiation of other pluripotent stem cells. Cell-cell induction is a common means of directing differentiation in the early embryo. Many potentially medically-useful cell types are influenced by inductive signals during normal embryonic development, including spinal cord neurons, cardiac cells, pancreatic beta cells, and definitive hematopoietic cells. Single cell-derived populations of cells capable of being propagated in vitro while maintaining an embryonic pattern of gene expression can be cultured in a variety of in vitro, in ovo, or in vivo culture conditions to induce the differentiation of other pluripotent stem cells to become desired cell or tissue types.

Induction may be carried out in a variety of methods that juxtapose the inducer cell with the target cell. By way of nonlimiting examples, the inducer cells may be plated in tissue culture and treated with mitomycin C or radiation to prevent the cells from replicating further. The target cells are then plated on top of the mitotically-inactivated inducer cells. Alternatively, single cell-derived inducer cells may be cultured on a removable membrane from a larger culture of cells or from an original single cell-derived colony and the target cells may be plated on top of the inducer cells or a separate membrane covered with target cells may be juxtaposed so as to sandwich the two cell layers in direct contact. The resulting bilayer of cells may be cultured in vitro, transplanted into a SPF avian egg, or cultured in conditions to allow growth in three dimensions while being provided vascular support (see, for example, international patent publication number WO2005068610, published Jul. 28, 2005, the disclosure of which is hereby incorporated by reference). The inducer cells may also be from a source of pluripotent stem cells, including hES or hED cells, in which a suicide construct has been introduced such that the inducer cells can be removed at will. Cell types useful in single cell-derived and oligoclonal cell-derived induction may include cases of induction well known in the art to occur naturally in normal embryonic development.

In certain embodiments of the invention, single cell-derived cells and oligoclonal cell-derived cells, derived by methods of this invention, are used as “feeder cells” to support the growth of other cell types, including pluripotent stem cells. The use of single cell-derived cells and oligoclonal cell-derived cells of the present invention as feeder cells alleviates the potential risk of transmitting pathogens from feeder cells derived from other mammalian sources to the target cells. The feeder cells may be inactivated, for example, by gamma ray irradiation or by treatment with mitomycin C, to limit replication and then co-cultured with the pluripotent stem cells.

In certain embodiments of the invention, the extracellular matrix (ECM) of single cell-derived and oligoclonal cell-derived cells, derived by methods of this invention, may be used to support less differentiated cells (see Stojkovic et al., Stem Cells (2005) 23(3):306-14). Certain cell types that normally require a feeder layer can be supported in feeder-free culture on a matrix (Roster et al., Dev Dyn. (2004) 229(2):259-74). The matrix can be deposited by preculturing and lysing a matrix-forming cell line (see WO 99/20741), such as the STO mouse fibroblast line (ATCC Accession No. CRL-1503), or human placental fibroblasts.

In certain embodiments of the invention, the conditioned media of single cell-derived and oligoclonal cell-derived cell cultures may be collected, pooled, filtered and stored as conditioned medium. This conditioned medium may be formulated and used for research and therapy. Such conditioned medium may contribute to maintaining a less differentiated state and allow propagation of cells such as pluripotent stem cells. In certain embodiments of the invention, conditioned medium of single cell-derived and oligoclonal cell-derived cell cultures derived by the methods of this invention, can be used to induce differentiation of other cell types, including pluripotent stem cells. The use of conditioned medium of single cell-derived and oligoclonal cell-derived cell cultures may be advantageous in reducing the potential risk of exposing cultured cells to non-human animal pathogens derived from other mammalian sources (i.e., xenogeneic free) to the cells.

In another embodiment of the invention, single cell-derived and oligoclonal cell-derived paraxial mesoderm, neural crest mesenchyme, or somatopleuric mesoderm, derived by methods of this invention, can be used to induce embryonic ectoderm or single cell-derived embryonic ectoderm into keratinocytes for use in skin research and grafting for burns, wound repair, and drug discovery.

In another embodiment of the invention, the use of single cell-derived and oligoclonal cell-derived prechordal plate mesoderm, derived by methods of this invention, to induce embryonic ectoderm or single cell-derived and oligoclonal cell-derived embryonic ectoderm into neuroectodermal cells capable of generating CNS cells may be useful in neuron research and grafting for neurodegenerative diseases, and drug discovery. The single cell-derived and oligoclonal cell-derived prechordal plate mesoderm can be identified by transcript analysis as described herein through the expression of, for example, lim-1.

In another embodiment of the invention, the single cell-derived and oligoclonal cell-derived notochord mesodermal cells, derived by methods of this invention, are identified by their expression of brachyury. In normal development, notochordal cells induce the floor of the neural plate mesoderm (which induces the spinal chord) to make sonic hedgehog (“SHH”), a ventralizing signal, that induces the floor of the neural tube to express SHH as well, which induces the expression of FP1, FP2, and SC1 by the floor plate of the neural tube. Therefore, notochordal mesodermal cells can be used to induce neural plate ectodermal cells or neural tube neuroepithelial cells to differentiate into spinal cord neurons. Such neurons may be identified and confirmed by assaying the gene expression assays described herein for cells expressing FP1, FP2, or SC1. These cells expressing one or more of these markers could be useful in spinal cord regeneration.

Our discovery that various single cell-derived and oligoclonal cell-derived cells in early embryonic lineages may be propagated without the loss of their embryonic phenotype allows numerous types of mesodermal inducer cells to induce differentiation in embryonic ectoderm or endoderm. However, single cell-derived and oligoclonal cell-derived cells from endoderm and ectodermal lineages, derived by methods of this invention, may be useful in induction as well. For example, surface ectoderm and notochord express Shh and thereby induce somites to become sclerotome mesodermal cells that express M-twist and Pax-1 and surface ectoderm. Also, as another example, notochord expresses extracellular proteins of the Wnt family and thereby induces other somite mesodermal cells to become dermatome mesodermal cells that express gMHox, and dermo-1. Meanwhile, the myotome expresses N-myc and myogenin.

The juxtaposition of the inducer and target cells provides a useful in vitro model of differentiation that can be used for research into early embryonic differentiation, for drug screening, and studies of teratology. The target cells differentiated by the single cell-derived inducer cells may also be used for research, drug discovery, and cell-based therapy.

In certain embodiments of the invention, the single cell-derived and oligoclonal cell-derived cells, derived by methods of this invention, may be used to generate skin equivalents, as well as to reconstitute full-thickness human skin, according to the methods described in U.S. application Ser. No. 09/037,191, filed Mar. 9, 1998 (U.S. publication no. 20010048917, published Dec. 6, 2001); Ser. No. 10/013,124, filed Dec. 7, 2001 (U.S. publication no. 20020120950, published Aug. 29, 2002); Ser. No. 10/982,186, filed Nov. 5, 2004 (U.S. publication no. 20050118146, published Jun. 2, 2005); the disclosure of each of which is incorporated herein by reference. For example, the single cell-derived and oligoclonal cell-derived cells may be incorporated into a layered cell sorted tissue that includes a discrete first cell layer and a discrete second cell layer that are formed in vitro by the spontaneous sorting of cells from a homogenous cell mixture. The first cell layer may include any cell type, but preferably includes epithelial cells, in particular, keratinocytes. Other cell types that may be used in the first cell layer are CaCo2 cells, A431 cells, and HUC18 cells. The second cell layer may also include cells of any type, but preferably includes mesenchymal cells, in particular, fibroblasts. The layered cell sorted tissue possesses an epidermal-dermal junction that is substantially similar in structure and function to its native counterpart. That is, the tissue expresses the necessary integral proteins such as hemidesmosomes and collagen I, collagen IV, and collagen VII, to attach the epidermal and dermal layers with the proper basement membrane morphology. The single cell-derived and oligoclonal cell-derived cells may then sort to form an epidermal layer that contacts the connective tissue component. The layered cell sorted tissues comprising the single cell-derived and oligoclonal cell-derived cells may be used as a skin graft that could be used on graft sites such as traumatic wounds and burn injury.

In another embodiment of the invention, single cell-derived and oligoclonal cell-derived cells of this invention may be used as a means to identify and characterize genes that are transcriptionally activated or repressed as the cells undergo differentiation. For example, libraries of gene trap single cell-derived or oligoclonal cell-derived cells may be made by methods of this invention, and assayed to detect changes in the level of expression of the gene trap markers as the cells differentiate in vitro and in vivo. The methods for making gene trap cells and for detecting changes in the expression of the gene trap markers as the cells differentiate are reviewed in Durick et al. (Genome Res. (1999) 9:1019-25), the disclosure of which is incorporated herein by reference). The vectors and methods useful for making gene trap cells and for detecting changes in the expression of the gene trap markers as the cells differentiate are also described in U.S. Pat. No. 5,922,601 (Baetscher et al.), U.S. Pat. No. 6,248,934 (Tessier-Lavigne) and in U.S. patent publication No. 20040219563 (West et al.), the disclosures of which are also incorporated herein by reference. Methods for genetically modifying cells, inducing their differentiation in vitro, and using them to generate chimeric or nuclear-transfer cloned embryos and cloned mice are developed and known in the art. To facilitate the identification of genes and the characterization of their physiological activities, large libraries of gene trap cells having gene trap DNA markers randomly inserted in their genomes may be prepared. Efficient methods have been developed to screen and detect changes in the level of expression of the gene trap markers as the cells differentiate in vitro or in vivo. In vivo methods for inducing single cell-derived or oligoclonal cell-derived cells to differentiate further include injecting one or more cells into a blastocyst to form a chimeric embryo that is allowed to develop; fusing a stem cell with an enucleated oocyte to form a nuclear transfer unit (NTU), and culturing the NTU under conditions that result in generation of an embryo that is allowed to develop; and implanting one or more clonogenic differentiated cells into an immune-compromised or a histocompatible host animal (e.g., a SCID mouse, or a syngeneic nuclear donor) and allowing teratomas comprising differentiated cells to form. In vitro methods for inducing single cell-derived or oligoclonal cell-derived cells to differentiate further include culturing the cells in a monolayer, in suspension, or in three-dimensional matrices, alone or in co-culture with cells of a different type, and exposing them to one of many combinations of chemical, biological, and physical agents, including co-culture with one or more different types of cells, that are known to capable of induce or allow differentiation.

In another embodiment of the invention, cell types that do not proliferate well under any known cell culture conditions may be induced to proliferate such that they can be isolated clonally or oligoclonally according to the methods of this invention through the regulated expression of factors that overcome inhibition of the cell cycle, such as regulated expression of SV40 virus large T-antigen (Tag), or regulated E1a and/or E1b, or papillomavirus E6 and/or E7. To artificially stimulate the proliferation of such cell lines produced using the methods of the present invention, pluripotent stem cells such as hES cells may be transfected with a plasmid construct containing a temperature sensitive mutant of SV40 Tag regulated by a gamma-interferon promoter (Jat et al., Proc Natl Acad Sci USA 88:5096-5100 (1991)). The inducible Tag hES cells are then allowed to undergo a first round of differentiation with Tag in the uninduced state at the nonpermissive temperature of 37° C. and in medium lacking exogenous gamma-interferon in six differing conditions. For some cells that have potential for therapeutic or other commercial applications it may be desirable to remove the ectopic SV40 Tag DNA sequences. This may be accomplished by flanking the Tag and other undesirable DNA sequences with the recognition sequences for the Cre or FLP site specific recombinases (Sargent and Wilson, Recombination and Gene Targeting in Mammalian Cells. Current Research in Molecular Therapeutics, (1998) 1:584-590). When these recombinases are expressed in cells they efficiently catalyze recombination at a high frequency, specifically between DNA containing their respective recognition sequences. For example, genes flanked by the loxp recognition sequence for the Cre recombinase may be specifically deleted on intracellular transient expression of Cre recombinase.

For example, construction of H-2Kb-tsA58/neo and H-2Kb-tsA58/neo/loxp vectors may involve the 5′ flanking promoter sequences and the transcriptional initiation site of the mouse H-2Kb class1 gene being fused to the SV40 tsA58 early region coding sequences. The 4.2-kilobase (kb) EcoRI-Nru I fragment encompassing the H-2Kb promoter sequences are ligated to the 2.7-kb Bgl I-BamHI fragment derived from the tsA58 early region gene and pUC19 double-digested with EcoRI and BamHI. The Bgl I site is blunted by using the Klenow fragment of Escherichia coli DNA polymerase I to allow fusion to the Nru I site to generate the Tag expression vector pH-2Kb-tsA58 (Jat et al., Proc Natl Acad Sci USA 88:5096-5100 (1991)). To create a drug selectable Tag vector, the MC1NeoPolA expression cassette is isolated from the pMC1NeoPolA vector as a XhoI/SalI fragment and subcloned into SalI linearized H-2Kb-tsA58 vector to generate pH-2Kb-tsA58/neo. To create a pH-2Kb-tsA58/neo vector which has the pH-2Kb-tsA58/neo cassettes flanked by loxp site-specific recombination sequences, two loxp oligonucleotide duplexes are synthesized and ligated into pH-2Kb-tsA58/neo vector in the unique EcoRI and SalI sites that flank the expression cassettes and in an orientation that allow deletion of the expression cassettes on recombination. Each oligonucleotide duplex reconstructs a functional restriction site and an inactive restriction site such that the entire loxpH-2Kb-tsA58/neoloxp cassette can be removed intact by restriction endonuclease digestion with EcoRI and SalI. To construct this vector, a DNA oligonucleotide duplex molecule containing the loxp recognition sequence (Hoess et al., Proc Natl Acad Sci USA, (1982) 79(11): 3398-402) and single stranded ends complementary to restriction endonuclease EcoRI-cut DNA is ligated into EcoRI digested pH-2Kb-tsA58/neo vector to create the ploxpH-2Kb-tsA58/neo vector. A similar loxp oligonucleotide duplex containing single stranded ends complementary to restriction endonuclease SalI-cut DNA is ligated into SalI digested ploxpH-2Kb-tsA58/neo vector to create the ploxpH-2Kb-tsA58/neoloxp vector. Prior to transfection into H9 hES cells the pH-2Kb-tsA58/neo vector or ploxpH-2Kb-tsA58/neoloxp vector is linearized by restriction endonuclease digestion with EcoRI.

Transfection and establishment of transgenic cell lines may be performed by creating H9 hES cell lines or other ES cells with stably integrated temperature sensitive Tag by transfecting linearized plasmid vector by electroporation or using the chemical transfection reagent Exgene 500 transfection system (Frementas) as previously described (Eiges et al., Current Biol, 11:514-518 (2001), Zwaka and Thomson, Nat. Biotechnol. 21:319-321 (2003) and stable transfectants selected in the presence of the neomycin analog G418.

Transfection and establishment of transgenic cell lines may also be performed by chemical transfection. Human H9 ES cells or other ES cells are transfected with linearized pH-2Kb-tsA58/neo using the ExGen 500 transfection system (Fermentas). Transfection of human ES cells is carried out in 6-well tissue culture plates two days after plating on MEFs, using established conditions described above, and is performed as described by the manufacturer's protocol. Specifically, 2 ug of plasmid DNA plus 10 ul of the transfecting agent ExGen 500 is added to about 3×10⁵ cells/well in a final volume of 1 ml medium per well. The 6-well tissue culture plates are centrifuged at 280×g for 5 minutes and incubated at 37° C. in a humidified low oxygen incubator for an additional 45 min. Residual transfection agent is removed by washing the cells twice with PBS. The following day, cells are trypsinized and approximately 5×10⁵ cells are replated per 10 cm culture dish containing inactivated neomycin resistant MEF cells. Two days following replating, the neomycin analog G418 (200 ng/ml) is added to the growth medium. After approximately 10-14 days, G418 resistant colonies are observed. Single transgenic colonies are picked by a micropipette, dissociated into small clumps of cells, and transferred into a 24-well culture dish containing neomycin resistant MEF cells. The G418 resistant H9 cells are expanded before storage in liquid nitrogen or used for differentiation.

Transfection and establishment of transgenic cell lines may also be performed by electroporation. H9 hES cells or other ES cells are harvested by gentle trypsinization (0.05% mg/ml; Invitrogen, Carlsbad, Calif.), taking care to minimize dissociation into single cell suspensions. Cells are washed with MEF medium, and resuspended in 0.5 ml hES culture medium, not containing antibiotics, at a concentration of 1.5-3.0×10⁷ cells/ml. Immediately prior to electroporation, 40 μg of linearized vector DNA is added in a volume less than 80 ul, and 0.8 ml of the DNA/cell suspension is added to each electroporation cuvette (0.4 cm gap cuvette; BioRad, Hercules, Calif.). Cells are electroporated with a single 320 V, 200 uF pulse at room temperature using the BioRad Gene Pulser II. Electroporated cells are incubated for 10 minutes at room temperature and the contents of each cuvette plated at high density on a 10 cm culture dish seeded with neomycin resistant MEF cells. G418 selection (50 μg/ml, Invitrogen) is started 48 hours after electroporation. After approximately two weeks of G418 selection, surviving colonies are picked using a micropipette to dissociate nascent colonies into small cell clumps and transferred into 24-well tissue culture plates seeded with neomycin resistant MEF cells in hES medium containing 50 μg/ml G418. The G418 resistant colonies are expanded before individual analysis by PCR using primers specific for the neomycin resistance cassette and for the SV40 large T antigen, storage in liquid nitrogen, or used for differentiation. PCR positive clones are rescreened by Southern blot analysis for confirmation using genomic DNA isolated from G418 resistant clones and hybridizing with radiolabelled probes from the neomycin cassette or the SV40 large T antigen.

Inducible Tag-expressing cells are plated in a standard 6 well tissue culture plate on a feeder layer of mouse embryonic fibroblasts and allowed to grow for 9 days to confluence. The hES cell growth medium is replaced by any of the combinations of specialized media or other culture conditions described herein (see Table I) and the hES cells are allowed to differentiate under a variety of conditions and for variable periods of time as described herein.

The resulting heterogeneous mixture of cells is then rinsed with phosphate buffered saline, dissociated into single cells such as with trypsin (0.25% trypsin) and the differentiated cells plated out so as to allow clonal or oligoclonal growth as described herein. The differentiated cells are allowed to proliferate for 14-20 days under permissive temperature and the resulting colonies are cloned and plated in 24 well plates containing the same medium supplemented with gamma-interferon under the permissive temperature of 32.5° C. and extracellular matrix from which they were derived. The cloned colonies are expanded to obtain a stock of cells and the cell line stocks are cryopreserved. To determine the pattern of gene expression, the cells are shifted to the same medium reduced in serum concentration by 20-fold, free of gamma interferon, and at the nonpermissive temperature of 37° C. for five days.

Removal of H-2Kb-tsA58/Neo Vector Sequences from Cell Lines

To remove the H-2Kb-tsA58/neo expression cassettes from cells, cells are transfected with an expression cassette for the Cre, FLP, or equivalent recombinase, for example the pCX-NLS-Cre expression vector containing a nuclear localization signal fused in frame with Cre recombinase. Cells are transfected with Cre expression vector by electroporation or chemical transfection reagents, for example the ExGen 500 transfection system (Fermentas). Transfection of human ES-derived cells is carried out in 6-well tissue culture plates, using established conditions described above, and is performed as described by the manufacturer's protocol. Specifically, 2 μg of Cre expression vector DNA plus 10 μl of the transfecting agent ExGen 500 is added to about 3×10⁵ cells/well in a final volume of 1 ml medium per well. The 6-well tissue culture plates are centrifuged at 280×g for 5 minutes and incubated at 37° C. in a humidified low oxygen incubator for an additional 45 min. Residual transfection agent is removed by washing the cells twice with PBS. The following day, cells are trypsinized and replated at a density of approximately 1000 cells/10 cm culture dish or at a density of approximately 1 cell/well of a 96-well tissue culture plate. Each colony growing on 10 cm tissue culture plates are picked into individual wells of a 96-well plate several weeks after replating. Cells are screened by PCR for loss of H-2Kb-tsA58/neo sequences and by sensitivity to the drug G418. Loss of H-2Kb-tsA58/neo sequences are confirmed by southern analysis using ³²P labeled probes from the H-2Kb-tsA58/neo cassette (Sambrook and Russell Molecular Cloning A Laboratory Manual, 3^{rd} Edition, 2001, Cold Spring Harbor Press).

In another embodiment of the invention, the factors that override cell cycle arrest may be fused with additional proteins or protein domains and delivered to the cells. For example, factors that override cell cycle arrest may be joined to a protein transduction domain (PTD). Protein transduction domains, covalently or non-covalently linked to factors that override cell cycle arrest, allow the translocation of said factors across the cell membranes so the protein may ultimately reach the nuclear compartments of the cells. PTDs that may be fused with factors that override cell cycle arrest include the PTD of the HIV transactivating protein (TAT) (Tat 47-57) (Schwarze and Dowdy 2000 Trends Pharmacol. Sci. 21: 45-48; Krosl et al. 2003 Nature Medicine (9): 1428-1432). For the HIV TAT protein, the amino acid sequence conferring membrane translocation activity corresponds to residues 47-57 (Ho et al., 2001, Cancer Research 61: 473-477; Vives et al., 1997, J. Biol. Chem. 272: 16010-16017). These residues alone can confer protein translocation activity.

In another embodiment of the invention, the PTD and the cycle cycle arrest factor may be conjugated via a linker. The exact length and sequence of the linker and its orientation relative to the linked sequences may vary. The linker may comprise, for example, 2, 10, 20, 30, or more amino acids and may be selected based on desired properties such as solubility, length, steric separation, etc. In particular embodiments, the linker may comprise a functional sequence useful for the purification, detection, or modification, for example, of the fusion protein.

In another embodiment of the invention, single cell-derived or oligoclonal cell-derived cells of this invention may be reprogrammed to an undifferentiated state through novel reprogramming technique, as described in U.S. application No. 60/705,625, filed Aug. 3, 2005, U.S. application No. 60/729,173, filed Oct. 20, 2005; U.S. application No. 60/818,813, filed Jul. 5, 2006, the disclosures of which are incorporated herein by reference. Briefly, the cells may reprogrammed to an undifferentiated state using at least a two, preferably three-step process involving a first nuclear remodeling step, a second cellular reconstitution step, and finally, a third step in which the resulting colonies of cells arising from step two are characterized for the extent of reprogramming and for the normality of the karyotype and quality. In certain embodiments, the single cell-derived or oligoclonal cell-derived cells of this invention may be reprogrammed in the first nuclear remodeling step of the reprogramming process by remodeling the nuclear envelope and the chromatin of a differentiated cell to more closely resemble the molecular composition of an undifferentiated or a germ-line cell. In the second cellular reconstitution step of the reprogramming process, the nucleus, containing the remodeled nuclear envelope of step one, is then fused with a cytoplasmic bleb containing requisite mitotic apparatus which is capable, together with the transferred nucleus, of producing a population of undifferentiated stem cells such as ES or ED-like cells capable of proliferation. In the third step of the reprogramming process, colonies of cells arising from one or a number of cells resulting from step two are characterized for the extent of reprogramming and for the normality of the karyotype and colonies of a high quality are selected. While this third step is not required to successfully reprogram cells and is not necessary in some applications, the inclusion of the third quality control step is preferred when reprogrammed cells are used in certain applications such as human transplantation. Finally, colonies of reprogrammed cells that have a normal karyotype but not sufficient degree of programming may be recycled by repeating steps one and two or steps one through three.

In another embodiment of the invention, the single cell-derived and oligoclonal cell-derived cells may be used to generate ligands using phage display technology (see U.S. application No. 60/685,758, filed May 27, 2005, and PCT US2006/020552, filed May 26, 2006, the disclosures of which are hereby incorporated by reference).

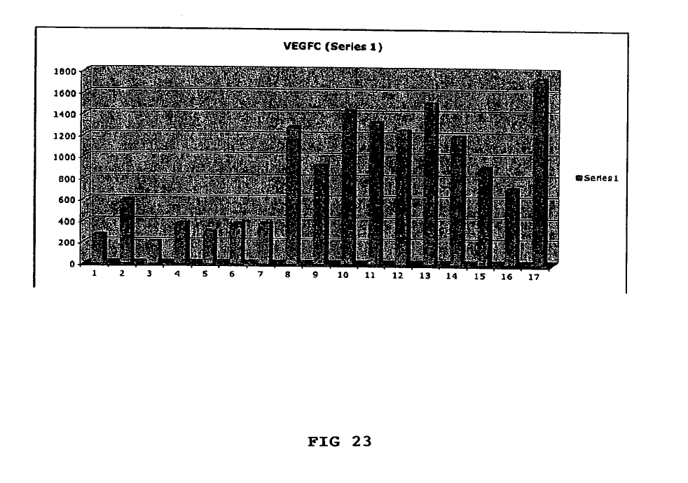

In another embodiment of the invention, the single cell-derived or oligoclonal cell-derived cells of this invention may express unique patterns of gene expression such as high levels of angiogenic and neurotrophic factors. Such cells may be useful for the delivery of these factors to tissues to promote vascularization or innervation where those responses are therapeutic. For example, in the case of the angiogenic factors, cell lines that express high levels of such factors including VEGFA, B, C, or D or angiopoietin-1 or -2 can be transplanted using delivery technologies appropriate to the target tissue to deliver cells that express said angiogenic factor(s) to induce angiogenesis for therapeutic effect. As an example, FIG. 23 depicts the relative gene expression of the angiogenic factor VEGFC in the cells derived from clones 1-17 of Series 1.

The expression of genes of the cells of this invention may be determined. Measurement of the gene expression levels may be performed by any known methods in the art, including but not limited to, microarray gene expression analysis, bead array gene expression analysis and Northern analysis. The gene expression levels may be represented as relative expression normalized to the ADPRT or GAPD housekeeping genes. Based on the gene expression levels, one would expect the expression of the corresponding proteins by the cells of the invention. For example, in the case of cell clone ACTC60 (or B-28) of Series 1, relatively high levels of DKK1, VEGFC and IL1R1 were observed. Therefore, the ability to measure the bioactive or growth factors produced by said cells may be useful in research and in the treatment of disease.

The formulation and dosage of said cells will vary with the tissue and the disease state but in the case of humans and most veterinary animals species, the dosage will be between 10²-10⁶ cells and the formulation can be, by way of nonlimiting example, a cell suspension in isosmotic buffer or a monolayer of cells attached to an layer of extracellular matrix such as contracted gelatin.

In the case of neutrophic factors, the cells made by the methods of this invention may be used to induce the innvervation of tissue such as to improve the sensory innervation of the skin in wound repair or regeneration, or other sensory or motor innervation. For example, cell clones may therefore be formulated for this use using delivery and formulation technologies well known in the art including by way of nonlimiting example, humans and veterinary animal applications where the dosage will be between 10²-10⁶ cells and the formulation can be, by way of nonlimiting example, a cell suspension in isosmotic buffer or a monolayer of cells attached to an layer of extracellular matrix such as contracted gelatin.

Such use of cells that promote angiogenesis or neurite outgrowth may further be combined with an adjunct therapy that includes young hemangioblasts or angioblasts in the case of angiogenesis or neuronal precursors of various kinds in the case of neurite outgrowth. Such combined therapy may have particular utility where the mere administration of angiogenic factors or neurite outgrowth promoting factors by themselves are not sufficient to generate a response due to the fact that there is a paucity of cells capable of responding to the stimulus.

In the case of angiogenesis, the senescence of the vascular endothelium or circulating endothelial precursor cells such as hemangioblasts may blunt the response to angiogenic stimulus. The co-administration of young hemangioblasts by various modalities known in the art based on the size of the animal and the target tissue along with cells capable of delivering an angiogenic stimulus will provide an improved angiogenic response. Such an induction of angiogenesis can be useful in promoting wound healing, the vascularization of tissues prone to ischemia such as aged myocardium, skeletal, or smooth muscle, skin (as in the case of nonhealing skin ulcers such as decubitus or stasis ulcers), intestine, kidney, liver, bone, or brain. [0194] In another embodiment of the invention, the expression of genes or proteins of the cells of this invention may be determined. Measurement of the gene expression levels may be performed by any known methods in the art, including but not limited to, microarray gene expression analysis, bead array gene expression analysis and Northern analysis. The gene expression levels may be represented as relative expression normalized to the ADPRT or GAPD housekeeping genes.

In another embodiment of the invention, the single cell-derived and oligoclonal cell-derived cells, derived by methods of this invention, may be injected into mice to raise antibodies to differentiation antigens. Antibodies to differentiation antigens would be useful for both identifying the cells to document the purity of populations for cell therapies, for research in cell differentiation, as well as for documenting the presence and fate of the cells following transplantation. In general, the techniques for raising antibodies are well known in the art.

A cell produced by the methods of this invention could produce large amounts of BMP3b, and this cell could therefore be useful in inducing bone.

In another embodiment of the invention, cells may produce large quantities of PTN (Accession number NM_002825.5), MDK (Accession number NM_002391.2), or ANGPT2 (Accession number NM_001147.1), or other angiogenesis factors and are therefore useful in inducing angiogenesis when injected in vivo as cell therapy, when mitotically inactivated and then injected in vivo, or when combined with a matrix in either a mitotically-inactivated or native state for use in inducing angiogenesis. PTN-producing cells described in the present invention are also useful when implated in vivo in either a native or mitotically-inactivated state for delivering neuro-active factors, such as preventing the apoptosis of neurons following injury to said neurons.

In another embodiment of the invention, the single cell-derived and oligoclonal cell-derived cells may be used for the purpose of generating increased quantities of diverse cell types with less pluripotentiality than the original stem cell type, but not yet fully differentiated cells. mRNA or miRNA can then be prepared from these cell lines and microarrays of their relative gene expression can be performed.

In another embodiment of the invention, the single cell-derived and oligoclonal cell-derived cells may be used in animal transplant models, e.g. transplanting escalating doses of the cells with or without other molecules, such as ECM components, to determine whether the cells proliferate after transplantation, where they migrate to, and their long-term differentiated fate in safety studies.

This invention contemplates using the cells derived from the methods of this invention in a number of ways. These cells may be used for research. These cells, their progenies, or cells differentiated from these cells may be used therapeutically, for example, for transplantation purposes. The growth factors secreted by cells may also be purified and used. These cells may serve as feeder cells for the derivation, production or maintenance of other cells, such as ES cells. The culture media from these cells may be used to induce differentiation of pluripotent stem cells in methods of this invention.

Unless otherwise defined, all technical and scientific terms used herein have the same meaning as commonly understood by one of ordinary skill in the art. In case of conflict, the present specification, including definitions, will control.

Further, unless otherwise required by context, singular terms shall include pluralities and plural terms shall include the singular.

Generally, nomenclatures used in connection with, and techniques of, cell and tissue culture, molecular biology, immunology, microbiology, genetics, developmental biology, cell biology described herein are those well-known and commonly used in the art.

Exemplary methods and materials are described below, although methods and materials similar or equivalent to those described herein can also be used in the practice or testing of the present invention.

All publications, patents, patent publications and other references mentioned herein are incorporated by reference in their entirety.

Throughout this specification and claims, the word “comprise,” or variations such as “comprises” or “comprising” will be understood to imply the inclusion of a stated integer or group of integers but not the exclusion of any other integer or group of integers.

### Biological Deposits

One cell line described in this application has been deposited with the American Type Culture Collection (“ATCC”; P.O. Box 1549, Manassas, Va. 20108, USA) under the Budapest Treaty. The B-28 cell line, also referred to as ACTC60 or clone 17 of Series 1, was deposited on Jun. 8, 2006 and has ATCC Accession No. PTA-7654, as described in Example 21 below.

### EXAMPLES

### Example 0.1

hES cells are grown to form embryoid bodies (EB) (see U.S. application No. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media are pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, colonies with a pattern of gene expression consistent with that of paraxial mesoderm and scarless skin repair are used as marker of cells useful in scarless skin repair. Alternatively, dermal fibroblasts can be isolated that express proteins for elastogenesis useful in inducing elastogenesis when transplanted in vivo.

### Example 2

hES cells are grown to form embryoid bodies (EB) (see U.S. application No. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, colonies with a pattern of gene expression consistent with that of endodermal cells are identified for use in liver cell, pancreatic beta cell, and intestinal cell transplantation.

### Example 3

hES cells are grown to form embryoid bodies (EB) (see U.S. application No. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, colonies with a pattern of gene expression consistent with that of ectodermal cells are identified for use in neuronal, and epidermal transplantation.

### Example 4

hES cells are grown to form embryoid bodies (EB) (see U.S. application No. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, colonies with a pattern of gene expression consistent with that of cardiac progenitors, stromal fibroblasts including but not limited to cardiac, liver, pancreatic, lung, dermal, renal, AGM region, and intestinal stromal cells are used for transplantation.

### Example 5

hED cells are allowed to differentiate without forming ES cell lines and without forming embryoid bodies and are differentiated for 10 days in DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are trypsinized to form a single cell suspension, the trypsin is neutralized with serum, and the cells are incubated for 15 minutes while gently agitating cells to keep them in suspension while allowing the re-expression of cell surface antigens that may have been removed by trypsin. The cells are then sorted by flow cytometry to select cells positive for endosialin (CD248) using antibody to the antigen. CD248 positive cells and/or other cells are dispersed one cell per well in a multiwell tissue culture plate. The cells are fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, the fibroblasts are used for cell induction, and for transplantation in dermal applications such as for promoting scarless wound healing.

### Example 6

hED cells are allowed to differentiate without forming ES cell lines and without forming embryoid bodies and are differentiated for 10 days in DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. Candidate cells differentiated for 4-8 days in 10% fetal bovine serum are trypsinized, the trypsin is neutralized. And the resulting single cell suspension is sorted by flow cytometry using techniques well known in the art using an antibody to AC4, an antigen known to sort neural crest cells. Single cells are plated at a density of a single cell per well using an automated cell deposition device (“ACDU”). The single cell-derived cultures that result are used for a number of research and therapeutic modalities that use neural crest cells, including the identification of cell cultures that display a dermal prenatal embryonic pattern of gene expression useful for transplantation into the face for regenerating elastic architecture in the dermis and for promoting scarless wound repair.

### Example 7

hED cells are allowed to differentiate without forming ES cell lines and without forming embryoid bodies and are differentiated for 10 days in DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are trypsinzied to form a single cell suspension. The trypsin is then neutralized with serum. And the cells are then incubated for 15 minutes while gently agitating to keep them in suspension, while allowing the re-expression of cell surface antigens that may have been removed by trypsin. The cells are then sorted by flow cytometry to select cells positive for endosialin (CD248) using antibody to the antigen. And the CD248 positive cells and/or other cells are dispersed one cell per well in a multiwell tissue culture plate. The cells are fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, the fibroblasts with a dermal progenitor pattern of gene expression are used to generate conditioned medium which is concentrated and applied topically in promoting scarless wound healing.

### Example 8

hES cells are grown to form embryoid bodies (EB) (see U.S. application No. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum to obtain a heterogeneous population of cells. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies expressing pigment, or pigmented clones, can then be expanded, cryopreserved, quality controlled, and their pattern of gene expression tested using gene expression arrays as is well known in the art.

In this example, colonies with a pattern of gene expression consistent with that of retinal pigment epithelial cells (“RPE”) are identified by examining the extracellular matrix of the cultured RPE cells for proteins of Bruch's membrane. This can be performed by techniques well known in the art, including, but not limited to, extracting the cells from the culture substrate with a detergent such as deoxycholate, and detecting the proteins that remain on said substrate using antibodies to the proteins of Bruch's membrane. The RPE cells that display a prenatal pattern of gene expression such that they deposit embryonic Bruch's membrane proteins can be identified in this manner, cryopreserved, and subsequently injected into the retina in association with degenerative diseases of the retina that have dysfunctional Bruch's membrane such that the injected RPE cells deposit new Bruch's membrane proteins and regenerate the membrane.

### Example 9

hES cells are grown to form embryoid bodies (EB) (see U.S. application No. 60/538,964, filed Jan. 23, 2004; Ser. No. 11/186,720, filed Jul. 20, 2005; PCT application nos. PCT/US05/002273, filed Jan. 24, 2005; PCT/US05/25860, filed Jul. 20, 2005, the disclosures of which are hereby incorporated by reference) and said embryoid bodies are plated in standard tissue culture vessels in the presence of DMEM media supplemented with 10% fetal bovine serum and pooled members of the FGF family FGF-2, FGF-8, FGF-15, FGF-17 at concentrations at the ED50 for each factor as is well known in the art to obtain a heterogeneous population of cells enriched in neuronal cell types. The media of said cultures is collected after 24 hours and the cultures are refed. The collected media is pooled, filtered through a 0.2 micron sterile filter and stored at 4° C. as conditioned medium. After a total of 10 days of differentiation, the differentiated cells are plated at limiting dilution, photographed to document the cell number in each well as well as the differentiated state of the cell, and fed the conditioned medium with biweekly refeeding, and cultured for two weeks in low ambient oxygen (5%), then microscopically analyzed for colony formation. The observed single cell-derived colonies, or clones, can then be expanded, cryopreserved, quality controlled, and their pattern of gene expression tested using gene expression arrays as is well known in the art.

In this example, colonies with a pattern of gene expression consistent with that of neuronal cells are useful in research and cell transplantation.

### Example 10

### Identification of Differentiated Tissues and Cells from Genetically Modified hES Cell Lines for Therapeutic Purposes

Master libraries of differentiated tissues and cell types from hES cells modified to prevent or reduce the severity of rejection by the host immune system may be ultimately used for therapeutic purposes. For example, dopaminergic neurons may be used to treat patients suffering from Parkinson's disease.

In this example, hES cells derived from 0 negative donors are first modified by gene targeting to delete the Major histocompatibility group loci HLA-A, HLA-B and HLA-D.

The same strategy for characterizing master libraries of differentiated hES cells is used to characterize cells that have been derived by directed differentiation. In this example, growth and analysis of dopaminergenic neurons are performed similar to Zeng et al., Stem Cells 22: 925-940 (2004). In brief, high throughput characterization of differentiated cells is performed by visually characterizing cell morphology and by microarray analysis of RNA transcripts to identify expression signatures specific for differentiated cells and tissues. Expression signatures by microarray analysis from differentiated cells and tissues are compared to existing microarray, SAGE, MPSS, and EST databases (Gene Expression Atlas, Affymetrix human Genechip U95A, http://expression.gnf.org; SAGEmap, http://www.ncbi.nlm.nih.gov/SAGE/; Tissuelnfo, http://icb.mssm.edu/crt/tissueinfowebservice.xml; UniGene, http://www.ncbi.nlm.nih.gov/UniGene/) to determine the cell or tissue type. Further additional characterization of differentiated cells and tissues may include immunocytochemistry for specific cell surface antigens, production of specific cell products, and 2D PAGE.

Growth of hESCs.

Briefly, hESCs are maintained on inactivated mouse embryonic fibroblast (MEF) feeder cells in Dulbecco's modified Eagle's medium/Ham's F12 (DMEM/F12, 1:1) supplemented with 15% fetal bovine serum (FBS), 5% knockout serum replacement (KSR), 2 mM nonessential amino acids, 2 mM L-glutamine, 50 μg/ml Penn-Strep (Invitrogen, Carlsbad, Calif., http://www.invitrogen.com), 0.1 mM β-mercaptoethanol (Specialty Media, Phillipsburg, N.J., http://www.specialtymedia.com), and 4 ng/ml basic fibroblast growth factor (bFGF; Sigma, St. Louis, http://www.sigmaaldrich.com). Cells are passaged by incubation in Cell Dissociation Buffer (Invitrogen), dissociated, and then seeded at approximately 20,000 cells/cm². Under such culture condition, the ES cells are passaged every 4-5 days.

ECM components are applied to the culture substrate either to promote the generation of a heterogeneous mixture of differentiated cell types (candidate cultures) and/or for the propagation step. Many ECM components include: Gelatin, or Collagens I, II, III, IV, V, VI, VII, VIII, IX, X, XI, XII, XIII, XIV, XV, XVI, XVII, XVIII and XIX.

Gelatin or specific collagens I-IX may be used to coat the culture substrate as follows. For short-term cultures of two days or less, the collagen solution is simply applied to the substrate and allowed to dry. The collagen solution is diluted 1:20 with 30% ethanol, spread over surface of sterile glass coverslip, and dried in a tissue culture hood. For long-term cultures or greater than two days, such as when culturing cell in the propagation step from a single cell or a small colony (oligoclonal propagation), the substrate can be first coated with polylysine or polyornithine. In this case, polylysine or polyornithine (MW or 30,000-70,000) at 0.1-1 mg/ml in 0.15 M borate buffer (pH 8.3) is filter sterilized and spread over the culture substrate. The covered substrate is incubated 2-24 hours at room temperature. The solution is then aspirated, washed three times with sterile water, and gelatin or specific collagens in solution (100 ug/ml in water) are added and incubated 4-16 hours. The solution is then aspirated, rinsed once with the medium to be used, and then seeded with cells in the medium used.

An alternative technique for long-term cultures generates a double layered collagen coating. The collagen solution as described above is spread on the substrate. This solution is immediately neutralized for 2 minutes with ammonium hydroxide vapors by placing the substrate in a covered dish containing filter paper wet with concentrated ammonium hydroxide. This will cause the collagen to gel. The substrate is then rinsed twice with sterile water and a thin film of the same solution is gently over the surface of the gelled collagen and air dried. The double layered collagen substrate is then used the same day for cell culture.

A polylysine-coated culture substrate can also be used as follows. A 0.01% solution of 150,000-300,000 molecular weight poly-D-lysine (Sigma P4832) is added to the culture vessel at about 0.5 mL per 25 cm² of surface area, incubated at 37° C. for 2-24 hours, removed, the substrate is rinsed twice with DPBS, and used immediately, or stored at 4° C.

Fibronectin may also be applied to the culture substrate. Fibronectin is an extracellular matrix constituent used for the culture of endothelial cells, fibroblasts, neurons and CHO cells. Briefly, stock solutions of fibronectin can be prepared by dissolving 1 mg/ml fibronectin in PBS, which is then filter sterilized and frozen in aliquots. The stock solution is diluted to 50-100 μg/ml in basal medium or PBS. Then, enough solution is added to pool over the surface of sterile glass coverslip. The coverslips can be incubated for 30-45 minutes at room temperature. The fibronectin solution is then aspirated to remove the excess fibronectin solution and the coverslips are then rinsed with media or PBS. Immediately thereafter, either cell suspension or growth media is added to prevent the fibronectin coating from drying.

Alternatively, laminin may be applied to the culture substrate. Laminin is an extracellular matrix constituent used for the culture of neurons, epithelial cells, leukocytes, myoblasts and CHO cells. Briefly, stock solutions of laminin can be prepared by dissolving 1 mg/ml laminin in PBS, which is then filter sterilized and frozen in aliquots. The stock solution is diluted to 10-100 μg/ml in basal medium or PBS. Then, enough solution is added to pool over the surface of sterile glass coverslip. The coverslips can be incubated for several hours at room temperature. The laminin solution is then aspirated to remove the excess laminin solution and the coverslips are then rinsed with media or PBS. Immediately thereafter, either cell suspension or growth media is added to prevent the fibronectin coating from drying. Furthermore, coating the glass coverslip first with polylysine or polyornithine followed by coating with laminin may increase the concentration of laminin applied using this method.

Neural Differentiation.

Neural differentiation of ES cells is induced by the mouse stromal cell line PA6 as described by Kawasaki et al., Neuron, 28:31-40 (2000), with some modifications. hESCs are cultured to form colonies on PA6 feeder cells in Glasgow minimum essential media (Invitrogen) supplemented with 10% KSR (Invitrogen), 1 mM pyruvate (Sigma), 0.1 mM nonessential amino acids, and 0.1 mM b-mercaptoethanol. ES cell colonies are grown at a density of 1,000 colonies per 3-cm dish. The medium is changed on days 4 and 6 and every day thereafter.

Immunocytochemistry.

Expression of stem cell and neuronal markers is examined by immunocytochemistry, and staining procedures are as described previously Zeng et al., Stem Cells, 21:647-653 (2003). Briefly, the ES cells are fixed with 4% paraformaldehyde and permeabilized with 0.1% Triton X-100. After blocking, the cells are incubated with primary antibody. The primary antibodies and the dilution used are as follows: Nestin and bromodeoxyurindine (BrdU [BD Pharmingen, San Diego, Calif., http://www.bdscience.com], 1:500 and 1:200); neural cell adhesion molecule (NCAM), synapsin, synaptophysin, and dopamine beta hydroxylase (DBH [Chemicon, Temecula, Ca, http://www.chemicon.com], 1:200, 1:20, 1:100, and 1:200); and neuron-specific class III beta tubulin (TuJ1) and tyrosine hydroxylase (TH [Sigma], 1:2000 and 1:2000, respectively). Localization of antigens is visualized by using respective secondary antibodies (Alexa fluor 594 or 488; Molecular Probes, Eugene, Oreg., http://www.probes.com).

Reverse Transcription-Polymerase Chain Reaction.

Total RNA is extracted from undifferentiated or differentiated cells using RNA STAT-60 (Tel-Test Inc., Friendswood, Tex.). cDNA is synthesized using a reverse transcription kit (RETROscript, Ambion, Austin, Tex.) with 100 ng total RNA in a 20-μl reaction according to the manufacturer's recommendations. RNase H 1 μl (Invitrogen) is added to each tube and incubated for 20 minutes at 37° C. before proceeding to the reverse transcription-polymerase chain reaction (RTPCR) analysis. For each PCR reaction, 0.5-μl cDNA template is used in a 50-μl reaction volume with the RedTaq DNA polymerase (Sigma). The cycling parameters are as follows: 94° C., 1 minute; 55° C., 1 minute; 72° C., 1 minute for 30 cycles. The PCR cycle is preceded by an initial denaturation of 3 minutes at 94° C. and followed by a final extension of 10 minutes at 72° C. Real-time PCR is used to quantify the levels of mRNA expression of Nurr1. PCR reactions are carried out using an Opticon instrument (MJ Research, Waltham, Mass.) and SYBR Green reagents (Roche Molecular Biochemicals, Indianapolis) according to the manufacturer's instructions. The content of Nurr1 is normalized to the content of the housekeeping gene cyclophilin. Standard curves are generated by cloning amplified products, using human cDNA as a template, into the PCR4 vector (TOPO TA cloning kit [Invitrogen]). The purified fragment solution is measured in a spectrophotometer, and the molecular number is calculated. Plasmid solutions are then used to generate serial dilutions. PCR analyses are conducted in triplicate for each sample. The primer pairs used for real-time PCR analyses are sequence verified. The acquisition temperature for each primer pair is 3° C. below the determined melting point for the PCR product being analyzed.

Detection of Dopamine.

hES cells are cultured on a PA6 cell layer for 3 weeks and rinsed twice with Hanks' balanced salt solution (HBSS). To induce depolarization, 56 mM KCl is added into the cells for 15 minutes. The medium is then collected and stabilized with 0.1 mM EDTA and analyzed for dopamine and DOPAC. Dopamine and DOPAC levels are measured using an HPLC coupled to an ESA Coulochem II Detector (Model 5200, ESA, Inc., Chelmsford, Mass.) with a dual-electrode microdialysis cell. Data are analyzed using an ESA data station (Model 501). Samples (20 μl) are injected by an autosampler (CMA 280) into a C-18 reverse-phase column (3 μm; particle size, 3μ 150 mm; Analytical MD-150 [ESA, Inc.]). The mobile phase for dopamine separation consists of 75 mM NaH2PO4, 1.5 mM 1-octanesulfonic acid-sodium salt, 10 μM EDTA, and 7% acetonitrile (pH 3.0, adjusted with H3PO4). Dopamine and DOPAC are quantified using the reducing (−250 mV) and oxidizing electrodes (350 mV), respectively, and then calculated as nanomolar concentration. The limit of detection is approximately 0.3 pg per injection.

Focused Microarray Analysis.

The nonradioactive GEArray™ Q series cDNA expression array filters for human stem cell genes pathway genes and mouse cytokine genes (Hs601 and MM-003N, SuperArray Inc, http://superarray.com) (Luo et al., Stem Cells, 21:575-587 (2003)) are used according to the manufacturer's protocol. The biotin 2′-deoxyuridine-5′-triphosphate (dUTP)-labeled cDNA probes are specifically generated in the presence of a designed set of gene-specific primers using total RNA (4 μg per filter) and 200 U MMLV reverse transcriptase (Promega, San Luis Obispo, Calif., http://www.promega.com). The array filters are hybridized with biotin-labeled probes at 60° C. for 17 hours. After that, the filters are washed twice with 2× standard saline citrate (SSC)/1% SDS and then twice with 0.1×SSC/1% SDS at 60° C. for 15 minutes each. Chemiluminescent detection steps are performed by incubation of the filters with alkaline phosphatase-conjugated streptavidin and CDP-Star substrate. Array membranes are exposed to Xray film. Quantification of gene expression on the array is performed with ScionImage software. cDNA microarray experiments are done twice with new filters and RNA isolated at different times. Results from the focused array are independently confirmed, and the array itself is validated (Wang et al., Exp Neurol 136:98-106 (1995)).

Of the 266 genes represented by the array, 50 genes are expressed in the induced neurons but not detected in undifferentiated cells. These include 14 markers for stem and differentiated cells, 22 growth factors and receptors, adhesion molecules, and cytokines, six extracellular matrix molecules, and eight others. In particular, Sox1, Map2, TrkC, and NT3 are expressed at higher levels in the differentiated cultures, which is consistent with results obtained by RT-PCR.

The expression of markers for dopaminergic neurons, as well as other neuronal markers, in hESC-derived differentiated cells is examined by immunocytochemistry, RT-PCR, and microarrays. The markers associated with the mature dopaminergic neuronal phenotype: TH, DAT, AADC, GTPCH, PCD, DHPR, and VMAT2 are expressed. The growth factor receptors TrkA, TrkB, TrkC, GFRA1, GFRA2, GFRA3, p75R, and c-ret and the Shh receptors Ptch and Smo are also present. Transcription factors Nurr1, Ptx3, Lmx1b, and Sox-1 associated with dopaminergic and neuronal differentiation are expressed by the PA6 cell-induced cells. Nurr1 is detectable in both undifferentiated hESCs and PA6-differentiated cells, but quantitative RT-PCR verified that a threefold increase in expression was associated with differentiation. DBH was not expressed in the TH-positive cells by immunostaining or RTPCR, and little or no NA was released by KCl stimulation, supporting the conclusion that PA6-induced hESC-differentiated cells are dopaminergic rather than noradrenergic. In addition to dopaminergic markers, cholinergic (ChAT and VAChT) and glutamatergic (GAC and KGA) markers were detected in the induced neurons, indicating the potential for generation of multiple neuronal types by this method. On the other hand, undifferentiated ES cell markers (hTERT, Oct3/4, Dppa5, and UTF-1) are not expressed in the differentiated cultures, indicating that undifferentiated hESCs do not persist in hESC cultures differentiated on PA6 cells.

### Example 11

Any pluripotent stem cells, such as ES cell lines and embryos, ICMs or blastomeres directly differentiated without making lines, may be used as the source of generating the cells of the present invention. Direct differentiation refers, for example, to methods of making downstream stem cells from an embryo without making ES cells (see U.S. patent publication no. 20050265976, published Dec. 1, 2005, and international patent publication no. WO0129206, published Apr. 26, 2001, the disclosures of which are hereby incorporated by reference herein). The resulting cells are “embryo-derived” (“ED”) cells, meaning cells made from embryos by directly differentiating them in vitro without making ES cell lines.

In this example, hES cells are derived from a single blastomere of a cryopreserved embryo wherein the original embryo is cryopreserved again and the blastomere is used to generate a female O-hES cell line with the HLA knockout. These hES cell colonies are differentiated using in situ colony differentiation by culturing them in conditions that induce differentiation without removing the colonies from their culture vessel, such as conditions that occur in the differentiation matrix shown in FIG. 1, in this example, condition #456 which is removal of LIF and the addition of 10% FBS. After various periods of time (1-100 days) in this example, 6 days, the cells are trypsinized and plated at limiting dilution such that most wells have a single cell. The wells are photodocumented to demonstrate a single cell is resident and that it does not have the morphological parameters of an ES cell. The plates are incubated in low ambient oxygen (5%) for ten days and microscopically analyzed for the presence of cell colonies. Colonies are photographed, trypsinized and passaged in the same media and characterized by gene expression as described below. Based on the type of tissue, the cells are lapelled by lentivirus carrying GFP or other markers such as beta galactosidase and injected into the corresponding tissue in an immunocompromised mouse to test engraftment.

### Example 12

Human blastocyst ICMs are isolated by immunosurgery and ICMs are plated in conditions to promote the direct differentiation of the ICM. In this example, the ICM-derived cells are from a nuclear transfer embryo that is female O- and HLA knockout. They are differentiated by culturing them in conditions that induce ICM in situ differentiation, such as conditions that occur in the differentiation matrix shown in FIG. 1, in this example, condition #456 which is removal of LIF and the addition of 10% FBS. After various periods of time (1-100 days) in this example, 6 days, the cells are trypsinized and plated at limiting dilution such that most wells have a single cell. The wells are photodocumented to demonstrate a single cell is resident and that it does not have the morphological parameters of an ES cell. The plates are incubated in low ambient oxygen (5%) for ten days and microscopically analyzed for the presence of cell colonies. Colonies are photographed, trypsinized and passaged in the same media and characterized by gene expression as described below. Based on the type of tissue, the cells are lapelled by lentivirus carrying GFP or other markers such as beta galactosidase and injected into the corresponding tissue in an immunocompromised mouse to test engraftment.

### Example 13

Colonies from the hES cell line ACT3 were differentiated using in situ colony differentiation by culturing the cells in conditions that induce differentiation without removing the colonies from their initial culture vessel, such as conditions that occur in the differentiation matrix shown in FIG. 1. In this example, the condition used was #456 which is removal of LIF-containing medium and the addition of DMEM medium containing 10% FBS. At various intervals of time (5, 7, and 9 days of exposure to differentiation medium), the cells are trypsinized, and plated onto 15 cm gelatinized plates and cultured for an additional 20 days to further induce differentiation into a heterogeneous mixture of early embryonic cell types as the final candidate culture. Therefore, in this example, the cells were differentiated into candidate cultures of heterogeneous differentiated cell types using two sequential differentiation-inducing conditions, one being condition #456 (removal of LIF and the addition of 10% FBS), and the second being #339 (grown in media without LIF with 10% FBS and grown on gelatin ECM).

The cells appeared largely fibroblastic, though heterogeneous in appearance and were then trypsinized and counted with a Coulter counter, and a volume containing 2,500 cells, 5,000 cells and 25,000 cells was introduced into gelatinized 15 cm tissue culture plates containing DMEM medium supplemented with 10% FBS, rocked twice counterclockwise, twice clockwise, twice vertically, twice horizontally to disperse the cells and subsequently incubated in 5% ambient oxygen undisturbed for two weeks.

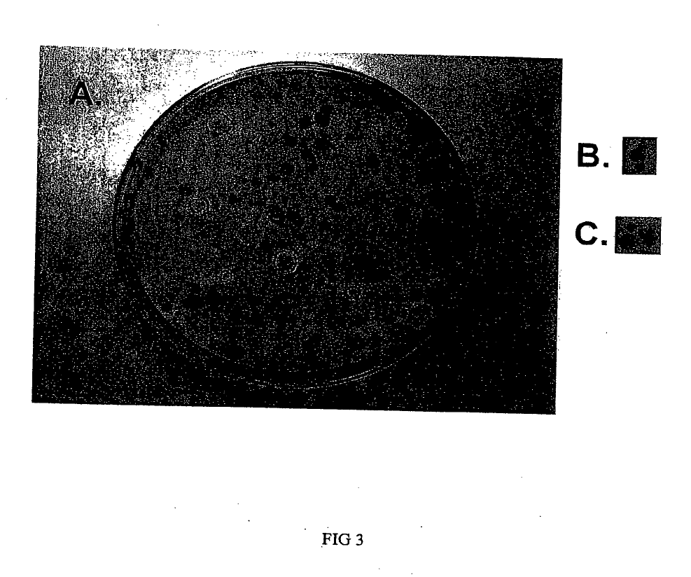

Clonal colonies were identified by phase contrast microscopy and those that are uniformly circular and well separated from surrounding colonies were marked for removal using cloning cylinders as is well known in the art. The dish of colonies at day 9 of in situ differentiation followed by 20 days of in vitro differentiation on gelatin and plated at 2,500 cell per dish was stained with crystal violet solution for 10 minutes, rinsed with water and is shown in FIG. 3.

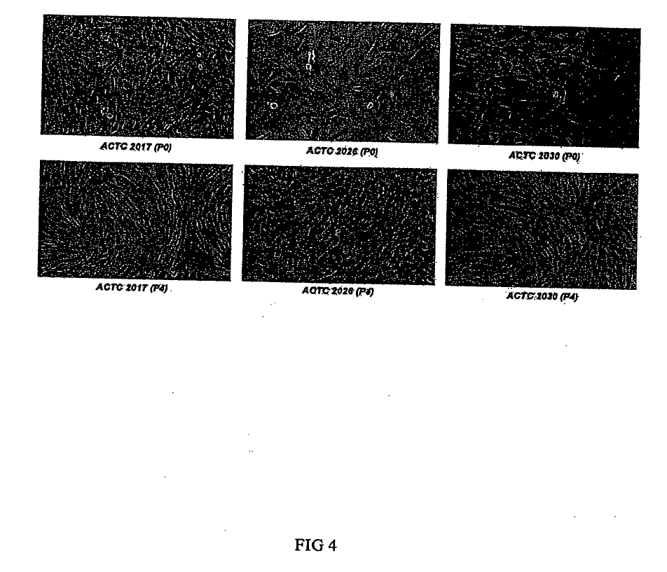

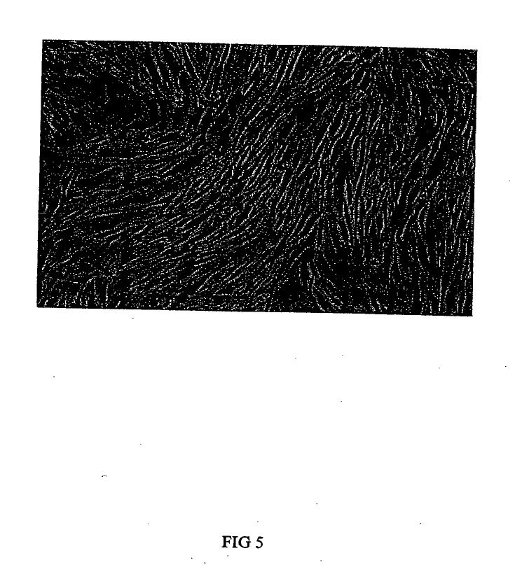

The trypsinized cells from within 61 cloning cylinders (P0) were then replated into gelatinized 24 well plates and incubated. Of 61 colonies isolated, 45 clonal populations became confluent in the 24 well plates (P1) and were then trypsinized and plated in 12 well gelatinized plates (P2). Of these, 44 wells became confluent and these were in turn trypsinized and plated in 6 well gelatinized plates (P3). Of these, 40 became confluent and were transferred to two six well gelatinized plates (P4). Of these, 34 became confluent and were trypsinized and plated in a 100 mm gelatinized tissue culture dish (P5). Of these, 16 became confluent and were trypsinized and transferred to gelatinized T75 flasks (P6). Representative phase contrast photographs of cells in the original clonal colony (P0) and after the fourth passage (P4) are shown in FIG. 4.

The cell cultures tested displayed a normal human karyotype. RNA was harvested from the cells in order to characterize the cell strains and the nature of their differentiated state. Other aliquots of cells were plated onto glass coverslips for immunocytochemical characterization of their differentiated state using antibodies to antigens such as are listed in Table V.

### Example 14

Colonies from the hES cell line ACTS were differentiated using in situ colony differentiation by culturing the cells in conditions that induce differentiation without removing the colonies from their initial culture vessel, such as conditions that occur in the differentiation matrix shown in FIG. 1. In this example, the condition used was #456, which is removal of LIF-containing medium and the addition of DMEM medium containing 10% FBS. The cells were differentiated for 7 days by exposure to differentiation medium, and viable, day 7 differentiated cells were determined via trypan blue exclusion method.

Day 7 differentiated cells were used in this experiment because the dermal progenitor clone B-2 (ACTC #59) was isolated from these differentiated cells. The cells were cultured in either DMEM with various concentrations of FBS or in specialized media.

For the culturing of cells in DMEM media with 3 different FBS concentrations, approximately 1,000 day 7 differentiated cells were plated in 15 cm gelatin-coated tissue culture plates containing DMEM media with either 5% FBS, 10% FBS or 20% FBS. Each media tested was carried out in replicates of 5 dishes per data point.

For the culturing of cells in specialized media, approximately 2,500 and 10,000 of day 7 differentiated cells were plated in 15-cm gelatin-coated tissue culture plates containing any one of the following cell selection/growth media in Table VI:

TABLE VI
Cell Selection and Growth Media

| | Media | Manufacturer | Catalog Number | Addition |
|---|---|---|---|---|
| 1 | Airway | PromoCell | C-21260 | Manufacturer |
| | Epithelial | | | Supplement |
| | Growth | | | |
| | Medium | | | |
| 2 | Epi-Life | Cascade | M-EPIcf/PRF-500 | LSGS (Low |
| | (LSGS) | | | Serum Growth |
| | Medium. | | | Supplement) |
| 3 | Neurobasal | Gibco | 12348-017 | B27 |
| | Medium - B27 | | | |
| 4 | Neurobasal | Gibco | 12348-017 | N2 |
| | Medium - N2 | | | |
| 5 | HepatoZyme- | Gibco | 17705-021 | None |
| | SFM | | | |
| 6 | Epi-Life | Cascade | M-EPIcf/PRF-500 | HKGS (Human |
| | (HKGS) | | | Keratinocyte |
| | Medium. | | | Growth |
| | | | | Supplement) |
| 7 | Endothelial | PromoCell | C-22221 | Manufacturer |
| | Cell Growth | | | Supplement |
| | Medium | | | |
| 8 | Endothelial | Gibco | 11111-044 | Epithelial |
| | Cell SFM | | | Growth |
| | | | | Factor, Basic |
| | | | | Fibroblast |
| | | | | Growth Factor |
| 9 | Skeletal | PromoCell | C-23260 | Manufacturer |
| | Muscle Growth | | | Supplement |
| | Medium | | | |
| 10 | Smooth Muscle | PromoCell | C-22262 | Manufacturer |
| | Basal Medium | | | Supplement |
| 11 | MesenCult | Stem Cell | 05041 | Manufacturer |
| | | Technologies | | Supplement |
| 12 | Melanocyte | PromoCell | C-24010 | Manufacturer |
| | Growth | | | Supplement |
| | Medium | | | |

The cell selection/growth media may preferentially select and sustain growth of particular cell phenotypes for which they were designed.

Each media tested was carried out with one plate of each cell concentration.

The day 7 differentiated cells cultured in either the DMEM/FBS or cell selection/growth media were allowed to grow for 7-10 days to form colonies, the colonies cloned and plated in 24-well gelatin-coated plates containing the same medium in which they were grown. The individual colonies are expanded to obtain a stock of cells and the cell line stocks are cryopreserved.

During the clonal expansion protocol, samples of the cell lines are taken for gene expression and immunophenotype analysis.

### Example 15

Cells from human ES (hES) cell line H-9 passage #48 were plated in a standard 6 well tissue culture plate on a feeder layer of mouse embryonic fibroblasts and allowed to grow for 9 days to confluence. The hES cell growth medium was replaced by 6 differentiation media as shown in Table VII, and the hES cells were allowed to differentiate for 3 days.

TABLE VII
Differentiation Media

| hES | | | | |
|---|---|---|---|---|
| Cell | Differentiation | | | |
| Well | Medium | | Catalog | |
| Number | Addition | Manufacturer | Number | Addition |
| 1 | Airway | PromoCell | C-21260 | Manufacturer |
| | Eiphelial | | | Supplement |
| | Growth | | | |
| | Medium | | | |
| 2 | Neurobasal | Gibco | 12348- | B-27 |
| | Medium - B27 | | 017 | |
| 3 | Epi-Life | Cascade | M- | LSGS (Low |
| | Medium - | | EPIcf/PRF- | Serum Growth |
| | LSGS | | 500 | Supplement) |
| 4 | Endothelial | PromoCell | C-22221 | Manufacturer |
| | Cell | | | Supplement |
| | Growth | | | |
| | Medium | | | |
| 5 | Skeletal | PromoCell | C-23260 | Manufacturer |
| | Muscle | | | Supplement |
| | Cell Growth | | | |
| | Medium | | | |
| 6 | DMEM + 10% | Hyclone | SH302285- | 10% fetal |
| | FBS | | 03 | bovine serum |

The cells were trypsinized using 0.05% trypsin and transferred to Corning 6-well, ultra low attachment tissue culture plates containing 12 embryoid body media as shown in Table VIII, and allowed to form embryoid bodies.

TABLE VIII
Embryoid Body Media

| | | Embryoid | | | |
|---|---|---|---|---|---|
| hES Cell | | Body Well | | | |
| Well | Differentiation | (Ultra Low | | | |
| (Original | Medium (Original | Attachment | Embyoid Body | | Catalog |
| Plate) | Plate) | Plate) | Media | Manufacturer | Number |
| Well 1 | Airway Eiphelial | 1 | Airway | PromoCell | C-21260 |
| | Medium | | Eiphelial | | |
| | | | Growth Medium | | |
| | | 2 | Epi-Life | Cascade | M- |
| | | | (LSGS) Medium | | EPIcf/PRF- |
| | | | | | 500 |
| Well 2 | Neurobasal | 3 | Neurobasal | Gibco | 12348-017 |
| | Medium - B27 | | Medium - B27 | | |
| | | 4 | Neurobasal | Gibco | 12348-017 |
| | | | Medium - N2 | | |
| Well 3 | Epi-Life (LSGS) | 5 | HepatoZyme- | Gibco | 17705-021 |
| | Medium. | | SFM | | |
| | | 6 | Epi-Life | Cascade | M- |
| | | | (HKGS) Medium | | EPIcf/PRF- |
| | | | | | 500 |
| Well 4 | Endothelial Cell | 7 | Endothelial | PromoCell | C-22221 |
| | Medium | | Cell Growth | | |
| | | | Medium | | |
| | | 8 | Endothelial | Gibco | 11111-044 |
| | | | Cell SFM | | |
| Well 5 | Skeletal Muscle | 9 | Skeletal | PromoCell | C-23260 |
| | Cell Medium | | Muscle Cell | | |
| | | | Growth Medium | | |
| | | 10 | Smooth Muscle | PromoCell | C-22262 |
| | | | Basal Medium | | |
| Well 6 | DMEM + 10% FBS | 11 | DMEM + 20% | Hyclone | SH302285- |
| | | | FBS | | 03 |
| | | 12 | Melanocyte | PromoCell | C-24010 |
| | | | Growth Media | | |

One well of differentiated hES cells were divided equally between 2 wells containing 2 different media and allowed to form embryoid bodies. For example, well number 1 of the original 6 well plate in which the hES cells were allowed to differentiate in Airway Eiphelial Medium for 3 days and then were trypsinized and half the cells are placed in a well of an ultra low attachment plate containing the same Airway Eiphelial Medium and the other half of the cells transferred to a second well of the ultra low attachment plate containing Epi-Life LSGS Medium.

The embryoid bodies were allowed to differentiate for 7-10 days, collected, washed in phosphate buffered saline, dissociated into single cells with trypsin (0.25% trypsin) and the differentiated cells plated out in extra cellular matrix coated 15 cm plates (see Table IX). The differentiated cells are allowed to proliferate for 7-20 days and the resulting colonies are cloned and plated in 24 well plates containing the same medium and extra cellular matrix from which they were derived. The cloned colonies are expanded to obtain a stock of cells and the cell line stocks are cryopreserved.

TABLE IX
Extracellular Matrix & Growth Medium

| | | Extra |
|---|---|---|
| | | Cellular |
| 15 cm Plate | Selection & Growth Media | Matrix |
| 1 | Airway Eiphelial Growth | Gelatin |
| | Medium | |
| 2 | Epi-Life (LSGS) Medium. | Collagen IV |
| 3 | Neurobasal Medium - B27 | Poly-lysine - |
| | | BioCoat |
| 4 | Neurobasal Medium - N2 | Poly-lysine - |
| | | BioCoat |
| 5 | HepatoZyme-SFM | Collagen IV |
| 6 | Epi-Life (HKGS) Medium. | Collagen IV |
| 7 | Endothelial Cell Growth | Gelatin |
| | Medium | |
| 8 | Endothelial Cell SFM | Gelatin |
| 9 | Skeletal Muscle Cell Growth | Gelatin |
| | Medium | |
| 10 | Smooth Muscle Basal Medium | Gelatin |
| 11 | DMEM + 20% FBS | Gelatin |
| 12 | Melanocyte Growth Medium | Gelatin |

During the clonal expansion protocol, samples of the cell lines are taken for gene expression and immunophenotype analysis.

### Example 16

Colonies from the hES cell line ACTS were differentiated using in situ colony differentiation by culturing them in conditions that induce differentiation without removing the colonies from their culture vessel, such as conditions that occur in the differentiation matrix shown in FIG. 1. In this example, the condition used was #456, which is removal of LIF and the addition of 10% FBS. At intervals of 5, 7, and 9 days after the colonies had begun to differentiate, the cells were trypsinized, and 25,000 cells were plated onto 15 cm gelatinized plates and cultured for an additional 20 days to further induce differentiation into a heterogeneous mixture of early embryonic cell types as the final candidate culture. These cells were then cryopreserved using DMSO as is well known in the art. The cells were subsequently thawed, cultured for two days on different ECMs (gelatin, plasma fibronectin, poly-D-lysine, and tenscin-C) and in chemically-defined, serum-free medium (Lifeline Lifeline Fibrolife Medium LM-0001). The cells were then trypsinized and counted with a Coulter counter, and a volume containing 5,000 cells in the case of day 5, and 1,000 cells in the case of days 7 and were introduced into 150 mm tissue culture dishes with the same medium and array of ECMs and subsequently incubated in 5% ambient oxygen undisturbed for two weeks with the exception of feeding after one week. Colonies are then identified by phase contrast microscopy, isolated, expanded, and characterized as described above in Example 13.

### Example 17

### Single Cell-Derived Cell Lines of Series 1

To derive the cells of Series 1, colonies from the hES cell line ACTS were routinely cultured in hES medium (KO-DMEM, 1× nonessential amino acids, 1× Glutamax-1, 55 uM beta-mercaptoethanol, 10% Serum Replacement, 10% Plasmanate, 10 ng/ml LIF, 4 ng/ml bFGF) and passaged by trypsinization. hES cells were plated at 500-10,000 cells per 15 cm dish. Three days after passaging, the cells were differentiated using colony in situ differentiation by the removal of LIF-containing medium and the addition of DMEM medium containing 10% FBS (Table I, conditions #456 and #1103). After various periods of time (5, 7, and 9 days of exposure to differentiation medium), the cells were trypsinized and plated onto 15 cm plates at low density of approximately 1,000 cells per cm² coated with the extracellular matrix protein Type I collagen (gelatin) (Table I, condition #339), and cultured for an additional 20 days to further induce differentiation in the same conditions in which they will subsequently be clonally expanded (the enrichment step). Series 1 cells were then trypsinized and counted with a Coulter counter, and the cells were plated at increasing dilutions with a volume containing 2,500 cells, 5,000 cells and 25,000 cells introduced into the 15 cm tissue culture plates and subsequently incubated in 5% ambient oxygen (Table I, condition #449) undisturbed for two weeks.

Clonal colonies were identified by phase contrast microscopy and those that are uniformly circular and well separated from surrounding colonies were marked for removal using cloning cylinders.

The trypsinized cells from within each cloning cylinder were then replated into collagen coated 24 well plates and incubated. Of 61 colonies isolated, 54 grew at a relatively rapid rate of approximately one doubling a day. The cells were karyotyped and determined to be normal human. Colonies were serially grown in gelatinized 24 well, 12 well, 6 well tissue culture plates, T25, T75, T150 flasks, and in some cases to 2 liter Roller Bottles (850 cm² surface area) before freezing and storing in liquid nitrogen. Of 61 colonies isolated from the cells of Series 1, 43 grew at a relatively rapid rate of approximately one doubling a day. Of these colonies, 19 cultures propagated to 150 cm² flasks and were then cryopreserved using 10% tissue grade DMSO in ethanol chambers and were assigned ACTC numbers (see Table X). All of those cell lines described in the present invention assigned ACTC numbers displayed the capacity for propagation in vitro. Those cell lines not given an ACTC number displayed a capacity for propagation from one cell to approximately 5×10⁵ cells but may or may not show the capacity for long-term propagation in vitro beyond that point. The cells were karyotyped and determined to be normal human. Cell morphologies and cell growth were monitored by phase contrast microscopy and recorded by photomicroscopy. Cells were cultured in 6 well tissue culture plates or 6 cm tissue culture Petri dishes prior to freezing to harvest mRNA for gene expression analysis using the Illumina human sentra-6 platform. The cell lines isolated are shown in the table below.

Series 1 Exp.

| Line Name | ACTC No. | Medium |
|---|---|---|
| 1 | | DMEM 10% Fetal Bovine Serum |
| 2 | | |
| 3 | | |
| 4 | | |
| 5 | | |
| 6 | | |
| B-1 | | |
| B-2 | 51 | |
| B-3 | 55 | |
| B-4 | 66 | |
| B-5 | | |
| B-6 | 56 | |
| B-7 | 53 | |
| B-9 | | |
| B-10 | | |
| B-11 | 58 | |
| B-12 | 65 | |
| B-13 | | |
| B-14 | 67 | |
| B-15 | 71 | |
| B-16 | 59 | |
| B-17 | 54 | |
| B-18 | | |
| B-19 | | |
| B-20 | | |
| B-21 | | |
| B-22 | | |
| B-23 | | |
| B-24 | | |
| B-25 | 57 | |
| B-26 | 50 | |
| B-27 | | |
| B-28 | 60 | |
| B-29 | 52 | |
| B-30 | 61 | |
| B-31 | | |
| B-32 | | |
| B-33 | | |
| B-34 | | |
| B-35 | | |
| 2-1 | 63 | |
| 2-2 | 62 | |
| 2-3 | 70 | |
| 2-4 | | |
| 4-1 | | |
| 4-2 | 69 | |
| 4-3 | | |
| 4-4 | | |
| 5-1 | | |
| 5-2 | | |
| 5-3 | | |
| 5-4 | 68 | |
| 5-5 | | |
| 6-1 | 64 | |

TOTAL COLONIES SERIES 1 = 54

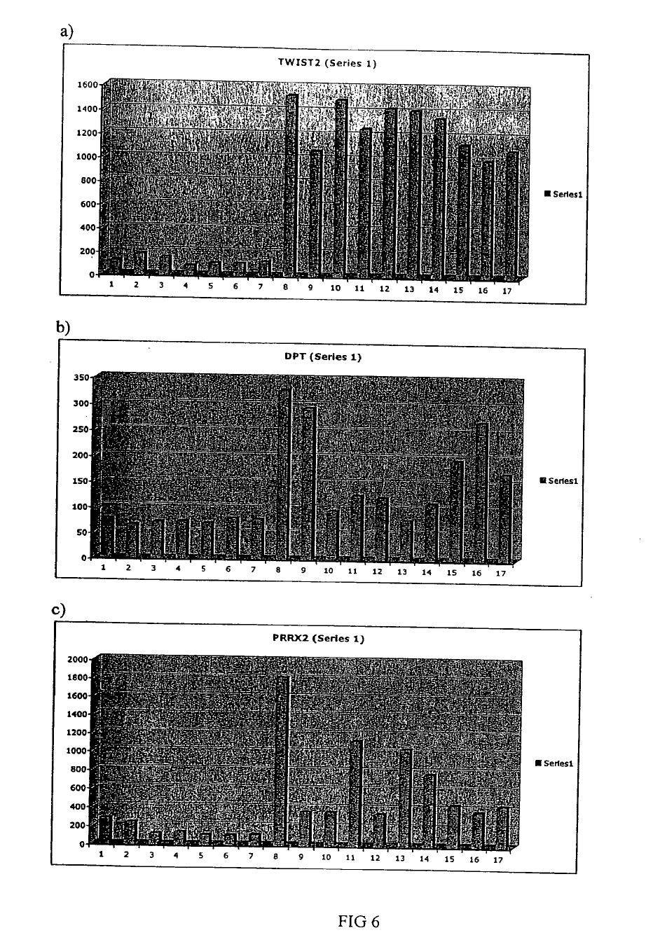

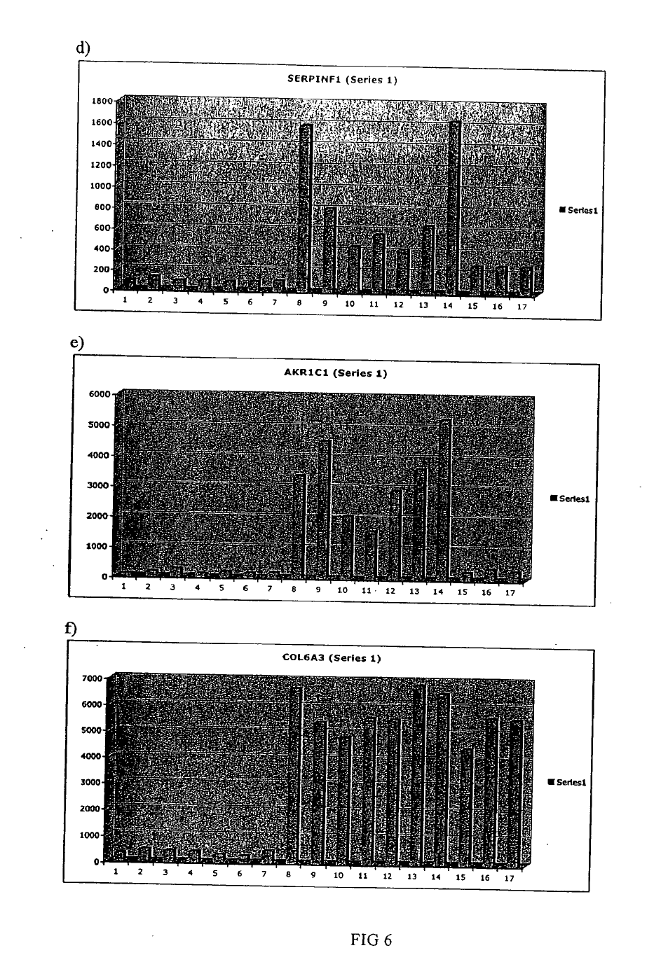

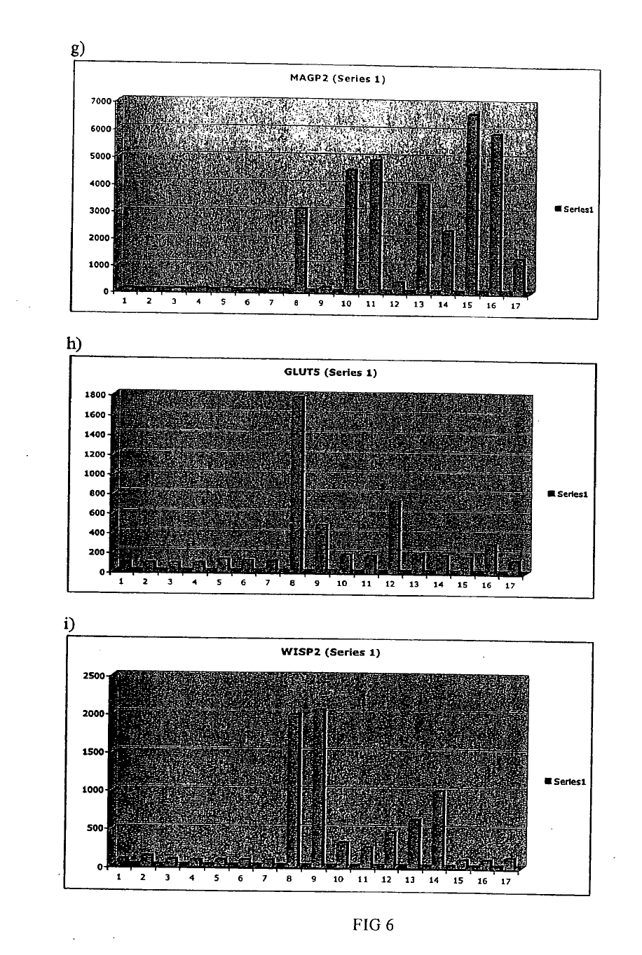

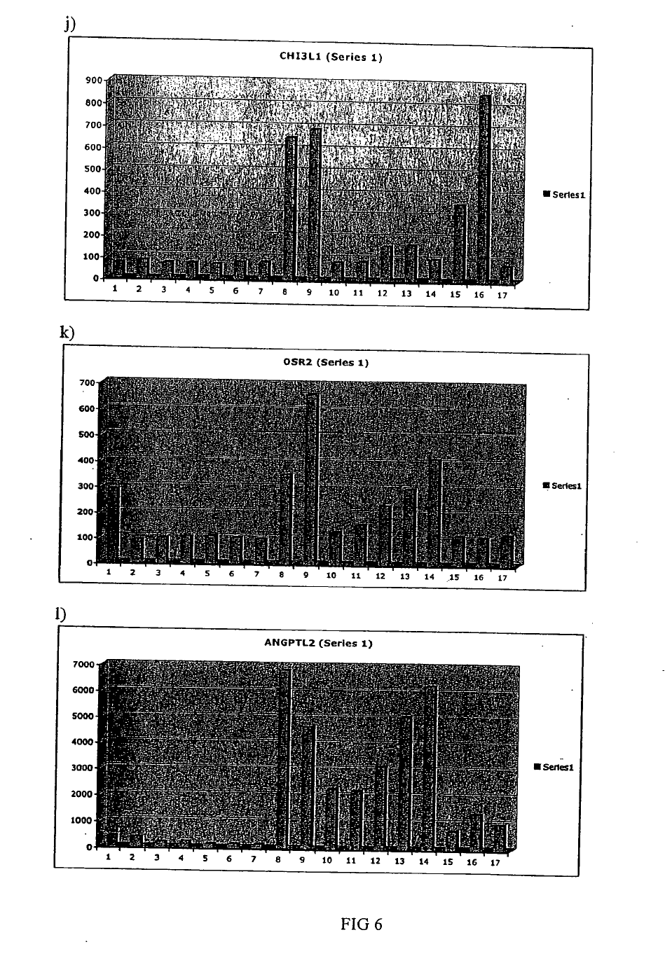

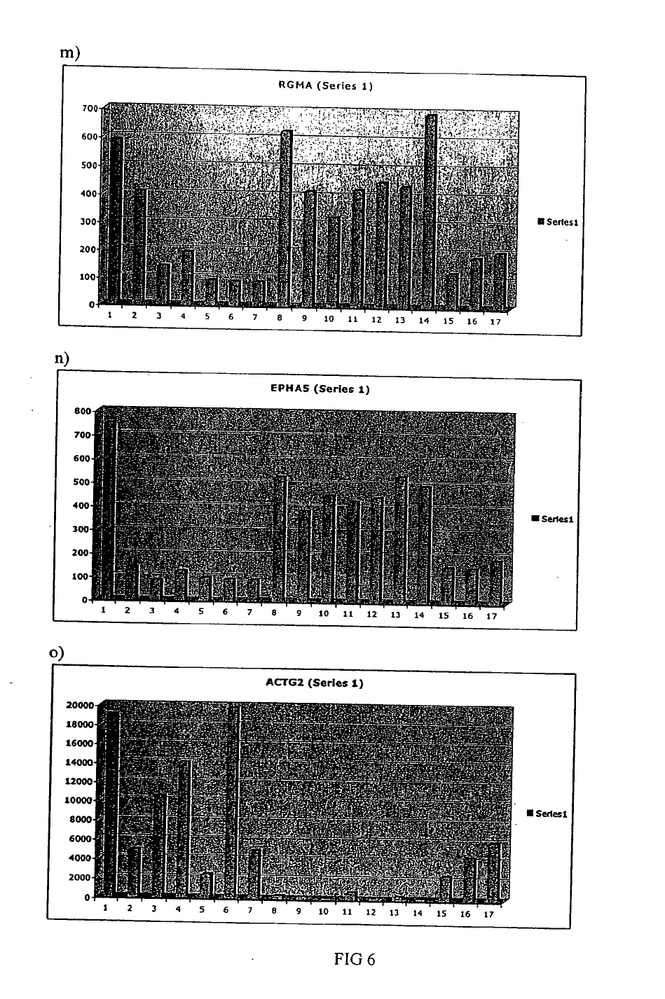

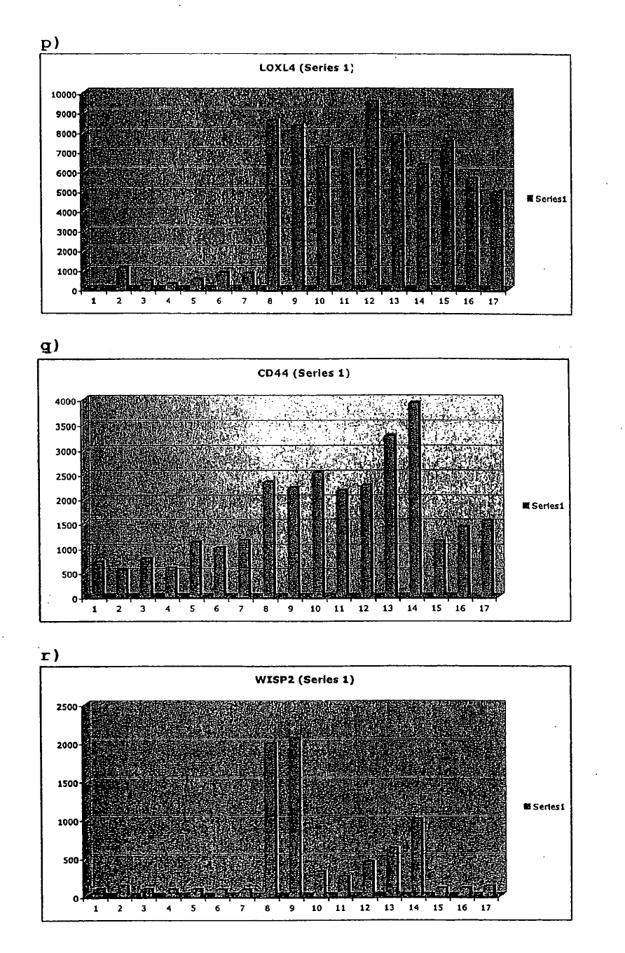

Of the first 17 colonies for which gene expression analysis was performed, clone 8 (B2 or ACTC51) of Series 1 displayed a pattern of gene expression consistent with dermal fibroblast progenitors with its expression of dermo-1 (TWIST2), dermatopontin (DPT), PRRX2 (which is a marker of fetal scarless wound repair (J Invest Dermatol 111(1):57-63 1998)), PEDF (SERPINF1), AKR1C1, collagen VI/alpha 3 (COL6A3), microfibril-associated glycoprotein 2 (MAGP2), which is a component of elastin-associated microfibrils, a component associated with elastogenesis Fibulin-1 (FBLN1). In developing prenatal skin, the MAGP2 protein is detected in the deep dermis and around hair follicles. The expression of MAGP2 has been reported to be up to six-fold higher in the prenatal state than postnatal and its expression precedes elastin synthesis in development (Gibson et al., J. Histochem. Cytochem. 46(8): 871-886 (1998)), GLUT5, WISP2, CHI3L1, Odd-Skipped Related 2 (OSR2), angiopoietin-like 2 (ANGPTL2), RGMA, EPHA5, the receptor for hyaluronic acid which promotes scarless wound repair (CD44), and a relative lack of the smooth muscle actins of a myofibroblast such as Actin Gamma 2 (ACTG2) (see FIG. 6).

In developing prenatal skin, the MAGP2 protein is detected in the deep dermis and around hair follicles. The expression of MAGP2 has been reported to be up to six-fold higher in the prenatal state than postnatal and its expression precedes elastin synthesis in development (Gibson et al., 1998).

Markers that uniquely identify dermal progenitors from this region of the developing dermis include the positive expression of TWIST2, DPT, PRRX2, MAGP2, and WISP2 at levels comparable to ADPRT as shown in FIG. 6, and the relative lack of expression of ACTG2 in relation to ADPRT as shown. A phase contrast photograph of the dermal fibroblast progenitors is shown in FIG. 5. All levels of gene expression were compared to the internal reference expression of the housekeeping ADPRT gene.

The relatively abundant expression of EPHA5 and RGMA in these dermal progenitors promote neuronal outgrowth and innervation of the forming tissues, are therefore useful in regenerating skin while promoting the innervation of the skin graft with sensory neurons and is an example of genes not expressed at comparable levels postnatally. The relatively abundant expression of angiopoietin-like2 (ANGPTL2) is another example of dermal cells with a prenatal pattern of gene expression, able to promote vascularization.

### Example 18

According to the methods described in Example 17, a number of other genes that are normally expressed more broadly in the embryo than postnatally were observed to be expressed by the clonogenic cells derived in this invention.

The following markers were uniquely expressed in our other cell lines that are normally expressed more broadly in the embryo than postnatally:

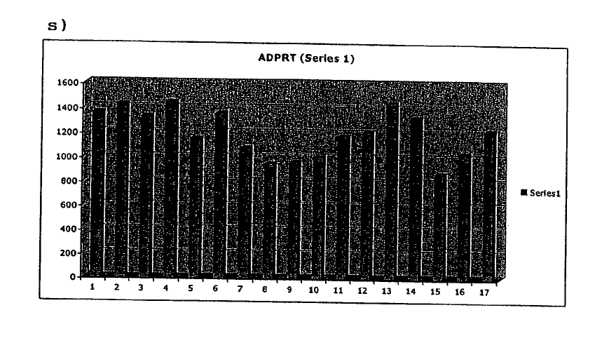

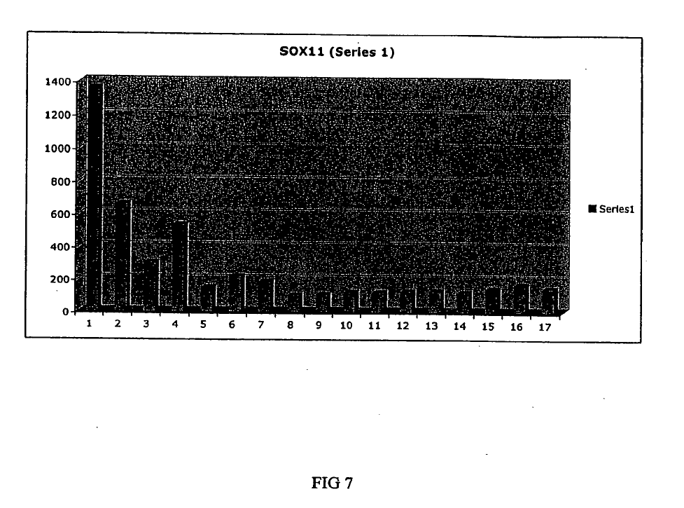

The SOX11 gene was expressed by the cells derived from clone 1 (B30 or ACTC61) of Series 1 (see FIG. 7 and Example 17). SOX11 is a gene which is largely expressed only in the CNS in adults, but has also been reported to be expressed in other places in the embryo, including the neural crest, mammary anlagen, ear fold, nose, and limb buds.

Some complement components, such as C3, MASP1, carboxypeptidases such as CPE and CPZ, like Furin activate prohormones and other proteins in early embryogenesis, but in the later fetal and adult stages of development, these complement components and other embryonic proteases are largely used only for the complement cascade or digestion. CPE (carboxypeptidase E) is a prohormone convertase like furin and is primarily CNS, neural crest, and expressed in the embryonic ribs, ganglia, in first branchial arch, embryonic heart, cartilage, primordial cells of cephalic bones, developing vertebral bodies, dorsal surface of tongue, and olfactory epithelium.

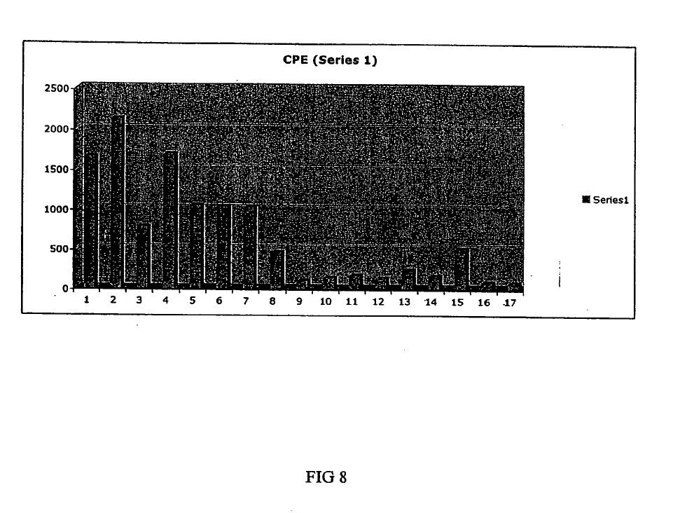

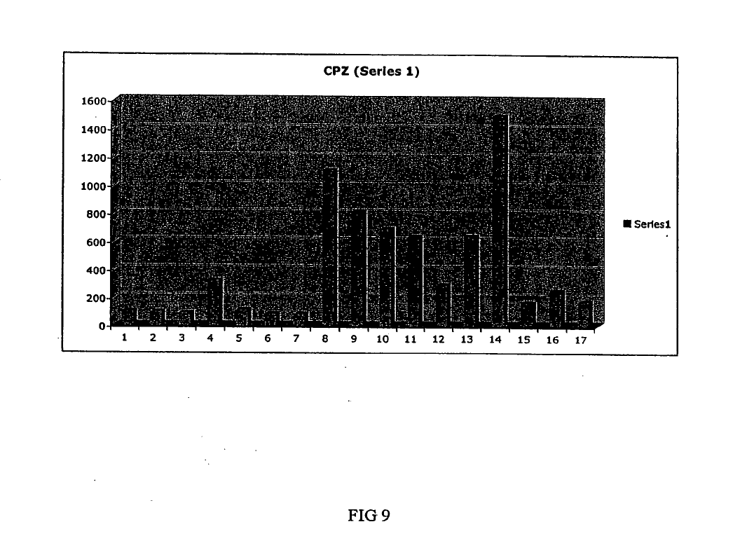

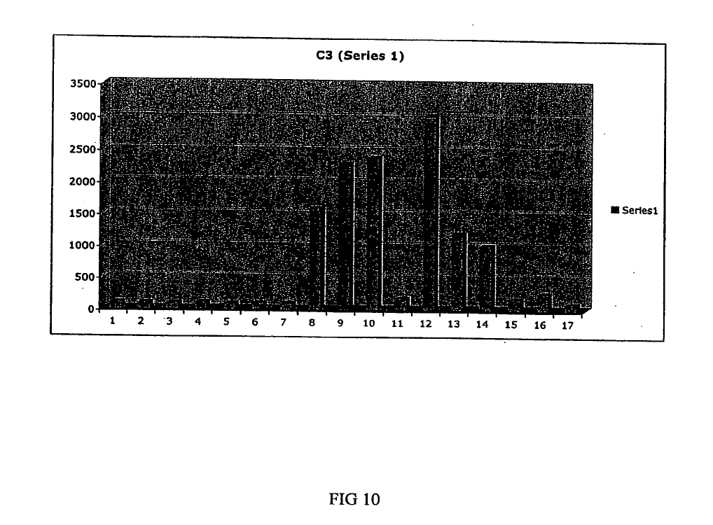

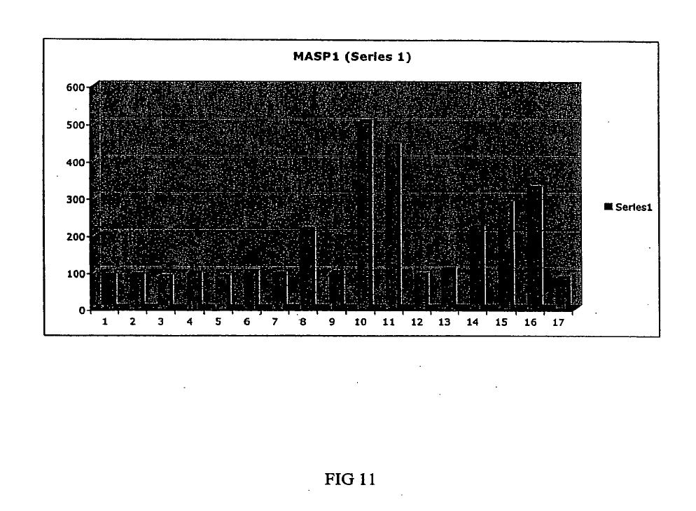

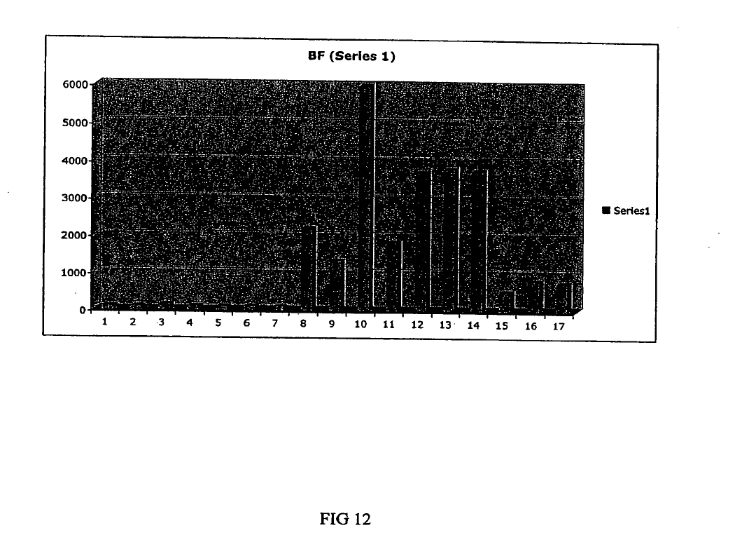

Examples of cells displaying this embryonic pattern of complement proteases and thereby capable of inducing tissue generation and regeneration were observed. The CPE gene was expressed by the cells derived from clones 1 (B30 or ACTC61), 2 (317 or ACTC54), 4 (B6 or ACTC56), 5 (4-1), 6 (4-3) and 7 (B-10) of Series 1 (see FIG. 8). The CPZ gene was expressed by the cells derived from clones 8 (B2 or ACTC51), 9 (B7 or ACTC53), 10 (B25 or ACTC57), 11 (B11 or ACTC58), 13 (326 or ACTC50) and 14 (6-1 or ACTC64) of Series 1 (see FIG. 9). The C3 gene was expressed by the cells derived from clones 8 (B2 or ACTC51), 9 (B7 or ACTC53), 10 (B25 or ACTC57) and 12 (B3 or ACTC55) of Series 1 (see FIG. 10). The MASP1 gene was expressed by the cells derived from clones 8 (B2 or ACTC51), 10 (B25 or ACTC57), 11 (B11 or ACTC58), 14 (6-1 or ACTC64), 15 (2-2 or ACTC62) and 16 (2-1 or ACTC63) of Series 1 (see FIG. 11). Finally, the BF gene was expressed by the cells derived from clones 10 (B25 or ACTC57), 12 (B3 or ACTC55), 13 (B26 or ACTC50) and 14 (6-1 or ACTC64) of Series 1 (see FIG. 12).

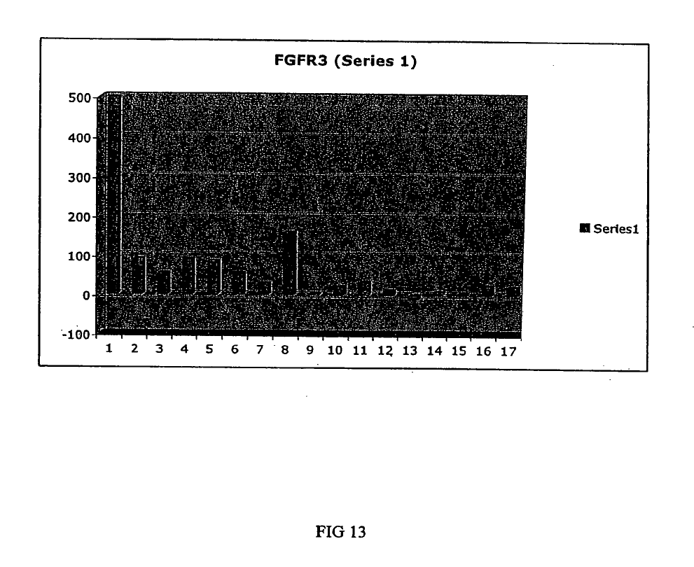

The FGFR3 (FGF Receptor 3) gene was expressed by the cells derived from clone 1 (B30 or ACTC61) of Series 1 (see FIG. 13). The FGFR3 (FGF Receptor 3) gene is expressed primarily in the CNS but also in other tissues during embryogenesis.

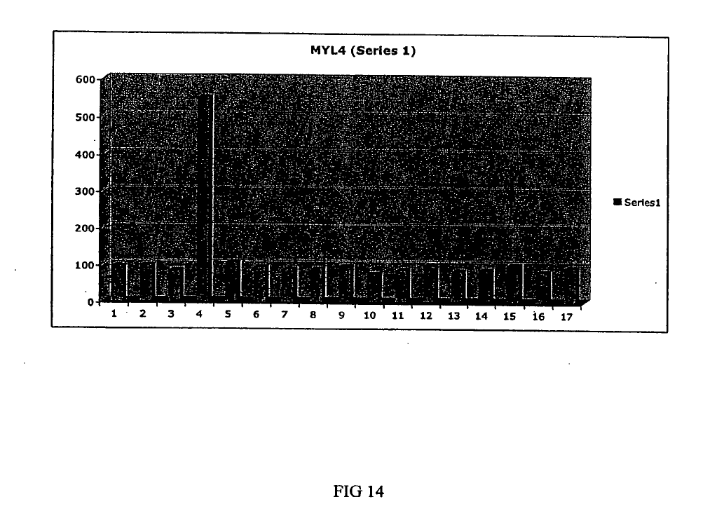

The MYL4 (myosin light chain 1) gene was also specifically expressed by the cells derived from clone (B6 or ACTC56) of Series 1 (see FIG. 14). MYL4 is an atrial/fetal isoform of the protein, indicating a muscle precursor of the first branchial arch that may be useful in research and for regenerating muscles of the derivatives of the first branchial arch such as muscles of the mandible.

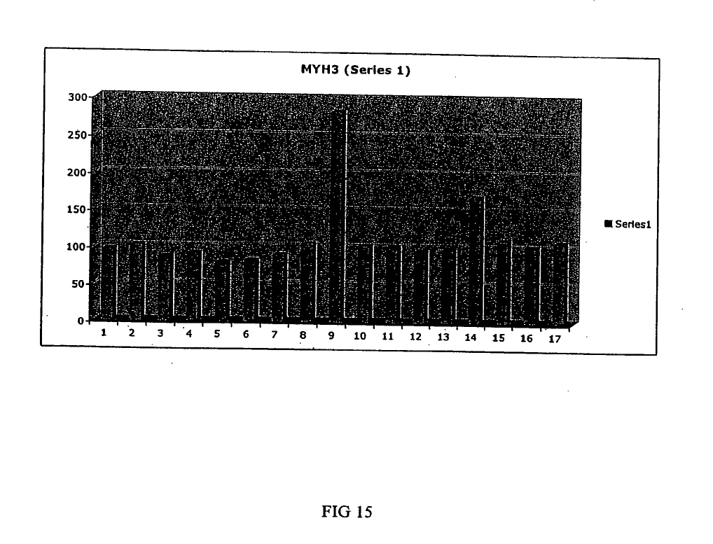

The MYH3 (myosin heavy chain polypeptide 3) gene was expressed by the cells derived from clone 9 (B7 or ACTC53) of Series 1 (see FIG. 15). Since the MYH2 gene is normally expressed in embryonic skeletal muscle, the overexpression of this gene by the cells derived from clone 9 suggests that these cells may be embryonic muscle precursor cells.

### Example 19

One of the important aspects of the clonogenic differentiated cell lines generated according to the methods of this invention is the observation that the original cell can be photodocumented not to have the morphology of an ES cell, and the resulting colony and subsequent cultures have vanishingly small likelihood of harboring undifferentiated ES cells. Since hES cells can only grow as colonies and as such, have unique and easily-recognized morphology as well as requiring special growth conditions, the likelihood for hES cells existing within the clonogenic differentiated cell lines is highly unlikely.

Since the characterization of cell formulations for therapy will require extensive documentation that the formulation does not include ES cells, the clonogenic differentiated cell lines with reduced or no contaminating ES cells can be used to determine the threshold concentrations of contaminating ES (or EC) cells tolerable in hES-based therapeutics.

A gradient of doses of hES cells (which lead to benign teratomas) and human EC (hEC) cells (EC being a malignant version of ES called teratocarcinoma cells) will be transplanted into SCID mice. The amount of hES and hEC cells will be transplanted at a gradient dose, with smaller and smaller doses of the ES and EC cells transplanted with the clonogenic differentiated cells generated according to the methods of this invention, until at the end of the gradient spectrum, only the clonogenic differentiated cells are being administered.

First, for the transplantation of hES, two SCID mice will be injected with 3×10⁶ hES cells (GFP-H1) in one leg quadricep muscle. The animals will be sacrificed after 60 days and histology will be performed on teratoma. The human cells can be identified by means of fluorescence and antibodies directed to human Class I HLA.

Second, for the transplantation of hES-derived clonogenic cells, two SCID mice will be transplanted with 3×10⁶ cells obtained from Example 13 or Example 17. The animals will then be sacrificed after 60 days and histology will be performed on teratoma, identifying human cells by means of fluorescence and antibody to human Class I HLA.

Finally, a gradient of doses of hES or hEC will be mixed with the clonogenic differentiated cells generated by the present invention at 0.01%, 0.1%, 1%, and 10% of the total cell number. The sensitivity of the assay to detect ES cells will be determined in the mass of tissue. Evidence of benign or malignant growth or metastasis will be determined.

Furthermore, the clonogenic differentiated cell lines can be mixed with GFP hES to allow visualization of the interaction of the cells with differentiating cells and tissues in a teratoma, thereby giving more insight into the nature and uses of the differentiated cell lines.

### Example 20

### Whole Body Imaging of Human Embryonic Stem Cells and Differentiated Progeny Cells in Mice

The locations and migration of human embryonic stem cells, and their differentiated progeny, in mouse tissues and cavities are identified by whole body imaging of mice injected with genetically modified hES cells, or their differentiated progeny, by technologies well know to those versed in the art. In this approach, cells that are genetically modified to express reporter genes are introduced into mice by injection directly into the target tissue, or introduced by intravenous or intraperitoneal injection. Cells may be genetically modified with a transgene encoding the Green Fluorescent protein (Yang, M. et al. (2000) Proc. Natl. Acad. Sci. USA, 97:1206-1211), or one of its derivatives, or modified with a transgene constructed from the Firefly (Photinus pyralis) luciferase gene (Fluc) (Sweeney, T. J. et al. (1999) Proc. Natl. Acad. Sci. USA, 96: 12044-12049), or with a transgene constructed from the Sea Pansey (Renilla reniformis) luciferase gene (Rluc) (Bhaumik, S., and Ghambhir, S. S. (2002) Proc. Natl. Acad. Sci. USA, 99:377-382). The reporter transgenes may be constitutively expressed using a “house-keeping gene” promoter such that the reporter genes are expressed in many or all cells at a high level, or the reporter transgenes may be expressed using a tissue specific or developmental stage specific gene promoter such that only cells that have located into particular niches and developed into specific tissues or cell types may be visualized.

Creation of Luciferase or GFP Expressing Clonogenic Cell Lines

Human ES cells or their differentiated progeny are first genetically modified with expression vectors containing reporter genes encoding the Firelfly luciferase gene (FLuc), Renilla luciferase gene (RLuc), or green fluorescence protein (GFP), or similar fluorescence proteins. These reporter gene vectors are available from commercial vendors as plasmid or retroviral vectors ready-for-use, or are engineered as proprietary expression vectors. There are several advantages to engineering proprietary reporter vectors for the applications described herein: tissue specific or developmental stage-specific promoters can be used to mark and identify specific classes or types of differentiated cells in vitro and in vivo; choice of plasmid or viral vector allows optimizing delivery of the reporter vector to cells; and construction of vectors with proprietary reporter genes not commercially available.

In this example, we describe the procedure for generating hES cells, or their differentiated progeny, including the dermal progenitor cells ACTC 59 (B2), containing the pFB-Luc retroviral vector (Stratagene, La Jolla, Calif.) stably integrated into the cellular genomic DNA. Luciferase levels and cell transduction efficiencies are determined by measuring luciferase activity in lysates of virus infected cells, by immunocytochemically staining cells for Luciferase expression, and by direct detection of luminescent cells in culture.

Transduction of Target Cells with a Viral Supernatant.

This transduction is performed to demonstrate that cell lines are able to be transduced, that the viral supernatants are able to be transduced, and to assess the quality of the viral supernatants.

Day 1: Preparing for Transduction

1. For both NIH3T3 positive control cells and target cells, including the dermal progenitor cells ACTC 59 (B2), seed 6 wells using 6-well tissue culture plates with 1×105 cells per well. This seeding density may vary with the target cell line; ˜20% confluency at the time of infection is desirable.

2. Return the plates to the 37° C. incubator overnight.

Day 2: Transducing the Target Cells

Prior to thawing the viral supernatant, the area around the cap should be carefully inspected for any sign of leakage, and thoroughly wiped with 70% ethanol. Media should be prepared and aliquoted into prelabeled Falcon® 2054 polystyrene tubes prior to thawing the virus.

1. Quickly thaw the pFB-Luc supernatant (nominal titer approximately 2×10⁷/ml) by rapid agitation in a 37° C. H2O bath. Screw caps should be removed in the hood only, and any fluid around the outside lip of the tube or the inside surface of the cap should be carefully wiped with a tissue wetted with 70% ethanol, and the tissue should be disposed of in the hood. Thawed virus should be temporarily stored on ice if not used immediately.

2. Prepare a dilution series from 1:10 to 1:10⁴ in growth medium (2.0 ml dilution per tube in 2054 tubes) supplemented with DEAE-dextran at a final concentration of 10 μg/ml (1:1000 dilution of the 10 mg/ml DEAE-dextran stock). Add 0.8-1.0 ml undiluted supernatant to an additional tube, and supplement with DEAE-dextran to 10 μg/ml.

3. Remove the plates containing the target cells (NIH3T3 cells and target cells) from the incubator.

4. Remove and discard the medium from the wells. For tubes containing undiluted supernatant and for each dilution, add 1.0 ml per well to both the NIH3T3 and target cell. Add 1.0 ml media (no virus) to the sixth well for an uninfected control. The remaining supernatant should be aliquoted and refrozen at −80° C. It should be noted that the titer will drop, resulting in a loss of <50% of the remaining infectious particles with each subsequent freeze-thaw cycle.

5. Return the plates to the 37° C. incubator and incubate for 3 hours.

6. After the 3 hour incubation, add an additional 1.0 ml growth medium to each well.

7. Return the plates to the 37° C. incubator and allow 24-72 hours for analysis of expression of the luciferase protein by luciferase assay, immunocytochemistry, or direct visualization of luminescent cells.

Luciferase Assay.

Transduction efficiencies of cells are determined by assaying lysates of virus infected cells for luciferase production. Luciferase may be assayed using commercially available kits. In this example, we describe measuring luciferase production using a Luciferase assay kit from Stratagene (La Jolla, Calif.).

Extracting Luciferase from Tissue Culture Cells.

The cell lysis buffer is designed to extract luciferase from mammalian tissue culture cells that are transfected with the luciferase reporter gene. The inclusion of 1% Triton® X-100 in the cell lysis buffer allows the direct lysis of many types of tissue culture cells, such as HeLa cells and fibroblasts. The quantities of the reagents given in this protocol are optimized for a 35-mm tissue culture plate having ˜9.4 cm2 of surface area in each well. The volume of the cell lysis buffer may be adjusted for tissue culture plates of other sizes.

1. Being careful not to dislodge any of the cells, remove the media from the tissue culture plate wells and wash the cells twice with 1×PBS.

2. Using a Pasteur pipet, remove as much PBS as possible from each well.

3. Make 1× cell lysis buffer (25 mM Tris-phosphate (pH 7.8), 2 mM DTT, 2 mM 1,2-diaminocyclohexane-N,N,N′,N′-tetraacetic acid, 10% glycerol, 1% Triton® X-100) by adding 4 milliliters of dH2O per milliliter of the 5× cell lysis buffer. Equilibrate the lysis buffer to room temperature before use.

4. Cover the cells by adding approximately 200-500 μl of 1× cell lysis buffer to each well.

5. Incubate the plate at room temperature for 15 minutes, swirling occasionally.

6. Scrape the cells and buffer from each well into separate microcentrifuge tubes. Place the tubes on ice.

7. Vortex the microcentrifuge tubes for 10-15 seconds. Spin the tubes in a microcentrifuge at 12,000×g for 15 seconds at room temperature or 2 minutes at 4° C.

8. Transfer the supernatant from each tube to a new microcentrifuge tube.

9. Immediately assay the supernatant for luciferase activity according to the protocol provided below or store the supernatant at −80° C. for later use.

It should be noted that each freeze-thaw cycle results in a significant loss of luciferase activity (as much as 50%).

Performing Luciferase Activity Assay.

The following protocol is based on a single-tube luminometer. Luminometers capable of assaying multiwell plates (e.g., 96-well plates) and sophisticated computer software to process large numbers of samples are also commercially available. Although both scintillation counters and photographic film can be used to detect the light emission, they are not as sensitive.

1. Prepare the luciferase substrate-assay buffer mixture by adding all of the assay buffer (10 ml) to the vial containing the lyophilized luciferase substrate and mixing well.

2. Divide the luciferase substrate-assay buffer mixture into aliquots of an appropriate size to avoid multiple freeze-thaw cycles. The luciferase substrate-assay buffer mixture is best if used within one month when stored at −20° C. or within one year when stored at −70° C. Avoid unnecessary freeze-thaw cycles. Protect the luciferase substrate-assay buffer mixture from light.

3. Allow the luciferase substrate-assay buffer mixture to reach room temperature. Allow the supernatant from step 9 in Extracting Luciferase from Tissue Culture Cells to reach room temperature.

4. Add 100 μl of the luciferase substrate-assay buffer mixture to a polystyrene tube that fits in the luminometer (e.g., a 5-ml BD Falcon polystyrene round bottom tube).

5. Add 5-20 μl of supernatant to the tube, mix gently, and immediately put the tube into the luminometer.

6. Begin measuring the light produced from the reaction ˜8 seconds after adding the supernatant using an integration time of 5-30 seconds.

Immunocytochemistry for Cells Expressing Luciferase.

An aliquot of viral transduced cells are cultured for 3 days after which cells were harvested and prepared on cytospin slides. Slides are stained with monoclonal antiluciferase antibody (Novus, Littleton, Colo.) 1:100 for 1 hour, followed by donkey polyclonal antibody to mouse IgG-FITC (Novus) 1:100 for 30 minutes. The slides are mounted with Vectashield medium with DAPI (4′,6-diamidino-2-phenylindole; Vector Laboratory, Burlingame, Calif.). Cultured nontransduced cells are used as negative controls.

Direct Imaging of Luciferase Expressing Cells.

Optimal conditions for DNA delivery are identified by adding luciferin (0.5 mg/ml final; Molecular Probes) to the cell culture medium and light emission is used to confirm expression of the reporter gene. Cultures are screened by using an intensified charge-coupled device camera (C2400-32, Hamamatsu Photonics, Hamamatsu City, Japan). Colonies of cells expressing light are expanded for xenotransplantation into mice.

Xenotransplantation of Cells into Mice.

Mice are anesthetized by i.p. injection of approximately 40 μl of a ketamine and xylazine (4:1) solutions and injected with approximately 3×10⁶ Luciferase expressing cells in 100 μl of PBS directly into the peritoneal cavity or injected via tail-vein. Injected mice are allowed to recover, maintained in a controlled environment and monitored weekly for 8 weeks to track the migration and final destination of Luciferase expressing cells using Xenogen IVIS Imaging System 3D Series bioluminescence imagers. Luciferase expressing ACTC59(B2) dermal progenitor cells are injected intradermally at doses of 1×10³, 1×10⁴, 1×10⁵, and 1×10⁶ cells in three animals over 4 injections per animal and engraftment and migration of the cells are tracked over three months using Xenogen IVIS Imaging System 3D Series bioluminescence imagers.

Whole Body Imaging of Luc-Marked Cells Injected in Mice.

Imaging of mice containing cells expressing Fluc reporter genes requires injection of mice with the cofactor Luciferin for light production and anesthetization prior to imaging. Mice are injected by an intraperitoneal route into the animal's lower left abdominal quadrant using 1 cc syringe fitted with a 25 gauge needle with a luciferin solution (15 mg/ml or 30 mg/kg, in PBS, dose of 150 mg/kg; D-Luciferin, Firefly, potassium salt, 1.0 g/vial, Xenogen Catalog #XR-1001) that is allowed to distribute in awake animals for about 5-15 minutes. The mice are placed into a clear plexiglass anesthesia box (2.5-3.5% isofluorane) that allows unimpeded visual monitoring of the animals; e.g. one can easily determine if the animals are breathing. The tube that supplies the anesthesia to the box is split so that the same concentration of anesthesia is plumbed to the anesthesia manifold located inside the imaging chamber. After the mice are fully anesthetized, they are transferred from the box to the nose cones attached to the manifold in the imaging chamber of a Xenogen IVIS Imaging System 3D Series imager, the door is closed, and the “Acquire” button (part of the Xenogen Living Image program) on the computer screen is activated. The imaging time is between one to five minutes per side (dorsal/ventral), depending on the experiment. When the mice are turned from dorsal to ventral (or vice versa), they can be visibly observed for any signs of distress or changes in vitality. The mice are again imaged (maximum five minutes), and the procedure is complete. The mice are returned to their cages where they awake quickly.

Alternatively, for mice containing cells expressing the RLuc reporter genes, an aqueous solution of the substrate coelenterazine (Biotium; 3.5 mg/kg) is injected via tail vein 10 minutes before imaging. The animals are then placed in a light-tight chamber, and a gray-scale body-surface reference image is collected with the chamber door slightly open. For this purpose, a low-light imaging system, comprised of an intensified charge-coupled device camera fitted with a 50-mm f1.2 Nikkor lens (Nikon) and a computer with image-analysis capabilities, is used. Subsequently, the door to the chamber is closed to exclude the room light that obscures the relatively dimmer luciferase bioluminescence. Photons emitted from luciferase within the animal and then transmitted through the tissue are collected and integrated for a period of 5 min. A pseudocolor image representing light intensity (blue least intense and red most intense) is generated on an Argus 20 image processor (Hamamatsu); images are transferred by using a plug-in module (Hamamatsu) to a computer (Macintosh 8100/100) running an image processing application (PHOTOSHOP, Adobe Systems, Mountain View, Calif.). Gray-scale reference images and pseudocolor images are superimposed by using the image-processing software, and annotations are added by using another graphics software package (CANVAS, version 5.0, Deneba, Miami, Fla.).

In whole body imaging approaches using GFP, and derivative, proteins, mice are anesthetized with pentobarbital (70 mg/kg body weight) placed in a warmed light box or directly on the microscope stage. A Leica fluorescence stereo microscope, model LZ12, equipped with a 50-W mercury lamp, is used for high-magnification imaging. Selective excitation of GFP is produced through a D425y60 band-pass filter and 470 DCXR dichroic mirror. Emitted fluorescence is collected through a long-pass filter GG475 (Chroma Technology, Brattleboro, Vt.) on a Hamamatsu C5810 3-chip cooled color charge-coupled device camera (Hamamatsu Photonics Systems, Bridgewater, N.J.). Images are processed for contrast and brightness and analyzed with the use of IMAGE PRO PLUS 3.1 software (Media Cybernetics, Silver Springs, Md.). Images of 1,024 3 724 pixels are captured directly on an IBM PC or continuously through video output on a high-resolution Sony VCR model SLV-R1000 (Sony, Tokyo). Imaging at lower magnification that visualizes the entire animal is carried out in a light box illuminated by blue light fiber optics (Lightools Research, Encinitas, Calif.) and imaged by using the thermoelectrically cooled color charge-coupled device camera, as described above.

### Example 21 hES-Derived Smooth Muscle Progenitors

Colonies from the hES cell line ACTS were differentiated using in situ colony differentiation by the removal of LIF-containing medium and the addition of DMEM medium containing 10% FBS. After various periods of time (5, 7, and 9 days of exposure to differentiation medium), the cells were trypsinized, and plated onto 15 cm plates coated with the extracellular matrix protein collagen, and cultured for an additional 20 days to further induce differentiation. The cells were then trypsinized and counted with a Coulter counter, and the cells were plated at increasing dilutions with a volume containing 2,500 cells, 5,000 cells and 25,000 cells introduced into the 15 cm tissue culture plates and subsequently incubated in 5% ambient oxygen undisturbed for two weeks.

Clonal colonies were identified by phase contrast microscopy and those that are uniformly circular and well separated from surrounding colonies were marked for removal using cloning cylinders. The trypsinized cells from within each cloning cylinder were then replated into collagen coated 24 well plates and incubated. Of 61 colonies isolated, 29 grew at a relatively rapid rate of approximately one doubling a day. The cells were karyotyped and determined to be normal human. A total genomic expression analysis using the Illumina system was performed on the cells.

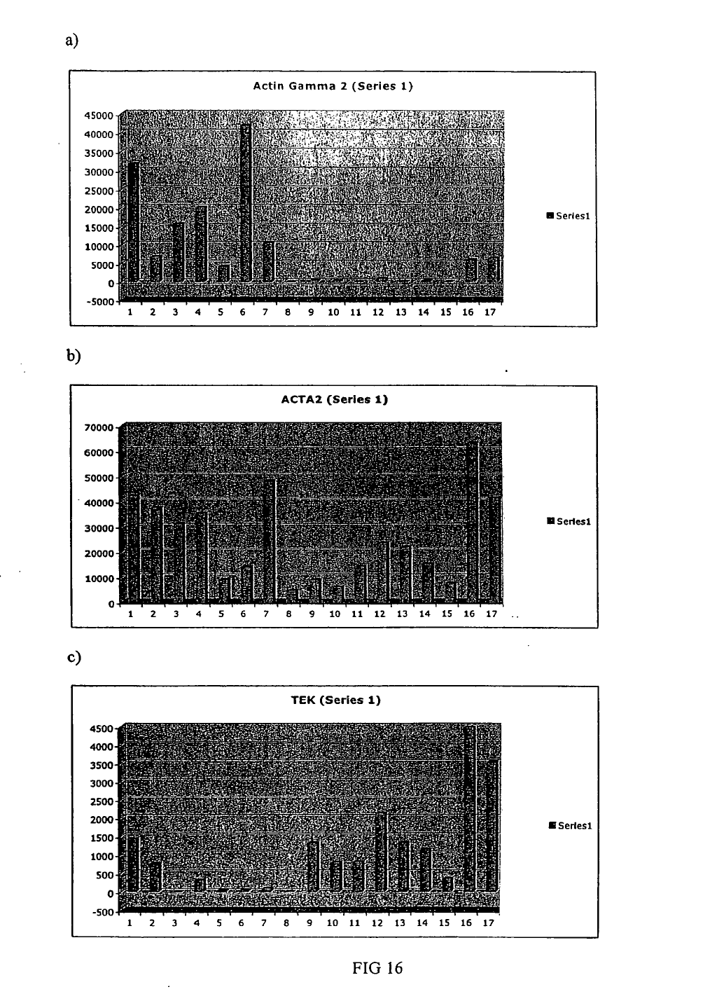

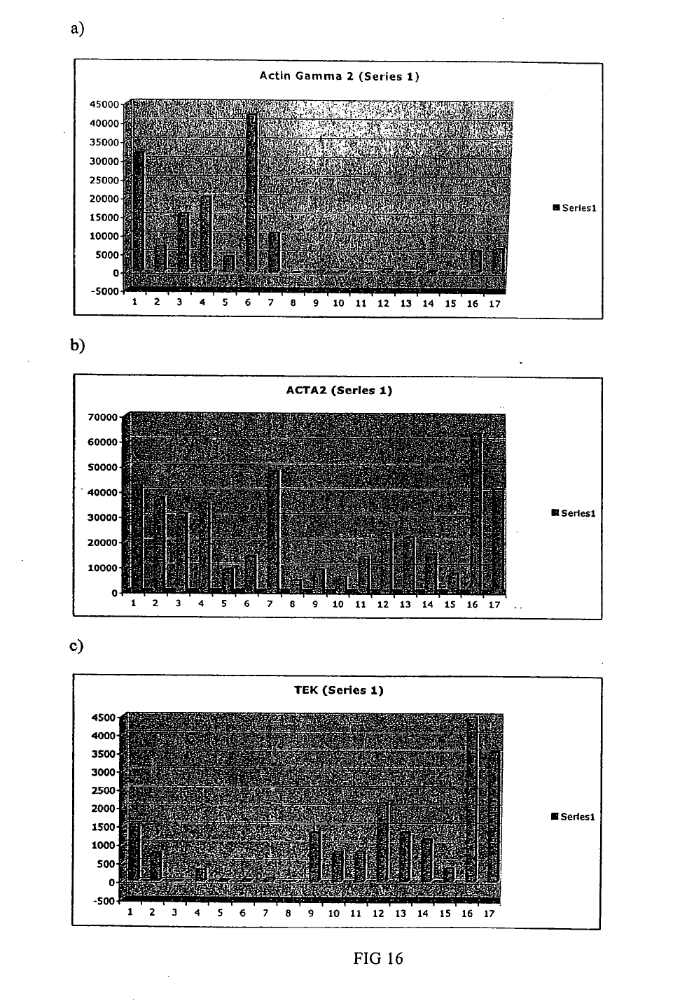

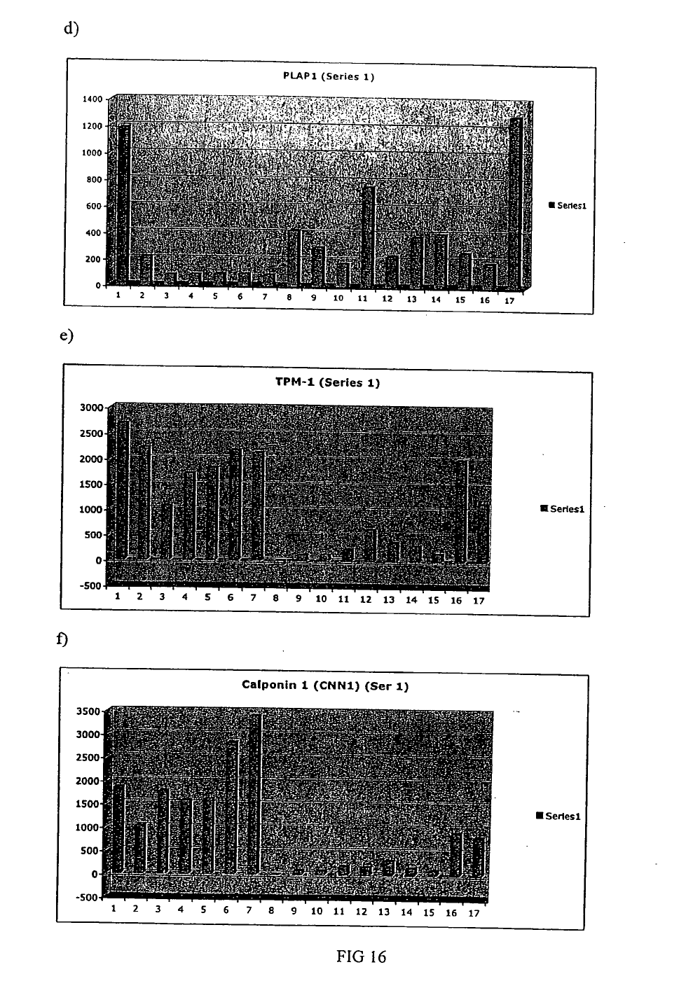

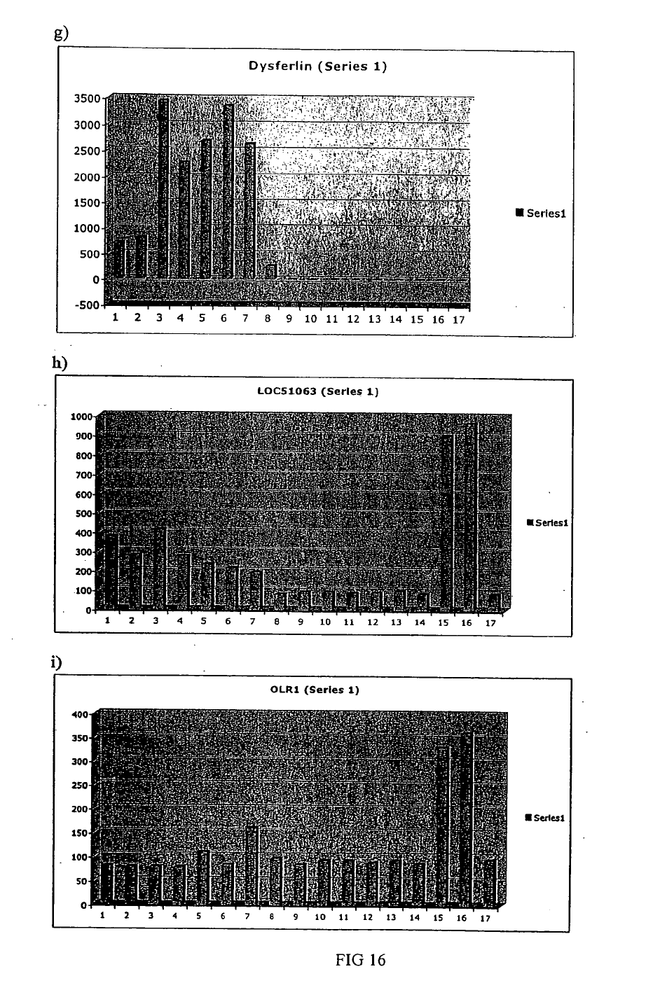

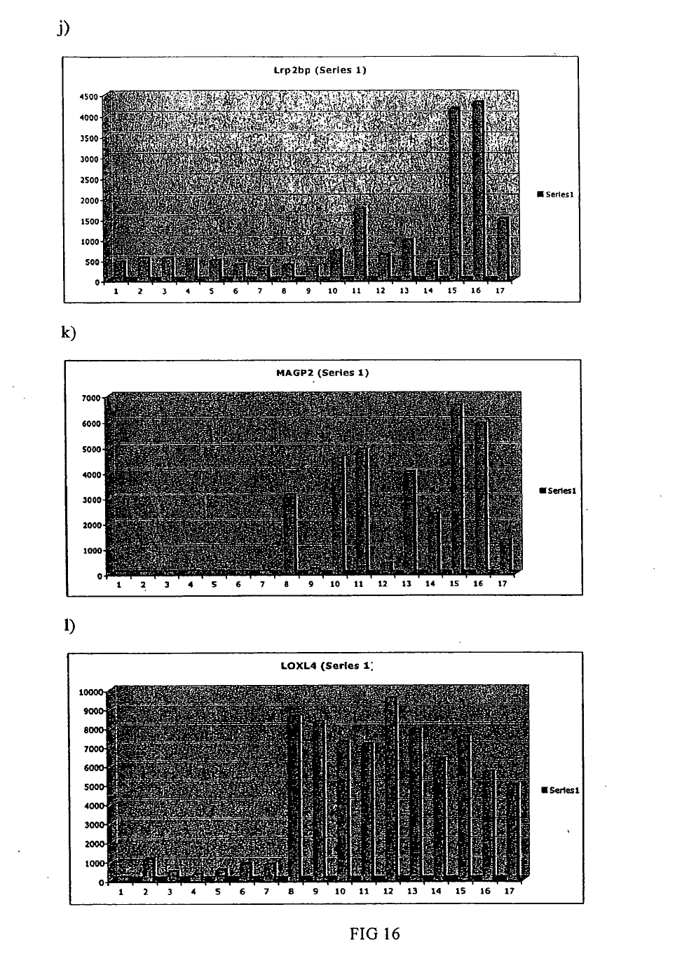

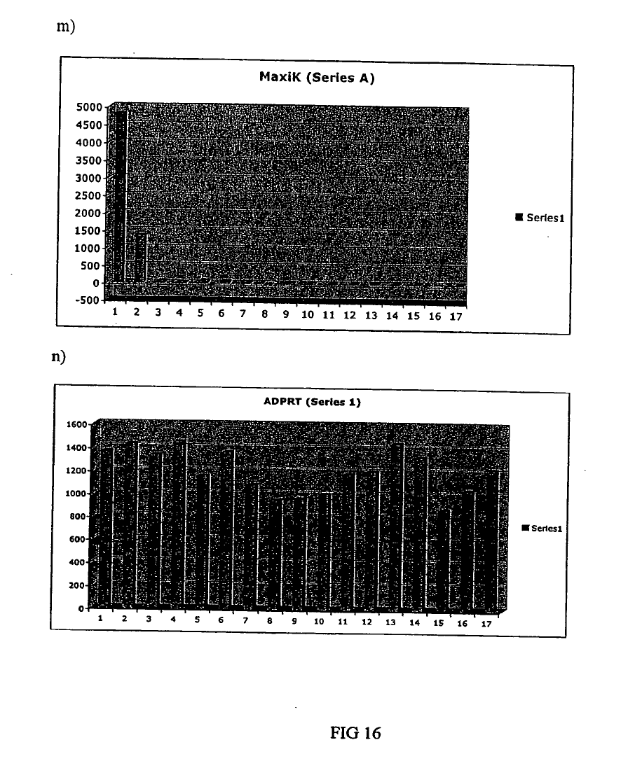

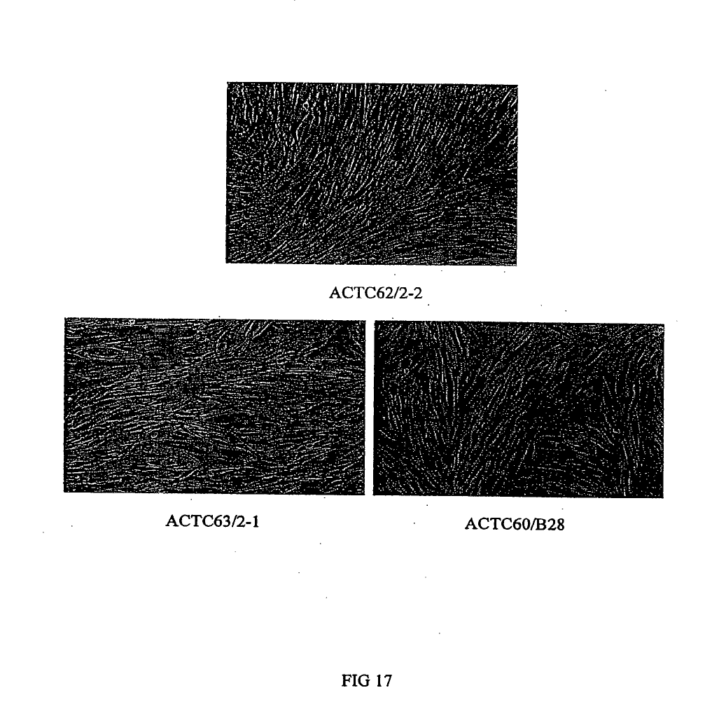

Clones 15 (2-2 or ACTC62), 16 (2-1 or ACTC63) and 17 (B28 or ACTC60) of Series 1 (see Example 17) displayed a pattern of gene expression consistent with smooth muscle progenitors and yet with numerous surprising genes being expressed with clones 15 and 16 of Series 1 displaying a pattern of large artery (aortic) vascular smooth muscle, and clone 17 of Series 1 showing a pattern of enteric smooth muscle in that the lines 15 and 16 expressed relatively high levels of expression of the smooth muscle actin gamma 2 (ACTAG2, Accession No. NM_001615.2, smooth muscle actin (ACTA2, Accession No. NM_001613.1), the endothelial receptor for angiopoietin-1 (TEK, Accession No. NM_000459.1), tropomyosin-1 (TPM-1, Accession No. NM_000366.4), calponin-1 (CNN1, Accession No. NM_001299.3), the unidentified gene L0051063, the oxidized low-density (lectin-like) receptor-1 (OLM1), LRP2 binding protein (Lrp2bp), MAGP2, LOXL4, and relatively low levels of expression of dysferlin, PLAP1, and MaxiK compared to the housekeeping gene ADPRT. The enteric smooth muscle clonogenic cell line 17 (also referred to as B-28 or ACTC60) showed markers for smooth muscle actin gamma 2, smooth muscle actin (ACTA2), the endothelial receptor for angiopoietin-1 (TEK), PLAP1, levels of tropomyosin-1 (TPM-1) comparable to fibroblast-like cells, calponin-1 (CNN1), LOXL4, MaxiK, and relatively low levels of expression of dysferlin, the unidentified gene L0051063, and OLR1, Lrp2bp compared to the housekeeping gene ADPRT. See FIG. 16. A phase contrast photograph of smooth muscle clonogenic cell lines is shown in FIG. 17.

The clonogenic cell line 17 of Series 1 (B-28 or ACTC60) (see Example 17) was deposited with the American Type Culture Collection (“ATCC”; P.O. Box 1549, Manassas, Va. 20108, USA) under the Budapest Treaty on Jun. 7, 2006, and have accession number ATCC PTA-7654. This cell line is an embryonic smooth muscle cell line with potential clinical application in heart disease, aneurysms and other age-related vascular disease, cancer, and intestinal disorders.

Large vascular smooth muscle cells with an embryonic (prenatal) pattern of gene expression with high levels of elastogenesis as shown herein have clinical utility in the treatment of vascular disease such as strengthening the arterial wall by direct injection, or by IV injection, allowing the cells to home to sites of vascular lesions such as atheromas or aneurysms. These cells could be modified to carry therapeutic transgenes to the sites of malignancy. These cells could be injected into cardiac or skeletal muscle to strengthen the muscle. Also, particular splicing isoforms of the OLR1 gene known in the art (Biocca et al, Circ. Res. 97(2): 152-158 (2005)) could be introduced to these cells and the cells could then be protective against myocardial infarction, or to be use in the engineering of tissued engineered vascular tissue. Enteric smooth muscle cells are useful in strengthening the wall of the intestine, improving contractility, or the tissue engineering of intestinal tissue.

### Example 22

### The Use of Hox Gene Expression to Identify Clonogenic Cell Lines Derived from Pluripotent Stem Cells Such as hES Cells

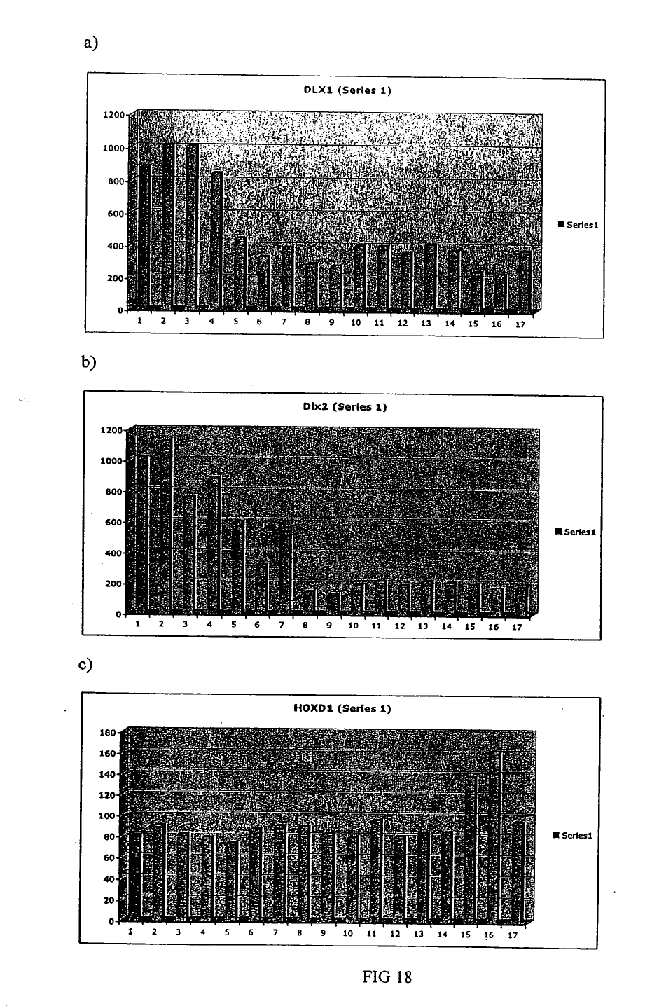

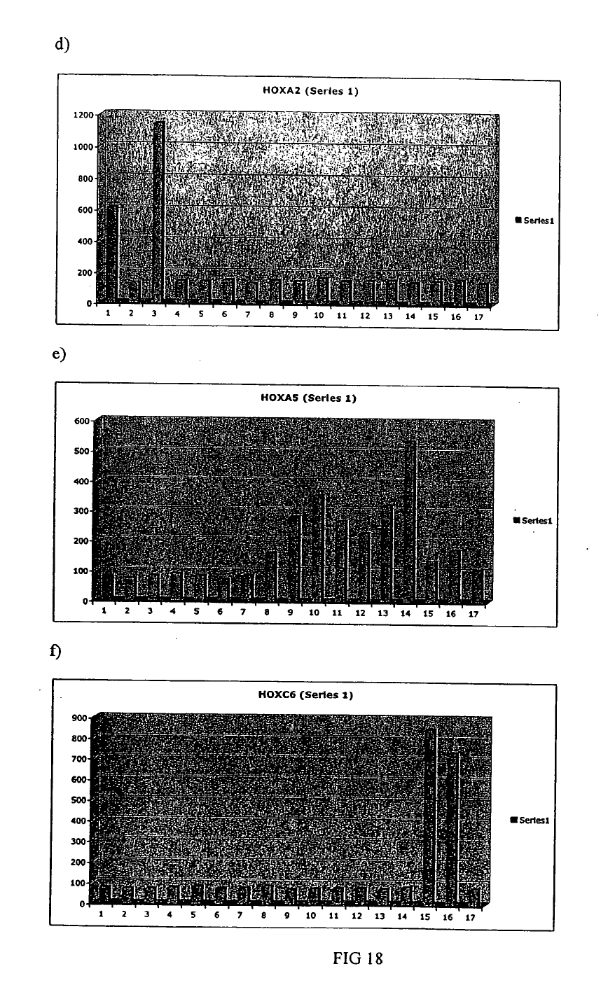

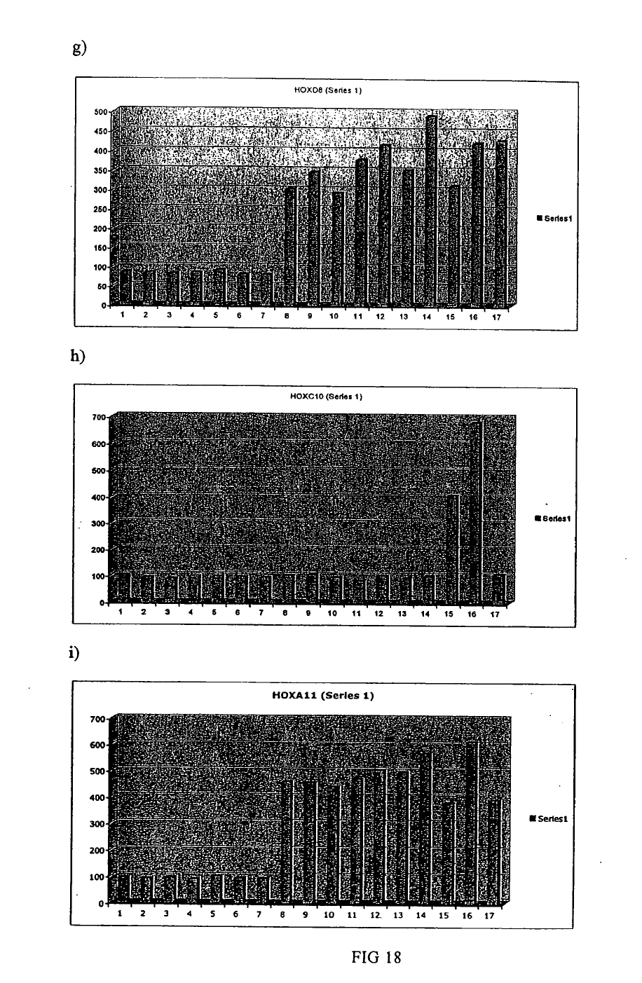

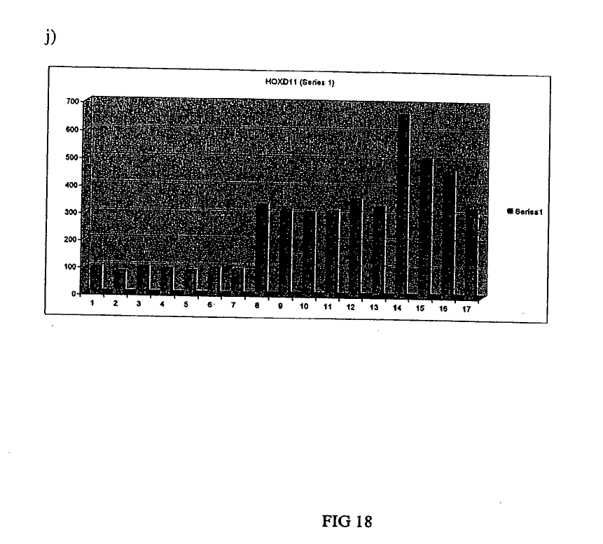

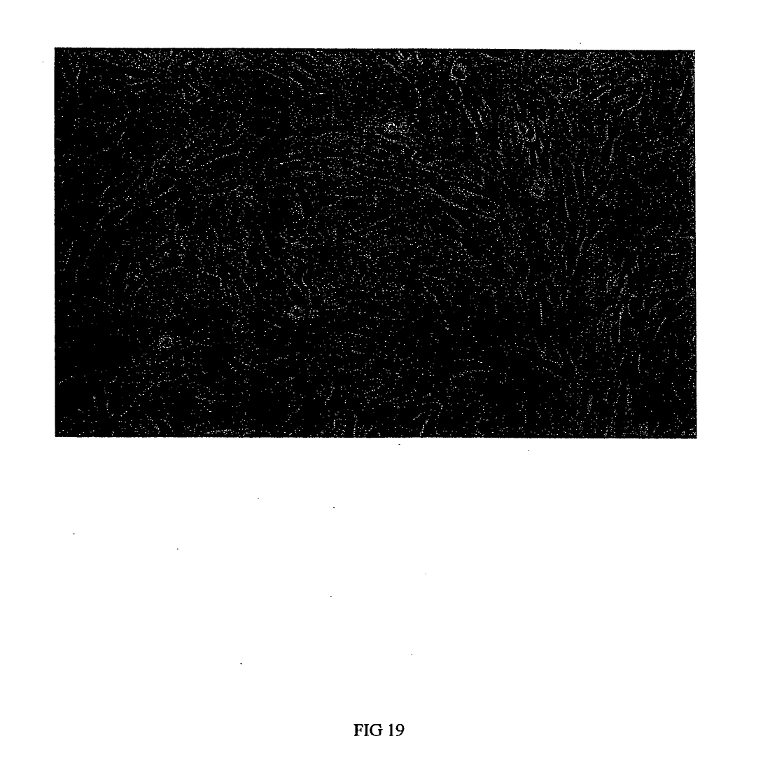

The expression of the Hox genes and other developmentally-regulated segmentation genes provide a useful marker of the origin of the clonogenic cell lines. This is generally not the case where the cells have a heterogeneous origin. By way of example, the cell clones described in example 17 above were compared for relative levels of genes such as the Hox genes and similar developmentally regulated segmentation genes. Those that displayed no expression are not shown. Shown in FIG. 18 are the expression of Dlx1, Dlx2. The expression of Dlx1 and Dlx2, but not Dlx3, Dlx5, Dlx6, or Dlx7, and the expression of HoxA2 and HoxB2 shows that cell clones 1, 3, and 7 of Series 1 (see Example 17) derive from the region of the third and fourth rhombomeres and would migrate to the region of about the dorsal first or more likely the second branchial arch. Clone 7 of Series 1 shows HoxB2 but not HoxA2 expression, confining the region of the cells to the junction of the third and fourth rhombomere. The smooth muscle cell clones 15 and 16 of Series 1 show HoxC6 and HoxC10 expression, consistent with these cells being of thoracic origin. The mesenchymal cell clones 8-14 of Series 1 including cell clone 8 with dermal progenitor characteristics, show HoxA10 and HoxA11 expression consistent with limb bud mesenchymal cells. Lastly, cell clone 17 of Series 1 with enteric smooth muscle characteristics has HoxA10 and HoxA11 expression but not HoxC6 or HoxC10 expression consistent with these cells deriving from somites in the lumbar region. The use of Hox and related developmentally-regulated segmentation genes to identify the nature of cell clones but also in matching the cells to the destination tissue insures that cells most suited for transplantation are obtained and used.

### Example 23

Induction of myocardial progenitors using inducer visceral endoderm cells. Visceral endoderm cells have an inductive effect on splanchnic mesoderm to differentiate into cells of the myocardial lineages. Pluripotent stem cells such as hES, hEC, hED, hEG or splanchnic mesoderm cells produced by the use of the methods of the present invention can be induced to differentiate into cells of cardiac lineages by juxtaposing said stem cells with visceral endoderm cells, including but not limited to cells expressing relatively high levels of AFP (Accession number NM_001134.1). In this example, hES cells are cultured as described herein, then three days following subculture, colonies are scraped from the dish and placed onto confluent cultures of visceral endoderm and cultured in PromoCell Skeletal Muscle Medium (Table I, condition #1112) or its equivalent for 2-6 weeks. Myocardial cells can be identified by the use of markers well known in the art, including the presence of myocardial myosin heavy chain MYH7 (accession number NM_000257.1).

### Example 24

hES cell colonies from one six well plate were grown to form embryoid bodies (EB) (see, e.g., U.S. application No. 60/538,964, filed Jan. 23, 2004, international patent publication no. WO05070011, published Aug. 4, 2005 and U.S. patent publication no. 20060018886, published Jan. 26, 2006, the disclosure of each of which is hereby incorporated by reference) and plated out to form epidermal keratinocytes that express a prenatal pattern of gene expression.

Specifically, colonies from the hES cell line H9 were differentiated by the removal of LIF-containing medium and the addition of DMEM medium containing 10% FBS. After 5 days of exposure to differentiation medium, the cells were trypsinized, and plated onto bacteriological plates and cultured for an additional 20 days to further induce differentiation as embryoid bodies. The cells were then trypsinized for 10 minutes with 0.25% trypsin/EDTA, neutralized with DMEM medium containing 10% FBS, counted with a Coulter counter, and the cells were plated at limiting dilutions from 5,000 plated cells, to 2,000 cells to 500 cells introduced into the 15 cm tissue culture plates with EpiLife medium (Cascade Biologics) Cat #M-EP/cf medium supplemented with calcium, LSGS (Cat #S-003-10) and recombinant collagen (Cat #R-011-K) per manufacturer's instructions. The cells were subsequently incubated in 5% ambient oxygen undisturbed for two weeks.

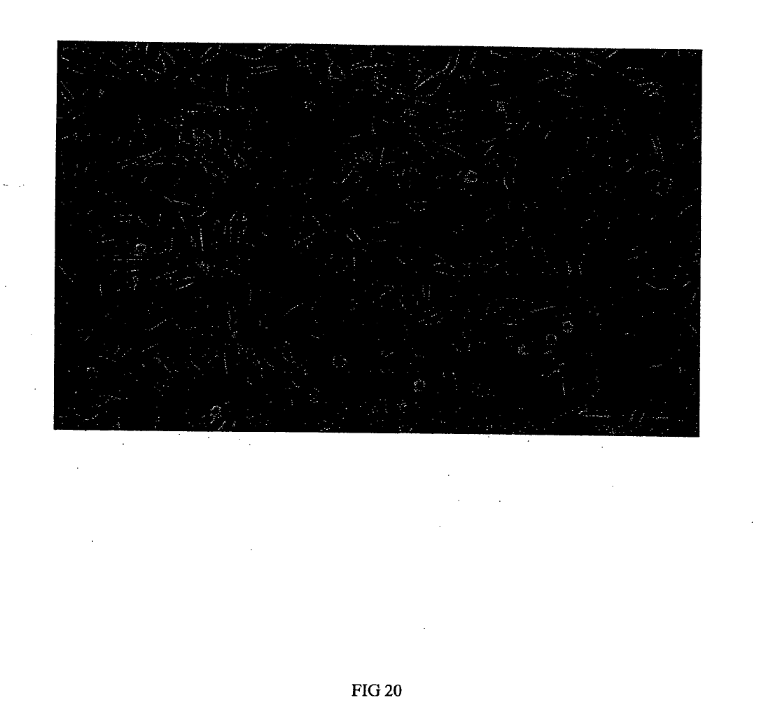

Clonal colonies were identified by phase contrast microscopy and those that are uniformly circular and well separated from surrounding colonies were marked for removal using cloning cylinders. A representative colony is shown in FIG. 20.

The trypsinized cells from within each cloning cylinder are then replated into collagen coated 24 well plates and incubated in the same medium until the cells reach confluency. Those that grow at a relatively rapid rate of approximately one doubling a day are then karyotyped to determine that they are normal human cells. A total genomic expression analysis using the Illumina system is then performed on the cells.

For improved wound repair, the keratinocytes with robust proliferative capacity are combined with dermal fibroblasts with a prenatal pattern of gene expression to produce skin equivalents capable of imparting a regenerative capacity to postnatal skin.

### Example 25

### Cranial Neural Crest Cells

Populations of neural crest cells of cranial, vagal, cardiac, or trunk origins can be derived according to the methods described in the present invention as these cells are formed in association with the differentiating central nervous system, neural tube and many differentiation conditions including in situ differentiation of hES, hEG, hEC or hED cells, embryoid bodies formed from hES, hEG, human EC or hED cells, or analogous differentiation systems that will form a complex mixture of neural tube-associated cells including the juxtaposition of neuroepithelium with inducing cells such as non-neural ectoderm (presumptive epidermis) in order to increase the number of neural crest progenitors or the administration of retinoic acid to shift the differentiation of neural crest types to a more caudal type. From heterogeneous mixtures of neural crest cells or neural crest progenitors, clonal or oligoclonal populations of the various neural crest cell types can be isolated according to the methods described in the present invention. Such cells may then be characterized through their pattern of gene expression or protein profiles to confirm their identity as neural crest cells. In the case of the human species and many species other than the laboratory mouse or chicken, the particular markers of various neural crest cells are not completely characterized.

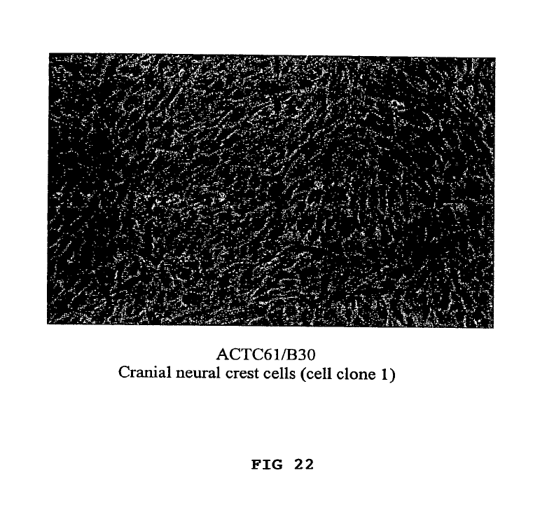

By way of nonlimiting example, example 17 of the present invention describes a method of obtaining clonal cranial neural crest cells from hES cells such as the hES cell line ACT3. Using the methods described in Example 17 above, single cell-derived cranial crest cells (also referred to as cell clone number 1 or ACTC61/B30 of Series 1) were generated. A phase contrast photograph of these cells at passage 7 is shown in FIG. 22.

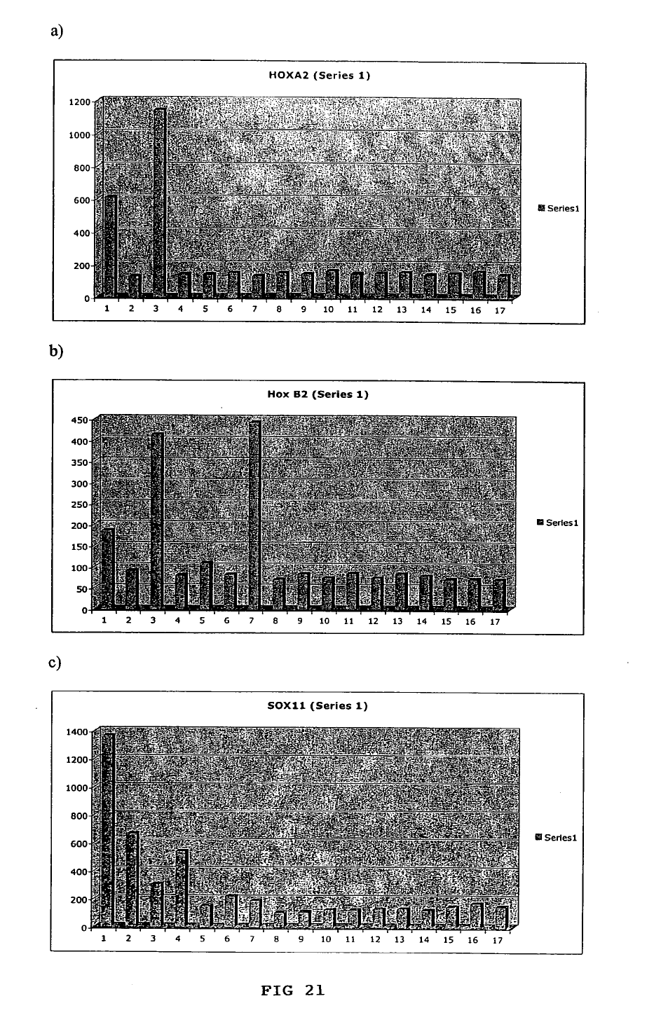

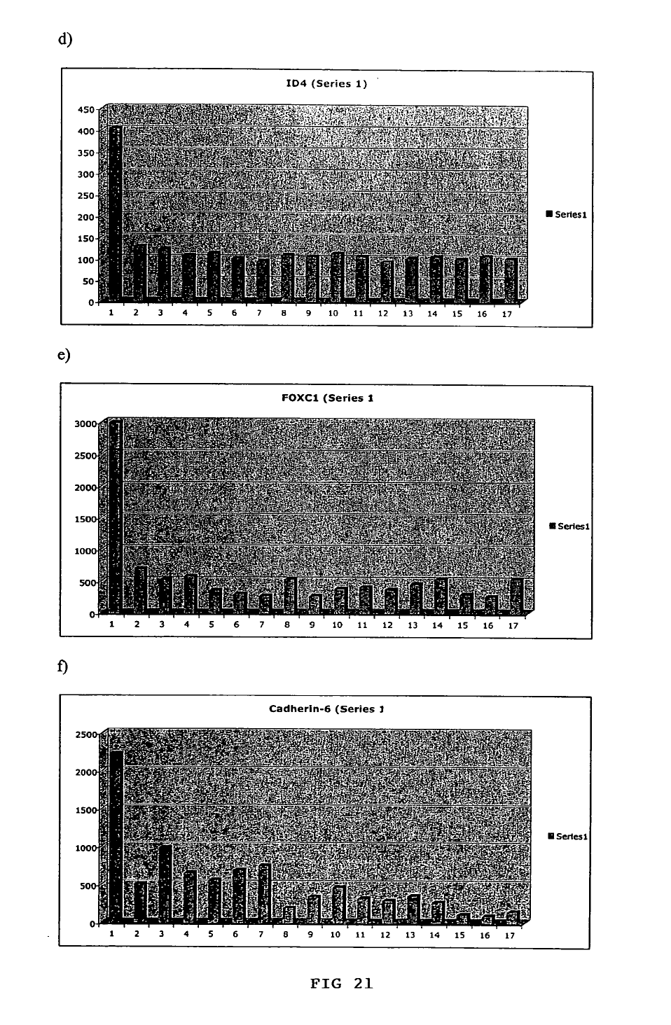

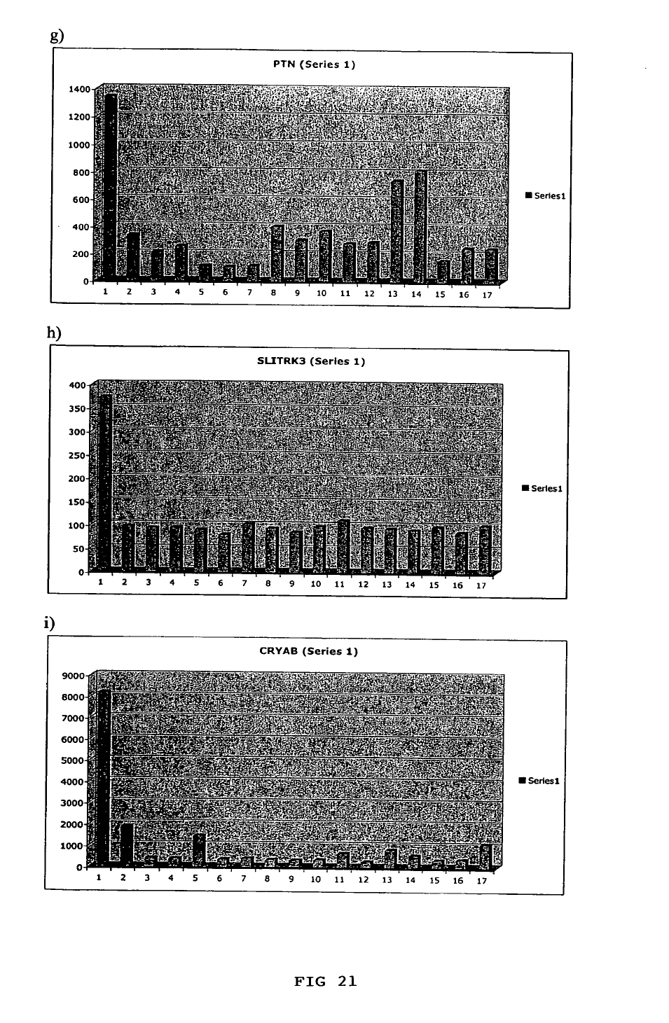

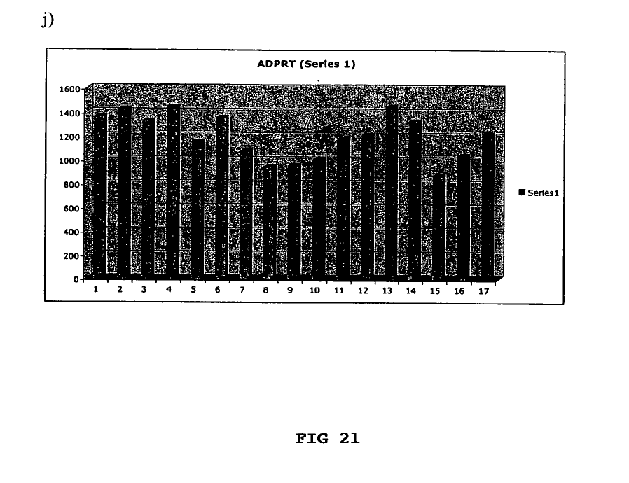

These cells displayed some but not all of the markers reported to correlate with mammalian cranial neural crest as well as novel and unexpected markers. The gene expression profile of cranial neural crest cell clone 1 is depicted in FIG. 21.

Cranial neural crest cells are well known to originate from the 1st-6th rhomomeres of the hindbrain. Depending on the rhombomere from which they originate, they differ in their expression of genes such as the HOX genes. Those originating from the third rhombomere express HOXA2 (Accession No. NM_006735.3) and HOXB2, unlike the neural crest cells isolated from mice that express high levels of Sox10 (Sieber-Blum (2004) Dev. Dyn. 231:258-269). Surprisingly, cell clone number 1 (ACTC61/B30) was negative for SOX10 expression (data not shown) but did express SOX11 (Accession No. NM_003108.3) (see FIG. 23). Similarly, cell clone number 1 of Series 1 (ACTC61/B30) did not express detectable levels of NCX (TLX2) expression, even though previous studies have reported that neural crest cells derived from mice and primates from ES cells are positive for this gene (Mizuseki et al (2003) PNAS 100(10):5823-5833) (data not shown). Other markers that distinguish the human cranial neural crest cell clone number 1 of Series 1 (ACTC61/B30) from other cell types include ID4 (Accession No. NM_001546.2), FOXC1 (Accession No. NM_001453.1), Cadherin-6 (Accession No. NM_004932.2), PTN (Accession No. NM_002825.5), SLITRK3 (Accession No. NM_014926.2), and CRYAB (Accession No. NM_0015885.1), as shown in FIG. 23. The relative expression levels of these markers normalized within the Series 1 data set are compared with the expression of the housekeeping ADPRT gene, as shown in FIG. 21.

The cranial neural crest cell clone 1 of Series 1 (ACTC61/B30) is also negative for HOXB1, HOXA3, HOXB3, HOXD3 and HOXB4 expression (data not shown). This further suggests that the cells originated from the third rhombomere and normally would have migrated into the second or third branchial arch largely at the level of the fourth rhombomere. Derivatives of the migrating cranial neural crest derived from the third and fifth rhombomeres stem from the region of the fourth rhombomere and migrate through the second branchial arch include bones such as the lesser horn of the hyoid bone, the stylohyoid ligament, the styloid process, and the stapes, muscles such as the buccinator, platysma, stapedius, stylohyoid, and the posterior belly of the digastric, and cranial nerve VII and are useful in regenerating numerous tissues as described herein.

Such cranial, vagal, cardiac or trunk neural crest cells can be used in a wide variety of applications in veterinary and human medicine for both research and therapeutic applications. By way of nonlimiting example, the cells may be used in either a nongenetically-modified or a genetically-modified form in cell-based assays for drug discovery, used to manufacture extracellular matrix materials or secreted factors such as cytokines, growth factors, and chemokines, or formulated and introduced into the bodies of humans or nonhuman animals in cell therapy to repair or regenerate tissues that these cells normally form in the embryo such as those listed above, or to deliver embryonic cytokines or growth factors such as to promote angiogenesis or neurite outgrowth as described herein.

The desired cell types can be differentiated from the neural crest stem cells by inducing differentiation and obtaining a population of cells enriched in a desired cell type, or by differentiating the neural crest cells into a heterogeneous mixture of downstream cell types and purifying out the desired cell type using techniques known in the art including genetic selection, or the use of affinity purification such as the use of antibodies or peptide ligands to antigens specific to the cell type of interest.

By way of nonlimiting examples, the methods to induce the differentiation of the neural crest cells may include the administration of 10 ng/mL of BMP2 for two weeks to generate chondrocytes, or 10 nM neuregulin-1 for two weeks to generate Schwann cells or peripheral neurons.

### Example 26

Some cell types do not proliferate well under any known cell culture conditions. To artificially stimulate the proliferation of such cells, the hES cell line H9 is transfected with a plasmid construct containing a temperature sensitive mutant of SV40 T antigen (Tag) regulated by a gamma-interferon promoter as described (Jat et al., Proc Natl Acad Sci USA 88:5096-5100 (1991)). The inducible Tag hES cells are then allowed to undergo a first step of differentiation with Tag in the uninduced state at the nonpermissive temperature of 37° C. and in medium lacking exogenous gamma-interferon in six differing conditions as follows.

Inducible Tag-expressing cells were plated in a standard 6 well tissue culture plate on a feeder layer of mouse embryonic fibroblasts and allowed to grow for 9 days to confluence. The hES cell growth medium was replaced by 6 extracellular matrix/growth media (see Table XI) and the hES cells were allowed to differentiate for 3 days.

The cells were trypsinized using 0.05% trypsin and transferred to Corning 6-well, ultra low attachment tissue culture plates containing the same differentiation medium. The embryoid bodies were allowed to differentiate for 7-10 days, collected, washed in phosphate buffered saline, dissociated into single cells with trypsin (0.25% trypsin) and the differentiated cells plated out in extra cellular matrix coated 15 cm plates (Table XI) in the same medium supplemented with gamma-interferon as described (Jat et al (1991) PNAS USA 88:5096-5100) under the permissive temperature of 32.5° C. The differentiated cells are allowed to proliferate for 14-20 days and the resulting colonies are cloned and plated in 24 well plates containing the same medium supplemented with gamma-interferon under the permissive temperature of 32.5° C. and extracellular matrix from which they were derived. The cloned colonies are expanded to obtain a stock of cells and the cell line stocks are cryopreserved. To determine the pattern of gene expression, the cells are shifted to the same medium reduced in serum concentration by 20-fold, free of gamma interferon, and at the nonpermissive temperature of 37° C. for five days.

TABLE XI
Extracellular Matrix & Growth Medium

| | | Extra |
|---|---|---|
| | | Cellular |
| 15 cm Plate | Selection & Growth Media | Matrix |
| 1 | Smooth Muscle Medium | Gelatin |
| 2 | Neurobasal Medium - B27 | Poly-lysine - |
| | | BioCoat |
| 3 | Epi-Life Medium - LSGS | Collagen IV |
| 4 | Endothelial Cell Growth | Gelatin |
| | Medium | |
| 5 | Skeletal Muscle Cell Growth | Gelatin |
| | Medium | |
| 6 | DMEM + 10% FBS | Gelatin |

During the clonal expansion protocol, samples of the cell lines are taken for gene expression and immunophenotype analysis.

### Example 27

### Production of ED Endoderm and Pancreatic Beta Cells

Isolated blastomeres or similar ED cells such as isolated morula or ICM cells are isolated, as described in U.S. provisional Application No. 60/839,622, filed Aug. 23, 2006, its disclosure is hereby incorporated by reference. These cells are then added onto mitotically-inactivated feeder cells that express high levels of NODAL or cell lines that express members of the TGF-beta family that activate the same receptor as NODAL such as CM02 cells that express relatively high levels of Activin-A, but low levels of Inhibins or follistatin. The cells are then incubated for a period of five days in DMEM medium with 0.5% human serum. After five days, the resulting cells which include definitive endodermal cells are purified by flow cytometry or other affinity-based cell separation techniques such as magnetic bead sorting using antibody specific to the CXCR4 receptor and then permeabilized and exposed to cellular extracts from isolated bovine pancreatic beta cells as described in U.S. patent publication 20050014258 (its disclosure being incorporated by reference). The resulting heterogeneous mixture of cells that has been induced toward beta cell differentiation is then cloned using techniques described herein. These cells are then directly differentiated into pancreatic beta cells or beta cell precursors using techniques known in the art for differentiating human embryonic stem cells into such cells or by culturing the hES cells on inducer cell mesodermal cell lines described herein.

### Example 28

Laser Capture Microscopy and Microarray Analysis of Whole Organism Tissues, hES, and Differentiated hES Cell Lines

The quantitation of gene expression in whole organism tissues, human embryonic stem cells, and their differentiated progeny, are accomplished by microarray technologies well know to those versed in the art. Tissue samples from biopsies and cell colonies containing differentiated hES cell progeny may be isolated using Laser Capture Microdissection (LCM) to capture small populations of cell for analysis (Baba, et al, 2006, Trans. Res. 148:103-113, Sluka, P. et al, 2002, Biol Repro 67:820-828). In this approach, total RNA is purified from target cells, cell colonies, or tissues and RNA prepared by linear amplification with T7 RNA polymerase such that there is a linear appearance of mRNA product in direct proportion to the amount of RNA template in the samples. These amplified samples are then fluorescently labeled and gene expression levels determined using microarray analysis.

### Selective Collection of Cells by LCM

Biopsy specimens are embedded in Tissue-Tek O.C.T. Compound (Miles, Inc., Elkhart, Ind.) and frozen in acetone chilled with dry ice. Ten micrometer frozen sections are produced, fixed in a 70% ethanol solution, and stained with hematoxylin and eosin. Cell clusters are selectively picked up by LCM (LM-100; Arcturus Engineering, Inc., Mountain View, Calif.) following the standard protocol as previously described (Emmert-Buck M R, Bonner R F, Smith P D, Chuaqui R F, Zhuang Z, Goldstein S R et al. Laser capture micro-dissection. Science (Wash. D.C.) 1996; 274:998-1001, Bonner R F, Emmert-Buck M R, Cole K, Pohida T, Chuaqui R, Goldstein S, et al. Laser capture dissection: molecular analysis of tissue. Science (Wash. D.C.) 1997; 278:1481-2). The entire sampling scheme is repeated three times from the same tissue. LCM is performed using a PixCell II laser capture microdissection microscope (Arcturus Engineering, Mountain View, Calif.), equipped with a fluorescence light source. Each section is pretreated with a PrepStrip tissue preparation strip (Arcturus) to remove loose debris and to flatten the tissue. Sections are then visualized using a 20× objective, and capture is performed using a 30-mm diameter laser spot size set at 20-30 mW with a pulse duration of 5 msec. Cells are captured using CapSure LCM caps (Arcturus) and stored in a desiccator prior to extraction of total RNA.

Extraction of Total RNA from BEC

Total RNA is isolated from the collected cells using a StrataPrep Total RNA Microprep Kit (Stratagene, La Jolla, Calif), according to the manufacturer's instructions. A preliminary examination is conducted to confirm the quality of the tissues as follows: Total RNA was extracted from the remaining portion of specimens using TRIzol (Gibco BR1, Rockville, Md.) and analyzed by electrophoresis in formaldehyde-agarose gels.

### Gene Amplification by T7 RNA Polymerase

Total RNA extracted from the collected cells is linearly amplified using T7 RNA polymerase, with a MessageAmp aRNA Kit (Ambion, Austin, Tex.). The applied procedure consists of reverse transcription with an oligo (dT) primer bearing a T7 promoter, and in vitro transcription of the resulting DNA with T7 RNA polymerase, generating hundreds to thousands of antisense cRNA copies of each mRNA per sample. To confirm the efficiency and accuracy of the gene amplification procedure, a preliminary examination is performed using a sample of human ovary total RNA (Stratagene, La Jolla, Calif.) as follows. First, 2 μg of human ovary total RNA is amplified twice by the gene amplification procedure. The resulting amount of amplified RNA is then determined and compared with that of the original. Secondly, the genetic composition of the amplified RNA is compared with that of the original by cRNA microarray analysis. cRNA probes are labeled with fluorescent dye, generated using an Illumina Total Prep RNA Labelling kit (Ambion, Inc, Austin, Tex.), from samples of (1) original human ovary total RNA, (2) RNA after refining poly(A)_mRNA (OligotexdT30, (Super) mRNA Purification Kit; Takara Bio, Inc.), (3) RNA after single amplification, and (4) RNA after amplifying twice. All samples are hybridized on a cRNA microarray (Illumina Human Sentrix 6 Beadchip, Illumina, Inc, San Diego, Calif.), and the fluorescence signals of the resulting spots are scanned by an Illumina 500 Beadstation. Correlations are examined by constructing scatter plots of the logarithms of the resulting fluorescent signals. The expression of each gene can be simultaneously analyzed through hybridization of the probes, which are prepared by using RNA obtained from human cells as a template. Control spots can be used to normalize the signal intensity between fluorescence-labeled probes and to determine the background level.

cRNA Microarray Analysis

cRNA probes are generated from the LCM generated RNA samples, amplified twice and labeled with fluorescent dye (Illumina Total Prep RNA Labelling kit, Ambion, Inc, Austin, Tex.). The labelled cRNA probes are then hybridized on an Illumina Human Sentrix-6 microarray and scanned as described above.

### Example 29

### Generation of Cell Lines Secreting the TAT-Tag Fusion Protein

### Construction of TAT-TAg Expression Plasmid

The SV40 large T antigen is amplified by polymerase chain reaction (PCR) with primers flanking the open reading frame. The 5′ PCR oligonucleotide sequence included DNA sequence complementary to the 5′ end of the SV40 large T antigen and DNA sequence encoding the TAT PTD (YGRKKRRQRRR). The PCR product was cloned into the pEF6/V5-His TOPO® TA vector (Invitrogen, Carlsbad, Calif.) according to the manufacturer instructions. Transcription is under the control of the hEF-1alpha promoter (hEF-1alpha) and the fusion protein (TAT-TAg) contains at its C-terminal end a myc and his epitope tags.

### Cell Culture, Transfection, and Replication Labeling

Human cell lines are grown as described above, by the supplying vendor or collaborator, or in DMEM supplemented with 10% fetal bovine serum, lx glutamax, and nonessential amino acids. To create cell lines secreting TAT-Tag, the human Hela cell line is transfected with the TAT-large T antigen construct using GenePorter Transfection Reagent (Gene Therapy Systems, San Diego, Calif.) by mixing 7 μg of plasmid DNA in 1 ml serum-free DMEM and mixing with 1 ml DMEM containing 35 μl GenePorter reagent. After aspirating medium from a 60 mm culture dish with Hela cells, this solution is added to the cells. After 5 hrs, 2 ml of DMEM containing 20% FCS is added. After another 48 hrs, the drug blasticidin is added to the cultures to select for stable Hela cell transfectants. Blasticidin resistant colonies are picked, expanded and the cell conditioned medium analyzed for the presence of the TAT-Tag fusion protein by immunoblotting cell extracts, conditioned medium and cell pellet as described below.

### Antibodies

The following primary antibodies are used: anti-myc tag mouse monoclonal antibody (clone 9E10); anti-his tag mouse monoclonal antibody (Dianova, Hamburg, Germany); anti-SV40 large T antigen mouse monoclonal antibody (PAB 101). For immunoblot analysis, horseradish peroxidase-conjugated anti-mouse IgG (Amersham, Buckinghamshire, U.K.) is used.

### Immunoblot Analysis

Transfected COS-7 cells are extracted for 30 min on ice in RIPA buffer. In brief, we analyze cell extracts and cell pellets by immunoblot using anti-myc tag mouse monoclonal antibody to detect the TAT-Tag fusion protein.

### Cell Co-Culture

TAT-Tag secreting Hela cell lines are used to treat growth medium appropriate for culture of the recipient cell lines. Briefly, TAT-Tag secreting Hela cells are cultured in growth medium. The medium is harvested by aspiration, filtered and applied to recipient cell cultures. Uptake of the TAT-Tag by recipient cells is monitored by immunoblotting as described above.

### Example 30

### Mitomycin C Treatment of Cells

1. Grow cells to confluence in 15 cm plates or T-150 flasks. 2. Inject 2 ml of sterile water (or PBS) into Mitomycin C (Sigma, Cat #M4287-2MG) vial and dissolve completely. Concentration of Mitomycin C is 1 mg/ml. Once prepared, Mitomycin C is good for about 2 weeks when stored at 4 degree C. 3. Prepare about 10 ml of warm medium for each plate or flask. Add 100 ul of Mitomycin C to each 10 ml of medium. Concentration of Mitomycin C is 10 ug/ml. 4. Aspirate medium from the plates or flasks and replace with the Mitomycin C medium (10 ml per plate or flask). Place in CO2 incubator at 37 degree C. for 3 hours. 5.

Aspirate Mitomycin C medium into disposal trap that containing bleach. Wash Mitomycin C treated cells 2-4 times with warm PBS. Aspirate PBS into bleach containing trap. 6. Trypsinize cells, neutralize the Trypsin with DMEM+10% FBS and count the number of cells with a Coulter Counter or hemacytometer. 7.

Determine the number of cells needed to cover the vessel of interest. For example, for mouse embryonic fibroblasts (MEF) feeder cells, at least 500K cells for one well of a 6 well plate are needed. This cell number could be increased by approximately 10-30% to account for cell death during the freezing process. 8.

Freeze the cells in aliquots convenient for later use. For example, MEF feeder cells can be frozen in aliquots for single wells (650K), 3 wells (1.75 million) or 6 wells (3.3 million). Freezing medium is the same medium used to grow the cells containing 10% dimethylsulfoxide (DMSO) and freezing solution should be cooled to 2-4 degree C. prior to use. Do not use DMSO freezing medium warmed to 37 degree C. Medium should contain at least 10% serum for best results. 9. Before discarding any unused Mitomycin C or vessels used in the inactivation procedure, treat with bleach.

### Example 31

### Differentiation of Directly Differentiated Embryo-Derived Cells into Hepatocytes

Human embryos are attached to collagen-coated tissue culture vessels and cells from the ICM are allowed to attach and spread in SR medium containing 1% DMSO. The cultures are fed daily with SR medium for 4 days and then exchanged into unconditioned SR medium containing both 1% DMSO and 2.5% Na-butyrate, with which they are fed daily for 6 days. They are then replated onto collagen, and cultured in a hepatocyte maturation medium containing: 30 ng/mL hEGF+1% DMSO 1% DMSO+10 ng/mL TGF-α+2.5 mM 30 ng/mL HGF+butyrate 2.5 mM butyrate (see U.S. Pat. No. 7,033,831).

The differentiated cells are allowed to grow for 7-10 days to form colonies, the colonies are cloned and plated in 24-well gelatin-coated plates containing the same medium in which they are grown. The individual colonies are expanded to obtain a stock of cells and the cell line stocks are cryopreserved.

During the clonal expansion protocol of step 2, samples of the cell lines are taken for gene expression and immunophenotype analysis.

### Example 32

### Differentiation of Directly-Differentiated Embryo-Derived Cells into Neuronal Cells

Human ICMs are isolated from blastocyst-staged embryos by immunosurgery as is well-known in the art, the ICMs are cultured on tissue culture plastic for five days in Gibco Neural Basal Medium, then placed in DMEM supplemented with 10% (by volume) fetal bovine serum (FBS). After resuspension in DMEM and 10% FBS, 1×10⁶ cells are plated in 5 ml DMEM plus 10% FBS plus 0.5 μM retinoic acid in a 60 mm Fisher brand bacteriological grade Petri dish. In such Petri dishes, embryonic stem cells cannot adhere to the dish, and instead adhere to each other, thus forming small aggregates of cells. Aggregation of cells aids in enabling proper cell differentiation. After two days, aggregates of cells are collected and resuspended in fresh DMEM plus 10% FBS plus 0.5 μM retinoic acid, and replated in Petri dishes for an additional two days. Aggregates, now induced four days with retinoic acid, are trypsinized to form a single-cell suspension, and plated in medium on poly-D-lysine-coated coated tissue culture grade dishes. The stem cell medium is formulated with Kaighn's modified Ham's F12 as the basal medium with the following supplements added: 15 μg/ml ascorbic acid 0.25% (by volume) calf serum 6.25 mg/ml insulin 6.25 μg/ml transferrin 6.25 μg/ml selenous acid 5.35 μg/ml linoleic acid 30 pg/ml thyroxine (T3) 3.7 ng/ml hydrocortisone 1. ng/ml Heparin 10 ng/ml somatostatin 10 ng/ml Gly-His-Lys (liver cell growth factor) 0.1 μg/ml epidermal growth factor (EGF) 50 μg/ml bovine pituitary extract (BPE) (see U.S. Pat. No. 6,432,711).

The differentiated cells are allowed to grow for 7-10 days to form colonies, the colonies are cloned and plated in 24-well gelatin-coated plates containing the same medium in which they are grown. The individual colonies are expanded to obtain a stock of cells and the cell line stocks are cryopreserved.

During the clonal expansion protocol, samples of the cell lines are taken for gene expression and immunophenotype analysis.

### Example 33

### Differentiation of Embryonic Bodies into Neuronal Cells

Human blastomeres are removed from 8 cell embryos and plated onto collagen-coated tissue culture vessels and cultured for two days in DMEM medium with 10% FBS. The cells are then removed by scraping and placed in Neural basal medium on bacteriological plates. Media is supplemented with the following growth factors: retinoic acid (Sigma): 10⁻⁷M (Bain et al (1995) or 10⁻⁶M (Bain et al., 1996); TGfβ1 (Sigma): 2 ng/ml (Slager et al., (1993) Dev. Genet., Vol. 14, pp. 212 224); and βNGF (New Biotechnology, Israel): 100 ng/ml (Wobus et al., 1988). After 21 days, EBs are plated on 5 μg/cm² collagen treated plates, either as whole EB's, or as single cells dissociated with trypsin/EDTA. The cultures are maintained for an additional week or 2 days respectively (see U.S. Pat. No. 7,045,353).

The differentiated cells are allowed to grow for 7-10 days to form colonies, the colonies are cloned according to the steps 2 (a) and 2 (b) of the present invention and plated in 24-well gelatin-coated plates containing the same medium in which they are grown. The individual colonies are expanded to obtain a stock of cells and the cell line stocks are cryopreserved.

During the clonal expansion protocol, samples of the cell lines are taken for gene expression and immunophenotype analysis.

TABLE I
Culture Variables
EGF Ligands

| | |
|---|---|
| 1) | Amphiregulin |
| 2) | Betacellulin |
| 3) | EGF |
| 4) | Epigen |
| 5) | Epiregulin |
| 6) | HB-EGF |
| 7) | Neuregulin-3 |
| 8) | NRG1 isoform GGF2 |
| 9) | NRG1 Isoform SMDF |
| 10) | NRG1-alpha/HRG1-alpha |
| 11) | TGF-alpha |
| 12) | TMEFF1/Tomoregulin-1 |
| 13) | TMEFF2 |
| 14) | EGF Ligands pooled (1-13 above) |

EGF R/ErbB Receptor Family

| | |
|---|---|
| 15) | EGF Receptor |
| 16) | ErbB2 |
| 17) | ErbB3 |
| 18) | ErbB4 |
| 19) | EGF/ErbB Receptors pooled (15-18 above) |

FGF Ligands

| | |
|---|---|
| 20) | FGF acidic |
| 21) | FGF basic |
| 22) | FGF-3 |
| 23) | FGF-4 |
| 24) | FGF-5 |
| 25) | FGF-6 |
| 26) | KGF/FGF-7 |
| 27) | FGF-8 |
| 28) | FGF-9 |
| 29) | FGF-10 |
| 30) | FGF-11 |
| 31) | FGF-12 |
| 32) | FGF-13 |
| 33) | FGF-14 |
| 34) | FGF-15 |
| 35) | FGF-16 |
| 36) | FGF-17 |
| 37) | FGF-18 |
| 38) | FGF-19 |
| 39) | FGF-20 |
| 40) | FGF-21 |
| 41) | FGF-22 |
| 42) | FGF-23 |
| 43) | FGF Ligands pooled (20-38 above) |

FGF Receptors

| | |
|---|---|
| 40) | FGF R1 |
| 41) | FGF R2 |
| 42) | FGF R3 |
| 43) | FGF R4 |
| 44) | FGF R5 |
| 45) | FGF Receptors pooled (40-44 above) |

FGF Regulators

| | |
|---|---|
| 46) | FGF-BP |

Hedgehogs

| | |
|---|---|
| 47) | Desert Hedgehog |
| 48) | Sonic Hedgehog |
| 49) | Indian Hedgehog |
| 50) | Hedgehogs pooled (47-49 above) |

Hedgehog Regulators

| | |
|---|---|
| 51) | Gas1 |
| 52) | Hip |
| 53) | Hedgehog Regulators pooled (51-52 above) |

IGF Ligands

| | |
|---|---|
| 54) | IGF-I |
| 55) | IGF-II |
| 56) | IGF Ligands pooled (54-55 above) |

IGF-I Receptor (CD221)

| | |
|---|---|
| 57) | IGF-I R |

GF Binding Protein (IGFBP) Family

| | |
|---|---|
| 58) | ALS |
| 59 | IGFBP-4 |
| 60) | CTGF/CCN2 |
| 61) | IGFBP-5 |
| 62) | Endocan |
| 63) | IGFBP-6 |
| 64) | IGFBP-1 |
| 65) | IGFBP-rp1/IGFBP-7 |
| 66) | IGFBP-2 |
| 67) | NOV/CCN3 |
| 68) | IGFBP-3 |
| 69) | GF Binding Protein Family pooled (58-68 above) |

Receptor Tyrosine Kinases

| | |
|---|---|
| 70) | Axl |
| 71) | C1q R1/CD93 |
| 72) | DDR1 |
| 73) | Flt-3 |
| 74) | DDR2 |
| 75) | HGF R |
| 76) | Dtk |
| 77) | IGF-II R |
| 78) | Eph |
| 79) | Insulin R/CD220 |
| 80) | EphA1 |
| 81) | M-CSF R |
| 82) | EphA2 |
| 83) | Mer |
| 84) | EphA3 |
| 85) | MSP R/Ron |
| 86) | EphA4 |
| 87) | MuSK |
| 88) | EphA5 |
| 89) | PDGF R alpha |
| 90) | EphA6 |
| 91) | PDGF R beta |
| 92) | EphA7 |
| 93) | Ret |
| 94) | EphA8 |
| 95) | ROR1 |
| 96) | EphB1 |
| 97) | ROR2 |
| 98) | EphB2 |
| 99) | SCF R/c-kit |
| 100) | EphB3 |
| 101) | Tie-1 |
| 102) | EphB4 |
| 103) | Tie-2 |
| 104) | EphB6 |
| 105) | TrkA |
| 106) | TrkB |
| 107) | TrkC |
| 108) | VEGF R1/Flt-1 |
| 109) | VEGF R2/Flk-1 |
| 110) | VEGF R3/Flt-4 |
| 111) | Receptor Tyrosine Kinases pooled (70-110 above) |

Proteoglycans

| | |
|---|---|
| 112) | Aggrecan |
| 113) | Lumican |
| 114) | Biglycan |
| 115) | Mimecan |
| 116) | Decorin |
| 117) | NG2/MCSP |
| 118) | Endocan |
| 119) | Osteoadherin |
| 120) | Endorepellin |
| 121) | Syndecan-1/CD138 |
| 122) | Glypican 2 |
| 123) | Syndecan-3 |
| 124) | Glypican 3 |
| 125) | Testican 1/SPOCK1 |
| 126) | Glypican 5 |
| 127) | Testican 2/SPOCK2 |
| 128) | Glypican 6 |
| 129) | Testican 3/SPOCK3 |
| 130) | Heparan sulfate proteoglycan |
| 131) | Heparin |
| 132) | Chondroitin sulfate proteoglycan |
| 133) | Hyaluronic acid |
| 134) | Dermatan sulfate proteoglycan |

Proteoglycan Regulators

| | |
|---|---|
| 135) | Arylsulfatase A/ARSA |
| 136) | HAPLN1 |
| 137) | Exostosin-like 2 |
| 138) | HS6ST2 |
| 139) | Exostosin-like 3 |
| 140) | IDS |
| 141) | Proteoglycan Regulators pooled (135-140 above) |

SCF, Flt-3 Ligand & M-CSF

| | |
|---|---|
| 142) | Flt-3 |
| 143) | M-CSF R |
| 144) | Flt-3 Ligand |
| 145) | SCF |
| 146) | M-CSF |
| 147) | SCF R/c-kit |
| 148) | Pooled factors (142-147 above) |

Activins

| | |
|---|---|
| 149) | Activin A |
| 150) | Activin B |
| 151) | Activin AB |
| 152) | Activin C |
| 153) | Pooled Activins (149-152 above) |

BMPs (Bone Morphogenetic Proteins)

| | |
|---|---|
| 154) | BMP-2 |
| 155) | BMP-3 |
| 156) | BMP-3b/GDF-10 |
| 157) | BMP-4 |
| 158) | BMP-5 |
| 159) | BMP-6 |
| 160) | BMP-7 |
| 161) | BMP-8 |
| 162) | Decapentaplegic |
| 163) | Pooled BMPs (154-162 above) |

GDFs (Growth Differentiation Factors)

| | |
|---|---|
| 164) | GDF-1 |
| 165) | GDF-2 |
| 166) | GDF-3 |
| 167) | GDF-4 |
| 168) | GDF-5 |
| 169) | GDF-6 |
| 170) | GDF-7 |
| 171) | GDF-8 |
| 172) | GDF-9 |
| 173) | GDF-10 |
| 174) | GDF-11 |
| 175) | GDF-12 |
| 176) | GDF-13 |
| 177) | GDF-14 |
| 178) | GDF-15 |
| 179) | GDFs pooled (164-178 above) |

GDNF Family Ligands

| | |
|---|---|
| 180) | Artemin |
| 181) | Neurturin |
| 182) | GDNF |
| 183) | Persephin |
| 184) | GDNF Ligands pooled (180-183 above) |

TGF-beta

| | |
|---|---|
| 185) | TGF-beta |
| 186) | TGF-beta 1 |
| 187) | TGF-beta 1.2 |
| 188) | TGF-beta 2 |
| 189) | TGF-beta 3 |
| 190) | TGF-beta 4 |
| 191) | TGF-beta 5 |
| 192) | LAP (TGF-beta 1) |
| 193) | Latent TGF-beta 1 |
| 194) | TGF-beta pooled (185-193 above) |

Other TGF-beta Superfamily Ligands

| | |
|---|---|
| 195) | Lefty |
| 196) | Nodal |
| 197) | MIS/AMH |
| 198) | Other TGF-beta Ligands pooled (195-197 above) |

TGF-beta Superfamily Receptors

| | |
|---|---|
| 199) | Activin RIA/ALK-2 |
| 200) | GFR alpha-1 |
| 201) | Activin RIB/ALK-4 |
| 202) | GFR alpha-2 |
| 203) | Activin RIIA |
| 204) | GFR alpha-3 |
| 205) | Activin RIIB |
| 206) | GFR alpha-4 |
| 207) | ALK-1 |
| 208) | MIS RII |
| 209) | ALK-7 |
| 210) | Ret |
| 211) | BMPR-IA/ALK-3 |
| 212) | TGF-beta RI/ALK-5 |
| 213) | BMPR-IB/ALK-6 |
| 214) | TGF-beta RII |
| 215) | BMPR-II |
| 216) | TGF-beta RIIb |
| 217) | Endoglin/CD105 |
| 218) | TGF-beta RIII |
| 219) | TGF-beta family receptors pooled (199-218 above) |

TGF-beta Superfamily Modulators

| | |
|---|---|
| 220) | Amnionless |
| 221) | GASP-2/WFIKKN |
| 222) | BAMBI/NMA |
| 223) | Gremlin |
| 224) | Caronte |
| 225) | NCAM-1/CD56 |
| 226) | Cerberus 1 |
| 227) | Noggin |
| 228) | Chordin |
| 229) | PRDC |
| 230) | Chordin-Like 1 |
| 231) | Chordin-Like 2 |
| 232) | Smad1 |
| 233) | Smad4 |
| 234) | Smad5 |
| 235) | Smad7 |
| 236) | Smad8 |
| 237) | CRIM1 |
| 238) | Cripto |
| 239) | Crossveinless-2 |
| 240) | Cryptic |
| 241) | SOST |
| 242) | DAN |
| 243) | Latent TGF-beta bp1 |
| 244) | TMEFF1/Tomoregulin-1 |
| 245) | FLRG |
| 246) | TMEFF2 |
| 247) | Follistatin |
| 248) | TSG |
| 249) | Follistatin-like 1 |
| 250) | Vasorin |
| 251) | GASP-1/WFIKKNRP |
| 252) | TGF Modulators pooled (220-251 above) |

VEGF/PDGF Family

| | |
|---|---|
| 253) | Neuropilin-1 |
| 254) | PlGF |
| 255) | PlGF-2 |
| 256) | Neuropilin-2 |
| 257) | PDGF |
| 258) | VEGF R1/Flt-1 |
| 259) | PDGF R alpha |
| 260) | VEGF R2/Flk-1 |
| 261) | PDGF R beta |
| 262) | VEGF R3/Flt-4 |
| 263) | PDGF-A |
| 264) | VEGF |
| 265) | PDGF-B |
| 266) | VEGF-B |
| 267) | PDGF-C |
| 268) | VEGF-C |
| 269) | PDGF-D |
| 270) | VEGF-D |
| 271) | PDGF-AB |
| 272) | VEGF/PDGF Family pooled (253-271 above) |

Dickkopf Proteins & Wnt Inhibitors

| | |
|---|---|
| 273) | Dkk-1 |
| 274) | Dkk-2 |
| 275) | Dkk-3 |
| 276) | Dkk-4 |
| 277) | Soggy-1 |
| 278) | WIF-1 |
| 279) | Pooled factors (273-278 above) |

Frizzled & Related Proteins

| | |
|---|---|
| 280) | Frizzled-1 |
| 281) | Frizzled-2 |
| 282) | Frizzled-3 |
| 283) | Frizzled-4 |
| 284) | Frizzled-5 |
| 285) | Frizzled-6 |
| 286) | Frizzled-7 |
| 287) | Frizzled-8 |
| 288) | Frizzled-9 |
| 289) | sFRP-1 |
| 290) | sFRP-2 |
| 291) | sFRP-3 |
| 292) | sFRP-4 |
| 293) | MFRP |
| 294) | Factors pooled (280-293 above) |

Wnt Ligands

| | |
|---|---|
| 295) | Wnt-1 |
| 296) | Wnt-2 |
| 297) | Wnt-3 |
| 298) | Wnt-3a |
| 299) | Wnt-4 |
| 300) | Wnt-5 |
| 301) | Wnt-5a |
| 302) | Wnt-6 |
| 303) | Wnt-7 |
| 304) | Wnt-8 |
| 305) | Wnt-8a |
| 306) | Wnt-9 |
| 307) | Wnt-10a |
| 308) | Wnt-10b |
| 309) | Wnt-11 |
| 310 | Wnt Ligands pooled (295-309 above) |

Other Wnt-related Molecules

| | |
|---|---|
| 311) | beta-Catenin |
| 312) | LRP-6 |
| 313) | GSK-3 |
| 314) | ROR1 |
| 315) | Kremen-1 |
| 316) | ROR2 |
| 317) | Kremen-2 |
| 318) | WISP-1/CCN4 |
| 319) | LRP-1 |
| 320) | Pooled factors (311-319 above) |

Other Growth Factors

| | |
|---|---|
| 321) | CTGF/CCN2 |
| 322) | NOV/CCN3 |
| 323) | EG-VEGF/PK1 |
| 324) | Osteocrin |
| 325) | Hepassocin |
| 326) | PD-ECGF |
| 327) | HGF |
| 328) | Progranulin |
| 329) | beta-NGF |
| 330) | Thrombopoietin |
| 331) | Pooled factors (321-330 above) |

Steroid Hormones

| | |
|---|---|
| 332) | 17beta-Estradiol |
| 333) | Testosterone |
| 334) | Cortisone |
| 335) | Dexamethasone |

Extracellular/Membrane Proteins

| | |
|---|---|
| 336) | Plasma Fibronectin |
| 337) | Tissue Fibronectin |
| 338) | Fibronectin fragments |
| 339) | Collagen Type I (gelatin) |
| 340) | Collagen Type II |
| 341) | Collagen Type III |
| 342) | Tenascin |
| 343) | Matrix Metalloproteinase 1 |
| 344) | Matrix Metalloproteinase 2 |
| 345) | Matrix Metalloproteinase 3 |
| 346) | Matrix Metalloproteinase 4 |
| 347) | Matrix Metalloproteinase 5 |
| 348) | Matrix Metalloproteinase 6 |
| 349) | Matrix Metalloproteinase 7 |
| 350) | Matrix Metalloproteinase 8 |
| 351) | Matrix Metalloproteinase 9 |
| 352) | Matrix Metalloproteinase 10 |
| 353) | Matrix Metalloproteinase 11 |
| 354) | Matrix Metalloproteinase 12 |
| 355) | Matrix Metalloproteinase 13 |
| 356) | ADAM-1 |
| 357) | ADAM-2 |
| 358) | ADAM-3 |
| 359) | ADAM-4 |
| 360) | ADAM-5 |
| 361) | ADAM-6 |
| 362) | ADAM-7 |
| 363) | ADAM-8 |
| 364) | ADAM-9 |
| 365) | ADAM-10 |
| 366) | ADAM-11 |
| 367) | ADAM-12 |
| 368) | ADAM-13 |
| 369) | ADAM-14 |
| 370) | ADAM-15 |
| 371) | ADAM-16 |
| 372) | ADAM-17 |
| 373) | ADAM-18 |
| 374) | ADAM-19 |
| 375) | ADAM-20 |
| 376) | ADAM-21 |
| 377) | ADAM-22 |
| 378) | ADAM-23 |
| 379) | ADAM-24 |
| 380) | ADAM-25 |
| 381) | ADAM-26 |
| 382) | ADAM-27 |
| 383) | ADAM-28 |
| 384) | ADAM-29 |
| 385) | ADAM-30 |
| 386) | ADAM-31 |
| 387) | ADAM-32 |
| 388) | ADAM-33 |
| 389) | ADAMTS-1 |
| 390) | ADAMTS-2 |
| 391) | ADAMTS-3 |
| 392) | ADAMTS-4 |
| 393) | ADAMTS-5 |
| 394) | ADAMTS-6 |
| 395) | ADAMTS-7 |
| 396) | ADAMTS-8 |
| 397) | ADAMTS-9 |
| 398) | ADAMTS-10 |
| 399) | ADAMTS-11 |
| 400) | ADAMTS-12 |
| 401) | ADAMTS-13 |
| 402) | ADAMTS-14 |
| 403) | ADAMTS-15 |
| 404) | ADAMTS-16 |
| 405) | ADAMTS-17 |
| 406) | ADAMTS-18 |
| 407) | ADAMTS-19 |
| 408) | ADAMTS-20 |
| 409) | Arg-Gly-Asp |
| 410) | Arg-Gly-Asp-Ser |
| 411) | Arg-Gly-Asp-Ser-Pro-Ala-Ser-Ser-Lys-Pro |
| 412) | Arg-Gly-Glu-Ser |
| 413) | Arg-Phe-Asp-Ser |
| 414) | SPARC |
| 415) | Cys-Asp-Pro-Gly-Tyr-Ile-Gly-Ser-Arg |
| 416) | Cys-Ser-Arg-Ala-Arg-Lys-Gln-Ala-Ala-Ser-Ile-Lys- |
| | Val-Ser-Ala-Asp-Arg |
| 417) | Elastin |
| 418) | Tropelastin |
| 419) | Gly-Arg-Gly-Asp-Ser-Pro-Lys |
| 420) | Gly-Arg-Gly-Asp-Thr-Pro |
| 421) | Laminin |
| 422) | Leu-Gly-Thr-Ile-Pro-Gly |
| 423) | Ser-Asp-Gly-Arg-Gly |
| 424) | Vitronectin |
| 425) | Superfibronectin |
| 426) | Thrombospondin |
| 427) | TIMP-1 |
| 428) | TIMP-2 |
| 429) | TIMP-3 |
| 430) | TIMP-4 |
| 431) | Fibromodulin |
| 432) | Flavoridin |
| 433) | Collagen IV |
| 434) | Collagen V |
| 435) | Collagen VI |
| 436) | Collagen VII |
| 437) | Collagen VIII |
| 438) | Collagen IX |
| 439) | Collagen X |
| 440) | Collagen XI |
| 441) | Collagen XII |
| 442) | Entactin |
| 443) | Fibrillin |
| 444) | Syndecan-1 |
| 445) | Keratan sulfate proteoglycan |

Ambient Oxygen

| | |
|---|---|
| 446) | 0.1-0.5% Oxygen |
| 447) | 0.5-1% Oxygen |
| 448) | 1-2% Oxygen |
| 449) | 2-5% Oxygen |
| 450) | 5-10% Oxygen |
| 451) | 10-20% Oxygen |

Animal Serum

| | |
|---|---|
| 452) | 0.1% Bovine Serum |
| 453) | 0.5% Bovine Serum |
| 454) | 1.0% Bovine Serum |
| 455) | 5.0% Bovine Serum |
| 456) | 10% Bovine Serum |
| 457) | 20% Bovine Serum |
| 458) | 10% Horse Serum |

Interleukins

| | |
|---|---|
| 459) | IL-1 |
| 460) | IL-2 |
| 461) | IL-3 |
| 462) | IL-4 |
| 463) | IL-5 |
| 464) | IL-6 |
| 465) | IL-7 |
| 466) | IL-8 |
| 467) | IL-9 |
| 468) | IL-10 |
| 469) | IL-11 |
| 470) | IL-12 |
| 471) | IL-13 |
| 472) | IL-14 |
| 473) | IL-15 |
| 474) | IL-16 |
| 475) | IL-17 |
| 476) | IL-18 |

Proteases

| | |
|---|---|
| 477) | MMP-1 |
| 478) | MMP-2 |
| 479) | MMP-3 |
| 480) | MMP-4 |
| 481) | MMP-5 |
| 482) | MMP-6 |
| 483) | MMP-7 |
| 484) | MMP-8 |
| 485) | MMP-9 |
| 486) | MMP-10 |
| 487) | MMP-11 |
| 488) | MMP-12 |
| 489) | MMP-13 |
| 490) | MMP-14 |
| 491) | MMP-15 |
| 492) | MMP-16 |
| 493) | MMP-17 |
| 494) | MMP-18 |
| 495) | MMP-19 |
| 496) | MMP-20 |
| 497) | MMP-21 |
| 498) | MMP-22 |
| 499) | MMP-23 |
| 500) | MMP-24 |
| 501) | Cathepsin B |
| 501) | Cathepsin C |
| 503) | Cathepsin D |
| 504) | Cathepsin G |
| 505) | Cathepsin H |
| 506) | Cathepsin L |
| 507) | Trypsin |
| 508) | Pepsin |
| 509) | Elastase |
| 510) | Carboxypeptidase A |
| 511) | Carboxypeptidase B |
| 512) | Carboxypeptidase G |
| 513) | Carboxypeptidase P |
| 514) | Carboxypeptidase W |
| 515) | Carboxypeptidase Y |
| 516) | Chymotrypsin |
| 517) | Plasminogen |
| 518) | Plasmin |
| 519) | u-type Plasminogen activator |
| 520) | t-type Plasminogen activator |
| 521) | Plasminogen activator inhibitor-1 |
| 522) | Carboxypeptidase Z |

Amino Acids

| | |
|---|---|
| 522) | Alanine |
| 523) | Arginine |
| 524) | Asparagine |
| 525) | Aspartic acid |
| 526) | Cysteine |
| 527) | Glutamine |
| 528) | Glutamic acid |
| 529) | Glycine |
| 530) | Histidine |
| 531) | Isoleucine |
| 532) | Leucine |
| 533) | Lysine |
| 534) | Methionine |
| 535) | Phenylalanine |
| 536) | Proline |
| 537) | Serine |
| 538) | Threonine |
| 539) | Tryptophan |
| 540) | Tyrosine |
| 541) | Valine |

Prostaglandins

| | |
|---|---|
| 542) | Prostaglandin A1 |
| 543) | Prostaglandin A2 |
| 544) | Prostaglandin B1 |
| 545) | Prostaglandin B2 |
| 546) | Prostaglandin D2 |
| 547) | Prostaglandin E1 |
| 548) | Prostaglandin E2 |
| 549) | Prostaglandin F1alpha |
| 550) | Prostaglandin F2alpha |
| 551) | Prostaglandin H |
| 552) | Prostaglandin I2 |
| 553) | Prostaglandin J2 |
| 554) | 6-Keto-Prostaglandin F1a |
| 555) | 16,16-Dimethyl-Prostaglandin E2 |
| 556) | 15d-Prostaglandin J2 |
| 557) | Prostaglandins pooled (542-556 above) |

Retinoid receptor agonists/Antagonists

| | |
|---|---|
| 558) | Methoprene Acid |
| 559) | All trans retinoic acid |
| 560) | 9-Cis Retinoic Acid |
| 561) | 13-Cis Retinoic Acid |
| 562) | Retinoid agonists pooled (558-561 above) |
| 563) | Retinoid antagonists |
| 564) | Retinoic acid receptor isotype RARalpha |
| 565) | Retinoic acid receptor isotype RARbeta |
| 566) | Retinoic acid receptor isotype RARgamma |
| 567) | Retinoic X receptor isotype RXRalpha |
| 568) | Retinoic X receptor isotype RXRbeta |
| 569) | Retinoic X receptor isotype RARgamma |

Miscellaneous Inducers

| | |
|---|---|
| 570) | Plant lectins |
| 571) | Bacterial lectins |
| 572) | forskolin |
| 573) | Phorbol myristate acetate |
| 574) | Poly-D-lysine |
| 575) | 1,25-dihydroxyvitamin D |
| 576) | Inhibin |
| 577) | Heregulin |
| 578) | Glycogen |
| 579) | Progesterone |
| 580) | IL-1 |
| 581) | Serotonin |
| 582) | Fibronectin - 45 kDa Fragment |
| 583) | Fibronectin - 70 kDa Fragment |
| 584) | glucose |
| 585) | beta mercaptoethanol |
| 586) | heparinase |
| 587) | pituitary extract |
| 588) | chorionic gonadotropin |
| 589) | adrenocorticotropic hormone |
| 590) | thyroxin |
| 591) | Bombesin |
| 592) | Neuromedin B |
| 593) | Gastrin-Releasing Peptide |
| 594) | Epinephrine |
| 595) | Isoproterenol |
| 596) | Ethanol |
| 597) | DHEA |
| 598) | Nicotinic Acid |
| 599) | NADH |
| 600) | Oxytocin |
| 601) | Vasopressin |
| 602) | Vasotocin |
| 603) | Angiotensin I |
| 604) | Angiotensin II |
| 605) | Angiotensin I Converting Enzyme |
| 606) | Angiotensin I Converting Enzyme Inhibitor |
| 607) | Chondroitinase AB |
| 608) | Chondroitinase C |
| 609) | Brain natriuretic peptide |
| 610) | Calcitonin |
| 611) | Calcium ionophore I |
| 612) | Calcium ionophore II |
| 613) | Calcium ionophore III |
| 614) | Calcium ionophore IV |
| 615) | Bradykinin |
| 616) | Albumin |
| 617) | Plasmonate |
| 618) | LIF |
| 619) | PARP inhibitors |
| 620) | Lysophosphatidic acid |
| 621) | (R)-METHANANDAMIDE |
| 622) | 1,25-DIHYDROXYVITAMIN D3 |
| 623) | 1,2-DIDECANOYL-GLYCEROL (10:0) |
| 624) | 1,2-DIOCTANOYL-SN-GLYCEROL |
| 625) | 1,2-DIOLEOYL-GLYCEROL (18:1) |
| 626) | 10-hydroxycamptothecin |
| 627) | 11,12-EPOXYEICOSATRIENOIC ACID |
| 628) | 12(R)-HETE |
| 629) | 12(S)-HETE |
| 630) | 12(S)-HPETE |
| 631) | 12-METHOXYDODECANOIC ACID |
| 632) | 13(S)-HODE |
| 633) | 13(S)-HPODE |
| 634) | 13,14-DIHYDRO-PGE1 |
| 635) | 13-KETOOCTADECADIENOIC ACID |
| 636) | 14,15-EPOXYEICOSATRIENOIC ACID |
| 637) | 1400W |
| 638) | 15(S)-HETE |
| 639) | 15(S)-HPETE |
| 640) | 15-KETOEICOSATETRAENOIC ACID |
| 641) | 17-Allylamino-geldanamycin |
| 642) | 17-OCTADECYNOIC ACID |
| 643) | 17-PHENYL-TRINOR-PGE2 |
| 644) | 1-ACYL-PAF |
| 645) | 1-HEXADECYL-2-ARACHIDONOYL-522) |
| 646) | GLYCEROL |
| 647) | 1-HEXADECYL-2-METHYLGLYCERO-3 PC |
| 648) | 1-HEXADECYL-2-O-ACETYL-GLYCEROL |
| 649) | 1-HEXADECYL-2-O-METHYL-GLYCEROL |
| 650) | 1-OCTADECYL-2-METHYLGLYCERO-3 PC |
| 651) | 1-OLEOYL-2-ACETYL-GLYCEROL |
| 652) | 1-STEAROYL-2-LINOLEOYL-GLYCEROL |
| 653) | 1-STEAROYL-2-ARACHIDONOYL-GLYCEROL |
| 654) | 2,5-ditertbutylhydroquinone |
| 655) | 24(S)-hydroxycholesterol |
| 656) | 24,25-DIHYDROXYVITAMIN D3 |
| 657) | 25-HYDROXYVITAMIN D3 |
| 658) | 2-ARACHIDONOYLGLYCEROL |
| 659) | 2-FLUOROPALMITIC ACID |
| 660) | 2-HYDROXYMYRISTIC ACID |
| 661) | 2-methoxyantimycin A3 |
| 662) | 3,4-dichloroisocoumarin |
| 663) | granzyme B inhibitor |
| 664) | 4-AMINOPYRIDINE |
| 665) | 4-HYDROXYPHENYLRETINAMIDE |
| 666) | 4-OXATETRADECANOIC ACID |
| 667) | 5(S)-HETE |
| 668) | 5(S)-HPETE |
| 669) | 5,6-EPOXYEICOSATRIENOIC ACID |
| 670) | 5,8,11,14-EICOSATETRAYNOIC ACID |
| 671) | 5,8,11-EICOSATRIYNOIC ACID |
| 672) | 5-HYDROXYDECANOATE |
| 673) | 5-iodotubercidin |
| 674) | 5-KETOEICOSATETRAENOIC ACID |
| 675) | 5′-N-Ethylcarboxamidoadenosine (NECA) |
| 676) | 6,7-ADTN HBr |
| 677) | 6-FORMYLINDOLO [3,2-B] CARBAZOLE |
| 678) | 7,7-DIMETHYLEICOSADIENOIC ACID |
| 679) | 8,9-EPOXYEICOSATRIENOIC ACID |
| 680) | 8-methoxymethyl-IBMX |
| 681) | 9(S)-HODE |
| 682) | 9(S)-HPODE |
| 683) | 9,10-OCTADECENOAMIDE |
| 684) | A-3 |
| 685) | AA-861 |
| 686) | acetyl (N)-s-farnesyl-1-cysteine |
| 687) | ACETYL-FARNESYL-CYSTEINE |
| 688) | Ac-Leu-Leu-Nle-CHO |
| 689) | ACONITINE |
| 690) | actinomycin D |
| 691) | ADRENIC ACID (22:4, n-6) |
| 692) | 1 mM |
| 693) | AG-1296 |
| 694) | AG1478 |
| 695) | AG213 (Tyrphostin 47) |
| 696) | AG-370 |
| 697) | AG-490 |
| 698) | AG-879 |
| 699) | AGC |
| 700) | AGGC |
| 701) | Ala-Ala-Phe-CMK |
| 702) | alamethicin |
| 703) | Alrestatin |
| 704) | AM 92016 |
| 704) | AM-251 |
| 706) | AM-580 |
| 707) | AMANTIDINE |
| 708) | AMILORIDE |
| 709) | Amino-1,8-naphthalimide [4-Amino-1,8-522) |
| | naphthalimide] |
| 710) | Aminobenzamide (3-ABA) [3-522) aminobenzamide (3- |
| | ABA)] |
| 711) | AMIODARONE |
| 712) | ANANDAMIDE (18:2, n-6) |
| 713) | ANANDAMIDE (20:3, n-6) |
| 714) | ANANDAMIDE (20:4, n-6) |
| 715) | ANANDAMIDE (22:4, n-6) |
| 716) | anisomycin |
| 717) | aphidicolin |
| 718) | ARACHIDONAMIDE |
| 719) | ARACHIDONIC ACID (20:4, n-6) |
| 720) | ARACHIDONOYL-PAF |
| 721) | aristolochic acid |
| 722) | Arvanil |
| 723) | ascomycin (FK-520) |
| 724) | B581 |
| 725) | BADGE |
| 726) | bafilomycin A1 |
| 727) | BAPTA-AM |
| 728) | BAY 11-7082 |
| 729) | BAY K-8644 |
| 730) | BENZAMIL |
| 731) | BEPRIDIL |
| 732) | Bestatin |
| 733) | beta-lapachone |
| 734) | Betulinic acid |
| 735) | bezafibrate |
| 736) | Blebbistatin |
| 737) | BML-190 |
| 738) | Boc-GVV-CHO |
| 739) | bongkrekic acid |
| 740) | brefeldin A |
| 741) | Bromo-7-nitroindazole [3-Bromo-7-nitroindazole] |
| 742) | Bromo-cAMP [8-Bromo-cAMP] |
| 743) | Bromo-cGMP [8-Bromo-cGMP] |
| 744) | bumetanide |
| 745) | BW-B 70C |
| 746) | C16 CERAMIDE |
| 747) | C2 CERAMIDE |
| 748) | C2 DIHYDROCERAMIDE |
| 749) | C8 CERAMIDE |
| 750) | C8 CERAMINE |
| 750) | C8 DIHYDROCERAMIDE |
| 751) | CA-074-Me |
| 753) | calpeptin |
| 754) | calphostin C |
| 755) | calyculin A |
| 756) | camptothecin |
| 757) | cantharidin |
| 758) | CAPE |
| 759) | capsacin(E) |
| 760) | capsazepine |
| 761) | CARBACYCLIN |
| 762) | castanospermine |
| 763) | CDC |
| 764) | Cerulenin |
| 765) | CGP-37157 |
| 766) | chelerythrine |
| 767) | CIGLITAZONE |
| 768) | CIMATEROL |
| 769) | CinnGEL 2Me |
| 770) | CIRAZOLINE |
| 771) | CITCO |
| 772) | CLOFIBRATE |
| 773) | clonidine |
| 774) | CLOPROSTENOL Na |
| 775) | clozapine |
| 776) | C-PAF |
| 777) | Curcumin |
| 778) | Cyclo [Arg-Gly-Asp-D-Phe-Val] |
| 779) | cycloheximide |
| 780) | protein synthesis inhibitor |
| 781) | cycloheximide-N-ethylethanoate |
| 782) | cyclopamine |
| 783) | CYCLOPIAZONIC ACID |
| 784) | cyclosporin A |
| 785) | cypermethrin |
| 786) | cytochalasin B |
| 787) | cytochalasin D |
| 788) | D12-PROSTAGLANDIN J2 |
| 789) | D609 |
| 790) | damnacanthal |
| 791) | DANTROLENE |
| 792) | decoyinine |
| 793) | Decylubiquinone |
| 794) | deoxymannojirimycin(1) |
| 795) | deoxynorjrimycin(1) |
| 796) | Deprenyl |
| 797) | DIAZOXIDE |
| 798) | dibutyrylcyclic AMP |
| 799) | dibutyrylcyclic GMP |
| 800) | DICHLOROBENZAMIL |
| 801) | DIHOMO-GAMMA-LINOLENIC ACID |
| 802) | DIHYDROSPHINGOSINE |
| 803) | DIINDOLYLMETHANE |
| 804) | DILTIAZEM |
| 805) | diphenyleneiodonium Cl |
| 806) | dipyridamole |
| 807) | DL-DIHYDROSPHINGOSINE |
| 808) | DL-PDMP |
| 809) | DL-PPMP |
| 810) | DOCOSAHEXAENOIC ACID (22:6 n-3) |
| 811) | DOCOSAPENTAENOIC ACID |
| 812) | DOCOSATRIENOIC ACID (22:3 n-3) |
| 813) | doxorubicin |
| 814) | DRB |
| 815) | E-4031 |
| 816) | E6 berbamine |
| 817) | E-64-d |
| 818) | Ebselen |
| 819) | EHNA HCl |
| 820) | EICOSA-5,8-DIENOIC ACID (20:2 n-12) |
| 821) | EICOSADIENOIC ACID (20:2 n-6) |
| 822) | EICOSAPENTAENOIC ACID (20:5 n-3) |
| 823) | EICOSATRIENOIC ACID (20:3 n-3) |
| 824) | ENANTIO-PAF C16 |
| 825) | epibatidine (+/−) |
| 826) | etoposide |
| 827) | FARNESYLTHIOACETIC ACID |
| 828) | FCCP |
| 829) | FIPRONIL |
| 830) | FK-506 |
| 831) | FLECAINIDE |
| 832) | FLUFENAMIC ACID |
| 833) | FLUNARIZINE |
| 834) | FLUPROSTENOL |
| 835) | FLUSPIRILINE |
| 836) | FPL-64176 |
| 837) | Fumonisin B1 |
| 838) | Furoxan |
| 839) | GAMMA-LINOLENIC ACID (18:3 n-6) |
| 840) | geldanamycin |
| 841) | genistein |
| 842) | GF-109203X |
| 843) | GINGEROL |
| 844) | Gliotoxin |
| 845) | GLIPIZIDE |
| 846) | GLYBURIDE |
| 847) | GM6001 |
| 848) | Go6976 |
| 849) | GRAYANOTOXIN III |
| 850) | GW-5074 |
| 851) | GW-9662 |
| 852) | H7] |
| 853) | H-89 |
| 854) | H9 |
| 855) | HA-1004 |
| 856) | HA1077 |
| 857) | HA14-1 |
| 858) | HBDDE |
| 859) | Helenalin |
| 860) | Hinokitiol |
| 861) | HISTAMINE |
| 862) | HNMPA-(AM)3 |
| 863) | Hoechst 33342 (cell permeable) (BisBenzimide) |
| 864) | Huperzine A [(−)-Huperzine A] |
| 865) | IAA-94 |
| 866) | IB-MECA |
| 867) | IBMX |
| 868) | ICRF-193 |
| 869) | Ikarugamyin |
| 870) | Indirubin |
| 871) | Indirubin-3′-monoxime |
| 872) | indomethacin |
| 873) | juglone |
| 874) | K252A |
| 875) | Kavain (+/−) |
| 876) | KN-62 |
| 877) | KT-5720 |
| 878) | L-744,832 |
| 879) | Latrunculin B |
| 880) | Lavendustin A |
| 881) | L-cis-DILTIAZEM |
| 882) | LEUKOTOXIN A (9,10-EODE) |
| 883) | LEUKOTOXIN B (12,13-EODE) |
| 884) | LEUKOTRIENE B4 |
| 885) | LEUKOTRIENE C4 |
| 886) | LEUKOTRIENE D4 |
| 887) | LEUKOTRIENE E4 |
| 888) | Leupeptin |
| 889) | LFM-A13 |
| 890) | LIDOCAINE |
| 891) | LINOLEAMIDE |
| 892) | LINOLEIC ACID |
| 893) | LINOLENIC ACID (18:3 n-3) |
| 894) | LIPOXIN A4 |
| 895) | L-NAME |
| 896) | L-NASPA |
| 897) | LOPERAMIDE |
| 898) | LY-171883 |
| 899) | LY-294002 |
| 900) | LY-83583 |
| 901) | Lycorine |
| 902) | LYSO-PAF C16 |
| 903) | Manoalide |
| 904) | manumycin A |
| 905) | MAPP, D-erythro |
| 906) | MAPP, L-erythro |
| 907) | mastoparan |
| 908) | MBCQ |
| 909) | MCI-186 |
| 910) | MDL-28170 |
| 911) | MEAD ACID (20:3 n-9) |
| 912) | MEAD ETHANOLAMIDE |
| 913) | methotrexate |
| 914) | METHOXY VERAPAMIL |
| 915) | Mevinolin (lovastatin) |
| 916) | MG-132 |
| 917) | Milrinone |
| 918) | MINOXIDIL |
| 919) | MINOXIDIL SULFATE |
| 920) | MISOPROSTOL, FREE ACID |
| 921) | mitomycin C |
| 922) | ML7 |
| 923) | ML9 |
| 924) | MnTBAP |
| 925) | Monastrol |
| 926) | monensin |
| 927) | MY-5445 |
| 928) | Mycophenolic acid |
| 929) | N,N-DIMETHYLSPHINGOSINE |
| 930) | N9-Isopropylolomoucine |
| 931) | N-ACETYL-LEUKOTRIENE E4 |
| 932) | NapSul-Ile-Trp-CHO |
| 933) | N-ARACHIDONOYLGLYCINE |
| 934) | NICARDIPINE |
| 935) | NIFEDIPINE |
| 936) | NIFLUMIC ACID |
| 937) | Nigericin |
| 938) | NIGULDIPINE |
| 939) | Nimesulide |
| 940) | NIMODIPINE |
| 941) | NITRENDIPINE |
| 942) | N-LINOLEOYLGLYCINE |
| 943) | nocodazole |
| 944) | N-PHENYLANTHRANILIC (CL) |
| 945) | NPPB |
| 946) | NS-1619 |
| 947) | NS-398 |
| 948) | NSC-95397 |
| 949) | OBAA |
| 950) | okadaic acid |
| 951) | oligomycin A |
| 952) | olomoucine |
| 953) | ouabain |
| 954) | PAF C16 |
| 955) | PAF C18 |
| 956) | PAF C18:1 |
| 957) | PALMITYLETHANOLAMIDE |
| 958) | Parthenolide |
| 959) | PAXILLINE |
| 960) | PCA 4248 |
| 961) | PCO-400 |
| 962) | PD 98059 |
| 963) | PENITREM A |
| 964) | pepstatin |
| 965) | PHENAMIL |
| 966) | Phenanthridinone [6(5H)-Phenanthridinone] |
| 967) | Phenoxybenzamine |
| 968) | PHENTOLAMINE |
| 969) | PHENYTOIN |
| 970) | PHOSPHATIDIC ACID, DIPALMITOYL |
| 971) | Piceatannol |
| 972) | pifithrin |
| 973) | PIMOZIDE |
| 974) | PINACIDIL |
| 975) | piroxicam |
| 976) | PP1 |
| 977) | PP2 |
| 978) | prazocin |
| 979) | Pregnenolone 16alpha carbonitrile |
| 980) | PRIMA-1 |
| 981) | PROCAINAMIDE |
| 982) | PROPAFENONE |
| 983) | propidium iodide |
| 984) | propranolol (S−) |
| 985) | puromycin |
| 986) | quercetin |
| 987) | QUINIDINE |
| 988) | QUININE |
| 989) | QX-314 |
| 990) | rapamycin |
| 991) | resveratrol |
| 992) | RETINOIC ACID, ALL TRANS |
| 993) | REV-5901 |
| 994) | RG-14620 |
| 995) | RHC-80267 |
| 996) | RK-682 |
| 997) | Ro 20-1724 |
| 998) | Ro 31-8220 |
| 999) | Rolipram |
| 1000) | roscovitine |
| 1001) | Rottlerin |
| 1002) | RWJ-60475-(AM)3 |
| 1003) | RYANODINE |
| 1004) | SB 202190 |
| 1005) | SB 203580 |
| 1006) | SB-415286 |
| 1007) | SB-431542 |
| 1008) | SDZ-201106 |
| 1009) | S-FARNESYL-L-CYSTEINE ME |
| 1010) | Shikonin |
| 1011) | siguazodan |
| 1012) | SKF-96365 |
| 1013) | SP-600125 |
| 1014) | SPHINGOSINE |
| 1015) | Splitomycin |
| 1016) | SQ22536 |
| 1017) | SQ-29548 |
| 1018) | staurosporine |
| 1019) | SU-4312 |
| 1020) | Suramin |
| 1021) | swainsonine |
| 1022) | tamoxifen |
| 1023) | Tanshinone IIA |
| 1024) | taxol = paclitaxel |
| 1025) | TETRAHYDROCANNABINOL-7-OIC ACID |
| 1026) | TETRANDRINE |
| 1027) | thalidomide |
| 1028) | THAPSIGARGIN |
| 1029) | Thiocitrulline [L-Thiocitrulline HCl] |
| 1030) | Thiorphan |
| 1031) | TMB-8 |
| 1032) | TOLAZAMIDE |
| 1033) | TOLBUTAMIDE |
| 1034) | Tosyl-Phe-CMK (TPCK) |
| 1035) | TPEN |
| 1036) | Trequinsin |
| 1037) | trichostatin-A |
| 1038) | trifluoperazine |
| 1039) | TRIM |
| 1040) | Triptolide |
| 1041) | TTNPB |
| 1042) | Tunicamycin |
| 1043) | tyrphostin 1 |
| 1044) | tyrphostin 9 |
| 1045) | tyrphostin AG-126 |
| 1046) | tyrphostin AG-370 |
| 1047) | tyrphostin AG-825 |
| 1048) | Tyrphostin-8 |
| 1049) | U-0126 |
| 1050) | U-37883A |
| 1051) | U-46619 |
| 1052) | U-50488 |
| 1053) | U73122 |
| 1054) | U-74389G |
| 1055) | U-75302 |
| 1056) | valinomycin |
| 1057) | Valproic acid |
| 1058) | VERAPAMIL |
| 1059) | VERATRIDINE |
| 1060) | vinblastine |
| 1061) | vinpocetine |
| 1062) | W7 |
| 1063) | WIN 55,212-2 |
| 1064) | Wiskostatin |
| 1065) | Wortmannin |
| 1066) | WY-14643 |
| 1067) | Xestospongin C |
| 1068) | Y-27632 |
| 1069) | YC-1 |
| 1070) | Yohimbine |
| 1071) | Zaprinast |
| 1072) | Zardaverine |
| 1073) | ZL3VS |
| 1074) | ZM226600 |
| 1075) | ZM336372 |
| 1076) | Z-prolyl-prolinal |
| 1077) | zVAD-FMK |
| 1078) | Ascorbate |
| 1079) | 5-azacytidine |
| 1080) | 5-azadeoxycytidine |
| 1081) | Hexamethylene bisacetamide (HMBA) |
| 1082) | Sodium butyrate |
| 1083) | Dimethyl sulfoxide. |
| 1084) | Goosecoid |
| 1085) | Glycogen synthase kinase-3 |
| 1086) | Galectin-1 |
| 1087) | Galectin-3 |

Cell Adhesion Molecules

| | |
|---|---|
| 1086) | Cadherin 1 (E-Cadherin) |
| 1087) | Cadherin 2 (N-Cadherin) |
| 1088) | Cadherin 3 (P-Cadherin) |
| 1089) | Cadherin 4 (R-Cadherin) |
| 1090) | Cadherin 5 (VE-Cadherin) |
| 1091) | Cadherin 6 (K-Cadherin) |
| 1092) | Cadherin 7 |
| 1093) | Cadherin 8 |
| 1094) | Cadherin 9 |
| 1095) | Cadherin 10 |
| 1096) | Cadherin 11 (OB-Cadherin) |
| 1097) | Cadherin 12 (BR-Cadherin) |
| 1098) | Cadherin 13 (H-Cadherin) |
| 1099) | Cadherin 14 (same as Cadherin 18) |
| 1100) | Cadherin 15 (M-Cadherin) |
| 1101) | Cadherin 16 (KSP-Cadherin) |
| 1102) | LI Cadherin |

Culture Media

| | |
|---|---|
| 1103) | DMEM (Dulbecco's Modified Eagle's Medium). |
| | HyClone Cat. No. SH30285.03 |
| 1104) | Airway Epithelial Growth Medium (PromoCell |
| | Cat. No. C-21260 with supplement Cat No. C-39160) |
| 1105) | Epi-Life (LSGS) Medium (Cascade Cat. No. |
| | M-EPIcf/PRF-500 with supplement Cat. No. S-003-10) |
| 1106) | Neural Basal Medium B-27 (Gibco Cat. No. |
| | 12348-017 with B-27 supplement Cat. No. 12587-010) |
| 1107) | Neural Basal Medium N-2 (Gibco Cat. No. |
| | 12348-017 with N-2 supplement Cat. No. 17502-048) |
| 1108) | HepatoZyme-SFM (Gibco Cat. No. 17705-021) |
| 1109) | Epi-Life (HKGS) Medium (Cascade Cat. No. |
| | M EPIcf/PRF-500 with supplement Cat. No. S-001-5) |
| 1110) | Endothelial Cell Growth Medium (PromoCell |
| | Cat. No. C-22221 with supplement Cat No. C-39221) |
| 1111) | Endothelial Cell SFM (Gibco Cat. No. 11111- |
| | 044 with basic fibroblast growth factor Cat. No. 13256- |
| | 029, epidermal growth factor, Cat. No. 13247-051 and |
| | fibronectin Cat. No. 33016-015) |
| 1112) | Skeletal Muscle Medium (PromoCell Cat. No. C- |
| | 23260 with supplement Cat. No. C-39360) |
| 1113) | Smooth Muscle Basal Medium (PromoCell Cat. |
| | No. C-22262 with supplement Cat. No. C-39262) |
| 1114) | MesenCult Medium (Stem Cell Technologies Cat. |
| | No. 05041 with supplement Cat. No. 05402) |
| 1115) | Melanocyte Growth Medium (PromoCell Cat. No. |
| | C 24010 with supplement Cat. No. C-39410) |
| 1116) | Ham's F-10 Medium |
| 1117) | Ham's F-12 Medium |
| 1118) | DMEM/Ham's F-12 50/50 mix |
| 1119) | Iscove's Modified Dulbecco's Medium (IMDM) |
| 1120) | Leibovitz's L-15 Medium |
| 1121) | McCoy's 5A Medium Modified |
| 1122) | RPMI 1640 Medium |
| 1123) | Glasgow's MEM (GMEM) |
| 1124) | Eagle's Medium |
| 1125) | Medium 199 |
| 1126) | MEM Eagle-Earle's |

Antibiotics

| | |
|---|---|
| 1127) | Penicillin |
| 1128) | Streptomycin |
| 1129) | Gentamycin |
| 1130) | Neomycin |
| 1131) | G418 |

Other Factors

| | |
|---|---|
| 1132) | Human plasma |
| 1133) | Chick embryo extract |
| 1134) | Human plasmanate |

TABLE II
Differentiated Cells and Tissues

```
Heart
  1) Ventricular myocardium
  2) Auricular myocardium
  3) Sinus node myocardium
  4) anterior, middle and posterior internodal tracts
  5) atrioventricular (AV) node
  6) His bundle
  7) right and left bundle branches
  8) anterior-superior and posterior-inferior divisions of the left bundle
  9) The Purkinje network
Musculo-Skeletal
10) Cartilage - Hyaline
11) Cartilage - Elastic
12) Cartilage - Fibrous
13) Bone - compact
14) Bone - cancellous
15) Intervertebral disc
16) Skeletal muscle
Nervous Tissues
17) Dopaminergic neurons of the substantia nigra
18) Autonomic - Parasympathetic
19) Autonomic - Sympathetic
20) Schwann cells
20) Cranial nerves
21) Myelinating - Schwann cells
22) Motor neurons
27) Outer neuroblastic layer of the developing retina
28) Inner neuroblastic layer of the developing retina
29) Outer nuclear layer of the retina
30) Outer plexiform layer of the retina
31) Inner nuclear layer of the retina
32) Inner plexiform layer of the retina
33) Ganglion cell layer of the retina
34) Thalamus
35) Hippocampus
36) Hypothalamus
37) Cerebral cortex
Respiratory System
38) Trachea
39) Tracheobronchial epithelium
40) Brochi
41) Lungs
42) Type I pneumocytes
43) Type II pneumocytes
Endocrine System
44) Pancreatic beta cells
45) Anterior pituitary
46) Neural pituitary
46) Adrenal cortex
47) Adrenal medulla
48) Thyroid gland
49) Parathyroid gland
Vascular System
50) Aorta
51) Pulmonary vein
52) capillaries
53) Vascular endothelium
54) Vascular smooth muscle
55) Pericytes
56) Adventitial cells
Hematopoietic system
55) Hematopoietic stem cells
56) Lymphoid progenitors
57) B lymphocytes
58) T lymphocytes
59) Myeloid progenitors
Integumentary system
60) Dermis
61) Epidermis
62) Hair follicles
63) Sebaceous glands
63) Sweat glands
64) Subcutaneous adipose tissue
Urinary System
65) Kidney
66) Renal tubule epithelial cells
67) Renal cortex
68) Ureters
69) Bladder
70) Urethra
Gastrointestinal system
71) Oral epithelium
72) Cheek epithelium
72) Teeth
72) Esophagus
72) Gastric mucosa
73) Jejunum
74) Ileum
75) Duodenum
76) Colon
77) Pancreas
78) Hepatic parenchymal cells
79) Hepatic Stellate (Ito) cells
Sensory systems
79) Olfactory epithelium
24) Inner ear
25) Lens
26) Cornea
23) Sensory neurons
25) Eye
26) Retinal pigment epithelium
```

TABLE III
Differentiating Cell Types (includes SPF chick embryonic
tissues, nonhuman animal embryonic/fetal cells and tissues,
and human embryonic/fetal cells and tissues

```
Endoderm - Embryonic
  1) Definitive endodermal (entodermal) cells
  2) Foregut endodermal cells
  3) Midgut endodermal cells
  4) Hindgut endodermal cells
  5) Ventral pancreatic bud cells
Mesoderm - Embryonic
  6) Intraembryonic mesodermal cells
  7) Prechordal plate mesodermal cells
  8) Notochordal plate mesodermal cells
  9) Notochord mesodermal cells
10) Paraxial mesodermal cells
11) Intermediate mesodermal cells
12) Lateral plate mesodermal cells
13) Splanchnopleuiric mesodermal cells
14) Somatopleuric mesodermal cells
15) Somitomeric mesodermal cells
16) Somite mesodermal cells
17) Cervical somite mesodermal cells
18) Thoracic somite mesodermal cells
19) Lumbar somite mesodermal cells
20) Sacral somite mesodermal cells
21) Sclerotome mesodermal cells
22) Myotome mesodermal cells
23) Epimere myotome mesodermal cells
24) Hypomere myotome mesodermal cells
25) Dermatome mesodermal cells
26) Angioblasts
27) Mural progenitor cells
28) Vascular smooth muscle cells
29) Pericytes
30) Myoepithelial cells
31) Enteric (intestinal) smooth muscle cells
32) Limb bud mesenchyme
33) Osteoblasts
34) Synoviocytes
35) Hemangioblasts
36) Angioblasts
37) Skeletal muscle myoblasts
38) cardiogenic mesoderm
39) Endocardial primordial cells
40) Epi-myocardial primordial cells
41) Dorsal mesocardial cells
Ectoderm - Embryonic
42) cranial neural crest
43) cardiac neural crest
44) vagal neural crest
45) trunk neural crest
Extraembryonic Cells
46) Hypoblast (primary endoderm)
47) Extraembryonic endodermal cells
49) Amnioblasts
49) Syncytiotrophoblasts
50) Cytotrophoblasts
51) Extraembryonic mesodermal cells
```

TABLE IV
Teratogens

Abovis
Acebutolol
Acebutolol hydrochloride
Acemetacin
Acepreval
Acetaldehyde
Acetamide
5-Acetamide-1,3,4-thiadiazole-2-sulfonamide
Acetazolamide sodium
Acetic acid methylnitrosaminomethyl ester
Acetohydroxamic acid
Acetonitrile
3-(alpha-Acetonyl-para-nitrobenzyl)-4-hydroxy-coumarin
para-Acetophenetidide
17-Acetoxy-19-nor-17-alpha-pregn-4-EN-20-YN-3-one
Acetoxyphenylmercury
Acetoxytriphenylstannane
1-alpha-Acetylmethadol hydrochloride
Acetylsalicylic acid
Acetyltryptophan
Acid red 92
4,-(9-Acridinylamino) methanesulphon-meta-anisidide
Acriflavin hydrochloride
Acrylic acid
Acrylonitrile
Actihaemyl
Actinomycin
Actinomycin C
Actinomycin D
Acyclovir
Acyclovir sodium salt
Adalat
1-Adamantanamine hydrochloride
Adapin
Adenine
Adenosine-3,-(alpha-amino-para-
methoxyhydrocinnamamido)-3,-deoxy-n,n-dimethyl
Adipic acid bis (2-ethylhexyl) ester
Adipic acid dibutyl ester
Adipic acid di(2-hexyloxyethyl) ester
Adobiol
Adona trihydrate
1-Adrenaline chloride
Adrenocorticotrophic hormone
Adriamycin
Aflatoxin
Aflatoxin B1
Afridol blue
Agent orange
Alclometasone dipropionate
Alcohol sulphate
Aldactazide
Aldecin
Aldimorph
Aldrin
alpha-Alkenesulfonic acid
Alkyl dimethylbenzyl ammonium chloride
3-(Alkylamino) propionitrile
Alkylbenzenesulfonate
Allantoxanic acid, potassium salt
Alloxan
Allyl chloride
Allyl glucosinolate
Allyl isothiocyanate
6-Allyl-6,7-dihydro-5h-dibenz (c,e) azepine phosphate
Allylestrenol
(4-Allyloxy-3-chlorophenyl) acetic acid
Alternariol
Alternariol monomethyl ether and alternariol (1:1)
Alternariol-9-methyl ether
Aluminum aceglutamide
Aluminum chloride
Aluminum chloride hexahydrate
Aluminum lactate
Aluminium (III) nitrate, nonahydrate (1:3:9)
Aluminium potassium sulfate, dodecahydrate
Ambroxol hydrochloride
Ametycin
Amfenac sodium monohydrate
Amicardine
N1-Amidinosulfanilamide
Amidoline
5-((2-Aminoacetamido) methyl)-1-(4-chloro-2-
(orthochlorobenzoyl) phenyl)-n,n-dimethyl-1H-S-
triazole-3-carboxamide, hydrochloride, dihydrate
Aminoacetonitrile bisulfate
Aminoacetonitrile sulfate
2-Aminobenzimidazole
2-Amino-6-benzimidazolyl phenylketone
Aminobenzylpenicillin
5-Amino-1-bis (dimethylamide) phosphoryl-3-phenyl-
1,2,4-triazole
2-Amino-5-bromo-6-phenyl-4 (1h)-pyrimidinone
4-Amino-2-(4-butanoylhexahydro-1h-1,4-diazepin-1-yl)-
6,7-dimethoxyquinazoline hydrochloride
2-Amino-5-butylbenzimidazole
5-Amino-1,6-dihydro-7h-v-triazolo (4,5-d) pyrimidin-7-
one
3-(2-aminoethyl) indol-5-ol
3-(2-aminoethyl) indol-5-ol creatinine sulfate
trans-4-Aminoethylcyclohexane-1-carboxylic acid
Aminoglutethimide
2-Amino-3-hydroxybenzoic acid
8-Amino-7-hydroxy-3,6-napthalenedisulfonic acid, sodium
salt
4-Amino-n-(6-methoxy-3-pyridazinyl)-benzenesulfonamide
3-Amino-4-methylbenzenesulfonylcyclohexylurea
2-Amino-6-(1,-methyl-4,-nitro-5,-imidazolyl)
mercaptopurine
1-(4-Amino-2-methylpyrimidin-5-yl)methyl-3-(2-
chloroethyl)-3-nitrosourea
2-Amino-4-(methylsulfinyl) butyric acid
5-Amino-2-napthalenesulfonic acid sodium salt
6-Aminonicotinamide
2-Amino-4-nitroaniline
4-Amino-2-nitroaniline
Aminonucleoside puromycin
2-Aminophenol
3-Aminophenol
4-Aminophenol
meta-Aminophenol, chlorinated
7-(d-alpha-aminophenylacetamido)
desacetoxycephalosporanic acid
3-Aminopropionitrile
beta-Aminopropionitrile fumarate
Aminopropyl aminoethylthiophosphate
3-(2-Aminopropyl) indole
Aminopteridine
2-Aminopurine-6-thiol
Aminopyrine sodium sulfonate
Aminopyrine-barbital
5-Amino-2-beta-d-ribofuranosyl-as-triazin-3-(2H)-one
4-Amino-2,2,5,5-tetrakis (trifluoromethyl)-3-
imidazoline
2-Amino-1,3,4-thiadiazole
2-Amino-1,3,4-thiadiazolehydrochloride
2-Amino-1,3,4-thiadiazole-5-sulfonamide sodium salt
1-Amino-2-(4-thiazolyl)-5-benzimidazolecarbamic acid
isopropyl ester
Amitriptyline-n-oxide
Amitrole
Ammonium vanadate
Amosulalol hydrochloride
Amoxicillin trihydrate
dl-Amphetamine sulfate
Ampicillin trihydrate
Amrinone
Amsacrine lactate
Amygdalin
Anabasine
Anatoxin I
Androctonus amoreuxi venom
Androfluorene
Androfurazanol
Androstanazol
Androstenediol dipropionate
Androstenedione
Androstenolone
Androstestone-M
Angel dust
Angiotonin
Anguidin
Aniline violet
6-(para-anilinosulfonyl) metanilamide
2-Anthracenamine
Antibiotic BB-K8
Antibiotic BB-K8 sulfate
Antibiotic BL-640
Antibiotic MA 144A1
Antimony oxide
Apholate
9-beta-d-Arabino furanosyl adenine
Arabinocytidine
Ara-C palmitate
Araten phosphate
Arathane
1-Arginine monohydrochloride
Aristocort
Aristocort acetonide
Aristocort diacetate
Aristolic acid
Aristospan
Aromatol
Arotinoic acid
Arotinoic methanol
Arotinoid ethyl ester
Arsenic
ortho-Arsenic acid
Arsenic acid, disodium salt, heptahydrate
Arsenic acid, sodium salt
Arsenic trioxide
Asalin
1-Ascorbic acid
1-Asparaginase
Atrazine
Atromid S
Atropine
Atropine sulfate (2:1)
Auranofin
Aureine
1-Aurothio-d-glucopyranose
Ayush-47
Azabicyclane citrate
Azactam
Azacytidine
Azaserine
Azathioprine
Azelastine hydrochloride
1-2-Azetidinecarboxylic acid
Azinphos methyl
Azo blue
Azo ethane
Azosemide
Azoxyethane
Azoxymethane
Baccidal
Bacmecillinam
Bal
Barbital sodium
Barium ferrite
Barium fluoride
Bayer 205
Baythion
Befunolol hydrochloride
Bendacort
Bendadryl hydrochloride
Benedectin
Benomyl
Benzarone
d-Benzedrine sulfate
Benzenamine hydrochloride
Benzene
Benzene hexachloride-g-isomer
1-Benzhydryl-4-(2-(2-hydroxyethoxy)ethyl)piperazine
Benzidamine hydrochloride
2-Benzimidazolecarbamic acid
1-(2-Benzimidazolyl)-3-methylurea
1,2-Benzisothiazol-3 (2H)-one-1,1-dioxide
1,2-Benzisoxazole-3-methanesulfonamide
Benzo (alpha) pyrene
Benzo (e) pyrene
Benzoctamine hydrochloride
para-Benzoquinone monoimine
Benzothiazole disulfide
2-Benzothiazolethiol
2-Benzothiazolyl-N-morpholinosulfide
2-(meta-Benzoylphenyl) propionic acid
2-Benzylbenzimidazole
Benzyl chloride
Benzyl penicillinic acid sodium salt
Beryllium chloride
Beryllium oxide
Bestrabucil
Betamethasone
Betamethasone acetate and betamethasone phosphate
Betamethasone benzoate
Betamethasone dipropionate
Betamethasone disodium phosphate
Betel nut
Betnelan phosphate
BHT (food grade)
Bindon ethyl ether
Binoside
4-Biphenylacetic acid
2-Biphenylol
2-Biphenylol, sodium salt
3-(4-Biphenylylcarbonyl) propionic acid
2,2-Bipyridine
Bis(para-acetoxyphenyl)-2-methylcylcophexylidenemethane
4,4-Bis(1-amino-8-hydroxy-2,4-disulfo-7-napthylazo)-
3,3,-bitolyl,tetrasodium salt
1,4-Bis(3-bromopropionyl)-piperazine
1,3-Bis(carbamoylthio)-2-(N,N-dimethylamino)propane
hydrochloride
trans-N,N,-Bis(2-chlorobenzyl)-1,4 cyclohexanebis
(methylamine) dihydrochloride
Bis(2-chloroethyl) amine hydrochloride
4,-(Bis (2-chloroethyl) amino) acetanilide
4,-(Bis (2-chloroethyl) amino)-2-fluoro acetanilide
dl-3-(para-(Bis (2-chloroethyl) amino) phenyl)alanine
Bis(beta-chloroethyl) methylamine
Bis(2-chloroethyl) methylamine hydrochloride
Bis (2-chloroethyl) sulfide
N,N,-Bis (2-chloroethyl)-N-nitrosourea
N,N,-Bis (2-chloroethyl)-para-phenylenediamine
Bis (para-chlorophenyl) acetic acid
2,2-Bis (ortho, para-chlorophenyl)-1,1,1-
trichloroethane
1,1-Bis (para-chlorophenyl)-2,2,2-trichloroethanol
Bis (beta-cyanoetyl) amine
Bis (dichloroacetyl)-1,8-diaminooctane
3,5-Bis-dimethylamino-1,2,4-dithiazolium chloride
Bis (dimethyldithiocarbamato) zinc
(((3,5-Bis(1,1-dimethylethyl)-4-
hydroxyphenyl)methyl)thio)acetic acid 2-ethylhexyl
ester
Bis (dimethylthiocarbamoyl) sulfate
2,4-Bis (ethylamino)-6-chloro-s-triazine
Bis (ethylmercuri) phosphate
Bis-HM-A-TDA
Bishydroxycoumarin
Bis (4-hydroxy-3-coumarin) acetic acid ethyl ester
1,4-Bis ((2-((2-hydroxyethyl) amino) ethyl) amino)-
9,10-athracenedione diacetate
Bis (isooctyloxycarbonylmethylthio) dioctyl stannane
Bis (2-methoxy ethyl) ether
Bisphenol A
1,4-Bis (phenyl amino) benzene
Bis (tributyl tin) oxide
2-(3,5-Bis (trifluoromethyl) phenyl)-N-methyl-
hydrazinecarbothioamide (9CI)
Bladex
Bleomycin sulfate
Bomt
Bracken fern, dried
Bradykinin
Bredinin
Bremfol
Bromacil
Bromazepam
Bromocriptine
Bromocriptine mesilate
5-Bromo-2,-deoxyuridine
2-Bromo-d-lysergic acid diethylamide
6-Bromo-1,2-napththoquinone
Bromoperidol
Bromophenophos
4-Bromophenyl chloromethyl sulfone
Buclizine dihydrochloride
Budesonide
Bunitrolol hydrochloride
Buprenorphine hydrochloride
1,3-Butadiene
Butamirate citrate
1,4-Butanediamine
1,4-Butanediol dimethyl sulfonate
4-Butanolide
Butobarbital
Butoctamide semisuccinate
Butorphanol tartrate
Butoxybenzyl hyoscyamine bromide
2-Butoxyethanol
para-Butoxyphenylacetohydroxamic acid
Butriptyline
Bromoperidol
Bromophenophos
4-Bromophenyl chloromethyl sulfone
Buclizine dihydrochloride
Budesonide
Bunitrolol hydrochloride
Buprenorphine hydrochloride
1,3-Butadiene
Butamirate citrate
1,4-Butanediamine
1,4-Butanediol dimethyl sulfonate
4-Butanolide
Butobarbital
Butoctamide semisuccinate
Butorphanol tartrate
Butoxybenzyl hyoscyamine bromide
2-Butoxyethanol
para-Butoxyphenylacetohydroxamic acid
Butriptyline
n-Butyl acetate
n-Butyl alcohol
sec-Butyl alcohol
tert-Butyl alcohol
alpha,-((tert-Butyl amino) methyl)-4-hydroxy-meta-
xylene-alpha,alpha-diol
Butyl carbamate
Butyl carbobutoxymethyl phthalate
Butyl dichlorophenoxyacetate
Butyl ethyl acetic acid
Butyl flufenamate
n-Butyl glycidyl ether
n-Butyl mercaptan
n-Butyl-3,ortho-acetyl-12-b-13-alpha-dihydrojervine
1-(tert-Butylamino)-3-(2-chloro-5-methylphenoxy)-2-
propanol hydrochloride
alpha-Butylbenzenemethanol
5-Butyl-2-benzimidazolecarbamic acid methyl ester
5-Butyl-1-cylcohexylbarbituric acid
2-sec-Butyl-4,6-dinitrophenol
4-Butyl-1,2-diphenyl-3,5-dioxo pyrazolidine
n-Butyl-N-nitroso-1-butamine
N-Butyl-N-nitroso ethyl carbamate
n-Butylnitrosourea
1-Butyl-2′,6′-pipecoloxylidide
1-Butyl-3-sulfanilyl urea
1-Butyl-3-(para-tolyl sulfonyl) urea
1-Butyl-3-(para-tolylsulfonyl) urea, sodium salt
Butyl-2,4,5-trichlorophenoxyacetate
1-Butyryl-4-(phenylallyl) piperazine hydrochloride
Buzepide methiodide
Cadmium
Cadmium (II) acetate
Cadmium chloride
Cadmium chloride, dihydrate
Cadmium compounds
Cadmium oxide
Cadmium sulfate (1:1)
Cadmium sulfate (1:1) hydrate (3:8)
Cadralazine
Caffeic acid
Caffeine
Calcium EbrA complex
Calcium fluoride
Calcium phosphonomycin hydrate
Calcium trisodium diethylene triamine pentaacetate
Calcium valproate
Calcium-N-2-ethylhexyl-beta-oxybutyramide semisuccinate
Cambendazole
Camphorated oil
Candida albicans glycoproteins
Cannabidiol
Cannabinol
Cannabis
Cap
Caprolactam
Captafol
Captan
Carbamates
Carbaryl
Carbendazim and sodium nitrite (5:1)
Carbidopa
Carbinilic acid isopropyl ester
Carbofuran
Carbon dioxide
Carbon disulfide
Carbon monoxide
Carbon tetrachloride
Carboprost tromethamine
Cargutocin
Carmetizide
Carmofur
1-Carnitine hydrochloride
Carnosine
Carzinophilin
Cassava, manihot utilissima
Catatoxic steroid No. 1
d-Catechol
CAZ pentahydrate
Cefamandole sodium
Cefotaxime sodium
Cefazedone
Cefazolin sodium salt
Cefmetazole
Cefmetazole sodium
Cefroxadin
Cefuroxim
Celestan-depot
Cellryl
Cellulose acetate monophthalate
Centbucridine hydrochloride
Centchroman
Cephalothin
Cervagem
Cesium arsenate
Cethylamine hydrofluoride
alpha-Chaconine
Chenodeoxycholic acid
Chlodithane
Chlorambucil
Chloramphenicol
Chloramphenicol monosuccinate sodium salt
Chloramphenicol palmitate
Chlorcyclizine hydrochloride
Chlorcyclizine hydrochloride A
Chlorcyclohexamide
Chlordane
Chlorimipramine
Chlorinated camphene
Chlorinated dibenzo dioxins
Chlorisopropamide
Chlormadinon
para-Chloro dimethylaminoazobenzene
2-Chloroadenosine
1-(3-Chloroallyl)-3,5,7-triaza-1-azoniaadamantane
chloride
3-Chloro-4-aminoaniline
1-((para-(2-(Chloro-ortho-
anisamido)ethyl)phenyl)sulfonyl)-3-cylcohexyl urea
Chlorobenzene
ortho-Chlorobenzylidene malononitrile
1-para-Chlorobenzyl-1H-indazole-3-carboxylic acid
7-Chloro-5-(ortho-chlorophenyl)-1,3-dihydro-3-hydroxy-
2H-1,4-benzodiazepin-2-one
Chlorocylcine
6-Chloro-5-Cyclohexyl-1-indancarboxylic acid
6-Chloro-5-(2,3-dichlorophenoxy)-2-methylthio-
benzimidazole
5-Chloro-2-(2-(diethylamino)ethoxy)benzanilide
7-Chloro-1,3-dihydro-5-phenyl,2H-1,4-benzodiazepin-2-
one
Chloroethyl mercury
1-(2-Chloroethyl)-3-cylcohexyl-1-nitrosourea
1-Chloro-3-ethyl-1-penten-4-YN-3-OL
Chloroform
4-Chloro-N-furfuryl-5-sulfamoylanthranilic acid
Chlorogenic acid
endo-4-Chloro-N-(hexahydro-4,7-methanoisoindol-2-YL)-3-
sulfamoylbenzamide
(−)-N-((5-Chloro-8-hydroxy-3-methyl-1-OXO-7-
isochromanyl) carbonyl)-3-phenylalanine
5-Chloro-7-iodo-8-quinolinol
(4-Chloro-2-methylphenoxy) acetic acid
2-(4-Chloro-2-methylphenoxy) propanoic acid (R) (9CI)
4-Chloro-2-methylphenoxy-alpha-propionic acid
7-Chloro-1-methyl-5-phenyl-1H-1,5-benzodiazepine-
2,4(3H,5H)-dione
2-Chloro-11-(4-methylpiperazino) dibenzo (b,f) (1,4)
thiazepine
4-((5-Chloro-2-OXO-3(2H)-benzothiazolyl)acetyl)-1-
piperazineethanol
4-(3-(2-Chlorophenothiazin-10-YL)propyl)-1-
piperazineethanol
4-Chlorophenylalanine
1-(para-Chloro-alpha-phenylbenzyl)-4-(2-((2-
hydroxyethoxy) ethyl)piperazine)
1-(meta-Chlorophenyl)-3-N,N-dimethylcarbamoyl-5-
methoxypyrazole
3-(para-Chlorophenyl)-1,1,dimethylurea
5,(2-Chlorophenyl)-7-ethyl-1-methyl-1,3-dihydro-2H-
thieno (2,3-e) (1,4) diazepin-2-one
N-3-Chlorophenylisopropylcarbamate
3-(4-Chlorophenyl)-1-methoxy-1-methylurea
2-(ortho-Chlorophenyl)-2-(methylamino)cyclohexanone
hydrochloride
3-(para-Chlorophenyl)-1-methyl-1-(1-methyl-2-propynyl)
urea
4-(para-Chlorophenyl)-2-phenyl-5-thiazoleacetic acid
1-(para-Chlorophenylsulfonyl)-3-propylurea
para-Chlorophenyl-2,4,5-trichlorophenyl sulfone
4-Chlorophenyl-2,4,5-trichlorophenylazosulfide mixed
with 1,1-bis(4-chlorophenyl)ethanol
Chloropromazine
Chloropromazine hydrochloride
Chloroquine
Chloroquine diphosphate
N-(3-Chloro-ortho-tolyl) anthranilic acid
2-((4-Chloro-ortho-tolyl)oxy)propionic acid potassium
salt
Chloro(triethylphosphine)gold
Chlorovinylarsine dichloride
4-Chloro-3,5-xylenol
Chlorphentermine
g-(4-(para-Chlorphenyl)-4-hydroxiperidino)-para-
fluorbutyrophenone
Cholecalciferol
Cholesterol
Cholestyramine
Chorionic gonadotropin
Chromium chloride
Chromium (VI) oxide (1:3)
Chromium trichloride hexahydrate
Chromomycin A3
C.I. 45405
C.I. Direct blue 1, tetrasodium salt
C.I. Direct blue 6, tetrasodium salt
C.I. Direct blue 14, tetrasodium salt
C.I. Direct blue 15, tetrasodium salt
Cilostazol
Cinoxacin
Citreoviridin
Citrinin
Citrus hystrix DC., fruit peel extract
Clavacin
Clindamycin-2-palmitate monohydrochloride
Clindamycin-2-phosphate
Cloazepam
Clobetasone butyrate
Cloconazole hydrochloride
Clofedanol hydrochloride
Clofexamide phenylbutazone
Clomiphene
racemic-Clomiphene citrate
trans-Clomiphene citrate
Clonidine hydrochloride
Clonixic acid
Cloxazolazepam
Clozapine
Coagulase
Cobalt (III) acetylacetonate
Cobalt (II) chloride
Corn oil
Corticosterone
Corticosterone acetate
Cortisol
Cortisone
Cortisone-21-acetate
Cottonseed oil (unhydrogenated)
Coumarin
Cravetin
meta-Cresol
Cumoesterol
S-1-Cyano-2-hydroxy-3-butene
Cyanotrimethylandrostenolone
Cycasin
Cyclocytidine hydrochloride
Cycloguanyl
Cyclohexanamine hydrochloride
Cycloheximide
Cyclohexylamine
Cyclohexylamine sulfate
2-(Cyclohexylamino)ethanol
N-Cyclohexyl-2-benzothiazolesulfenamide
4-(4-Cyclohexyl-3-chlorophenyl)-4-oxobutyric acid
1-Cyclohexyl-3-para-tolysulfonylurea
Cyclonite
Cyclopamine
Cyclophosphamide hydrate
Cyclophosphoramide
alpha-Cyclopiazonic acid
5-(Cyclopropylcarbonyl)-2-benzimidazolecarbamic acid
methyl ester
Cyprosterone acetate
Cysteine-germanic acid
Cytochalasin B
Cytochalasin E
Cytostasan
Cytoxal alcohol
Cytoxyl amine
Demeton-O + Demeton-S
Demeton-O-methyl
Demetrin
Denopamine
11-Deoxo-12-beta,13-alpha-dihydro-11-alpha-
hydroxyjervine
11-Deoxojervine-4-EN-3-one
2,-Deoxy-5-fluorouridine
2-Deoxyglucose
2,-Deoxy-5-iodouridine
4-Deoxypyridoxol hydrochloride
Dephosphate bromofenofos
Depofemin
Depo-medrate
N-Desacetylthiocolchicine
Desoxymetasone
2-Desoxyphenobarbital
Detergents, Liquid containing AES
Detergents, Liquid containing LAS
Dexamethasone acetate
Dexamethasone 17,21-dipropionate
Dexamethasone palmitate
Dextran 1
Dextran 70
Dextropropoxyphene napsy
alpha-DFMO
Diabenor
Diacetylmorphine hydrochloride
Dialifor
Diamicron
2,4-Diamino-6-methyl-5-phenylpyrimidine
2,4-Diamino-5-phenyl-6-ethylpyrimidine
2,4-Diamino-5-phenyl-6-propylpyrimidine
2,4-Diamino-5-phenylpyrimidine
2,5-Diaminotoluene dihydrochloride
Diazepam
Diazinon
6-Diazo-5-oxonorleucine
Diazoxide
Dibekacin
5H-Dibenz (b,f) azepine-5-carboxamide
5H-Dibenz (b,f) azepine, 3-chloro-5-(3-(4-carbamoyl-
4-piperidinopiperine
Dibenz (b,f) (1,4) oxazepine
Dibenzacepin
Dibenzyline hydrochloride
1,2-Dibromo-3-chloropropane
3,5-Dibromo-4-hydroxyphenyl-2-ethyl-3-benzofuranyl
ketone
Dibromomaleinimide
1,6-Dibromomannitol
Dibutyl phthalate
N,N-Di-n-butylformamide
Dibutyryl cyclic amp
Dicarbadodecaboranylmethylethyl sulfide
Dicarbadodecaboranylmethylpropyl sulfide
1-(2,4-Dichlorbenzyl)indazole-3-carboxylic acid
Dichloroacetonitrile
(ortho-((2,6-Dichloroanilino)phenyl) acetic acid sodium
salt
ortho-Dichlorobenzene
para-Dichlorobenzene
4,5-Dichloro-meta-benzenedisulfonamide
2,2,-Dichlorobiphenyl
Dichloro-1,3-butadiene
1,4-Dichloro-2-butene
2,2-Dichloro-1,1-difluorethyl methyl ether
5,5-Dichloro-2,2,-dihydroxy-3,3,-dinitrobiphenyl
1,1-Dichloroethane
2,3-Dichloro-N-ethylmaleinimide
Dichloromaleimide
Dichloro-N-methylmaleimide
2,4-Dichloro-4,-nitrodiphenyl ether
2,4-Dichlorophenol
(2,4-Dichlorophenoxy) acetic acid butoxyethyl ester
(2,4-Dichlorophenoxy) acetic acid dimethylamine
4-(2,4-Dichlorophenoxy) butyric acid
2-(2,4-Dichlorophenoxy) propionic acid
(+)-2-(2,4-Dichlorophenoxy) propionic acid
3,4-Dichlorophenoxyacetic acid
2,4-Dichlorophenoxyacetic acid propylene glycol butyl
ether ester
2-(2,6-Dichlorophenylamino)-2-imidazoline
3,6-Dichloro-2-pyridinecarboxylic acid
Dichlorvos
Dicyclohexyl adipate
Dicyclohexyl-18-crown-6
Dicyclopentadienyldichlorotitanium
7,8-Didehydroretinoic acid
Dieldrin
Diethyl carbitol
Diethyl carbonate
Diethyl mercury
Diethyl phthalate
Diethyl sulfate
2-(Diethylamino)-2′,6′-acetoxylidide
2-Diethylamino-2′,6′-acetoxylidide hydrochloride
ortho-(Diethylaminoethoxy) benzanilide
2-(2-(Diethylamino)ethoxy)-5-bromobenzanilide
2-(2-(Diethylamino)ethoxy)-2,-chloro-benzanilide
2-(2-(Diethylamino)ethoxy)-3,-chloro-benzanilide
2-(2-(Diethylamino)ethoxy)-3,-chloro-methylbenzanilide
(para-2-Diethylaminoethoxyphenyl)-1-phenyl-2-para-
anisylethanol
1-(2-(Diethylamino)ethyl)reserpine
7-Diethylamino-5-methyl-s-triazolo(1,5-alpha)
pyrimidine
N,N-Diethylbenzenesulfonamide
Diethylcarbamazine
Diethylcarbamazine acid citrate
Diethyldiphenyl dichloroethane
Diethylene glycol
Diethylene glycol monomethyl ether
1,2-Diethylhydrazine
1,2-Diethylhydrazine dihydrochloride
N,N-Diethyllsergamide
N,N-Diethyl-4-methyl-3-oxo-5-alpha-4-azaandrostane-17-
beta-carboxamide
3,3-Diethyl-1-(meta-pyridyl)triazene
a,a-Diethyl-(E)-4,4,-stilbenediol bis(dihydrogen
phosphate)
a,a-Diethyl-4,4,-stilbenediol disodium salt
Diethylstilbesterol
Diethylstilbestrol dipalmitate
Diethylstilbestrol dipropionate
Diflorasone diacetate
Diflucortolone valerate
dl-alpha-Difluoromethylornithine
5-(2,4-Difluorophenyl) salicylic acid
Difluprednate
Digoxin
Dihydantoin
Dihydrocodeinone bitartrate
Dihydrodiethylstilbestrol
3,4-Dihydro-6-(4-(3,4-dimethoxybenzoyl)-1-piperazinyl)-
2(1H)-quinolinone
5,6-Dihydro-N-(3-(dimethylamino)propyl)-11H-
dibenz(b,e)azepine
10,11-Dihydro-5-(3-(dimethylamino)propyl)-5H-
dibenz(b,f)azepine hydrochloride
5,6-Dihydro-para-dithiin-2,3-dicarboximide
12,b,13,alpha-Dihydrojervine
10,11-Dihydro-5-(3-(methylamino)propyl)-5H-
dibenz(b,f)azepine hydrochloride
1,7-Dihydro-6H-purin-6-one
7,8-Dihydroretinoic acid
Dihydrostreptomycin
4-Dihydrotestosterone
3-alpha,17-beta-Dihydroxy-5-alpha-androstane
3-alpha,7-beta-Dihydroxy-6-beta-cholan-24-OIC acid
1 alpha,25-Dihydroxycholecalciferol
3,4-Dihydroxy-alpha-((isopropylamino)methyl)benzyl
alcohol
1-Dihydroxyphenyl-1-alanine
1-(−)-3-(3,4-Dihydroxyphenyl)-2-methylanine
17R,21-alpha-Dihydroxy-4-propylajmalanium hydrogen
tartrate
DI(2-Hydroxy-n-propyl) amine
Diisobutyl adipate
Diisobutyl phthalate
alpha-(2-(Diisopropylamino)ethyl)-alpha-phenyl-2-
pyridineacetamide
Dilantin
Dilaudid
Diltiazem hydrochloride
Dimatif
Dimethoxy ethyl phthalate
1,2-Dimethoxyethane
3,6-Dimethoxy-4-sulfanilamidopyridazine
Dimethyl adipate
O,O-Dimethyl methylcarbamoylmethyl phosphordithioate
Dimethyl phthalate
Dimethyl sulfate
Dimethyl sulfoxide
O,S-Dimethyl phosphoramidothioate
N,N-Dimethylacetamide
O,O-Dimethyl-S-(2-(acetylamino)ethyl) dithiophosphate
4-(Dimethylamine)-3,5-XYLYL-N-methylcarbamate
Dimethylaminoantipyrine
4-Dimethylaminoazobenzene
para-Dimethylaminobenzenediazosodium sulphonate
5-(3-(Dimethylamino)propyl)-2-hydroxy-10,11-dihydro-5H-
dibenz(b,f)azephine
11-(3-Dimethylaminopropylidene-6,11-
dihydrodibenzo(b,e)thiepine hydrochloride
10-(2-(Dimethylamino)propyl)phenothiazine
Dimethylbenzanthracene
1,1-Dimethylbiguanide
1-(2-(1,3-Dimethyl-2-butenylidene)hydrazino)phthalazine
Dimethyldicetylammonium chloride
9,9-Dimethyl-10-dimethylaminopropylacridan hydrogen
tartrate
6-alpha,21-Dimethylethisterone
N-(5-(((1,1-Dimethylethyl)amino)sulfonyl)-1,3,4-
thiadiazol-2-YL)acetamide monsodium salt
N,N-Dimethyl-para((para-fluorophenyl)azo)aniline
Dimethylformamide
1,1-Dimethylhydrazine
1,2-Dimethylhydrazine
2,6-Dimethylhydroquinone
Dimethylimipramine
1,3-Dimethylisothiourea
1,3-Dimethylnitrosourea
3,3-Dimethyl-1-phenyltriazene
Dimethylthiomethylphosphate
N,N-Dimethyl-4-(para-tolylazo)aniline
5-(3,3-Dimethyl-1-triazeno)imidazole-4-carboxamide
citrate
2,6-Dimethyl-4-tridecylmorpholine
1,3-Dimethylurea
2,4-Dinitroaniline
4,6-Dinitro-ortho-cresol ammonium salt
2,6-Dinitro-N,N-dipropyl-4-(trifluoromethyl)benzenamine
2,4-Dinitrophenol
2,4-Dinitrophenol sodium salt
Dinitrosopiperazine
2,4-Dinitrotoluene
2,6-Dinitrotoluene
Dinoprost methyl ester
Dinoprostone
n-Dioctyl phthalate
Dioxane
meta-Dioxane-4,4-dimethyl
1,4-Di-N-oxide of dihydroxymethylquinoxaline
1,3-Dioxolane-4-methanol
3-(2-(1,3-Dioxo-2-methylindanyl)) glutarimide
3-(2-(1,3-Dioxo-2-phenyl-4,5,6,7-tetrahydro-4,7-
dithiaindanyl)) glutarimide
2-(2,6-Dioxopiperiden-3YL)phthalimide
N-(2,6-Dioxo-3-piperidyl)phthalimidine
1,3-Dioxo-2-(3-pyridylmethylene)indan
Diphenylamine
Diphenylguanidine
Diphenylhydantoin and phenobarbital
3-(3,3-Diphenylpropylamino)propyl-3′,4′,5′-
trimethoxybenzoate hydrochloride
Dipropyl adipate
Diquat
DI-sec-octyl phthalate
Disodium ethylene-1,2-bisidithiocarbamate
Disodium etidronate
Disodium inosinate
Disodium methanearsenate
Disodium molybdate dihydrate
Disodium phosphonomycin
Disodium selenate
Disulfiram
Dithane M-45
2,2-Dithiobis(pyridine-1-oxide)magnesium sulfate
trihydrate
2,2-Dithiodipyridine-1,1,-dioxide
Diuron
alpha-DFMO
Dobutamine hydrochloride
Domperidone
Dopamine
Dopamine hydrochloride
Doriden
Doxifluridine
Doxycycline
1-Dromoran tartrate
Duazomycin
Durabolin
Duricef
Dydrogesterone
Dye C
Econazole nitrate
Eflornithine hydrochloride
Elasiomycin
Elavil
Elavil hydrochloride
Elymoclavine
EM 255
Emoquil
Emorfazone
Enalapril maleate
Enavid
Endosulfan
Endrin
Enflurane
Enoxacin
Epe
Ephedrine
Epichlorohydrin
Epidehydrocholesterin
2-alpha,3-alpha-Epithio-5-alpha-androstan-17-beta-OL
4,5-Epithiovaleronitrile
EPN
Epocelin
1,2-Epoxyethylbenzene
Eraldin
Ergochrome AA (2,2)-5-beta,6-alpha,10-beta-5′,6′-
alpha,1-,-beta
Ergocornine methanesulfonate (salt)
Ergotamine tartrate
Ergoterm TGO
Erythromycin
Escherichia coli endotoxin
Escin
beta-Escin
Escin, sodium salt
Estradiol
Estradiol dipropionate
Estradiol polyester with phosphoric acid
Estradiol-17-valerate
Estradiol-3-benzoate
Estradiol-3-benzoate mixed with progesterone (1:14
moles)
Estradiol-17-caprylate
Estramustin phosphate sodium
Estra-1,3,5(10)-triene-17-beta-diol-17-
tetrahydropyranyl ether
Estriol
Estrone
Ethanolamine
Ethinamate
Ethinyl estradiol
Ethinyl estradiol and norethindrone acetate
17-alpha-Ethinyl-5,10-estrenolone
dl-Ethionine
Ethisterone and diethylstilbestrol
6-Ethoxy-2-benzothiazolesulfonamide
2-Ethoxyethanol
2-Ethoxyethyl acetate
Ethyl alcohol
Ethyl all-trans-9-(4-methoxy-2,3,6-trimethylphenyl)-
3,7-dimethyl-2,4,6,8-nonatetraenoate
Ethyl apovincaminate
Ethyl benzene
Ethyl (2,4-dichlorophenoxy) acetate
Ethyl fluclozepate
Ethyl hexylene glycol
Ethyl mercury chloride
Ethyl methacrylate
Ethyl methanesulfonate
Ethyl methyl 1,4-dihydro-2,6-dimethyl-4-(meta-
nitrophenyl)-3,5-pyridinedicarboxylate
Ethyl morphine hydrochloride dihydrate
Ethyl thiourea
alpha-((Ethylamino)methyl)-meta-hydroxybenzyl alcohol
2-Ethylamino-1,3,4-thiadiazole
1-Ethyl-1,4-dihydro-7-methyl-4-oxo-1,8-naphthyridine-3-
carboxylic acid
Ethyl-S-dimethylaminoethyl methylphosphonothiolate
Ethyl-N,N-dimethyl carbamate
Ethylene bis(dithiocarbamato)) zinc
Ethylene chlorohydrin
1,2-Ethylene dibromide
Ethylene dichloride
Ethylene glycol
Ethylene glycol diethyl ether
Ethylene glycol methyl ether
Ethylene oxide
Ethylenebis (dithiocarbamato) manganese and zinc
acetate (50:1)
Ethylenediamine hydrochloride
Ethylenediaminetetraacetic acid
Ethylenediaminetetraacetic acid, disodium salt
Ethyleneimine
Ethylestrenol
2-Ethylhexanol
Ethyl-para-hydroxyphenyl ketone
Ethylmercuric phosphate
Ethyl-N-methyl carbamate
Ethyl-2-methyl-4-chlorophenoxyacetate
5-Ethyl-N-methyl-5-phenylbarbituric acid
2-Ethyl-2-methylsuccinimide
1-Ethyl-4-(2-morpholinoethyl)-3,3-diphenyl-2-
pyrrolidinone
N-Ethyl-N-nitrosobiuret
1-Ethyl-1-nitrosourea
Ethylnorgestrienone
17-Ethyl-19-nortestosterone
N-Ethyl-para-(phenylazo) aniline
5-Ethyl-5-phenylbarbituric acid
1-5-Ethyl-5-phenylhydantoin
3-Ethyl-5-phenylhydantoin
5-(2-Ethylphenyl)-3-(3-methoxyphenyl)-s-triazole
2-Ethylthioisonicotinamide
Ethyltrichlorphon
Ethyl-3,7,11-trimethyldodeca-2,4-dienoate
Ethylurea and sodium nitrite (1:1)
Ethylurea and sodium nitrite (2:1)
Ethynodiol
Ethynylestradiol mixed with norethindrone
2-alpha-Ethynyl-alpha-nor-17-alpha-pregn-20-YNE-2-
beta,17-beta-diol
Etizolam
Etoperidone
ETP
E. typhosa lipopolysaccharide
False hellebore
Famfos
Famotidine
FD&C red No. 2
FD&C yellow NO. 5
Feldene
Fencahlonine
Fenestrel
Fenoprofen calcium dihydrate
Fenoterol hydrobromide
Fenthion
Fenthiuram
Ferbam
Ferrous sulfate
Fertodur
Fiboran
Firemaster BP-6
Firemaster FF-1
Flavoxate hydrochloride
Flomoxef sodium
Floxapen sodium
Flubendazole
Flucortolone
Flunarizine dihydrochloride
Flunisolide
Flunitrazepam
Fluoracizine
N-Fluoren-2-YL acetamide
Fluorobutyrophenone
Fluorocortisone
5-Fluoro-2,-deoxycytidine
3-Fluoro-4-dimethylaminoazobenzene
Fluorohydroxyandrostenedione
2-Fluoro-alpha-methyl-(1,1,-biphenyl)-4-acetic acid 1-
(acetyloxy) ethyl ester
4,-Fluoro-4-(4-methylpiperidino)butyrophenone
hydrochloride
3-Fluoro-4-phenylhydratropic acid
5-Fluoro-1-(tetrahydrofuran-2-YL)uracil
Fluorouracil
Flutamide
Flutazolam
Flutoprazepam
Flutropium bromide hydrate
Folic acid
Fominoben hydrochloride
Fonazine mesylate
Formaldehyde
Formamide
Formhydroxamic acid
Formoterol fumarate dihydrate
N-Formyl-N-hydroxyglycine
N-Formyljervine
Forphenicinol
Fortimicin A
Fortimicin A sulfate
Fotrin
Fulvine
Fumidil
Furapyrimidone
Furazosin hydrochloride
2-(2-Furyl)-3-(5-nitro-2-furyl)acrylamide
Fusarenone X
Fusaric acid calcium salt
Fusariotoxin T 2
Fusidine
Fyrol FR 2
Gabexate mesylate
Galactose
Gastrozepin
Gentamycin
Gentamycin sulfate
Gentisic acid
Germanium dioxide
Gestoral
Gindarine hydrochloride
Glucagon
2-(beta-d-Glucopyranosyloxy) isobutyronitrile
d-Glucose
Gludiase
Glutaraldehyde
Glutril
Glycidol
Glycinonitrile
Glycinonitrile hydrochloride
Glycol ethers
Glycyrrhizic acid, ammonium salt
Gold sodium thiomalate
Gonadotropin releasing hormone agonist
Gossypol acetic acid
Grisofulvin
Guanabenz acetate
Guanazodine
Guanfacine hydrochloride
Guanine-3-N-oxide
Guanosine
HBK
Haloanisone
Halofantrine hydrochloride
Haloperidol decanoate
Halopredone acetate
Halothane
Haloxazolam
HCDD
Heliotrine
Hematoidin
Heptamethylphenylcyclotetrasiloxane
Heptyl phthalate
Heroin
Hexabromonaphthalene
Hexachlorobenzene
2,2′,4,4′,5′5′-Hexachloro-1,1,-biphenyl
3,3′,4,4′,5,5′-Hexachlorobiphenyl
Hexachlorobutadiene
Hexachlorocyclopentadiene
1,2,3,4,7,8-Hexachlorodibenzofuran
Hexachlorophene
4,5,6,7,8,8-Hexachlor-D1,5-tetrahydro-4,7-methanoinden
1-Hexadecanamine
Hexadecyltrimethylammonium bromide
Hexafluoroacetone
Hexafluoro acetone trihydrate
Hexamethonium bromide
Hexamethylmelamine
n-Hexane
1,6-Hexanediamine
2-Hexanone
Hexocyclium methylsulfate
Hexone
Hexoprenaline dihydrochloride
Hexoprenaline sulfate
n-Hexyl carborane
Histamethizine
Histamine diphosphate
Homofolate
Human immunoglobin COG-78
Hyaluronic acid, sodium salt
Hycanthone methanesulfonate
Hydantoin
Hydralazine
Hydralazine hydrochloride
Hydrazine
Hydrochlorbenzethylamine dimaleate
Hydrochloric acid
Hydrocortisone sodium succinate
Hydrocortisone-21-acetate
Hydrocortisone-17-butyrate
Hydrocortisone-17-butyrate-21-propionate
Hydrocortisone-21-phosphate
Hydrofluoric acid
10-beta-Hydroperoxy-17-alpha-ethynyl-4-estren-17-beta-
OL-3-one
Hydroquinone-beta-d-glucopyranoside
N-Hydroxy ethyl carbamate
4,-Hydroxyacetanilide
N-Hydroxy-N-acetyl-2-aminofluorene
N-Hydroxyadenine
6-N-Hydroxyadenosine
3-alpha-Hydroxy-17-androston--one
17-beta-Hydroxy-5-beta-androstan-3-one
3-Hydroxybenzoic acid
para-Hydroxybenzoic acid ethyl ester
5-(alpha-Hydroxybenzyl)-2-benzimidazolecarbamic acid
methyl ester
1-Hydroxycholecalciferol
Hydroxydimethylarsine oxide
Hydroxydimethylarsine oxide, sodium salt
9-Hydroxyellipticine
2-(2-Hydroxyethoxy)ethyl-N-(alpha,alpha,alpha-
trifluoro-meta-tolyl)anthranilate
Hydroxyethyl starch
beta-Hydroxyethylcarbamate
1-Hydroxyethylidene-1,1-diphosphonic acid
17-beta-Hydroxy-7-alpha-methylandrost-5-ENE-3-one
7-Hydroxymethyl-12-methylbenz(alpha)anthracene
1-Hydroxymethyl-2-methylditmide-2-oxide
5-Hydroxymethyl-4-methyluracil
2-Hydroxymethylphenol
5-(1-Hydroxy-2-((1-methyl-3-phenylpropyl)amino)ethyl)
salicyclamide hydrochloride
N-(Hydroxymethyl)phthalimide
3-(1-Hydroxy-2-piperidinoethyl)-5-phenylisoxazole
citrate
2-Hydroxy-N-(3-(meta-
(piperidinomethyl)phenoxy)propyl)acetamide acetate
(ester hydrochloride)
Hydroxyprogesterone caproate
beta-(N-(3-Hydroxy-4-pyridone))-alpha-aminopropionic
acid
4-Hydroxysalicylic acid
5-Hydroxytetracycline
5-Hydroxytetracycline hydrochloride
17-beta-Hydroxy-4,4,17-alpha-trimethyl-androst-5-
ENE(2,3-d) isoxazole
Hydroxytriphenylstannane
dl-Hydroxytryptophan
5-Hydroxy-1-tryptophan
dl-Hydroxytryptophan
5-Hydroxy-1-tryptophan
Hydroxyurea
3-Hydroxyxanthine
Hydroxyzine pamoate
Hyoscine hydrobromide
Hypochlorous acid
Hypoglycine B
Ibuprofen piconol
Ifenprodil tartrate
IMET 3106
4-Imidazo (1,2-alpha) pyridin-2-YL-alpha-
methylbenzeneacetic acid
Imidazole mustard
2-Imidazolidinethione
2-Imidazolidinethione mixed with sodium nitrite
2-Imino-5-phenyl-4-oxazolidinone
Improsulfan tosylate
Indacrinone
Indanazoline hydrochloride
1,3-Indandione
Indapamide
Indeloxazine hydrochloride
Inderal
Indium
Indium nitrate
1H-Indole-3-acetic acid
Indole-3-carbinol
Indomethacin
Inolin
Insulin
Insulin protamine zinc
Iocarmate meglumine
Iodoacetic acid
Iopramine hydrochloride
Iotroxate meglumine
Ipratropium bromide
Iron-dextran complex
Iron nickel zinc oxide
Iron-poly (sorbitol-gluconic acid) complex
Iron-sorbitol
Isoamygdalin
Isoamyl 5,6-dihydro-7,8-dimethyl-4,5-dioxo-4H-pyrano
(3,2-c) quinoline-2-carboxylate
Isobutyl methacrylate
para-Isobutylhydratropic acid
Isocarboxazid
Isodecyl methacrylate
Isodonazole nitrate
Isoflurane
Isonicotinic acid hydrazide
Isonicotinic acid-2-isopropylhydrazide
Isooctyl-2,4-dichlorophenoxyacetate
Isophosphamide
Isoprenaline hydrochloride
Isoprenyl chalcone
Isopropyl alcohol
Isopropyl-2,4-D ester
Isopropylidine azastreptonigrin
4,4,-Isopropylidenediphenol, polymer with 1-chloro-2,3-
epoxypropane
Isopropylmethanesulfonate
Isosafrole-n-octylsulfoxide
Isothiacyanic acid, ethylene ester
Isothiocyanic acid, phenyl ester
Isothiourea
Jervine
Jervine-3-acetate
Josamycin
Kanamycin
Kanamycin sulfate (1:1) salt
KAO 264
Karminomycin
Kepone
Kerlone
Ketamine
Ketoprofen sodium
Ketotifen fumarate
KF-868
Khat leaf extract
KM-1146
KPE
Lactose
Latamoxef sodium
Lead
Lead (II) acetate
Lead chloride
Lead (II) nitrate (1:2)
Lecithin iodide
Lenampicillin hydrochloride
Lendormin
Lente insulin
Lentinan
Leptophos
1-Leucine
Leurocristine
Leurocristine sulfate (1:1)
Levamisole hydrochloride
Levorin
Levothyroxine sodium
Librium
d-Limonene
Linear alkylbenzenesulfonate, sodium salt
Linoleic acid (oxidized)
Liothyronine
Lipopolysaccharide, escherichia coli
Lipopolysaccharide, from B. Abortus Bang.
Lithium carbonate (2:1)
Lithium carmine
Lithium chloride
Lividomycin
Lobenzarit disodium
Locoweed
Lofetensin hydrochloride
Lucanthone metabolite
Luteinizing hormone antiserum
Luteinizing hormone-releasing hormone
Luteinizing hormone-releasing hormone, diacetate (salt)
Luteinizing hormone-releasing hormone, diacetate,
tetrahydrate
Lyndiol
Lysenyl hydrogen maleate
d-Lysergic acid diethylamide tartrate
Lysergide tartrate
Lysine
Mafenide acetate
Magnesium glutamate hydrobromide
Magnesium sulfate (1:1)
Malathion
Maleimide
Malotilate
Maltose
Manganese (II) chloride
Manganese (II) ethylenebis (dithiocarbamate)
Manganese (II) sulfate (1:1)
Maprotiline hydrochloride
Marezine hydrochloride
Maytansine
Mazindol
Mec
Meclizine dihydrochloride
Meclizine hydrochloride
Medemycin
Medrogestone
Medroxyprogesterone
Medroxyprogesterone acetate
Medullin
Melengestrol acetate
Mentha arvensis, oil
Mepiprazole dihydrochloride
Mepyrapone
Mequitazine
2-Mercapto-1-methylimidazole
1-(d-3-Mercapto-2-methyl-1-oxopropyl)-1-proline (S,S)
N-(2-Mercapto-2-methylpropanoyl)-1-cysteine
6-Mercaptopurine monohydrate
6-Mercaptopurine 3-N-oxide
Mercaptopurine ribonucleoside
d,3-Mercaptovaline
Mercuric acetate
Mercuric oxide
Mercury
Mercury (II) chloride
Mercury (II) iodide
Mercury methylchloride
Merthiolate sodium
Mervan ethanolamine salt
Mescaline
Mesoxalylurea monohydrate
Mestranol mixed with norethindrone
Metalutin
Metaproterenol sulfate
Methadone
Methadone hydrochloride
dl-Methadone hydrochloride
Methallyl-19-nortestosterone
Methaminodiazepoxide hydrochloride
1-Methamphetamine hydrochloride
Methaqualone hydrochloride
Methedrine
dl-Methionine
l-Methionine
Methionine sulfoximine
Methofadin
Methophenazine difumarate
Methotrexate
Methotrexate sodium
Methoxyacetic acid
3-Methoxycarbonylaminophenyl-N-3,-methylphenylcarbamate
Methoxychlor
5-Methoxyindoleacetic acid
4-(6-Methoxy-2-naphthyl)-2-butanone
(+)-2-(Methoxy-2-naphthyl)-propionic acid
2-(3-Methoxyphenyl)-5,6-dihydro-s-triazolo (5,1-alpha)
isoquinoline
2-(para-(6-Methoxy-2-phenyl-3-
indenyl)phenoxy)triethylamine hydrochloride
2-(para-(para-Methoxy-alpha-
phenylphenethyl)phenoxy)triethylamine hydrochloride
N1-(3-Methoxy-2-pyrazinyl)sulfanilamide
Methyl alcohol
Methyl azoxymethyl acetate
Methyl benzimidazole-2-YL carbamate
2-Methyl butylacrylate
Methyl chloride
Methyl chloroform
Methyl (beta)-11-alpha-16-dihydroxy-16-methyl-9-
oxoprost-13-EN-1-OATE
Methyl ethyl ketone
Methyl hydrazine
Methyl isocyanate
Methyl mesylate
Methyl methacrylate
Methyl (methylthio) mercury
Methyl parathion
Methyl pentachlorophenate
Methyl phenidyl acetate
Methyl salicylate
Methyl thiourea
Methyl urea and sodium nitrite
Methylacetamide
Methyl-5-benzoyl benzimidazole-2-carbamate
1-Methyl-2-benzylhydrazine
1-Methyl-5-chloroindoline methylbromide
Methylchlortetracycline
3-Methylcholanthrene
N-Methyl-4-cyclochexene-1,2-dicarboximide
N-Methyl-N-desacetylcolchicine
N-Methyl-dibromomaleinimide
beta-Methyldigoxin
17-alpha-Methyldihydrotestosterone
N-Methyl-3,6-dithia-3,4,5,6-tetrahydrophthalimide
Methylene chloride
Methylene dimethanesulfonate
N,N,-Methylenebis(2-amino-1,3,4-thiadiazole)
2-Methylenecyclopropanylalanine
Methylergonovine maleate
3-(1-Methylethyl)-1H-2,1,3-benzothiazain-4(3H)-one-2,2-
dioxide
4-Methylethylenethiourea
3-Methyl-5-ethyl-5-phenylhydantoin
3-Methylethynylestradiol
x-Methylfolic acid
N-Methylformamide
Methylhesperidin
(alpha-(2-Methylhydrazino)-para-toluoyl)urea,
monohydrobromide
4-Methyl-7-hydroxycoumarin
Methyl-ortho-(4-hydroxy-3-methoxycinnamoyl) reserpate
2-Methyl-1,3-indandione
N-Methyljervine
N-Methyllorazepam
Methylmercuric dicyandiamide
Methylmercuric phosphate
Methylmercury
Methylmercury hydroxide
1-Methyl-6-(1-methylallyl)-2,5-dithiobiurea
d-3-Methyl-N-methylmorphinan phosphate
N-Methyl-alpha-methyl-alpha-phenylsuccinimide
2-Methyl-1,4-naphthoquinone
2-Methyl-5-nitroimidazole-1-ethanol
N-Methyl-N′-nitro-N-nitrosoguanidine
4-(N-Methyl-N-nitrosamino)-1-(3-pyridyl)-1-butanone
N-Methyl-N-nitrosoaniline
N-Methyl-N-nitrosoethylcarbamate
N-Methyl-N-nitroso-1-propanamine
N-Methyl-N-nitrosourea
(3-Methyl-4-oxo-5-piperidino-2-thiazolidinylidene)
acetic acid ethyl ester
10-Methylphenothiazine-2-acetic acid
N-Methyl-para-(phenylazo) aniline
3-Methyl-2-phenylmorpholine hydrochloride
N-Methyl-2-phenyl-succinimide
Methyl-4-phthalimido-dl-glutaramate
N-Methyl-2-phthalimidoglutarimide
N-Methylpyrrolidone
Methylsulfonyl chloramphenicol
17-Methyltestosterone
N-Methyl-3,4,5,6-tetrahydrophthalimide
Methylthioinosine
6-Methylthiouracil
6-Methyluracil
Metiapine
Meticrane
Metoprine
Metoprolol tartrate
Metrizamide
Mexiletine hydrochloride
Mezinium methyl sulfate
Mezlocillin
Mibolerone
Miconazole nitrate
Micromycin
Midodrine
Mikelan
Miloxacin
Miltown
Mineral oil
Mineral oil, petroleum extracts, heavy naphthenic
distillate solvent
Mirex
Mithramycin
MN-1695
Mobilat
Molybdenum
Monoethylhexyl phthalate
Monoethylphenyltriazene
8-Monohydro mirex
Monosodium glutamate
Morphine hydrochloride
Morphine sulfate
Morphocycline
Moxestrol
Moxnidazole
Mucopolysaccharide, polysulfuric acid ester
Muldamine
Mycosporin
Nafoxidine hydrochloride
Naftidrofuryl oxalate
Naja nigricollis venom
Naloxone hydrochloride
Naphthalene
beta-Naphthoflavone
1-Naphthol
Navaron
Neem oil
Nembutal sodium
Neocarzinostatin
Neoprene
Neoproserine
Neosynephrine
Netilmicin sulfate
Nickel
Nickel carbonyl
Nickel compounds
Nickel subsulfide
Nickelous chloride
Nicotergoline
Nicotine
Nicotine tartrate (1:2)
N-Nicotinoyltryptamide
Nipradilol
Nisentil
Nitric acid
Nitrilotriacetic acid trisodium salt monohydrate
Nitrobenzene
Nitrofurantoin
Nitrofurazone
4-((5-Nitrofurfurylidene)amino)-3-methylthiomorpholine-
1,1-dioxide
Nitrogen dioxide
Nitrogen oxide
Nitroglycerin
1-(2-Nitroimidazol-1-YL-3-methoxypropan-2-OL
Nitromifene citrate
2-Nitropropane
4-Nitroquinoline-N-oxide
Nitroso compounds
N-Nitroso compounds
N-Nitrosobis(2-oxopropyl)amine
Nitrosocimetidine
N-Nitrosodiethylamine
N-Nitrosodimethylamine
N-Nitrosodi-N-propylamine
N-Nitroso-N-ethyl aniline
N-Nitroso-N-ethylurethan
N-Nitroso-N-ethylvinylamine
N-Nitrosohexahydroazepine
N-Nitrosoimidazolidinethione
N-Nitrosopiperidine
1-(Nitrosopropylamino)-2-propanol
N-Nitroso-N-propylurea
Nizofenone fumarate
Norchlorcyclizine
Norchlorcyclizine hydrochloride
1-Norepinephrine
19-Norethisterone
Norethisterone enanthate
Norgestrel
1-Norgestrel
19-Norpregn-4-ENE-3,20-dione
19-Nor-17-alpha-pregn-5(10)-EN-20-YNE-3-alpha,17-diol
19-Nor-17-alpha-pregn-5(10)-EN-20-YNE-3-beta,17-diol
19-Nor-17-alpha-pregn-4-EN-20-YN-17-OL
Novadex
Nutmeg oil, east indian
Nystatin
Ochratoxin
Ochratoxin A sodium salt
Octabromodiphenyl
Octachlorodibenzodioxin
Octoclothepine
Ofloxacin
Oleamine
Oleylamine hydrofluoride
Oncodazole
Ophthazin
Orgoteins
Orphenadrine hydrochloride
Oxaprozin
Oxatimide
Oxazolazepam
Oxepinac
Oxfendazole
Oxibendazole
Oxiranecarboxylic acid, 3-(((3-methyl-1-(((3-
methylbutyl)amino) carbonyl)-,ethyl ester, (2S-(2-
alpha-3-beta)R*)))
N-(2-Oxo-3,5,7-cylcoheptatrien-1-YL)aminooxoacetic acid
ethyl ester
2-(3-Oxo-1-indanylidene)-1,3-indandione
Oxolamine citrate
N-(2-Oxo-3-piperidyl)phthalimide
Oxybutynin chloride
Oxymorphinone hydrochloride
beta-Oxypropylpropylnitrosamine
Ozone
Padrin
Palm oil
Panoral
d-Pantethine
Pantocrin
Papain
Papaverine chlorohydrate
Paradione
Paramathasone acetate
Paraquat dichloride
Parathion
Paraxanthine
Pavisoid
PE-043
Penfluridol
Penicillic acid
Penitrem A
Pentachlorobenzene
2,3,4,7,8-Pentachlorodibenzofuran
Pentachloronitrobenzene
Pentachlorophenol
Pentafluorophenyl chloride
Pentazocine hydrochloride
Pentostatin
Pentothal
Pentothal sodium
Pentoxyphylline
Perchloroethylene
Perdipine
Perfluorodecanoic acid
Periactin hydrochloride
Periactinol
Perphenazine hydrochloride
Pharmagel A
1,10-Phenanthroline
Phenazin-5-oxide
Phenethyl alcohol
Phenfluoramine hydrochloride
Phenol
4-Phenoxy-3-(pyrrolidinyl)-5-sulfamoylbenzoic acid
Phenyl salicylate
Phenylacetic acid
(Phenylacetyl) urea
1-Phenylalanine
17-beta-Phenylaminocarbonyloxyoestra-1,3,5(10)-triene-
3-methyl ether
para-(Phenylazo)aniline
2-Phenyl-5-benzothiazoleacetic acid
1-Phenyl-3,3-diethyltriazene
2-Phenyl-5,5-dimethyl-tetrahydro-1,4-oxazine
hydrochloride
1-Phenyl-2-(1′,1′-diphenylpropyl-3′-amino)propane
4-Phenyl-1,2-diphenyl-3,5-pyrazolidinedione
meta-Phenylenediamine
2-Phenylethylhydrazine
Phenylmethylcylosiloxane, mixed copolymer
N-Phenylphthalimidine
Phenyl-2-pyridylmethyl-beta-N,N-dimethylaminoethyl
ether succinate
2-(Phenylsulfonylamino)-1,3,4-thiadiazole-5-sulfonamide
1-Phenyl-2-thiourea
Phomopsin
Phorbol myristate acetate
Phosphonacetyl-1-aspartic acid
Phosphoramide mustard cyclohexylamine salt
Phthalazinol
Phthalic anhydride
Phthalimide
Phthalimidomethyl-O,O-dimethyl phosphorodithioate
N-Phthaloly-1-aspartic acid
N-Phthalylisoglutamine
Physostigmine sulfate
Phytohemagglutinin
Picloram
Pilocarpine monohydrochloride
Pimozide
2,6-Piperazinedione-4,4,-propylene dioxopiperazine
Piperidine
3-Piperidine-1,1-diphenyl-propanol-(1)
methanesulphonate
Piperin
Piperonyl butoxide
Pipethanate ethylbromide
Pipram
Pituitary growth hormone
Plafibride
cis-Platinous diammine dichloride
Platinum thymine blue
Podophyllin
Podophyllotoxin
Polybrominated biphenyls
Polychlorinated biphenyl (Aroclor 1248)
Polychlorinated biphenyl (Aroclor 1254)
Polychlorinated biphenyl (Kanechlor 300)
Polychlorinated biphenyl (Kanechlor 400)
Polychlorinated biphenyl (Kanechlor 500)
Polyoxyethylene sorbitan monolaurate
Potassium bichromate
Potassium canrenoate
Potassium chromate (VI)
Potassium clavulanate
Potassium cyanide
Potassium fluoride
Potassium iodide
Potassium nitrate
Potassium nitrite (1:1)
Potassium perchlorate
Potassium thiocyanate
Potato blossoms, glycoalkaloid extract
Potato, green parts
Pranoprofen
Prednisolone succinate
Prednisone 21-acetate
Predonin
9-beta,10-alpha-Pregna-4,6-diene-3,20-dione and 17-
alpha-hydroxypregn-4-ENE-3,2 ortho-dione (9:10)
5-alpha-17-alpha-Pregna-2-EN-20-YN-17-OL, acetate
Premarin
Primaquine phosphate
Primobolan
Prinadol hydrobromide
Procarbazine
Procarbazine hydrochloride
Procaterol hydrochloride
Prochlorpromazine
Progesterone
Prolinomethyltetracycline
Promethazine hydrochloride
Propadrine hydrochloride
Propane sultone
1,3-Propanediamine
1,2-Propanediol
Propanidide
3-Propanolamine
Proparthrin
Propazone
Propiononitrile
Propoxur
2-Propoxyethyl acetate
d-Propoxyphene hydrochloride
Propyl carbamate
Propyl cellosolve
n-Propyl gallate
Propylene glycol diacetate
Propylene glycol monomethyl ether
Propylene oxide
2-Propylpentanoic acid
2-Propylpiperidine
6-Propyl-2-thiouracil
Propylthiouracil and iodine
2-Propylvaleramide
2-Propylvaleric acid sodium salt
Prostaglandin A1
Prostaglandin E1
Prostaglandin E2 sodium salt
Prostaglandin F1-alpha
Prostaglandin F2-alpha
Prostaglandin F2-alpha-tham
Protizinic acid
Proxil
Pseudolaric acid A
Pseudolaric acid B
Purapuridine
Purine-6-thiol
Pyrantel pamoate
Pyrazine-2,3-dicarboxylic acid imide
Pyrazole
Pyrbuterol hydrochloride
Pyridinamine (9CI)
2,3-Pyridinedicarboximide
3,4-Pyridinedicarboximide
1-(Pyridyl-3)-3,3-dimethyl triazene
1-Pyridyl-3-methyl-3-ethyltriazene
5-(para-(2-Pyridylsulfamoyl)phenylazo)salicyclic acid
Pyrimidine-4,5-dicarboxylic acid imide
N1-2-Pyrimidinyl-sulfanilamide
Pyrogallol
Pyronaridine
N-(1-Pyrrolidinylmethyl)-tetracycline
Quaalude
Quercetin
Quinine
2-Quinoline thioacetamide hydrochloride
Ralgro
Refosporen
Reptilase
Reserpine
Retinoid etretin
all-trans-Retinylidene methyl nitrone
Rhodamine 6G extra base
2-beta-d-Ribofuranosyl-as-triazine-3,5(2H,4H)-dione
1-beta-d-Ribofuranosyl-1,2,4-triazole-3-carboxamide
Ricin
Rifamycin AMP
Rifamycin SV
Ripcord
Ritodrine hydrochloride
Rizaben
Robaveron
Ronnel
Rose bengal sodium
Rotenone
Rowachol
Rowatin
R Salt
Rubratoxin B
Rythmodan
Salicyclaldehyde
Salicyclamide
Salicyclic acid
Salicyclic acid, compounded with morpholine (1:1)
ortho-Salicylsalicylic acid
Salipran
Salmonella enteritidis endotoxin
Sarkomycin
SCH 20569
Scopolamine
Sefril
Selenium
Selenodiglutathione
Semicarbazide hydrochloride
Serum gonadotropin
Sfericase
Silicone 360
Sisomicin
S. Marcescens lipopolysaccharide
Smoke condensate, cigarette
Smokeless tobacco
Sodium para-aminosalicylate
Sodium arsenite
Sodium benzoate
Sodium bicarbonate
Sodium chloride
Sodium chlorite
Sodium chondroitin polysulfate
Sodium cobaltinitrite
Sodium colistinemethanesulfonate
Sodium cyanide
Sodium cyclamate
Sodium dehydroacetic acid
Sodium dichlorocyanurate
Sodium diethyldithiocarbamate
Sodium diphenyldiazo-bis(alpha-naphthylaminesulfonate)
Sodium fluoride
Sodium (E)-3-(para-(1H-imidazol-1-methyl)phenyl)-2-
propenoate
Sodium iodide
Sodium lauryl sulfate
Sodium luminal
Sodium nigericin
Sodium nitrite
Sodium nitrite and carbendazime (1:1)
Sodium nitrite and 1-citrulline (1:2)
Sodium nitrite and 1-(methylethyl) urea
Sodium nitroferricyanide
Sodium pentachlorophenate
Sodium picosulfate
Sodium piperacillin
Sodium retinoate
Sodium saccharin
Sodium salicylate
Sodium selenite
Sodium selenite pentahydrate
Sodium sulfate (2:1)
Sodium d-thyroxine
Sodium tolmetin dihydrate
Sodium-2,4-dichlorophenoxyacetate
(22s,25r)-5-alpha-Solanidan-3-beta-OL
Solanid-5-ENE-3-beta, 12-alpha-diol
(22s,25r)-Solanid-5-EN-3-beta-OL
Solanine
Solcoseryl
Spectogard
Spiclomazine hydrochloride
Spiramycin
Spiroperidol
SRC-II, heavy distillate
1-ST-2121
Sterculia foetida oil
Steroids
Stimulexin
Streptomycin
Streptomycin and dihydrostreptomycin
Streptomycin sesquisulfate
Streptomycin sulphate
Streptonigran
Streptonigrin methyl ester
Streptozoticin
STS 557
Styrene
Subtigen
Succinic anhydride
Succinonitrile
Sucrose
Sulfadiazine silver salt
Sulfadimethoxypyrimidine
Sulfadimethyldiazine
Sulfamonomethoxin
Sulfamoxole-trimethoprim mixture
Sulfanilamide
6-Sulfanilamido-2,4-dimethoxypyrimidine
5-Sulfanilamido-3,4-dimethyl-isoxazole
Sulfanilylurea
N-Sulfanylacetamide
alpha-Sulfobenzylpenicillin disodium
Sulfur dioxide
Sulfuric acid
Suloctidyl
Sultopride hydrochloride
Supercortyl
Superprednol
Surgam
Surital sodium
Surmontil maleate
Suxibuzone
Sweet pea seeds
Sygethin
meta-Synephrine hydrochloride
Synephrine tartrate
Synsac
2,4,5-T
T-1982
T-2588
Tagamet
Tarweed
TCDD
Tellurium
Tellurium dioxide
Temephos
Tenormin
Terbutaline sulphate
Terodiline hydrochloride
Testosterone
Testosterone heptanoate
Testosterone propionate
1,1,3,3-Tetrabutylurea
2,3,7,8-Tetrachlododibenzofuran
Tetrachloroacetone
1,1,3,3-Tetrachloroacetone
3,3′,4,4′-Tetrachloroazoxbenzene
1,2,3,4-Tetrachlorobenzene
3,3′,4,4′-Tetrachlorobiphenyl
2,4,5,6-Tetrachlorophenol
Tetracycline
Tetracycline hydrochloride
Tetraethyl lead
1-trans-D9-Tetrahydrocannabinol
2-(para-(1,2,3,4-Tetrahydro-2-(para-
chlorophenyl)naphthyl) phenoxy) triethyl amine
2,3,4,5-Tetrahydro-2,8-dimethyl-5-(2-(6-methyl-3-
pyridyl)ethyl)-1H-pyrid 0-(4,3-beta) indole
Tetrahydro-3,5-dimethyl-4H,1,3,5-oxadiazine-4-thione
5,6,7,8-Tetrahydrofolic acid
2-(1,2,3,4-Tetrahydro-1-naphthylamino)-2-imidazoline
hydrochloride
4,-O-Tetrahydropyranyladriamycin hydrochloride
para-(1,1,3,3-Tetramethylbutyl)phenol, polymer with
ethylene oxide and formaldehyde
2,2,9,9-Tetramethyl-1,10-decanediol
Tetramethyl lead
Tetramethylsuccinonitrile
Tetramethylthiourea
1,1,3,3-Tetramethylurea
Tetranicotylfructose
Tetrapotassium hexacyanoferrate
Tetrasodium fosfestrol
Tetrazosin hydrochloride dihydrate
Thalidomide
Thallium acetate
Thallium chloride
Thallium compounds
Thallium sulfate
Thebaine hydrochloride
para-(2-Thenoyl) hydratropic acid
Theobromine
Theobromine sodium salicylate
Theophylline
1-(Theophyllin-7-YL)ethyl-2-(2-(para-chlorophenoxy)-2-
methylpropionate
Thiamine chloride
2-(Thiazol-4-YL) benzimidazole
2-(4-Thiazolyl)-5-benzimidazolecarbamic acid methyl
ester
Thioacetamide
Thioinosine
Thiotriethylenephosphoramide
2-Thiouracil
Thiram
Thymidine
Thyroid
1-Thyroxin
Thyroxine
Tiapride hydrochloride
Ticarcillin sodium
Ticlodone
Timepidium bromide
Timiperone
Tinactin
Tindurin
Tinidazole
Tinoridine hydrochloride
Tiquizium bromide
2,4,5-T isooctyl ester
Titanium (wet powder)
Tizanidine hydrochloride
Tobacco
Tobacco leaf, nicotiana glauca
Tobramycin
Todralazine hydrochloride hydrate
Togal
Tolmetine
Toluene
para-Toluenediamine sulfate
ortho-Toluidine
Tormosyl
2,4,5-T propylene glycol butyl ether ester
Traxanox sodium pentahydrate
Triaminoguanidine nitrate
para,para,-Triazenylenedibenzenesulfonamide
Triazolam
Trichloroacetonitrile
1,2,4-Trichlorobenzene
Trichloroethylene
2,4,4,-Trichloro-2,-hydroxydiphenyl ether
(2,2,2-Trichloro-1-hydroxyethyl) dimethylphosphonate
N-(Trichloromethylthio)phthalimide
4-(2,4,5-Trichlorophenoxy) butyric acid
alpha-(2,4,5-Trichlorophenoxy) propionic acid
Trichloropropionitrile
Triclopyr
Tricosanthin
Tridemorph
Tridiphane
Triethyl lead chloride
Triethylenetetramine
2,2,2-Trifluoroethyl vinyl ether
3,-Trifluoromethyl-4-dimethylaminoazobenzene
Trifluoromethylperazine
2-(8,-Trifluoromethyl-4,-quinolylamino)benzoic acid,
2,3-dihydroxy propyl ester
Trifluperidol
Triglyme
Trimebutine maleate
(beta)-Trimethoquinol
Trimethoxazine
5-(3,4,5-Trimethoxybenzyl)-2,4-diaminopyrimidine
Trimethyl lead chloride
Trimethyl phosphate
Trimethyl phosphite
3,3,5-Trimethyl-2,4-diketooxazolidine
Trimethylenedimethanesulfonate
exo-Trimethylenenorbornane
1,1,3-Trimethyl-3-nitrosourea
1,3,5-Trimethyl-2,4,6-tris(3,5-DI-tert-butyl-4-
hydroxybenzyl) benzene
Triparanol
Tris
Tris (1-aziridinyl)-para-benzoquinone
Tris-(1-aziridinyl) phosphine oxide
Trisaziridinyltriazine
Tris (1-methylethylene) phosphoric triamide
Tritolyl phosphate
Tropacaine hydrochloride
1-Tryptophan
TSH-releasing hormone
Tungsten
dl-meta-Tyrosine
1-Tyrosine
Ubiquinone 10
Uracil
Uracil mixture with tegafur (4:1)
Uranyl acetate dihydrate
Urapidil
Urbacide
Urbason soluble
Urethane
Urfamicin hydrochloride
Uridion
Urokinase
Valbazen
Valison
Vanadium pentoxide (dust)
Vasodilan
Vasodilian
Vasodistal
Vasotonin
Venacil
Ventipulmin
Veratramine
Veratrine
Veratrylamine
Vincaleukoblastine
Vincaleukoblastine sulfate (1:1) (salt)
Vinyl chloride
Vinyl pivalate
Vinyl toluene
Vinylidene chloride
R-5-Vinyl-2-oxazolidinethione
Viomycin
Vipera berus venom
Viriditoxin
Visken
Vistaril hydrochloride
Vitamin A
Vitamin A acetate
Vitamin A acid
13-cis-Vitamin A acid
Vitamin A palmitate
Vitamin B7
Vitamin B12 complex
Vitamin B12, methyl
Vitamin D2
Vitamin K
Vitamin MK 4
Volidan
Vomitoxin
Wait's green mountain antihistamine
Warfarin
Warfarin sodium
White spirit
Xamoterolfumarate
Xanax
Xanthinol nicotinate
Xylene
meta-Xylene
ortho-Xylene
para-Xylene
Xylostatin
N-(2,3-Xylyl)anthranilic acid
Ytterbium chloride
Zaroxolyn
Zearalenone
Zimelidine dihydrochloride
Zinc carbonate (1:1)
Zinc chloride
Zinc (II) EbrA complex
Zinc oxide
Zinc (N,N,-propylene-1,2-bis(dithiocarbamate))
Zinc pyridine-2-thiol-1-oxide
Zinc sulfate
Zoapatle, crude leaf extract
Zoapatle, semi-purified leaf extract
Zotepine
Zygosporin A
Zyloprim

TABLE V
Antibodies Used to Determine the Differentiated Status of Cells

| | | |
|---|---|---|
| Antibody | Antigen | Cell Specificity |

Panel I: Undifferentiated Cells

| | |
|---|---|
| SSEA-1 | human ES/ICM |
| SSEA-4 | human ES/ICM |
| TRA-1-60 | human ES/ICM |
| TRA-1-81 | human ES/ICM |
| SOX-2 | human ES/ICM |
| Oct-4 | human ES/ICM |
| Nanog | human ES/ICM |

Panel II: Broad Differentiated Cell Characterization

| | | |
|---|---|---|
| | Cxcr4 | Definitive endoderm |
| | Vimentin | Connective tissue |
| | | cell/primitive |
| neuroepithelium | Cytokeratins | Epithelial cell |
| | Neurofilaments | Neurons |
| | L,M,H | |

Panel III: Narrow Differentiated Cell Characterization

| | | |
|---|---|---|
| Ectoderm | Nestin | Neural progenitor |
| | S-100 | Neuroectoderm |
| | CD56 | Neuroectoderm |
| | CD57 | Neuroectoderm |
| | CD99 | Neuroectoderm |
| | Neuron-specific | Neuroectoderm |
| | enolase | |
| | Microtubule | Dendritic neurons |
| | Basic Protein | |
| | (MAP 2) | |
| | GFAP | Astrocytes |
| | CD133 | Neural stem cells |
| | Myelin basic | Oligodendrocytes |
| | Protein | |
| | Neural | Differentiated neurons |
| | Tubulin | |
| | Noggin | Neurons |
| Mesoderm | Bone | Mesenchymal Progenitors |
| | morphogenic | |
| | protein receptor | |
| | Fetal liver | Endothelial progenitor |
| | kinase-1 (Flk1) | |
| | Smooth muscle | Smooth muscle |
| | myosin | |
| | VE-Cadherin | Smooth muscle |
| | Desmin | Muscle cell |
| (multinucleate) | Bone-specific | Osteoblast |
| | alkaline phosphatase | |
| | Osteocalcin | Osteoblast |
| | CD34 | Hematopoietic/muscle |
| | | satellite/Endothelial |
| | CD44 | Mesenchymal progenitors |
| | c-kit | Hematopoietic and |
| | | mesenchymal progenitors |
| | Stem cell | Hematopoietic/mesenchymal |
| | antigen-1 | |
| | (sca-1) | |
| | Stro-1 | Bone marrow |
| | | stromal/Mesenchymal |
| | | stem cells |
| | Collagen II | Chondrocytes |
| | Collagen IV | Chondrocytes |
| | CD29 | Stromal cells |
| | CD44 | Stromal cells |
| | CD73 | Stromal cells |
| | CD166 | Stromal cells |
| | Brachyury | Mesoderm (Notochord) |
| Endoderm | Sox17 | Visceral and definitive |
| | | Endoderm |
| | Goosecoid (+) | Definitive endoderm |
| | Goosecoid (−) | Visceral endoderm |
| | Albumin | Hepatocytes |
| | B-1 Integrin | Hepatocytes |

TABLE X
Single Cell-Derived Cell Lines of Series 1
Series 1 Exp.

| Line Name | ACTC No. | Medium |
|---|---|---|
| 1 | | DMEM 10% |
| 2 | | Fetal Bovine |
| 3 | | Serum |
| 4 | | |
| 5 | | |
| 6 | | |
| B-1 | | |
| B-2 | 51 | |
| B-3 | 55 | |
| B-4 | 66 | |
| B-5 | | |
| B-6 | 56 | |
| B-7 | 53 | |
| B-9 | | |
| B-10 | | |
| B-11 | 58 | |
| B-12 | 65 | |
| B-13 | | |
| B-14 | 67 | |
| B-15 | 71 | |
| B-16 | 59 | |
| B-17 | 54 | |
| B-18 | | |
| B-19 | | |
| B-20 | | |
| B-21 | | |
| B-22 | | |
| B-23 | | |
| B-24 | | |
| B-25 | 57 | |
| B-26 | 50 | |
| B-27 | | |
| B-28 | 60 | |
| B-29 | 52 | |
| B-30 | 61 | |
| B-31 | | |
| B-32 | | |
| B-33 | | |
| B-34 | | |
| B-35 | | |
| 2-1 | 63 | |
| 2-2 | 62 | |
| 2-3 | 70 | |
| 2-4 | | |
| 4-1 | | |
| 4-2 | 69 | |
| 4-3 | | |
| 4-4 | | |
| 5-1 | | |
| 5-2 | | |
| 5-3 | | |
| 5-4 | 68 | |
| 5-5 | | |
| 6-1 | 64 | |

TOTAL COLONIES SERIES 1 = 54

What is claimed is:

1. A method for deriving cells from pluripotent stem cells wherein said derived cells possess reduced differentiation potential than said pluripotent stem cells, comprising the steps of:
(a) selecting all or a subset of differentiation conditions from a plurality of differentiation conditions that may result in the differentiation of said pluripotent stem cells;
(b) exposing said pluripotent stem cells to said selected all or a subset of differentiation conditions from step (a) for various time periods resulting in a heterogeneous population of cells comprising cells with reduced differentiation potential than said pluripotent stem cells;
(c) plating said heterogeneous population of cells to isolate a number of individual cultures of cells or a number of individual cultures of cells that are oligoclonal, wherein one or more of said cultures comprise cells with reduced differentiation potential than said pluripotent stem cells and wherein each of said individual cultures having only one cell may be propagated into a pure clonal culture of cells and wherein each of said individual cultures of cells having cells that are oligoclonal may be propagated into a larger number of cells; and
(d) propagating one or more (or all) of said individual cultures of cells in conditions selected to promote the propagation of said one or more (or all) of said individual cultures of cells.

2. A method for deriving cells from embryoid bodies derived from pluripotent stem cells wherein said derived cells possess reduced differentiation potential than said embryoid bodies derived from pluripotent stem cells, comprising the steps of:
(a) selecting all or a subset of differentiation conditions from a plurality of differentiation conditions that may result in the differentiation of said embryoid bodies derived from pluripotent stem cells;
(b) exposing said embryoid bodies derived from pluripotent stem cells to said selected all or a subset of differentiation conditions from step (a) for various time periods resulting in a heterogeneous population of cells comprising cells with reduced differentiation potential than said pluripotent stem cells;
(c) plating said heterogeneous population of cells to isolate a number of individual cultures of cells, each culture having only one cell or cells that are oligoclonal, wherein one or more of said cultures comprise cells with reduced differentiation potential than said pluripotent stem cells and wherein each of said individual cultures having only one cell may be propagated into a pure clonal culture of cells and wherein each of said individual cultures of cells that are oligoclonal may be propagated into a larger number of cells; and
(d) propagating one or more (or all) of said individual cultures of cells in conditions selected to promote the propagation of said one or more (or all) of said individual cultures of cells.

3. A method for deriving cells from pluripotent stem cells, wherein said derived cells possess reduced differentiation potential than said pluripotent stem cells, comprising the steps of:
(a) exposing said pluripotent stem cells in various differentiation conditions for various time periods resulting in a heterogeneous population of cells comprising cells with reduced differentiation potential than said pluripotent stem cells;
(b) plating said heterogeneous population of cells to isolate a number of individual cultures of cells or a number of individual cultures of cells that are oligoclonal, wherein one or more of said cultures comprise cells with reduced differentiation potential than said pluripotent stem cells and wherein each of said individual cultures having only one cell may be propagated into a pure clonal culture of cells and wherein each of said individual cultures of cells having cells that oligoclonal may be propagated into a larger number of cells; and
(c) propagating one or more (or all) of said individual cultures of cells in conditions selected to promote the propagation of said one or more (or all) of said individual cultures of cells.

4. A method for deriving cells from embryoid bodies derived from pluripotent stem cells, wherein said derived cells possess reduced differentiation potential than said pluripotent stem cells, comprising the steps of:
(a) exposing said embryoid bodies derived from pluripotent stem cells in various differentiation conditions for various time periods resulting in a heterogeneous population of cells comprising cells with reduced differentiation potential than said pluripotent stem cells;
(b) plating said heterogeneous population of cells to isolate a number of individual cultures of cells, each culture having only one cell or an oligoclonal number of cells, wherein one or more of said cultures comprise cells with reduced differentiation potential than said pluripotent stem cells and wherein each of said individual cultures having only one cell may be propagated into a pure clonal culture of cells and wherein each of said individual cultures of cells that are oligoclonal may be propagated into a larger number of cells; and
(c) propagating one or more (or all) of said individual cultures of cells in conditions selected to promote the propagation of said one or more (or all) of said individual cultures of cells.

5. The method according to any one of claims 1-4, further comprising a step of disaggregating said heterogeneous population of cells prior to plating.

6. The method according to claim 5, wherein said disaggregating step is performed by trypsinizing the heterogenous population of cells.

7. The method according to any one of claims 1-4, wherein, in said plating step, said heterogeneous population of cells is plated at limiting dilution.

8. The method according to claim 5, wherein, in said plating step, said heterogeneous population of cells, after disaggregation, is plated at limiting dilution.

9. The method according to claim 7 or claim 8, wherein limiting dilution is performed in multiwell dishes.

10. The method according to any one of claims 1-4, wherein, in said plating step, said heterogeneous population of cells is plated at low density.

11. The method according to claim 10, wherein said heterogeneous population of cells plated at low density is plated on semisolid media.

12. The method according to any one of claims 1-4, wherein said heterogeneous population of cells in step (b) are plated in juxtaposition with feeder or inducer cells.

13. The method according to any one of claims 1-4, wherein said heterogeneous population of cells form embryoid bodies prior to plating.

14. The method according to any one of claims 1-4, wherein said pluripotent stem cells are differentiated in vitro, in vivo, or in ovo.

15. The method according to any one of claims 1-4, wherein said heterogeneous population of cells are plated as single isolated cells at low density in a semisolid media.

16. The method according any one of claims 1-4, wherein, in said plating step, said heterogenous population of cells are plated as single isolated cells at low density in a hanging drop culture.

17. The method according to claim 16, further comprising the step of culturing said single isolated cells as an aggregate.

18. The method according to any one of claims 1-4, wherein said heterogeneous population of cells is cultured at low cellular density such that colonies of proliferating cells derived from a single cell can be easily identified and isolated.

19. The method according to any one of claims 1-4, wherein the cells in said individual cultures, or progeny thereof, are documented by genotype or phenotype.

20. The method according to any one of claims 1-4, wherein the cells in said individual cultures, or progeny thereof, are documented by photography.

21. The method according to any one of claims 1-4, wherein the cells in said individual cultures, or progeny thereof, are documented by immunocytochemistry.

22. The method according to any one of claims 1-4, wherein the cells in said individual cultures, or progeny thereof, are documented by hybridization of probes with RNA or cDNA transcript.

23. The method according any one of claims 1-4, wherein said pluripotent stem cells are selected from the group consisting of ES cells, EG cells, EC cells and ED cells.

24. The method according to claim 23, wherein said ED cells are selected from the group consisting of morula cells and inner cell mass cells.

25. The method according any one of claim 1, 2, 3, 4, 23 or 24, wherein said pluripotent stem cells are human cells.

26. The method according to any one of claims 1-4, wherein said pluripotent stem cells are genetically modified such that the MHC genes are deleted.

27. The method according to any one of claims 1-4, wherein said pluripotent stem cells are genetically modified such that the MHC genes are first deleted and then alleles of the MHC gene family are restored such that these stem cells are hemizygous or homozygous for one allele of the MHC gene family.

28. The method according to any one of claims 1-4, wherein said pluripotent stem cells are derived from the direct differentiation of embryonic cells without the derivation of embryonic stem cell line.

29. The method according to any one of claims 1-4, wherein said pluripotent stem cells are derived from the reprogramming of somatic cell through the exposure of said somatic cell to the cytoplasm of an undifferentiated cell.

30. The method according to any one of claims 1-4, wherein one of more cells in said individual cultures of cells are selected from the group consisting of endodermal cells, ectodermal cells and mesodermal cells.

31. The method according to any one of claims 1-4, wherein one of more cells in said individual cultures of cells are neuroglial precursor cells.

32. The method according to any one of claims 1-4, wherein one of more cells in said individual cultures of cells are hepatic cells or hepatic precursor cells.

33. The method according to any one of claims 1-4, wherein one of more cells in said individual cultures of cells are chondrocyte or chondrocyte precursor cells.

34. The method according to any one of claims 1-4, wherein one of more cells in said individual cultures of cells are myocardial or myocardial precursor cells.

35. The method according to any one of claims 1-4, wherein one of more cells in said individual cultures of cells are smooth muscle or skeletal muscle precursor cells.

36. The method according to claim 35, wherein said smooth muscle or skeletal muscle precursor cells are selected from the group consisting of somatic muscle precursor cells, muscle satellite stem cells and myoblast cells.

37. The method according to any one of claims 1-4, wherein one of more cells in said individual cultures of cells are gingival fibroblast or gingival fibroblast precursor cells.

38. The method according to any one of claims 1-4, wherein one of more cells in said individual cultures of cells are pancreatic beta cells or pancreatic beta precursor cells.

39. The method according to any one of claims 1-4, wherein one of more cells in said individual cultures of cells are dermal fibroblasts with prenatal patterns of gene expression.

40. The method according to any one of claims 1-4, wherein one of more cells in said individual cultures of cells are retinal precursor cells.

41. The method according to any one of claims 1-4, wherein one of more cells in said individual cultures of cells are hemangioblasts.

42. The method according to any one of claims 1-4, wherein said pluripotent stem cells are human pluripotent stem cells.

43. The method according to any one of claims 1-4, wherein said pluripotent stem cells are derived from a library of human embryonic stem cells, wherein said library of human embryonic stem cells comprises stem cells, each of which is hemizygous or homozygous for at least one MHC allele present in a human population, wherein each member of said library of stem cells is hemizygous or homozygous for a different set of MHC alleles relative to the remaining members of the library.

44. The method according to claim 45, wherein said library of human embryonic stem cells comprises stem cells that are hemizygous or homozygous for all MHC alleles present in a human population.

45. A method of treating a patient suffering a condition or disease such that said patient is in need of cell-based therapy, comprising the steps of:
(a) obtaining said patient;
(b) identifying MHC proteins expressed on the surface of said patient's cells;
(c) providing a library of human cells that have reduced differentiation potential than said human embryonic stem cells made according to the method of claim 43 or 44;
(d) selecting the human cells in said library that match said patient's MHC proteins on said patient's cells and that are appropriate for treating said patient's condition or disease that renders said patient in need of cell-based therapy and optionally further differentiating said human cell;
(e) administering said human cells from step (d) to said patient.

46. The method according to claim 45, wherein said method is performed in a regional center.

47. The method according to claim 46, wherein said regional center is a hospital.

48. The method according to any one of claims 1-4, wherein in the exposing step said pluripotent stem cells are exposed to said differentiation conditions for 1-100 days.

49. The method according to any one of claims 1-4, further comprising the step of determining the lineage of the derived cells.

50. A method of treating a patient suffering a condition or disease such that said patient is in need of cell-based therapy, comprising the step of administering a cell derived from a method according to any one of claims 1-4 or progeny thereof that are further differentiated.

51. The method according to any one of claims 1-4, wherein one or more of said derived cells secrete growth factors.

52. The method according to any one of claims 1-4, wherein the culture medium of one or more of said derived cells is used as a differentiation condition in any one of claims 1-4.

53. The method according to any one of claims 1-4, wherein one or more of said derived cells secrete growth factors.

54. The method according to any one of claims 1-4, wherein the culture medium of one or more of said derived cells is used as a differentiation condition in any one of claims 1-4.

55. The method according to any one of claims 1-4, wherein said pluripotent stem cells or embryoid bodies derived therefrom are exposed to a variety of differentiating conditions.

56. The method according to any one of claim 1, 2, 3, 4 or 54, wherein plating step is performed at various time intervals after exposing to the differentiating conditions.
